(19) United States
(12) Patent Application Publication
(10) Pub. No.: US 2024/0230671 A9
(43) Pub. Date: Jul. 11, 2024

(54) COMPOSITIONS AND METHODS FOR IDENTIFICATION OF CROSS-REACTIVE ALLERGENIC PROTEINS AND TREATMENT OF ALLERGIES

(71) Applicant: Alladapt Immunotherapeutics, Inc., Menlo Park, CA (US)

(72) Inventors: Preeti Lal, Burlingame, CA (US); Ashley Dombkowski, Woodside, CA (US); Dana McClintock, San Mateo, CA (US)

(21) Appl. No.: 18/546,766

(22) PCT Filed: Feb. 24, 2022
(86) PCT No.: PCT/US2022/017700

§ 371 Date: Aug. 16, 2023

Related U.S. Application Data
(60) Provisional application No. 63/235,992, filed on Aug. 23, 2021.
(60) Provisional application No. 63/153,235, filed on Feb. 24, 2021.
(60) Provisional application No. 63/153,245, filed on Feb. 24, 2021.
(60) Provisional application No. 63/153,250, filed on Feb. 24, 2021.

(51) Int. Cl. G01N 33/68; A61K 9/00; A61K 39/35; A61K 39/36; A61P 37/08

(52) U.S. Cl. CPC G01N 33/6893; A61K 9/0053; A61K 39/35; A61K 39/36; A61P 37/08; G01N 33/6854; G01N 33/6878; A61K 2039/70; G01N 2800/24; G01N 2800/52

(57) ABSTRACT

The disclosure relates generally to methods of treating allergies in subjects, methods of monitoring treatment of allergies in subjects, methods of diagnosing allergies in subjects, methods for determining whether subjects are candidates for allergy treatments, methods and kits for detecting allergies or allergens, methods for identifying cross-reactive allergens, antibodies that bind allergenic proteins and methods of identifying the same, and multiple allergen compositions and methods of preparing the same.

### CROSS REFERENCE

This application claims the benefit of and priority to U.S. Provisional Patent Application No. 63/153,235, filed on Feb. 24, 2021; U.S. Provisional Patent Application No. 63/153,245, filed on Feb. 24, 2021; U.S. Provisional Patent Application No. 63/153,250, filed on Feb. 24, 2021; and U.S. Provisional Patent Application No. 63/235,992, filed on Aug. 23, 2021, the entire disclosure of each of which is hereby incorporated by reference in its entirety for all purposes.

### BACKGROUND

Allergy is a disorder of the immune system characterized by the occurrence of allergic reactions to normally harmless environmental substances. Allergies are caused by allergens, which may be present in a wide variety of sources, including but not limited to pollens or other plant components, dust, molds or fungi, foods, additives, latex, transfusion reactions, animal or bird danders, insect venoms, radiocontrast medium, medications or chemicals. Common allergic reactions include eczema, hives, hay fever, asthma, and reactions to venoms. Mild allergies like hay fever are highly prevalent in the human population and cause symptoms such as allergic conjunctivitis, itchiness, and runny nose. In some people, severe allergies to environmental or dietary allergens or to medication may result in life-threatening anaphylactic reactions and potentially death, if left untreated.

A food allergy is an adverse immune response to a food allergen, e.g., a food protein. Common food allergens are found in shellfish, peanuts, tree nuts, fish, milk, eggs, soy and fresh fruits such as strawberries, mangoes, bananas, and apples. Immunoglobulin E (IgE)-mediated food allergies are classified as type-I immediate hypersensitivity reactions. These allergic reactions have an acute onset (from seconds to one hour) and the accompanying symptoms may include angioedema (soft tissue swelling of the eyelids, face, lips, tongue, larynx and trachea), hives, itching of the mouth, throat, eyes, or skin, gastrointestinal symptoms such as nausea, vomiting, diarrhea, stomach cramps, or abdominal pain, rhinorrhea or nasal congestion, wheezing, shortness of breath, or difficulty swallowing, and even anaphylaxis, a severe, whole-body allergic reaction that can result in death.

While only 3% of adults suffer from food allergies, 8% of children under four years of age have a food allergy. This has been reported to be doubling every 10 years for certain nuts. It is estimated that over $24 billion is spent per year on health care costs for food allergic reactions, largely due to about 90,000 visits to the ER per year in the U.S. due to food induced anaphylactic symptoms. Moreover, there are still deaths that occur every year due to fatal food allergies.

Multi-allergic subjects are becoming increasingly common in the United States. Recent data show that approximately 30% to 60% of food-allergic subjects were allergic to multiple food. In a clinical trial, approximately two-thirds of peanut-allergic subjects were also allergic to 1 or more additional food sources. In nationwide epidemiological surveys of US adults and children, subjects who are reactive to foods other than peanuts show high rate of allergy to multiple foods. In a separate study, about half of multi-allergic subjects were found to be allergic to unique combinations of food allergies. Allergies to multiple foods increase the risk of anaphylaxis (due to accidental ingestion), increase anxiety, and worsen the disabling nature of food allergies for patients and their families, with significant impact on quality of life. Children with multiple food allergies are also more likely to experience an allergic reaction (0.7 to 3.4 reactions per year) compared with children with a single food allergy (0.2 reactions per year).

Current options in the management of foods that trigger food allergy include oral immunotherapy for specific foods, dietary avoidance of the allergenic food, and education of the patient/family on acute management of an allergic reaction. Despite efforts towards strict food allergen avoidance, accidental exposure continues to be a major concern.

The presence of cross-reactivity or co-recognition remains a confounding issue in the field of allergy. Cross-reactivity occurs when an individual has antibodies raised against a particular allergenic protein and these antibodies bind to another, usually structurally similar protein.

There remains an unmet medical need for methods, compositions, and kits for prediction and detection of cross-reactive allergens. There also remains an unmet need for new methods for treatment, prevention and monitoring individuals for clinically adverse effects from exposure to cross-reactive allergens and the resulting cross-reactive immune response, and for inducing parallel desensitization or tolerization in individuals having or at risk of having cross-reactivity to or co-recognition of multiple food allergenic proteins.

### SUMMARY

The disclosure relates generally to methods of treating allergies in subjects, methods of monitoring treatment of allergies in subjects, methods of diagnosing allergies in subjects, methods for determining whether subjects are candidates for allergy treatments, methods and kits for detecting allergies or allergens, methods for identifying cross-reactive allergens, antibodies that bind cross-reactive allergenic proteins and methods of identifying the same, and multiple allergen compositions and methods of preparing the same.

For example, in one aspect, provided herein is a method of treating a subject for two or more allergies. A contemplated method comprises: (a) identifying a subject as having a first allergy to a first allergenic protein; (b) identifying the subject as having a second allergy to a second allergenic protein that is immunologically cross-reactive with the first allergenic protein; and/or (c) administering a multiple allergen oral immunotherapy to the subject (for example, a multiple allergen oral immunotherapy that does not contain the second allergenic protein).

In certain embodiments, identifying a subject as having a first allergy includes evaluating the subject's clinical history of allergy and/or identifying a subject as having a second allergy does not include evaluating the subject's clinical history of allergy. In certain embodiments, identifying a subject as having a second allergy comprises assessing the presence of IgE antibodies in the subject that bind to a first allergenic protein, and/or assessing the presence of a T-cell epitope within a first allergenic protein.

In certain embodiments, a first allergy is a shrimp, cod, salmon, hen's egg, cow's milk, peanut, sesame, soy, pecan, cashew, hazelnut, walnut, pistachio, almond, or wheat allergy. In certain embodiments, a second allergy is a prawn, kiwi, brazil nut, lobster, celery, coconut, crab, apple, buckwheat, clam, pear, lentil, mussel, tomato, garden pea, oyster, stone fruit (e.g., apricot, peach, cherry), yellow mustard, abalone, birch tree, mung bean, squid, alder tree, lupin, octopus, white oak tree, Korean pine, house dust mite, cockroach, or yellow fever mosquito allergy.

In certain embodiments, a first and/or second allergenic protein is selected from the group consisting of proteins listed in Tables 4-7 and Tables 10-12.

A contemplated multiple allergen oral immunotherapy may, for example, comprise two or more of: a shrimp allergen, a cod allergen, a salmon allergen, a hen's egg allergen, a cow's milk allergen, a peanut allergen, a sesame allergen, a soy allergen, a pecan allergen, a cashew allergen, a hazelnut allergen, a walnut allergen, a pistachio allergen, an almond allergen, and a wheat allergen. In certain embodiments, a multiple allergen oral immunotherapy comprises a shrimp allergen, a cod allergen, a salmon allergen, a hen's egg allergen, a cow's milk allergen, a peanut allergen, a sesame allergen, a soy allergen, a pecan allergen, a cashew allergen, a hazelnut allergen, a walnut allergen, a pistachio allergen, an almond allergen, and a wheat allergen. In certain embodiments, a multiple allergen oral immunotherapy comprises one or more allergenic proteins listed in Table 2.

In another aspect, provided herein is a method of treating an allergy to kiwifruit, brazil nut, coconut, buckwheat, lentil, garden pea, yellow mustard, mung bean, lupin, and/or Korean pine in a subject in need thereof. A contemplated method comprises administering to a subject a soybean, sesame, pistachio, pecan, hazelnut, walnut, cashew, and/or almond allergen (for example, administering to a subject a multiple allergen oral immunotherapy comprising a soybean, sesame, pistachio, pecan, hazelnut, walnut, cashew, and/or almond allergen). In certain embodiments, a contemplated method does not include administering to a subject a kiwifruit, brazil nut, coconut, buckwheat, lentil, garden pea, yellow mustard, mung bean, lupin, and/or Korean pine allergen (for example, the method comprises administering to a subject a multiple allergen oral immunotherapy that does not include a kiwifruit, brazil nut, coconut, buckwheat, lentil, garden pea, yellow mustard, mung bean, lupin, and/or Korean pine allergen).

In another aspect, provided herein is a method for treating a subject in need thereof having an allergy to an allergenic protein listed in the first column (titled Allergen 1) of any one of Tables 10-12 (or an allergy to a food product including the allergenic protein). A contemplated method comprises administering to a subject an allergenic protein listed in the corresponding row of the second column (titled Allergen 2) of Tables 10-12, or a food product including the allergenic protein (for example, administering to the subject a multiple allergen oral immunotherapy comprising the allergenic protein listed in the corresponding row of the second column of Tables 10-12). In certain embodiments, a contemplated method does not include administering to the subject the allergenic protein listed in the first column (titled Allergen 1) of Tables 10-12, or a food product including the allergenic protein (for example, the method comprises administering to the subject a multiple allergen oral immunotherapy that does not include the allergenic protein listed in the first column of Tables 10-12).

In another aspect, provided herein is a method for treating a subject in need thereof having an allergy to an allergenic peptide listed in the third column (titled Peptide) of any one of Tables 13-23 (or an allergy to a protein or a food product including the allergenic peptide). A contemplated method comprises administering to the subject an allergenic protein listed in the corresponding row of the sixth column (titled Cross-Reactive Proteins) of Tables 13-23, or a food product including the allergenic protein (for example, administering to the subject a multiple allergen oral immunotherapy comprising the allergenic protein listed in the corresponding row of the sixth column of Tables 13-23). In certain embodiments, a contemplated method does not include administering to the subject the allergenic peptide listed in the third column (titled Peptide) of Tables 13-23, or a protein or food product including the allergenic peptide (for example, the method comprises administering to the subject a multiple allergen oral immunotherapy that does not include the allergenic peptide listed in the third column of Tables 13-23).

In another aspect, provided herein is a method for treating a subject in need thereof having an allergy to an allergenic protein listed in the sixth column (titled Cross-Reactive Proteins) of any one of Tables 13-23 (or an allergy to a food product including the allergenic protein). A contemplated method comprises administering to the subject an allergenic peptide listed in the corresponding row of the third column (titled Peptide) of Tables 13-23, or a protein or food product including the allergenic peptide (for example, administering to the subject a multiple allergen oral immunotherapy comprising the allergenic peptide listed in the corresponding row of the third column of Tables 13-23). In certain embodiments, a contemplated method does not include administering to the subject the allergenic protein listed in the sixth column (titled Cross-Reactive Proteins) of Tables 13-23, or a food product including the protein (for example, the method comprises administering to the subject a multiple allergen oral immunotherapy that does not include the allergenic protein listed in the sixth column of Tables 13-23).

In another aspect, provided herein is a method for detecting development of tolerance to and/or monitoring the efficacy of a multiple allergen oral immunotherapy in a subject being administered the multiple allergen oral immunotherapy and in need thereof. A contemplated method comprises: (a) obtaining a biological sample from a subject (for example, before, after, or during treatment of the subject); (b) contacting the biological sample with a reference allergenic protein present in the multiple allergen oral immunotherapy and/or an other protein that is immunologically cross reactive to the reference allergenic protein; (c) measuring an amount of IgE, IgG, or T-cells that bind to the reference allergenic protein and/or the other protein; and/or (d) comparing the amount of IgE, IgG or T-cells with a previous measurement of the amount of IgE, IgG or T-cells. Tolerance and/or efficacy may, for example, be indicated when the amount of IgE, IgG or T-cells, is less than the previous amount of IgE, IgG or T-cells.

Contemplated reference allergenic proteins include, for example, proteins elected from the group consisting of a shrimp allergen, a cod allergen, a salmon allergen, a hen's egg allergen, a cow's milk allergen, a peanut allergen, a sesame allergen, a soy allergen, a pecan allergen, a cashew allergen, a hazelnut allergen, a walnut allergen, a pistachio allergen, an almond allergen, and a wheat allergen. In certain embodiments, a reference allergenic protein is listed in Table 2.

Contemplated other proteins include, for example, proteins in the same protein family as a reference allergenic protein (for example, a protein family listed in Table 3), proteins listed in Tables 4-7 or Tables 10-12, or any other proteins identified by methods disclosed herein.

In certain embodiments, a reference allergenic protein and/or an other protein are coupled to a solid support to form one or more protein-solid support complexes. Protein-solid support complexes may, for example, be further bound to a labeling reagent.

In another aspect, provided herein is a method for detecting development of tolerance to a protein listed in the second column (titled Allergen 2) of any one of Tables 10-12, and/or monitoring the efficacy of a multiple allergen oral immunotherapy in treating an allergy to a protein listed in the second column of any one of Tables 10-12. A contemplated method comprises: (a) obtaining a biological sample from the subject (for example, before, after, or during treatment of the subject); (b) contacting the biological sample with a protein listed in the corresponding row of the first column (titled Allergen 1) of Tables 10-12; (c) measuring an amount of IgE, IgG, or T-cells that bind to the protein listed in the corresponding row of the first column of Tables 10-12; and/or (d) comparing the amount of IgE, IgG or T-cells with a previous measurement of an amount of IgE, IgG or T-cells. Tolerance and/or efficacy may, for example, be indicated when the amount of IgE, IgG or T-cells, is less than the previous amount of IgE, IgG or T-cells.

In another aspect, provided herein is a method for detecting development of tolerance to a peptide listed in the third column (titled Peptide) of any one of Tables 13-23, and/or monitoring the efficacy of a multiple allergen oral immunotherapy in treating an allergy to a peptide listed in the third column of any one of Tables 13-23. A contemplated method comprises: (a) obtaining a biological sample from the subject (for example, before, after, or during treatment of the subject); (b) contacting the biological sample with a protein listed in the corresponding row of the sixth column (titled Cross-Reactive Proteins) of Tables 13-23; (c) measuring an amount of IgE, IgG, or T-cells that bind to the protein listed in the corresponding row of the sixth column of Tables 13-23; and/or (d) comparing the amount of IgE, IgG or T-cells with a previous measurement of an amount of IgE, IgG or T-cells. Tolerance and/or efficacy may, for example, be indicated when the amount of IgE, IgG or T-cells, is less than the previous amount of IgE, IgG or T-cells.

In another aspect, provided herein is a method for detecting development of tolerance to a protein listed in the sixth column (titled Cross-Reactive Proteins) of any one of Tables 13-23, and/or monitoring the efficacy of a multiple allergen oral immunotherapy in treating an allergy to a protein listed in the sixth column of any one of Tables 13-23. A contemplated method comprises: (a) obtaining a biological sample from the subject (for example, before, after, or during treatment of the subject); (b) contacting the biological sample with a peptide listed in the corresponding row of the third column (titled Peptide) of Tables 13-23; (c) measuring an amount of IgE, IgG, or T-cells that bind to the peptide listed in the corresponding row of the third column of Tables 13-23; and/or (d) comparing the amount of IgE, IgG or T-cells with a previous measurement of an amount of IgE, IgG or T-cells. Tolerance and/or efficacy may, for example, be indicated when the amount of IgE, IgG or T-cells, is less than the previous amount of IgE, IgG or T-cells.

In another aspect, provided herein is a detection kit, for example, for a biological cross-reactivity IgE assay. A contemplated detection kit comprises six or more distinct peptides or proteins each bound to a solid support, wherein each protein or peptide is selected from the group consisting of: (i) proteins listed in Table 2; (ii) proteins listed in Tables 10-12; and/or (iii) peptides listed in Tables 13-23. In certain embodiments, a contemplated kit includes an IgE or IgG labeling reagent; a binding buffer; a wash buffer; a detection buffer; and/or instructions for use.

In another aspect, provided herein is a method of assessing the presence or absence of cross-reactive IgE antibodies or T-cells in a biological sample. A contemplated method comprises: (a) contacting the biological sample with a reference allergenic protein; (b) measuring the binding of IgE antibodies or T-cells to the reference allergenic proteins; and/or (c) identifying any proteins that are immunologically cross-reactive to the reference allergenic protein; to assess the presence of one or more cross-reactive IgE antibodies or T-cells. In certain embodiments, a method does not include measuring binding of IgE antibodies or T-cells to proteins that are immunologically cross-reactive to the reference allergenic protein. In certain embodiments, a reference allergenic protein is immobilized on a substrate.

In another aspect, provided herein is a method for testing whether a subject is a candidate for a multiple allergen oral immunotherapy. A contemplated method comprises: detecting in a biological sample from a subject the presence of IgE antibodies or T-cells that bind to a reference allergenic protein in a multiple allergen oral immunotherapy and an other protein that is immunologically cross-reactive with the reference protein, wherein if IgE antibodies or T-cells are present in the biological sample, the subject is a candidate for the multiple allergen oral immunotherapy. A subject may, for example, have not been identified as having an allergy to an other protein.

In another aspect, provided herein is a method for diagnosing a subject as having an allergy to an allergic protein listed in the first column (titled Allergen 1) of any one of Tables 10-12, or a food product including the allergenic protein in the first column of any one of Tables 10-12. A contemplated method comprises evaluating the subject, or a biological sample from the subject, for an allergic response to an allergenic protein listed in the corresponding row of the second column (titled Allergen 2) of Tables 10-12, or a food product including the allergenic protein in the corresponding row of the second column of Tables 10-12.

In another aspect, provided herein is a method for diagnosing a subject as having an allergy to an allergic peptide listed in the third column (titled Peptide) of any one of Tables 13-23, or a protein or food product including the allergic peptide listed in the third column of any one of Tables 13-23. A contemplated method comprises evaluating the subject, or a biological sample from the subject, for an allergic response to an allergenic protein listed in the corresponding row of the sixth column (titled Cross-Reactive Proteins) of Tables 13-23, or a food product including the allergenic protein listed in the corresponding row of the sixth column of Tables 13-23.

In another aspect, provided herein is a method for diagnosing a subject as having an allergy to an allergic protein listed in the sixth column (titled Cross-Reactive Proteins) of any one of Tables 13-23, or a food product including the allergic protein listed in the sixth column of any one of Tables 13-23. A contemplated method comprises evaluating the subject, or a biological sample from the subject, for an allergic response to an allergenic peptide listed in the corresponding row of the third column (titled Peptide) of Tables 13-23, or a protein or food product including the allergenic peptide listed in the corresponding row of the third column of Tables 13-23.

Exemplary solid supports or substrates include a membrane, a filter, a chip, a slide (such as a microscopic slide), an array (such as a microarray), a wafer, a fiber, a magnetic or a non-magnetic bead, a gel, a tubing, a plate (such as a microtiter plate), a microtiter strip, a polymer, a microparticle, a sphere, a microsphere, a nanosphere, a particle, a microparticle, a nanoparticle, or a capillary.

Exemplary biological samples include whole blood, serum, plasma, sputum, blood cells (e.g., peripheral blood mononuclear cells (PBMC), T-cells, B-cells, basophils, etc.), immune cells, a tissue sample, a biopsy sample, urine, tears, peritoneal fluid, pleural fluid, breast duct fluid, breast exudate, breast milk, breast fluids, saliva, semen, mucous, lymph, cytosol, ascites, amniotic fluid, bladder washes, and bronchioalveolar lavage.

In another aspect, provided herein is a method for identifying a protein that is immunologically cross-reactive to a reference allergenic protein, the method comprising: (a) identifying an other protein that is in the same protein domain family as the reference allergenic protein; and/or (b) calculating pairwise identity and/or similarity between an amino acid sequence (for example, a full-length amino acid sequence) of the reference allergenic protein and an other protein, so as to determine whether the other protein is cross-reactive to the reference allergenic protein.

In certain embodiments, step (b) comprises: (i) providing a dataset comprising the amino acid sequence of the reference allergenic protein and the other protein; (ii) inputting the dataset into a computer readable medium; (iii) performing a bioinformatic step to calculate pairwise identity and similarity between the reference allergenic protein and the other protein; and/or (iv) identifying the other protein as cross-reactive to the reference allergenic protein based on pairwise identity and similarity between the reference allergenic protein and the other protein.

A bioinformatic step may, for example, comprise applying an algorithm to determine homology between the reference allergenic protein and the other protein based on pairwise identity and similarity, wherein the algorithm is selected from the group consisting of GAP, BESTFIT, FASTA, TFASTA, BLAST, BLASTP, TBLASTN, FASTDB, ALIGN, CLUSTALW, and CLUSTAL-Omega, including any version thereof. In certain embodiments, a bioinformatic step comprises applying an algorithm comprising (identity+similarity)/2 to determine a cross-reactivity score, for example, an other protein is considered cross-reactive to a reference allergenic protein if the other protein has a cross-reactivity score of ≥0.5 with respect to the reference allergenic protein. In certain embodiments, an other protein is considered cross-reactive to a reference allergenic protein if the other protein has ≥70% homology to the reference allergenic protein.

A contemplated method may, for example, comprise, prior to step (a), identifying the reference allergenic protein as a protein in an allergenic food source, and/or mapping the reference allergenic protein to a protein domain family. A contemplated method may, for example, comprise identifying a T-cell epitope peptide, a B-cell epitope peptide, and/or an MHC-binding epitope peptide in the allergenic protein that is cross-reactive to a T-cell epitope peptide, a B-cell epitope peptides and/or an MHC-binding epitope in the other protein. In certain embodiments, a contemplated method further comprises determining the MHC binding potential of the T-cell or B-cell peptide. A contemplated method may, for example, comprise identifying proteins that are immunologically cross-reactive to each reference allergenic protein present in a food source to generate an allergenic map (AllerMap) of the food source.

In certain embodiments, a contemplated reference allergenic protein is present in a multiple allergen oral immunotherapy. For example, a reference allergenic protein may be selected from the group consisting of a shrimp allergen, a cod allergen, a salmon allergen, a hen's egg allergen, a cow's milk allergen, a peanut allergen, a sesame allergen, a soy allergen, a pecan allergen, a cashew allergen, a hazelnut allergen, a walnut allergen, a pistachio allergen, an almond allergen, and a wheat allergen, or be selected from the group consisting of allergenic proteins listed in Table 2. In certain embodiments, a contemplated other protein is selected from the group consisting of proteins listed in Tables 4-7 or Tables 10-12.

In certain embodiments, a contemplated protein family is selected from the group consisting of: Tropomyosin, EF hand family (Beta-parvalbumin), ATP:guanido phosphotransferase (Arginine Kinase), Triosephosphate isomerase, Fructose bisphosphate aldolase class I, Alpha/beta casein, Lipocalin, Kappa-casein, Transferrin, C-type lysozyme/alpha-lactalbumin family, Serum albumin, Enolase, Serpin serine protease inhibitor, Kazal-type serine protease inhibitor, Collagen, Lipoprotein, Prolamin superfamily, Cupin, Profilin, Bet v 1 family, Oleosin, Isoflavone reductase family, Plant defensin, 60S acidic ribosomal protein, Heat shock protein Hsp70, Fe/Mn superoxide dismutase, Papain-like cysteine protease, Thaumatin-like protein, Unclassified, GILT family, Hevein-like and class I/11 chitinase, Zinc finger, C3HC4 type (RING finger), SCAI family, Peroxidase, Serpin serine protease inhibitor, Thionin, Thioredoxin, Transcription elongation factor 1, Lipid transfer protein, and Triosephosphate isomerase.

The disclosure also provides methods of treating an allergy to a food source including an allergenic Cupin family protein (e.g., a protein identified in Table 5) in a subject in need thereof. A contemplated method comprises administering to the subject a multiple allergen oral immunotherapy composition comprising a food source including a cross-reactive allergenic Cupin family protein identified herein.

For example, provided herein is a method of treating an allergy to one or more of soybean, sesame, pistachio, pecan, hazelnut, walnut, cashew, peanut and almond in a subject in need thereof, the method comprising administering to the subject a multiple allergen oral immunotherapy composition comprising two or more of soybean, sesame, pistachio, pecan, hazelnut, walnut, cashew, peanut, almond, kiwifruit, brazil nut, coconut, buckwheat, lentil, garden pea, yellow mustard, mung bean, lupin, and/or korean pine, optionally wherein the multiple allergen oral immunotherapy composition does not comprise soybean, sesame, pistachio, pecan, hazelnut, walnut, cashew, peanut and/or almond. Provided herein is a method of treating an allergy to soybean in a subject in need thereof, the method comprising administering to the subject a multiple allergen oral immunotherapy composition comprising one or more of kiwifruit, brazil nut, coconut, buckwheat, lentil, garden pea, yellow mustard, mung bean, lupin, and korean pine, optionally wherein the multiple allergen oral immunotherapy composition does not comprise soybean. Provided herein is a method of treating an allergy to soybean in a subject in need thereof, the method comprising administering to the subject a multiple allergen oral immunotherapy composition comprising two or more of sesame, pistachio, pecan, hazelnut, walnut, cashew, peanut, almond, kiwifruit, brazil nut, coconut, buckwheat, lentil, garden pea, yellow mustard, mung bean, lupin, and korean pine, optionally wherein the multiple allergen oral immunotherapy composition does not comprise soybean. Provided herein is a method of treating an allergy to sesame in a subject in need thereof, the method comprising administering to the subject a multiple allergen oral immunotherapy composition comprising one or more of kiwifruit, brazil nut, coconut, buckwheat, lentil, garden pea, yellow mustard, mung bean, lupin, and korean pine, optionally wherein the multiple allergen oral immunotherapy composition does not comprise sesame. Provided herein is a method of treating an allergy to sesame in a subject in need thereof, the method comprising administering to the subject a multiple allergen oral immunotherapy composition comprising two or more of soybean, pistachio, pecan, hazelnut, walnut, cashew, peanut, almond, kiwifruit, brazil nut, coconut, buckwheat, lentil, garden pea, yellow mustard, mung bean, lupin, and korean pine, optionally wherein the multiple allergen oral immunotherapy composition does not comprise sesame. Provided herein is a method of treating an allergy to pistachio in a subject in need thereof, the method comprising administering to the subject a multiple allergen oral immunotherapy composition comprising one or more of kiwifruit, brazil nut, coconut, buckwheat, lentil, garden pea, yellow mustard, mung bean, lupin, and korean pine, optionally wherein the multiple allergen oral immunotherapy composition does not comprise pistachio. Provided herein is a method of treating an allergy to pistachio in a subject in need thereof, the method comprising administering to the subject a multiple allergen oral immunotherapy composition comprising two or more of soybean, sesame, pecan, hazelnut, walnut, cashew, peanut, almond, kiwifruit, brazil nut, coconut, buckwheat, lentil, garden pea, yellow mustard, mung bean, lupin, and korean pine, optionally wherein the multiple allergen oral immunotherapy composition does not comprise pistachio. Provided herein is a method of treating an allergy to pecan in a subject in need thereof, the method comprising administering to the subject a multiple allergen oral immunotherapy composition comprising one or more of kiwifruit, brazil nut, coconut, buckwheat, lentil, garden pea, yellow mustard, mung bean, lupin, and korean pine, optionally wherein the multiple allergen oral immunotherapy composition does not comprise pecan. Provided herein is a method of treating an allergy to pecan in a subject in need thereof, the method comprising administering to the subject a multiple allergen oral immunotherapy composition comprising two or more of soybean, sesame, pistachio, hazelnut, walnut, cashew, peanut, almond, kiwifruit, brazil nut, coconut, buckwheat, lentil, garden pea, yellow mustard, mung bean, lupin, and korean pine, optionally wherein the multiple allergen oral immunotherapy composition does not comprise pecan. Provided herein is a method of treating an allergy to hazelnut in a subject in need thereof, the method comprising administering to the subject a multiple allergen oral immunotherapy composition comprising one or more of kiwifruit, brazil nut, coconut, buckwheat, lentil, garden pea, yellow mustard, mung bean, lupin, and korean pine, optionally wherein the multiple allergen oral immunotherapy composition does not comprise hazelnut. Provided herein is a method of treating an allergy to hazelnut in a subject need thereof, the method comprising administering to the subject a multiple allergen oral immunotherapy composition comprising two or more of soybean, sesame, pistachio, pecan, walnut, cashew, peanut, almond, kiwifruit, brazil nut, coconut, buckwheat, lentil, garden pea, yellow mustard, mung bean, lupin, and korean pine, optionally wherein the multiple allergen oral immunotherapy composition does not comprise hazelnut. Provided herein is a method of treating an allergy to walnut in a subject in need thereof, the method comprising administering to the subject a multiple allergen oral immunotherapy composition comprising one or more of kiwifruit, brazil nut, coconut, buckwheat, lentil, garden pea, yellow mustard, mung bean, lupin, and korean pine, optionally wherein the multiple allergen oral immunotherapy composition does not comprise walnut. Provided herein is a method of treating an allergy to walnut in a subject need thereof, the method comprising administering to the subject a multiple allergen oral immunotherapy composition comprising two or more of soybean, sesame, pistachio, pecan, hazelnut, cashew, peanut, almond, kiwifruit, brazil nut, coconut, buckwheat, lentil, garden pea, yellow mustard, mung bean, lupin, and korean pine, optionally wherein the multiple allergen oral immunotherapy composition does not comprise walnut. Provided herein is a method of treating an allergy to cashew in a subject in need thereof, the method comprising administering to the subject a multiple allergen oral immunotherapy composition comprising one or more of kiwifruit, brazil nut, coconut, buckwheat, lentil, garden pea, yellow mustard, mung bean, lupin, and korean pine, optionally wherein the multiple allergen oral immunotherapy composition does not comprise cashew. Provided herein is a method of treating an allergy to cashew in a subject need thereof, the method comprising administering to the subject a multiple allergen oral immunotherapy composition comprising two or more of soybean, sesame, pistachio, pecan, hazelnut, walnut, peanut, almond, kiwifruit, brazil nut, coconut, buckwheat, lentil, garden pea, yellow mustard, mung bean, lupin, and korean pine, optionally wherein the multiple allergen oral immunotherapy composition does not comprise cashew. Provided herein is a method of treating an allergy to peanut in a subject in need thereof, the method comprising administering to the subject a multiple allergen oral immunotherapy composition comprising one or more of kiwifruit, brazil nut, coconut, buckwheat, lentil, garden pea, yellow mustard, mung bean, lupin, and korean pine, optionally wherein the multiple allergen oral immunotherapy composition does not comprise peanut. Provided herein is a method of treating an allergy to peanut in a subject need thereof, the method comprising administering to the subject a multiple allergen oral immunotherapy composition comprising two or more of soybean, sesame, pistachio, pecan, hazelnut, walnut, cashew, almond, kiwifruit, brazil nut, coconut, buckwheat, lentil, garden pea, yellow mustard, mung bean, lupin, and korean pine, optionally wherein the multiple allergen oral immunotherapy composition does not comprise peanut. Provided herein is a method of treating an allergy to almond in a subject in need thereof, the method comprising administering to the subject a multiple allergen oral immunotherapy composition comprising one or more of kiwifruit, brazil nut, coconut, buckwheat, lentil, garden pea, yellow mustard, mung bean, lupin, and korean pine, optionally wherein the multiple allergen oral immunotherapy composition does not comprise almond. Provided herein is a method of treating an allergy to almond in a subject need thereof, the method comprising administering to the subject a multiple allergen oral immunotherapy composition comprising two or more of soybean, sesame, pistachio, pecan, hazelnut, walnut, cashew, peanut, kiwifruit, brazil nut, coconut, buckwheat, lentil, garden pea, yellow mustard, mung bean, lupin, and korean pine, optionally wherein the multiple allergen oral immunotherapy composition does not comprise almond.

Similarly, the disclosure also provides methods of treating an allergy to a food source including an allergenic Tropomyosin family protein (e.g., a protein identified in Table 6) or an allergenic Bet v 1 family protein (e.g., a protein identified in Table 7) in a subject in need thereof. A contemplated method comprises administering to the subject a multiple allergen oral immunotherapy composition comprising a food source including a cross-reactive allergenic Tropomyosin family protein or a cross-reactive allergenic Bet v 1 family protein identified herein, respectively.

The disclosure also provides methods of treating an allergy to a first Cupin family protein (e.g., a protein identified in FIG. 12), or a food source including the first Cupin family protein, in a subject in need thereof. A contemplated method comprises administering to the subject a second Cupin family protein that is cross-reactive with the first Cupin family protein (e.g., a protein that in FIG. 12 is connected to the first Cupin family protein by a line) or a food source including the second Cupin family protein.

For example, provided herein is a method of treating an allergy to an Ana o 1 protein, or a food source comprising the Ana o 1 protein, in a subject in need thereof, the method comprising administering to the subject: a Cor a 11 protein, or a food source comprising the Cor a 11 protein; a Pis v 3 protein, or a food source comprising the Pis v 3 protein; and/or a Ses i 3 protein, or a food source comprising the Ses i 3 protein; optionally wherein the method does not comprise administering to the subject the Ana o 1 protein or a food source comprising the Ana o 1 protein. Provided herein is a method of treating an allergy to an Ana o 2 protein, or a food source comprising the Ana o 2 protein, in a subject in need thereof, the method comprising administering to the subject: an Ara h 3 protein, or a food source comprising the Ara h 3 protein; a Car i 4 protein, or a food source comprising the Car i 4 protein; a Cor a 9 protein, or a food source comprising the Cor a 9 protein; a Gly m 6 protein, or a food source comprising the Gly m 6 protein; a Jug n 4 protein, or a food source comprising the Jug n 4 protein; a Jug r 4 protein, or a food source comprising the Jug r 4 protein; a Pis v 2 protein, or a food source comprising the Pis v 2 protein; a Pis v 5 protein, or a food source comprising the Pis v 5 protein; a Pru du 6 protein, or a food source comprising the Pru du 6 protein; a Ses i 6 protein, or a food source comprising the Ses i 6 protein; a Ses i 7 protein, or a food source comprising the Ses i 7 protein; a Ber e 2 protein, or a food source comprising the Ber e 2 protein; a Fag e 1 protein, or a food source comprising the Fag e 1 protein; a Fag t 13s g protein, or a food source comprising the Fag t 13s g protein; and/or a Sin a 2 protein, or a food source comprising the Sin a 2 protein; optionally wherein the method does not comprise administering to the subject the Ana o 2 protein or a food source comprising the Ana o 2 protein. Provided herein is a method of treating an allergy to an Ara h 1 protein, or a food source comprising the Ara h 1 protein, in a subject in need thereof, the method comprising administering to the subject: a Car i 2 protein, or a food source comprising the Car i 2 protein; a Gly m 5 protein, or a food source comprising the Gly m 5 protein; a Jug r 2 protein, or a food source comprising the Jug r 2 protein; a Ses i 3 protein, or a food source comprising the Ses i 3 protein; a Len c 1 protein, or a food source comprising the Len c 1 protein; a Lup an 1 protein, or a food source comprising the Lup an 1 protein; a Lup a v protein, or a food source comprising the Lup a v protein; a Pis s 1 protein, or a food source comprising the Pis s 1 protein; a Pis s 2 protein, or a food source comprising the Pis s 2 protein; and/or a Vig r 2 protein, or a food source comprising the Vig r 2 protein; optionally wherein the method does not comprise administering to the subject the Ara h 1 protein or a food source comprising the Ara h 1 protein. Provided herein is a method of treating an allergy to an Ara h 3 protein, or a food source comprising the Ara h 3 protein, in a subject in need thereof, the method comprising administering to the subject: a Car i 4 protein, or a food source comprising the Car i 4 protein; a Cor a 9 protein, or a food source comprising the Cor a 9 protein; a Jug n 4 protein, or a food source comprising the Jug n 4 protein; a Jug r 4 protein, or a food source comprising the Jug r 4 protein; a Pis v 2 protein, or a food source comprising the Pis v 2 protein; a Pis v 5 protein, or a food source comprising the Pis v 5 protein; a Pru du 6 protein, or a food source comprising the Pru du 6 protein; a Ses i 6 protein, or a food source comprising the Ses i 6 protein; a Ses i 7 protein, or a food source comprising the Ses i 7 protein; a Fag e 1 protein, or a food source comprising the Fag e 1 protein; a Fag t 13s g protein, or a food source comprising the Fag t 13s g protein; and/or an Ana o 2 protein, or a food source comprising the Ana o 2; optionally wherein the method does not comprise administering to the subject the Ara h 3 protein or a food source comprising the Ara h 3 protein. Provided herein is a method of treating an allergy to a Ber e 2 protein, or a food source comprising the Ber e 2 protein, in a subject in need thereof, the method comprising administering to the subject: a Fag e 1 protein, or a food source comprising the Fag e 1 protein; a Fag t 13s g protein, or a food source comprising the Fag t 13s g protein; an Ana o 2 protein, or a food source comprising the Ana o 2 protein; a Car i 4 protein, or a food source comprising the Car i 4 protein; a Cor a 9 protein, or a food source comprising the Cor a 9 protein; a Gly m 6 protein, or a food source comprising the Gly m 6 protein; a Jug n 4 protein, or a food source comprising the Jug n 4 protein; a Jug r 4 protein, or a food source comprising the Jug r 4 protein; a Pis v 2 protein, or a food source comprising the Pis v 2 protein; a Pis v 5 protein, or a food source comprising the Pis v 5 protein; a Pru du 6 protein, or a food source comprising the Pru du 6 protein; a Ses i 6 protein, or a food source comprising the Ses i 6 protein; and/or a Ses i 7 protein, or a food source comprising the Ses i 7 protein; optionally wherein the method does not comprise administering to the subject the Ber e 2 protein or a food source comprising the Ber e 2 protein. Provided herein is a method of treating an allergy to a Car i 2 protein, or a food source comprising the Car i 2 protein, in a subject in need thereof, the method comprising administering to the subject: a Cor a 11 protein, or a food source comprising the Cor a 11 protein; a Gly m 5 protein, or a food source comprising the Gly m 5 protein; a Jug r 2 protein, or a food source comprising the Jug r 2 protein; a Pis v 3 protein, or a food source comprising the Pis v 3 protein; a Ses i 3 protein, or a food source comprising the Ses i 3 protein; a Len c 1 protein, or a food source comprising the Len c 1 protein; a Lup an 1 protein, or a food source comprising the Lup an 1 protein; a Lup a v protein, or a food source comprising the Lup a v protein; a Pin k 2 protein, or a food source comprising the Pin k 2 protein; a Pis s 2 protein, or a food source comprising the Pis s 2 protein; a Vig r 2 protein, or a food source comprising the Vig r 2 protein; and/or an Ara h 1 protein, or a food source comprising the Ara h 1 protein; optionally wherein the method does not comprise administering to the subject the Car i 2 protein or a food source comprising the Car i 2 protein. Provided herein is a method of treating an allergy to a Car i 4 protein, or a food source comprising the Car i 4 protein, in a subject in need thereof, the method comprising administering to the subject: a Cor a 9 protein, or a food source comprising the Cor a 9 protein; a Gly m 6 protein, or a food source comprising the Gly m 6 protein; a Jug n 4 protein, or a food source comprising the Jug n 4 protein; a Jug r 4 protein, or a food source comprising the Jug r 4 protein; a Pis v 2 protein, or a food source comprising the Pis v 2 protein; a Pis v 5 protein, or a food source comprising the Pis v 5 protein; a Pru du 6 protein, or a food source comprising the Pru du 6 protein; a Ses i 6 protein, or a food source comprising the Ses i 6 protein; a Ses i 7 protein, or a food source comprising the Ses i 7 protein; a Ber e 2 protein, or a food source comprising the Ber e 2 protein; a Fag e 1 protein, or a food source comprising the Fag e 1 protein; a Fag t 13s g protein, or a food source comprising the Fag t 13s g protein; a Sin a 2 protein, or a food source comprising the Sin a 2 protein; an Ana o 2 protein, or a food source comprising the Ana o 2 protein; and/or an Ara h 3 protein, or a food source comprising the Ara h 3 protein; optionally wherein the method does not comprise administering to the subject the Car i 4 protein or a food source comprising the Car i 4 protein. Provided herein is a method of treating an allergy to a Cor a 11 protein, or a food source comprising the Cor a 11 protein, in a subject in need thereof, the method comprising administering to the subject: a Jug r 2 protein, or a food source comprising the Jug r 2 protein; a Pis v 3 protein, or a food source comprising the Pis v 3 protein; a Len c 1 protein, or a food source comprising the Len c 1 protein; a Pin k 2 protein, or a food source comprising the Pin k 2 protein; a Pis s 1 protein, or a food source comprising the Pis s 1 protein; a Pis s 2 protein, or a food source comprising the Pis s 2 protein; an Ana o 1 protein, or a food source comprising the Ana o 1 protein; and/or a Car i 2 protein, or a food source comprising the Car i 2 protein; optionally wherein the method does not comprise administering to the subject the Cor a 11 protein or a food source comprising the Cor a 11 protein. Provided herein is a method of treating an allergy to a Cor a 9 protein, or a food source comprising the Cor a 9 protein, in a subject in need thereof, the method comprising administering to the subject: a Gly m 6 protein, or a food source comprising the Gly m 6 protein; a Jug n 4 protein, or a food source comprising the Jug n 4 protein; a Jug r 4 protein, or a food source comprising the Jug r 4 protein; a Pis v 2 protein, or a food source comprising the Pis v 2 protein; a Pis v 5 protein, or a food source comprising the Pis v 5 protein; a Pru du 6 protein, or a food source comprising the Pru du 6 protein; a Ses i 6 protein, or a food source comprising the Ses i 6 protein; a Ses i 7 protein, or a food source comprising the Ses i 7 protein; a Ber e 2 protein, or a food source comprising the Ber e 2 protein; a Fag e 1 protein, or a food source comprising the Fag e 1 protein; a Fag t 13s g protein, or a food source comprising the Fag t 13s g protein; a Sin a 2 protein, or a food source comprising the Sin a 2 protein; an Ana o 2 protein, or a food source comprising the Ana o 2 protein; an Ara h 3 protein, or a food source comprising the Ara h 3 protein; and/or a Car i 4 protein, or a food source comprising the Car i 4 protein; optionally wherein the method does not comprise administering to the subject the Cor a 9 protein or a food source comprising the Cor a 9 protein. Provided herein is a method of treating an allergy to a Fag e 1 protein, or a food source comprising the Fag e 1 protein, in a subject in need thereof, the method comprising administering to the subject: a Fag t 13s g protein, or a food source comprising the Fag t 13s g protein; an Ana o 2 protein, or a food source comprising the Ana o 2 protein; an Ara h 3 protein, or a food source comprising the Ara h 3 protein; a Car i 4 protein, or a food source comprising the Car i 4 protein; a Cor a 9 protein, or a food source comprising the Cor a 9 protein; a Gly m 6 protein, or a food source comprising the Gly m 6 protein; a Jug r 4 protein, or a food source comprising the Jug r 4 protein; a Pis v 2 protein, or a food source comprising the Pis v 2 protein; a Pis v 5 protein, or a food source comprising the Pis v 5 protein; a Pru du 6 protein, or a food source comprising the Pru du 6 protein; a Ses i 7 protein, or a food source comprising the Ses i 7 protein; and/or a Ber e 2 protein, or a food source comprising the Ber e 2 protein; optionally wherein the method does not comprise administering to the subject the Fag e 1 protein or a food source comprising the Fag e 1 protein. Provided herein is a method of treating an allergy to a Fag t 13s g protein, or a food source comprising the Fag t 13s g protein, in a subject in need thereof, the method comprising administering to the subject: an Ana o 2 protein, or a food source comprising the Ana o 2 protein; an Ara h 3 protein, or a food source comprising the Ara h 3 protein; a Car i 4 protein, or a food source comprising the Car i 4 protein; a Cor a 9 protein, or a food source comprising the Cor a 9 protein; a Gly m 6 protein, or a food source comprising the Gly m 6 protein; a Jug r 4 protein, or a food source comprising the Jug r 4 protein; a Pis v 2 protein, or a food source comprising the Pis v 2 protein; a Pis v 5 protein, or a food source comprising the Pis v 5 protein; a Pru du 6 protein, or a food source comprising the Pru du 6 protein; a Ses i 7 protein, or a food source comprising the Ses i 7 protein; a Ber e 2 protein, or a food source comprising the Ber e 2 protein; and/or a Fag e 1 protein, or a food source comprising the Fag e 1 protein; optionally wherein the method does not comprise administering to the subject the Fag t 13s g protein or a food source comprising the Fag t 13s g protein. Provided herein is a method of treating an allergy to a Gly m 5 protein, or a food source comprising the Gly m 5 protein, in a subject in need thereof, the method comprising administering to the subject: a Jug r 2 protein, or a food source comprising the Jug r 2 protein; a Len c 1 protein, or a food source comprising the Len c 1 protein; a Lup an 1 protein, or a food source comprising the Lup an 1 protein; a Lup a v protein, or a food source comprising the Lup a v protein; a Pis s 1 protein, or a food source comprising the Pis s 1 protein; a Pis s 2 protein, or a food source comprising the Pis s 2 protein; a Vig r 2 protein, or a food source comprising the Vig r 2 protein; an Ara h 1 protein, or a food source comprising the Ara h 1 protein; and/or a Car i 2 protein, or a food source comprising the Car i 2 protein; optionally wherein the method does not comprise administering to the subject the Gly m 5 protein or a food source comprising the Gly m 5 protein. Provided herein is a method of treating an allergy to a Gly m 6 protein, or a food source comprising the Gly m 6 protein, in a subject in need thereof, the method comprising administering to the subject: a Jug n 4 protein, or a food source comprising the Jug n 4 protein; a Jug r 4 protein, or a food source comprising the Jug r 4 protein; a Pis v 2 protein, or a food source comprising the Pis v 2 protein; a Pru du 6 protein, or a food source comprising the Pru du 6 protein; a Ses i 7 protein, or a food source comprising the Ses i 7 protein; a Ber e 2 protein, or a food source comprising the Ber e 2 protein; a Fag e 1 protein, or a food source comprising the Fag e 1 protein; a Fag t 13s g protein, or a food source comprising the Fag 13s g protein; an Ana o 2 protein, or a food source comprising the Ana o 2 protein; a Car i 4 protein, or a food source comprising the Car i 4 protein; and/or a Cor a 9 protein, or a food source comprising the Cor a 9 protein; optionally wherein the method does not comprise administering to the subject the Gly m 6 protein or a food source comprising the Gly m 6 protein. Provided herein is a method of treating an allergy to a Jug n 4 protein, or a food source comprising the Jug n 4 protein, in a subject in need thereof, the method comprising administering to the subject: a Jug r 4 protein, or a food source comprising the Jug r 4 protein; a Pis v 2 protein, or a food source comprising the Pis v 2 protein; a Pis v 5 protein, or a food source comprising the Pis v 5 protein; a Pru du 6 protein, or a food source comprising the Pru du 6 protein; a Ses i 6 protein, or a food source comprising the Ses i 6 protein; a Ses i 7 protein, or a food source comprising the Ses i 7 protein; a Ber e 2 protein, or a food source comprising the Ber e 2 protein; a Sin a 2 protein, or a food source comprising the Sin a 2 protein; an Ana o 2 protein, or a food source comprising the Ana o 2 protein; an Ara h 3 protein, or a food source comprising the Ara h 3 protein; a Car i 4 protein, or a food source comprising the Car i 4 protein; a Cor a 9 protein, or a food source comprising the Cor a 9 protein; and/or a Gly m 6 protein, or a food source comprising the Gly m 6 protein; optionally wherein the method does not comprise administering to the subject the Jug n 4 protein or a food source comprising the Jug n 4 protein. Provided herein is a method of treating an allergy to a Jug r 2 protein, or a food source comprising the Jug r 2 protein, in a subject in need thereof, the method comprising administering to the subject: a Ses i 3 protein, or a food source comprising the Ses i 3 protein; a Len c 1 protein, or a food source comprising the Len c 1 protein; a Pin k 2 protein, or a food source comprising the Pin k 2 protein; a Pis s 1 protein, or a food source comprising the Pis s 1 protein; a Pis s 2 protein, or a food source comprising the Pis s 2 protein; a Vig r 2 protein, or a food source comprising the Vig r 2 protein; an Ara h 1 protein, or a food source comprising the Ara h 1 protein; a Car i 2 protein, or a food source comprising the Car i 2 protein; a Cor a 11 protein, or a food source comprising the Cor a 11 protein; and/or a Gly m 5 protein, or a food source comprising the Gly m 5 protein; optionally wherein the method does not comprise administering to the subject the Jug r 2 protein or a food source comprising the Jug r 2 protein. Provided herein is a method of treating an allergy to a Jug r 4 protein, or a food source comprising the Jug r 4 protein, in a subject in need thereof, the method comprising administering to the subject: a Pis v 2 protein, or a food source comprising the Pis v 2 protein; a Pis v 5 protein, or a food source comprising the Pis v 5 protein; a Pru du 6 protein, or a food source comprising the Pru du 6 protein; a Ses i 6 protein, or a food source comprising the Ses i 6 protein; a Ses i 7 protein, or a food source comprising the Ses i 7 protein; a Ber e 2 protein, or a food source comprising the Ber e 2 protein; a Fag e 1 protein, or a food source comprising the Fag e 1 protein; a Fag t 13s g protein, or a food source comprising the Fag t 13s g protein; a Sin a 2 protein, or a food source comprising the Sin a 2 protein; an Ana o 2 protein, or a food source comprising the Ana o 2 protein; an Ara h 3 protein, or a food source comprising the Ara h 3 protein; a Car i 4 protein, or a food source comprising the Car i 4 protein; a Cor a 9 protein, or a food source comprising the Cor a 9 protein; a Gly m 6 protein, or a food source comprising the Gly m 6 protein; and/or a Jug n 4 protein, or a food source comprising the Jug n 4 protein; optionally wherein the method does not comprise administering to the subject the Jug r 4 protein or a food source comprising the Jug r 4 protein. Provided herein is a method of treating an allergy to a Len c 1 protein, or a food source comprising the Len c 1 protein, in a subject in need thereof, the method comprising administering to the subject: a Lup an 1 protein, or a food source comprising the Lup an 1 protein; a Lup a v protein, or a food source comprising the Lup a v protein; a Pis s 1 protein, or a food source comprising the Pis s 1 protein; a Pis s 2 protein, or a food source comprising the Pis s 2 protein; a Vig r 2 protein, or a food source comprising the Vig r 2 protein; an Ara h 1 protein, or a food source comprising the Ara h 1 protein; a Car i 2 protein, or a food source comprising the Car i 2 protein; a Cor a 11 protein, or a food source comprising the Cor a 11 protein; a Gly m 5 protein, or a food source comprising the Gly m 5 protein; a Jug r 2 protein, or a food source comprising the Jug r 2 protein; and/or a Ses i 3 protein, or a food source comprising the Ses i 3 protein; optionally wherein the method does not comprise administering to the subject the Len c 1 protein or a food source comprising the Len c 1 protein. Provided herein is a method of treating an allergy to a Lup an 1 protein, or a food source comprising the Lup an 1 protein, in a subject in need thereof, the method comprising administering to the subject: a Lup a v protein, or a food source comprising the Lup a v protein; a Pis s 1 protein, or a food source comprising the Pis s 1 protein; a Pis s 2 protein, or a food source comprising the Pis s 2 protein; a Vig r 2 protein, or a food source comprising the Vig r 2 protein; an Ara h 1 protein, or a food source comprising the Ara h 1 protein; a Car i 2 protein, or a food source comprising the Car i 2 protein; a Gly m 5 protein, or a food source comprising the Gly m 5 protein; a Ses i 3 protein, or a food source comprising the Ses i 3 protein; and/or a Len c 1 protein, or a food source comprising the Len c 1 protein; optionally wherein the method does not comprise administering to the subject the Lup an 1 protein or a food source comprising the Lup an 1 protein. Provided herein is a method of treating an allergy to a Lup a v protein, or a food source comprising the Lup a v protein, in a subject in need thereof, the method comprising administering to the subject: a Pis s 1 protein, or a food source comprising the Pis s 1 protein; a Pis s 2 protein, or a food source comprising the Pis s 2 protein; a Vig r 2 protein, or a food source comprising the Vig r 2 protein; an Ara h 1 protein, or a food source comprising the Ara h 1 protein; a Car i 2 protein, or a food source comprising the Car i 2 protein; a Gly m 5 protein, or a food source comprising the Gly m 5 protein; a Len c 1 protein, or a food source comprising the Len c 1 protein; and/or a Lup an 1 protein, or a food source comprising the Lup an 1 protein; optionally wherein the method does not comprise administering to the subject the Lup a v protein or a food source comprising the Lup a v protein. Provided herein is a method of treating an allergy to a Pin k 2 protein, or a food source comprising the Pin k 2 protein, in a subject in need thereof, the method comprising administering to the subject: a Car i 2 protein, or a food source comprising the Car i 2 protein; a Cor a 11 protein, or a food source comprising the Cor a 11 protein; a Jug r 2 protein, or a food source comprising the Jug r 2 protein; and/or a Pis v 3 protein, or a food source comprising the Pis v 3 protein; optionally wherein the method does not comprise administering to the subject the Pin k 2 protein or a food source comprising the Pin k 2 protein. Provided herein is a method of treating an allergy to a Pis s 1 protein, or a food source comprising the Pis s 1 protein, in a subject in need thereof, the method comprising administering to the subject: a Pis s 2 protein, or a food source comprising the Pis s 2 protein; a Vig r 2 protein, or a food source comprising the Vig r 2 protein; an Ara h 1 protein, or a food source comprising the Ara h 1 protein; a Car i 2 protein, or a food source comprising the Car i 2 protein; a Cor a 11 protein, or a food source comprising the Cor a 11 protein; a Gly m 5 protein, or a food source comprising the Gly m 5 protein; a Jug r 2 protein, or a food source comprising the Jug r 2 protein; a Ses i 3 protein, or a food source comprising the Ses i 3 protein; a Len c 1 protein, or a food source comprising the Len c 1 protein; a Lup an 1 protein, or a food source comprising the Lup an 1 protein; and/or a Lup a v protein, or a food source comprising the Lup a v protein; optionally wherein the method does not comprise administering to the subject the Pis s 1 protein or a food source comprising the Pis s 1 protein. Provided herein is a method of treating an allergy to a Pis s 2 protein, or a food source comprising the Pis s 2 protein, in a subject in need thereof, the method comprising administering to the subject: a Vig r 2 protein, or a food source comprising the Vig r 2 protein; an Ara h 1 protein, or a food source comprising the Ara h 1 protein; a Car i 2 protein, or a food source comprising the Car i 2 protein; a Cor a 11 protein, or a food source comprising the Cor a 11 protein; a Gly m 5 protein, or a food source comprising the Gly m 5 protein; a Jug r 2 protein, or a food source comprising the Jug r 2 protein; a Ses i 3 protein, or a food source comprising the Ses i 3 protein; a Len c 1 protein, or a food source comprising the Len c 1 protein; a Lup an 1 protein, or a food source comprising the Lup an 1 protein; a Lup a v protein, or a food source comprising the Lup a v protein; and/or a Pis s 1 protein, or a food source comprising the Pis s 1 protein; optionally wherein the method does not comprise administering to the subject the Pis s 2 protein or a food source comprising the Pis s 2 protein. Provided herein is a method of treating an allergy to a Pis v 2 protein, or a food source comprising the Pis v 2 protein, in a subject in need thereof, the method comprising administering to the subject: a Pis v 5 protein, or a food source comprising the Pis v 5 protein; a Pru du 6 protein, or a food source comprising the Pru du 6 protein; a Ses i 6 protein, or a food source comprising the Ses i 6 protein; a Ses i 7 protein, or a food source comprising the Ses i 7 protein; a Ber e 2 protein, or a food source comprising the Ber e 2 protein; a Fag e 1 protein, or a food source comprising the Fag e 1 protein; a Fag t 13s g protein, or a food source comprising the Fag t 13s g protein; an Ana o 2 protein, or a food source comprising the Ana o 2 protein; an Ara h 3 protein, or a food source comprising the Ara h 3 protein; a Car i 4 protein, or a food source comprising the Car i 4 protein; a Cor a 9 protein, or a food source comprising the Cor a 9 protein; a Gly m 6 protein, or a food source comprising the Gly m 6 protein; a Jug n 4 protein, or a food source comprising the Jug n 4 protein; and/or a Jug r 4 protein, or a food source comprising the Jug r 4 protein; optionally wherein the method does not comprise administering to the subject the Pis v 2 protein or a food source comprising the Pis v 2 protein. Provided herein is a method of treating an allergy to a Pis v 3 protein, or a food source comprising the Pis v 3 protein, in a subject in need thereof, the method comprising administering to the subject: a Ses i 3 protein, or a food source comprising the Ses i 3 protein; a Pin k 2 protein, or a food source comprising the Pin k 2 protein; an Ana o 1 protein, or a food source comprising the Ana o 1 protein; a Car i 2 protein, or a food source comprising the Car i 2 protein; and/or a Cor a 11 protein, or a food source comprising the Cor a 11 protein; optionally wherein the method does not comprise administering to the subject the Pis v 3 protein or a food source comprising the Pis v 3 protein. Provided herein is a method of treating an allergy to a Pru du 6 protein, or a food source comprising the Pru du 6 protein, in a subject in need thereof, the method comprising administering to the subject: a Ses i 6 protein, or a food source comprising the Ses i 6 protein; a Ses i 7 protein, or a food source comprising the Ses i 7 protein; a Ber e 2 protein, or a food source comprising the Ber e 2 protein; a Fag e 1 protein, or a food source comprising the Fag e 1 protein; a Fag t 13s g protein, or a food source comprising the Fag t 13s g protein; a Sin a 2 protein, or a food source comprising the Sin a 2 protein; an Ana o 2 protein, or a food source comprising the Ana o 2 protein; an Ara h 3 protein, or a food source comprising the Ara h 3 protein; a Car i 4 protein, or a food source comprising the Car i 4 protein; a Cor a 9 protein, or a food source comprising the Cor a 9 protein; a Gly m 6 protein, or a food source comprising the Gly m 6 protein; a Jug n 4 protein, or a food source comprising the Jug n 4 protein; a Jug r 4 protein, or a food source comprising the Jug r 4 protein; a Pis v 2 protein, or a food source comprising the Pis v 2 protein; and/or a Pis v 5 protein, or a food source comprising the Pis v 5 protein; optionally wherein the method does not comprise administering to the subject the Pru du 6 protein or a food source comprising the Pru du 6 protein. Provided herein is a method of treating an allergy to a Ses i 3 protein, or a food source comprising the Ses i 3 protein, in a subject in need thereof, the method comprising administering to the subject: a Len c 1 protein, or a food source comprising the Len c 1 protein; a Lup an 1 protein, or a food source comprising the Lup an 1 protein; a Pis s 1 protein, or a food source comprising the Pis s 1 protein; a Pis s 2 protein, or a food source comprising the Pis s 2 protein; an Ana o 1 protein, or a food source comprising the Ana o 1 protein; an Ara h 1 protein, or a food source comprising the Ara h 1 protein; a Car i 2 protein, or a food source comprising the Car i 2 protein; a Jug r 2 protein, or a food source comprising the Jug r 2 protein; and/or a Pis v 3 protein, or a food source comprising the Pis v 3 protein; optionally wherein the method does not comprise administering to the subject the Ses i 3 protein or a food source comprising the Ses i 3 protein. Provided herein is a method of treating an allergy to a Ses i 6 protein, or a food source comprising the Ses i 6 protein, in a subject in need thereof, the method comprising administering to the subject: a Ber e 2 protein, or a food source comprising the Ber e 2 protein; a Fag t 13s g protein, or a food source comprising the Fag t 13s g protein; an Ana o 2 protein, or a food source comprising the Ana o 2 protein; an Ara h 3 protein, or a food source comprising the Ara h 3 protein; a Car i 4 protein, or a food source comprising the Car i 4 protein; a Cor a 9 protein, or a food source comprising the Cor a 9 protein; a Jug n 4 protein, or a food source comprising the Jug n 4 protein; a Jug r 4 protein, or a food source comprising the Jug r 4 protein; a Pis v 2 protein, or a food source comprising the Pis v 2 protein; a Pis v 5 protein, or a food source comprising the Pis v 5 protein; and/or a Pru du 6 protein, or a food source comprising the Pru du 6 protein; optionally wherein the method does not comprise administering to the subject the Ses i 6 protein or a food source comprising the Ses i 6 protein. Provided herein is a method of treating an allergy to a Ses i 7 protein, or a food source comprising the Ses i 7 protein, in a subject in need thereof, the method comprising administering to the subject: a Ber e 2 protein, or a food source comprising the Ber e 2 protein; a Fag e 1 protein, or a food source comprising the Fag e 1 protein; a Fag t 13s g protein, or a food source comprising the Fag t 13s g protein; a Sin a 2 protein, or a food source comprising the Sin a 2 protein; an Ana o 2 protein, or a food source comprising the Ana o 2 protein; an Ara h 3 protein, or a food source comprising the Ara h 3 protein; a Car i 4 protein, or a food source comprising the Car i 4 protein; a Cor a 9 protein, or a food source comprising the Cor a 9 protein; a Gly m 6 protein, or a food source comprising the Gly m 6 protein; a Jug n 4 protein, or a food source comprising the Jug n 4 protein; a Jug r 4 protein, or a food source comprising the Jug r 4 protein; a Pis v 2 protein, or a food source comprising the Pis v 2 protein; a Pis v 5 protein, or a food source comprising the Pis v 5 protein; and/or a Pru du 6 protein, or a food source comprising the Pru du 6 protein; optionally wherein the method does not comprise administering to the subject the Ses i 7 protein or a food source comprising the Ses i 7 protein. Provided herein is a method of treating an allergy to a Sin a 2 protein, or a food source comprising the Sin a 2 protein, in a subject in need thereof, the method comprising administering to the subject: an Ana o 2 protein, or a food source comprising the Ana o 2 protein; a Car i 4 protein, or a food source comprising the Car i 4 protein; a Cor a 9 protein, or a food source comprising the Cor a 9 protein; a Jug n 4 protein, or a food source comprising the Jug n 4 protein; a Jug r 4 protein, or a food source comprising the Jug r 4 protein; a Pis v 5 protein, or a food source comprising the Pis v 5 protein; a Pru du 6 protein, or a food source comprising the Pru du 6 protein; and/or a Ses i 7 protein, or a food source comprising the Ses i 7 protein; optionally wherein the method does not comprise administering to the subject the Sin a 2 protein or a food source comprising the Sin a 2 protein. Provided herein is a method of treating an allergy to a Vig r 2 protein, or a food source comprising the Vig r 2 protein, in a subject in need thereof, the method comprising administering to the subject: an Ara h 1 protein, or a food source comprising the Ara h 1 protein; a Car i 2 protein, or a food source comprising the Car i 2 protein; a Gly m 5 protein, or a food source comprising the Gly m 5 protein; a Jug r 2 protein, or a food source comprising the Jug r 2 protein; a Len c 1 protein, or a food source comprising the Len c 1 protein; a Lup an 1 protein, or a food source comprising the Lup an 1 protein; a Lup a v protein, or a food source comprising the Lup a v protein; a Pis s 1 protein, or a food source comprising the Pis s 1 protein; and/or a Pis s 2 protein, or a food source comprising the Pis s 2 protein; optionally wherein the method does not comprise administering to the subject the Vig r 2 protein or a food source comprising the Vig r 2 protein.

Similarly, the disclosure also provides methods of treating an allergy to a first Tropomyosin family protein (e.g., a protein identified in FIG. 13), or a food source including the first Tropomyosin family protein, in a subject in need thereof. A contemplated method comprises administering to the subject a second Tropomyosin family protein that is cross-reactive with the first Tropomyosin family protein (e.g., a protein that in FIG. 13 is connected to the first Tropomyosin family protein by a line) or a food source including the second Tropomyosin family protein. The disclosure also provides methods of treating an allergy to a first Bet v 1 family protein (e.g., a protein identified in FIG. 14), or a food source including the first Bet v 1 family protein, in a subject in need thereof. A contemplated method comprises administering to the subject a second Bet v 1 family protein that is cross-reactive with the first Bet v 1 family protein (e.g., a protein that in FIG. 14 is connected to the first Bet v 1 family protein by a line) or a food source including the second Bet v 1 family protein.

In another aspect, provided herein is a method of treating an allergy to one or more of salmon, shrimp, prawn, crab, lobster, crayfish, mussel, scallop, oyster, clam, abalone, squid, octopus, and nautilus in a subject in need thereof, the method comprising administering to the subject a multiple allergen oral immunotherapy composition comprising two or more of salmon, shrimp, prawn, crab, lobster, crayfish, mussels, scallop, oyster, clam, abalone, squid, octopus, and/or nautilus, optionally wherein the multiple allergen oral immunotherapy composition does not comprise one or more of salmon, shrimp, prawn, crab, lobster, crayfish, mussels, scallop, oyster, clam, abalone, squid, octopus, and nautilus.

In another aspect, provided herein is a method of treating an allergy to salmon in a subject in need thereof, the method comprising administering to the subject a multiple allergen oral immunotherapy composition comprising one or more of shrimp, prawn, crab, lobster, crayfish, mussel, scallop, oyster, clam, abalone, squid, octopus, and nautilus, optionally wherein the multiple allergen oral immunotherapy composition does not comprise salmon.

In another aspect, provided herein is a method of treating an allergy to shrimp in a subject in need thereof, the method comprising administering to the subject a multiple allergen oral immunotherapy composition comprising one or more of salmon, prawn, crab, lobster, crayfish, mussel, scallop, oyster, clam, abalone, squid, octopus, and nautilus, optionally wherein the multiple allergen oral immunotherapy composition does not comprise shrimp.

In another aspect, provided herein is a method of treating an allergy to prawn in a subject in need thereof, the method comprising administering to the subject a multiple allergen oral immunotherapy composition comprising one or more of salmon, shrimp, crab, lobster, crayfish, mussel, scallop, oyster, clam, abalone, squid, octopus, and nautilus, optionally wherein the multiple allergen oral immunotherapy composition does not comprise prawn.

In another aspect, provided herein is a method of treating an allergy to crab in a subject in need thereof, the method comprising administering to the subject a multiple allergen oral immunotherapy composition comprising one or more of salmon, shrimp, prawn, lobster, crayfish, mussel, scallop, oyster, clam, abalone, squid, octopus, and nautilus, optionally wherein the multiple allergen oral immunotherapy composition does not comprise crab.

In another aspect, provided herein is a method of treating an allergy to lobster in a subject in need thereof, the method comprising administering to the subject a multiple allergen oral immunotherapy composition comprising one or more of salmon, shrimp, prawn, crab, crayfish, mussel, scallop, oyster, clam, abalone, squid, octopus, and nautilus, optionally wherein the multiple allergen oral immunotherapy composition does not comprise lobster.

In another aspect, provided herein is a method of treating an allergy to crayfish in a subject in need thereof, the method comprising administering to the subject a multiple allergen oral immunotherapy composition comprising one or more of salmon, shrimp, prawn, crab, lobster, mussel, scallop, oyster, clam, abalone, squid, octopus, and nautilus, optionally wherein the multiple allergen oral immunotherapy composition does not comprise crayfish.

In another aspect, provided herein is a method of treating an allergy to mussel in a subject in need thereof, the method comprising administering to the subject a multiple allergen oral immunotherapy composition comprising one or more of salmon, shrimp, prawn, crab, lobster, crayfish, scallop, oyster, clam, abalone, squid, octopus, and nautilus, optionally wherein the multiple allergen oral immunotherapy composition does not comprise mussel.

In another aspect, provided herein is a method of treating an allergy to scallop in a subject in need thereof, the method comprising administering to the subject a multiple allergen oral immunotherapy composition comprising one or more of salmon, shrimp, prawn, crab, lobster, crayfish, mussel, oyster, clam, abalone, squid, octopus, and nautilus, optionally wherein the multiple allergen oral immunotherapy composition does not comprise scallop.

In another aspect, provided herein is a method of treating an allergy to oyster in a subject in need thereof, the method comprising administering to the subject a multiple allergen oral immunotherapy composition comprising one or more of salmon, shrimp, prawn, crab, lobster, crayfish, mussel, scallop, clam, abalone, squid, octopus, and nautilus, optionally wherein the multiple allergen oral immunotherapy composition does not comprise oyster.

In another aspect, provided herein is a method of treating an allergy to clam in a subject in need thereof, the method comprising administering to the subject a multiple allergen oral immunotherapy composition comprising one or more of salmon, shrimp, prawn, crab, lobster, crayfish, mussel, scallop, oyster, abalone, squid, octopus, and nautilus, optionally wherein the multiple allergen oral immunotherapy composition does not comprise clam.

In another aspect, provided herein is a method of treating an allergy to abalone in a subject in need thereof, the method comprising administering to the subject a multiple allergen oral immunotherapy composition comprising one or more of salmon, shrimp, prawn, crab, lobster, crayfish, mussel, scallop, oyster, clam, squid, octopus, and nautilus, optionally wherein the multiple allergen oral immunotherapy composition does not comprise abalone.

In another aspect, provided herein is a method of treating an allergy to squid in a subject in need thereof, the method comprising administering to the subject a multiple allergen oral immunotherapy composition comprising one or more of salmon, shrimp, prawn, crab, lobster, crayfish, mussel, scallop, oyster, clam, abalone, octopus, and nautilus, optionally wherein the multiple allergen oral immunotherapy composition does not comprise squid.

In another aspect, provided herein is a method of treating an allergy to octopus in a subject in need thereof, the method comprising administering to the subject a multiple allergen oral immunotherapy composition comprising one or more of salmon, shrimp, prawn, crab, lobster, crayfish, mussel, scallop, oyster, clam, abalone, squid, and nautilus, optionally wherein the multiple allergen oral immunotherapy composition does not comprise octopus.

In another aspect, provided herein is a method of treating an allergy to nautilus in a subject in need thereof, the method comprising administering to the subject a multiple allergen oral immunotherapy composition comprising one or more of salmon, shrimp, prawn, crab, lobster, crayfish, mussel, scallop, oyster, clam, abalone, squid, and octopus, optionally wherein the multiple allergen oral immunotherapy composition does not comprise nautilus.

In another aspect, provided herein is a method of treating an allergy to one or more of cod, salmon, shrimp, and carp in a subject in need thereof, the method comprising administering to the subject a multiple allergen oral immunotherapy composition comprising two or more of cod, salmon, shrimp, and carp, optionally wherein the multiple allergen oral immunotherapy composition does not comprise one or more of cod, salmon, shrimp, and carp.

In another aspect, provided herein is a method of treating an allergy to cod in a subject in need thereof, the method comprising administering to the subject a multiple allergen oral immunotherapy composition comprising one or more of salmon, shrimp, and carp, optionally wherein the multiple allergen oral immunotherapy composition does not comprise cod.

In another aspect, provided herein is a method of treating an allergy to salmon in a subject in need thereof, the method comprising administering to the subject a multiple allergen oral immunotherapy composition comprising two or more of cod, salmon, shrimp, and carp, optionally wherein the multiple allergen oral immunotherapy composition does not comprise salmon.

In another aspect, provided herein is a method of treating an allergy to shrimp in a subject in need thereof, the method comprising administering to the subject a multiple allergen oral immunotherapy composition comprising two or more of cod, salmon, shrimp, and carp, optionally wherein the multiple allergen oral immunotherapy composition does not comprise shrimp.

In another aspect, provided herein is a method of treating an allergy to carp in a subject in need thereof, the method comprising administering to the subject a multiple allergen oral immunotherapy composition comprising two or more of cod, salmon, shrimp, and carp, optionally wherein the multiple allergen oral immunotherapy composition does not comprise carp.

In another aspect, provided herein is a method of treating an allergy to one or more of egg, milk, and cheese in a subject in need thereof, the method comprising administering to the subject a multiple allergen oral immunotherapy composition comprising two or more of egg, milk, and cheese, optionally wherein the multiple allergen oral immunotherapy composition does not comprise one or more of egg, milk, and cheese.

In another aspect, provided herein is a method of treating an allergy to egg in a subject in need thereof, the method comprising administering to the subject a multiple allergen oral immunotherapy composition comprising one or both of milk and cheese, optionally wherein the multiple allergen oral immunotherapy composition does not comprise egg.

In another aspect, provided herein is a method of treating an allergy to milk in a subject in need thereof, the method comprising administering to the subject a multiple allergen oral immunotherapy composition comprising one or both of milk and cheese, optionally wherein the multiple allergen oral immunotherapy composition does not comprise milk.

In another aspect, provided herein is a method of treating an allergy to cheese in a subject in need thereof, the method comprising administering to the subject a multiple allergen oral immunotherapy composition comprising one or both of milk and cheese, optionally wherein the multiple allergen oral immunotherapy composition does not comprise cheese.

In another aspect, provided herein is a method of treating an allergy to one or more of wheat, corn, barley, and oat in a subject in need thereof, the method comprising administering to the subject a multiple allergen oral immunotherapy composition comprising two or more of wheat, corn, barley, and oat, optionally wherein the multiple allergen oral immunotherapy composition does not comprise one or more of wheat, corn, barley, and oat.

In another aspect, provided herein is a method of treating an allergy to wheat in a subject in need thereof, the method comprising administering to the subject a multiple allergen oral immunotherapy composition comprising one or more of corn, barley, and oat, optionally wherein the multiple allergen oral immunotherapy composition does not comprise wheat.

In another aspect, provided herein is a method of treating an allergy to corn in a subject in need thereof, the method comprising administering to the subject a multiple allergen oral immunotherapy composition comprising two or more of wheat, corn, barley, and oat, optionally wherein the multiple allergen oral immunotherapy composition does not comprise corn.

In another aspect, provided herein is a method of treating an allergy to barley in a subject in need thereof, the method comprising administering to the subject a multiple allergen oral immunotherapy composition comprising two or more of wheat, corn, barley, and oat, optionally wherein the multiple allergen oral immunotherapy composition does not comprise barley.

In another aspect, provided herein is a method of treating an allergy to oat in a subject in need thereof, the method comprising administering to the subject a multiple allergen oral immunotherapy composition comprising two or more of wheat, corn, barley, and oat, optionally wherein the multiple allergen oral immunotherapy composition does not comprise oat.

In another aspect, provided herein is a method of identifying an antibody (for example, an IgE or IgG antibody), for example, an antibody useful for detection of a cross-reactive allergenic protein. A contemplated method comprises: (a) identifying a cross-reactive T-cell or B-cell epitope peptide within the cross-reactive allergenic protein; and/or (b) identifying an IgE or IgG antibody that binds to the identified cross-reactive T-cell or B-cell epitope peptide.

In certain embodiments, step (a) comprises: (i) identifying a T-cell or B-cell epitope peptide within a reference allergenic protein; (ii) identifying a T-cell or B-cell epitope within an other protein that is in the same protein domain family as the reference allergenic protein; and/or (iii) calculating pairwise identity and/or similarity between the T-cell or B-cell epitope peptide within the reference allergenic protein and the T-cell or B-cell epitope within the other protein, so as to determine whether the T-cell or B-cell from the other protein is cross-reactive to the T-cell or B-cell from the reference allergenic protein.

In certain embodiments, step (i) comprises: selecting a peptide within the reference allergenic protein; and calculating pairwise identity and/or similarity between the peptide and a known T-cell or B-cell epitope, so as to determine whether the peptide is a T-cell or B-cell epitope.

A contemplated method may, for example, comprise determining the MHC binding potential of a T-cell epitope and/or mapping the cross-reactive T-cell or B-cell epitope peptide to the full-length sequence of the cross-reactive allergenic protein.

A cross-reactive allergenic protein and/or a reference protein may, for example, be selected from the group consisting of a shrimp allergen, a cod allergen, a salmon allergen, a hen's egg allergen, a cow's milk allergen, a peanut allergen, a sesame allergen, a soy allergen, a pecan allergen, a cashew allergen, a hazelnut allergen, a walnut allergen, a pistachio allergen, an almond allergen, and a wheat allergen, or be selected from the group consisting of allergenic proteins listed in Tables 2, 4-7, and 10-12.

In certain embodiments, a contemplated protein family is selected from the group consisting of: Tropomyosin, EF hand family (Beta-parvalbumin), ATP:guanido phosphotransferase (Arginine Kinase), Triosephosphate isomerase, Fructose bisphosphate aldolase class I, Alpha/beta casein, Lipocalin, Kappa-casein, Transferrin, C-type lysozyme/alpha-lactalbumin family, Serum albumin, Enolase, Serpin serine protease inhibitor, Kazal-type serine protease inhibitor, Collagen, Lipoprotein, Prolamin superfamily, Cupin, Profilin, Bet v 1 family, Oleosin, Isoflavone reductase family, Plant defensin, 60S acidic ribosomal protein, Heat shock protein Hsp70, Fe/Mn superoxide dismutase, Papain-like cysteine protease, Thaumatin-like protein, Unclassified, GILT family, Hevein-like and class I/II chitinase, Zinc finger, C3HC4 type (RING finger), SCAI family, Peroxidase, Serpin serine protease inhibitor, Thionin, Thioredoxin, Transcription elongation factor 1, and Triosephosphate isomerase.

In another aspect, provided herein is an antibody, e.g., an IgE or IgG antibody, identified by a disclosed method.

In another aspect, provided herein is an antibody, e.g., an IgE or IgG antibody, to a cross-reactive epitope sequence selected from the group consisting of: SEQ ID NOs: 1-11742. In another aspect, provided herein is an antibody, e.g., an IgE or IgG antibody, to an epitope sequence listed in any one of Tables 13-23.

In another aspect, provided herein is a method for detecting the presence or amount of a cross-reactive allergenic protein. A contemplated method comprises: (a) providing an antibody to a cross-reactive B-cell or T-cell epitope sequence within the cross-reactive allergenic protein; (b) contacting a sample (e.g., a biological sample) that may have the cross-reactive allergenic protein under conditions sufficient to permit binding of the antibody; and/or (c) detecting binding of the antibody, thereby to identify the presence or amount of the cross-reactive allergenic protein.

In certain embodiments, a cross-reactive allergenic protein is a kiwifruit, prawn, kiwi, brazil nut, lobster, celery, coconut, crab, apple, buckwheat, clam, pear, lentil, mussel, tomato, garden pea, oyster, stone fruit (e.g., apricot, peach, cherry), yellow mustard, abalone, birch tree, mung bean, squid, alder tree, lupin, octopus, white oak tree, Korean pine, house dust mite, cockroach, or yellow fever mosquito cross-reactive allergenic protein.

A contemplated antibody may include, for example, an IgE or IgG antibody, for example, an IgE or IgG antibody contemplated herein, for example an IgE or IgG antibody that binds to a cross-reactive epitope sequence selected from the group consisting of: SEQ ID NOs: 1-11742. A contemplated antibody may, for example, be immobilized on a substrate.

In another aspect, provided herein is kit (for example, a detection kit) comprising six or more antibodies (for example IgE or IgG antibodies) contemplated herein, each bound to a solid support. In certain embodiments, a contemplated kit includes an IgE or IgG labeling reagent; a binding buffer; a wash buffer; a detection buffer; and/or instructions for use.

Exemplary solid supports or substrates include a membrane, a filter, a chip, a slide (such as a microscopic slide), an array (such as a microarray), a wafer, a fiber, a magnetic or a non-magnetic bead, a gel, a tubing, a plate (such as a microtiter plate), a microtiter strip, a polymer, a microparticle, a sphere, a microsphere, a nanosphere, a particle, a microparticle, a nanoparticle, or a capillary.

In another aspect, provided herein is a method for preparing a multiple allergen oral immunotherapy composition that is capable of treating an allergy to a reference allergenic protein that is not present in the multiple allergen oral immunotherapy. A contemplated method comprises: (a) identifying an other protein that is the same protein domain family as the reference allergenic protein; (b) calculating pairwise identity and/or similarity between an amino acid sequence (for example, a full-length amino acid sequence) of the reference allergenic protein and an other protein, so as to determine whether the other protein is cross-reactive to the reference allergenic protein; and/or (c) selecting the other allergenic protein as a component in the multiple allergen oral immunotherapy if the other protein is cross-reactive to the reference allergenic protein. Alternatively, a contemplated method may comprise selecting the reference allergenic protein as a component in the multiple allergen oral immunotherapy so as to treat an allergy to the other allergenic protein.

In certain embodiments, step (b) comprises: (i) providing a dataset comprising the amino acid sequence of the reference allergenic protein and the other protein; (ii) inputting the dataset into a computer readable medium; (iii) performing a bioinformatic step to calculate pairwise identity and similarity between the reference allergenic protein and the other protein; and/or (iv) identifying the other protein as cross-reactive to the reference allergenic protein based on pairwise identity and similarity between the reference allergenic protein and the other protein.

A bioinformatic step may, for example, comprise applying an algorithm to determine homology between the reference allergenic protein and the other protein based on pairwise identity and similarity, wherein the algorithm is selected from the group consisting of GAP, BESTFIT, FASTA, TFASTA, BLAST, BLASTP, TBLASTN, FASTDB, ALIGN, CLUSTALW, and CLUSTAL-Omega, including any version thereof. In certain embodiments, a bioinformatic step comprises applying an algorithm comprising (identity+similarity)/2 to determine a cross-reactivity score, for example, an other protein is considered cross-reactive to a reference allergenic protein if the other protein has a cross-reactivity score of ≥0.5 with respect to the reference allergenic protein. In certain embodiments, an other protein is considered cross-reactive to a reference allergenic protein if the other protein has ≥70% homology to the reference allergenic protein.

A contemplated method may, for example, comprise, prior to step (a), identifying the reference allergenic protein as a protein in an allergenic food source, and/or mapping the reference allergenic protein to a protein domain family. A contemplated method may, for example, comprise identifying a T-cell epitope peptide, a B-cell epitope peptide, and/or an MHC-binding epitope peptide in the allergenic protein that is cross-reactive to a T-cell epitope peptide, a B-cell epitope peptides and/or an MHC-binding epitope in the other protein. In certain embodiments, a contemplated method further comprises determining the MHC binding potential of the T-cell or B-cell peptide.

In certain embodiments, a contemplated protein family is selected from the group consisting of: Tropomyosin, EF hand family (Beta-parvalbumin), ATP:guanido phosphotransferase (Arginine Kinase), Triosephosphate isomerase, Fructose bisphosphate aldolase class I, Alpha/beta casein, Lipocalin, Kappa-casein, Transferrin, C-type lysozyme/alpha-lactalbumin family, Serum albumin, Enolase, Serpin serine protease inhibitor, Kazal-type serine protease inhibitor, Collagen, Lipoprotein, Prolamin superfamily, Cupin, Profilin, Bet v 1 family, Oleosin, Isoflavone reductase family, Plant defensin, 60S acidic ribosomal protein, Heat shock protein Hsp70, Fe/Mn superoxide dismutase, Papain-like cysteine protease, Thaumatin-like protein, Unclassified, GILT family, Hevein-like and class I/II chitinase, Zinc finger, C3HC4 type (RING finger), SCAI family, Peroxidase, Serpin serine protease inhibitor, Thionin, Thioredoxin, Transcription elongation factor 1, and Triosephosphate isomerase.

A reference protein and/or an other allergenic protein may, for example, be selected from the group consisting of a shrimp allergen, a cod allergen, a salmon allergen, a hen's egg allergen, a cow's milk allergen, a peanut allergen, a sesame allergen, a soy allergen, a pecan allergen, a cashew allergen, a hazelnut allergen, a walnut allergen, a pistachio allergen, an almond allergen, and a wheat allergen, or be selected from the group consisting of proteins listed in Tables 2, 4-7, and 10-12.

In another aspect, provided herein is a multiple allergen oral immunotherapeutic composition prepared by a disclosed method.

In another aspect, provided herein is a multiple allergen oral immunotherapy composition comprising: (a) three or more Cupin family proteins; and (b) a pharmaceutically acceptable excipient. In certain embodiments, upon administration of the oral immunotherapy composition to a subject having an allergy or at risk of having an allergy to one or more of the three or more Cupin family proteins, the subject has increased resistance to the protein as compared to treatment with one Cupin family protein alone; and/or is also treated for an allergy to a different Cupin family protein. In a contemplated composition, the three or more Cupin family proteins and/or the different Cupin family member are selected from the group consisting of proteins listed in Table 5 and Table 10. In certain embodiments, the Cupin family proteins are selected from Pru du 6, Gly m 5, Gly m 6, and Gly m Bd, and upon administration the subject is also effectively treated for a Pis s 1, a Pis s 2 allergy and/or has more effective treatment for a Jug n 4 protein allergy as compared to administering a treatment with Jug n 4 alone.

In another aspect, provided herein is a multiple allergen oral immunotherapy composition comprising: (a) three or more Tropomyosin family proteins; and (b) a pharmaceutically acceptable excipient. In certain embodiments, upon administration of the oral immunotherapy composition to a subject having an allergy or at risk of having an allergy to one or more of the three or more Tropomyosin family proteins, the subject has increased resistance to the protein as compared to treatment with one Tropomyosin family protein alone; and/or is also treated for an allergy to a different Tropomyosin family protein. In a contemplated composition, the three or more Tropomyosin family proteins and/or the different Tropomyosin family member are selected from the group consisting of proteins listed in Table 6 and Table 11.

In another aspect, provided herein is a multiple allergen oral immunotherapy composition comprising: (a) three or more Bet v 1 family proteins; and (b) a pharmaceutically acceptable excipient. In certain embodiments, upon administration of the oral immunotherapy composition to a subject having an allergy or at risk of having an allergy to one or more of the three or more Bet v 1 family proteins, the subject has increased resistance to the protein as compared to treatment with one Bet v 1 family protein alone; and/or is also treated for an allergy to a different Bet v 1 family protein. In a contemplated composition, the three or more Bet v 1 family proteins and/or the different Bet v 1 family member are selected from the group consisting of proteins listed in Table 7 and Table 12.

In another aspect, provided herein is an allergen immunotherapeutic composition. A contemplated composition comprises at least 4 peptides, wherein the at least 4 peptides are each (i) from 9 to 25 amino acids in length (for example 15 amino acids in length), (ii) selected from the group consisting of a T-cell epitope peptide, a B-cell epitope peptide, and an MHC-binding peptide, and/or (iii) derived from an allergenic protein in a protein family selected from the group consisting of Tropomyosin, EF hand family (Beta-parvalbumin), ATP:guanido phosphotransferase (Arginine Kinase), Triosephosphate isomerase, Fructose bisphosphate aldolase class I, Alpha/beta casein, Lipocalin, Kappa-casein, Transferrin, C-type lysozyme/alpha-lactalbumin family, Serum albumin, Enolase, Serpin serine protease inhibitor, Kazal-type serine protease inhibitor, Collagen, Lipoprotein, Prolamin superfamily, Cupin, Profilin, Bet v 1 family, Oleosin, Isoflavone reductase family, Plant defensin, 60S acidic ribosomal protein, Heat shock protein Hsp70, Fe/Mn superoxide dismutase, Papain-like cysteine protease, Thaumatin-like protein, Unclassified, GILT family, Hevein-like and class I/II chitinase, Zinc finger, C3HC4 type (RING finger), SCAI family, Peroxidase, Serpin serine protease inhibitor, Thionin, Thioredoxin, Transcription elongation factor 1, and Triosephosphate isomerase.

In certain embodiments, contemplated peptides are each derived from a protein listed in Tables 2, 4-7, and 10-12. In certain embodiments, contemplated peptides each comprise, consist essentially of, or consist of the amino acid sequence of any one of SEQ ID NOs: 1-11742. In certain embodiments, contemplated peptides each comprise, consist essentially of, or consist of peptide sequences listed in any one of Tables 13-23.

A contemplated composition may, for example, be formulated for oral, parenteral, nasal, or topical administration.

### BRIEF DESCRIPTION OF THE DRAWINGS

FIG. 1 is a schematic depiction of an exemplary method for identification of cross-reactive allergenic proteins.

FIG. 2 is a schematic depiction of an exemplary allergenic map for a food source (“AllerMap”). All allergenic proteins present in a food source, all protein families to which these allergenic proteins belong, and all cross-reactive allergenic proteins not in the food source may be identified, for example, as described in FIG. 1. Then, all allergenic proteins in the food source and cross-reactive allergenic proteins may be assembled into an allergenic map for that food source. One or more AllerMaps may be used to create an allergenic profile of a subject.

FIG. 3 is a schematic depiction of an exemplary method for identification of cross-reactive epitopes, e.g., MHC-binding T-cell epitopes and B-cell epitopes that are shared by or common to allergenic proteins within a protein family.

FIG. 4 is a 2-dimensional matrix illustrating the homology and cross-reactivity between any two full-length proteins (based on amino acid sequence) within the Cupin protein family. Minimal homology observed was 14%. The size of the dots is indicative of the homology and the cross-reactivity between any protein pair.

FIG. 5 is a 2-dimensional matrix illustrating the homology and cross-reactivity between any two full-length proteins (based on amino acid sequence) within the Tropomyosin protein family. Minimal homology observed was <50%. The size of the dots is indicative of the homology and the cross-reactivity between any protein pair.

FIG. 6 is a 2-dimensional matrix illustrating the homology and cross-reactivity between any two full-length proteins (based on amino acid sequence) within the Bet v 1 protein family. Minimal homology observed was 28%. The size of the dots is indicative of the homology and the cross-reactivity between any protein pair.

FIG. 7 is a 2-dimensional matrix illustrating the homology and cross-reactivity between any two full-length proteins (based on amino acid sequence) within the Profilin protein family. Minimal homology observed was >about 23%. The size of the dots is indicative of the homology and the cross-reactivity between any protein pair.

FIG. 8 is a 2-dimensional matrix illustrating the homology and cross-reactivity between any two full-length proteins (based on amino acid sequence) within the EF Hand protein family. Minimal homology observed was >50%. The size of the dots is indicative of the homology and the cross-reactivity between any protein pair.

FIGS. 9A-9E depict an MHC-II-binding T-cell epitope cluster alignment for each of the indicated allergenic proteins present in the Cupin protein family. The X-axis corresponds to full-length amino acid sequence. Regions to which the MHC-II binding T-cell epitopes align are indicated. Alignment was performed using BlastP algorithm.

FIGS. 10A-10V depict an MHC-II-binding T-cell epitope cluster alignment for each of the indicated allergenic proteins present in the Tropomyosin protein family. The X-axis corresponds to full-length amino acid sequence. Regions to which the MHC-II binding T-cell epitopes align are indicated. Alignment was performed using BlastP algorithm.

FIGS. 11A-11D depict an WIC-II-binding T-cell epitope cluster alignment for each of the indicated allergenic proteins present in the Bet v 1 protein family. The X-axis corresponds to full-length amino acid sequence. Regions to which the MHC-II binding T-cell epitopes align are indicated. Alignment was performed using BlastP algorithm.

FIGS. 12A-12EE depict Circos plots that represent cross-reactive MHC-II-binding T-cell epitopes shared by various proteins within the Cupin protein family. A line originating from one protein radiating to another protein indicates cross-reactive MHC-II-binding T-cell epitopes that are shared by the two proteins. FIG. 12EE depicts a map of cross-reactivity of all Cupin protein family members.

FIG. 13A-1311 depict Circos plots that represent cross-reactive MHC-II-binding T-cell epitopes shared by various proteins within the Tropomyosin protein family. A line originating from one protein radiating to another protein indicates cross-reactive MHC-II-binding T-cell epitopes that are shared by the two proteins.

FIG. 14A-14BB depict Circos Plots that represent cross-reactive MHC-II-binding T-cell epitopes shared by various proteins within the Bet v 1 protein family. A line originating from one protein radiating to another protein indicates cross-reactive MHC-II-binding T-cell epitopes that are shared by the two proteins.

### DETAILED DESCRIPTION

Food allergy diagnosis remains challenging. Most standard methods are unable to differentiate sensitization from clinical allergy and cross-reactivity confounds diagnosis of allergies in many subjects. In many cases clinicians and allergists are unable to make a final diagnosis due to the presence of unidentified cross-reactivity between allergens. Much of the efforts in the prevention and/or treatment of allergies and intolerance to foods have focused on individual allergenic foods (e.g., peanuts) wherein vaccines and diagnostics are directed to allergens in a single food source (e.g., ara h1 or ara h 2 allergenic proteins in peanuts). Response to allergens are also typically focused on IgE antibodies that bind to epitopes on single allergens. The ability to identify cross-reactive allergenic proteins to, and between, allergenic food proteins would greatly aid in the design of various tests and immunotherapeutics for treatment and prevention of allergies and food intolerance in subjects. Identifying cross-reactive epitopes would also aid in the development of diagnostic tests, vaccines, peptide therapies, and specific antibodies which bind to such cross-reactive epitopes.

Accordingly, the disclosure relates, in part, to in silico methods for identification of cross-reactive allergenic proteins. A cross-reactive allergenic protein may be any allergenic protein that has cross-reactivity with at least one other allergenic protein. In the context of the present disclosure, an other allergenic protein is typically a protein in an allergenic source (e.g., an allergenic food source) to which a subject may be allergic and typically belongs to the same protein family as its cross-reactive allergenic counterpart. In some cases, a cross-reactive allergenic protein is an allergenic protein that is cross-reactive with an allergenic protein present in a whole food source that is included in a multiple allergen oral immunotherapeutic composition.

The disclosure also relates, in part, to in silico methods for identification of allergenic cross-reactive epitopes. Cross-reactive epitopes are epitopes that are shared by, or common to, two or more allergenic proteins, one of which may be an allergenic protein present in a food source of interest, for example, a food source present in a multiple allergen oral immunotherapeutic composition. Allergenic cross-reactive epitopes can be mapped to cross-reactive allergenic proteins and allergenic proteins of interest that, for example, are present in a food source or an immunotherapeutic composition. Allergenic cross-reactive epitopes of the present disclosure include T-cell epitopes and other epitopes that bind to at least one Major Histocompatibility Complex (MHC)/Human Leukocyte Antigen (HLA) molecule, or variants thereof. Allergenic cross-reactive epitopes of the present disclosure also include B-cell epitopes. It is contemplated that certain MHC-II-binding T-cell epitopes identified by methods disclosed herein bind a large number of MHC-II molecules which may be advantageous in the development of immunotherapeutics. Allergenic cross-reactive epitopes may, for example, be used in development and preparation of vaccines, peptide therapies, or diagnostics.

The disclosure also relates, in part, to methods of mapping all food allergenic proteins in various protein families to other cross-reactive allergenic proteins to create an allergen map for each food source (an AllerMap). A contemplated AllerMap includes not only the allergenic proteins present in such foods but also all cross-reactive allergenic proteins in other food sources, and other allergenic sources such as inhalants, pollens, spores, etc. It is contemplated that such AllerMaps may, for example, serve as a ready reference for subjects, their families and caregivers, as well as clinicians, allergists, and other health professionals.

The disclosure also relates, in part, to methods, compositions, and kits for identifying, treating, preventing, and/or monitoring food allergies and/or food intolerances in subjects having or at risk of having such food allergies and/or food intolerances. Contemplated methods may, for example, take advantage of cross-reactivity between allergenic proteins in various foods as identified by methods of the present disclosure.

The disclosure also relates, in part, to multiple allergen compositions (for example, multiple allergen immunotherapeutic compositions). A multiple allergen composition may, for example, comprise two or more proteins or two or more whole food sources such that the multiple allergen composition comprises one or more allergenic proteins that contain cross-reactive epitopes shared by or common to one or more cross-reactive allergenic proteins that are present in other food allergens and/or non-food allergens. For example, it is contemplated that parallel tolerization to (i) allergens present in a multiple allergen compositions, and (ii) cross-reactive allergens that are not present in a multiple allergen composition, may be observed upon administration of the multiple allergen composition. A contemplated mixed allergen composition may, for example, increase the resistance of a subject to a cross-reactive allergen, thereby forestalling or reducing the sensitization or allergy development in the subject.

The disclosure also relates, in part, to peptides (for example, T cell epitope peptides) and compositions and kits including such peptides, and their use in the treatment of allergy.

The disclosure also relates, in part, to methods, compositions, and kits for identification and detection of specific binding partners (e.g., antibodies, B-cell receptor expressing cells, and the like) that bind to allergenic proteins, or epitopes within allergenic proteins (for example, epitopes identified by the methods disclosed herein, for example, epitopes that are present on two or more cross-reactive allergenic proteins). Contemplated binding partners may, for example, be useful in the detection or quantification of particular cross-reactive allergens or treatment of allergic disease.

### I. Definitions

As used herein, the terms “a,” “an,” and “the” include plural reference unless the context dictates otherwise.

As used herein, an “antigen” refers any immunogenic moiety or agent (generally a macromolecule) which can elicit an immunological response in an individual mediated by antibodies, specific immunologically-competent cells, or both. The term may be used to refer to an individual macromolecule or to a homogenous or heterogeneous population of antigenic macromolecules. An “allergen” is an antigen which can initiate a state of hypersensitivity, or which can provoke an immediate hypersensitivity reaction in an individual already sensitized to such an allergen. The term “allergenic antigen” may be used interchangeably herein with “antigen” as it pertains to allergens. Allergens are commonly proteins or chemicals bound to proteins which have the property of being allergenic; however, allergens can also include organic or inorganic materials derived from a variety man-made or natural sources such as plant materials, metals, ingredients in cosmetics or detergents, latexes, or the like. Allergens may have little or no intrinsic toxicity by themselves, but cause pathological conditions due to their ability to elicit an IgE-associated immunological response, and upon their subsequent exposure, due to ability to elicit IgE- and/or T-cell-dependent hypersensitivity reactions. As used herein, an allergen may refer to an allergenic food source (for example, a whole or complete food containing allergenic proteins) or may refer directly to an allergenic protein, as will be evident from its context. Exemplary allergens (food source and their allergenic protein) are discussed in further detail in the sections below.

As used herein, “primary allergen” refers to an allergenic source or an allergenic protein in an allergenic source (e.g., an allergenic food source) to which an individual, subject or a patient has an existing allergy.

As used herein, the term “antibody” is meant to refer to immunoglobulin molecules (e.g., any type, including IgG, IgE, IgM, IgD, and IgA, and any class, such as IgG1, IgG2, IgG3, IgG4, IgA1 and IgA2) isolated from nature or prepared by recombinant means or chemically synthesized. The terms “antibody” and “immunoglobulin” can be used interchangeably throughout the specification, unless indicated otherwise. As used herein, “antibody” includes any fragment and analog thereof. A “fragment” or “analog” of an antibody comprises at least the antigen-binding or variable regions of the antibody. Examples of antibody fragments/analogs include Fab, Fab′, F(ab)2, F(ab′)2, F(ab)3, Fv (typically the VL and VH domains of a single arm of an antibody), single-chain Fv (scFv), dsFv, Fd fragments (typically the VH and CH1 domain), and dAb (typically a VH domain) fragments; VH, VL, VhH, and V-NAR domains; minibodies, diabodies, triabodies, tetrabodies, and kappa bodies (see, e.g., Ill et al., Protein Eng 1997; 10: 949-57); camel or llama IgG; IgNAR; and multispecific antibody fragments formed from antibody fragments, and one or more isolated CDRs or a functional paratope, where isolated CDRs or antigen-binding residues or polypeptides can be associated or linked together so as to form a functional antibody fragment. Various types of antibody fragments have been described or reviewed in, e.g., Holliger and Hudson (2005) Nat. Biotechnol. 23:1126-1136; International (PCT) Patent Application Publication No. WO2005/040219; and U.S. Patent Application Publication Nos. 2005/0238646 and 2002/0161201.

As used herein, the term “affinity” refers to a measure of the strength of binding between two members of a binding pair, for example, an antibody and an epitope and an epitope and a MHC-I or II haplotype. Kd is the dissociation constant and has units of molarity. The affinity constant is the inverse of the dissociation constant. Affinity may be determined experimentally, for example by surface plasmon resonance (SPR) using commercially available Biacore SPR units (GE Healthcare) or in silico by methods such as those described herein. Affinity may also be expressed as an IC50 or inhibitory concentration 50, that concentration at which 50% of a ligand (e.g., a peptide) is displaced.

As used herein, the terms “specific binding” or “specifically binding,” for example, when used in reference to the interaction of an antibody and a protein or peptide or the interaction of an epitope and an MEW haplotype means that the interaction is dependent upon the presence of a particular structure (e.g., an antigenic determinant or epitope).

As used herein, the term “epitope” refers to one or several portions or antigenic determinants (which may define a linear or a conformational epitope) of an antigenic protein or peptide which is/are specifically recognized and bound by an antibody, IgE, IgG, IgM, IgA etc., or a portion thereof (Fab′, Fab2′, etc.) or a receptor presented at the cell surface of a B or T cell lymphocyte, and which is able, by such binding, to induce an immune response.

As used herein, the term “B-cell epitope” refers to a peptide sequence that is recognized and bound by a B-cell receptor. A B-cell epitope may be a linear peptide or may comprise several discontinuous sequences which together are folded to form a structural epitope.

As used herein, the term “T-cell epitope” refers to a peptide sequence that is recognized and bound by a major histocompatibility protein molecule or a variant thereof in a configuration recognized by a T-cell receptor. In particular embodiments, a T cell epitope is an epitope recognized by an MHC class II molecule, which includes a sequence of +/−9 amino acids which fits in the groove of the MHC II molecule. Within a peptide sequence representing a T cell epitope, the amino acids in the epitope may be numbered P1 to P9, amino acids N-terminal of the epitope may be numbered P-1, P-2 and so on, and amino acids C terminal of the epitope may be numbered P+1, P+2 and so on.

The terms “allergy”, “allergic diseases”, “allergic disorders”, “food intolerances” “epitope-sensitive disorder, conditions and/or diseases” as used herein refer to diseases characterized by hypersensitivity reactions of the immune system to specific substances called allergens (such as pollen, stings, inhalants, venom, drugs, or foods, for example, whole foods, allergenic proteins, T-cell epitopes, B-cell epitopes, and/or MHC-binding T-cell epitopes present in allergenic proteins). Allergy is the ensemble of signs and symptoms observed whenever atopic individual subjects encounter an allergen to which they have been sensitized, which may result in the development of various diseases and symptoms, in particular respiratory diseases and symptoms such as bronchial asthma, autoimmunity, urticaria, etc. “Hypersensitivity” is an undesirable (damaging, discomfort-producing and sometimes fatal) reaction produced in an individual upon exposure to an antigen to which it has become sensitized; “Immediate hypersensitivity” depends of the production of IgE antibodies and is therefore equivalent to allergy.

As used herein, “immune cell” refers to any immune cell capable of providing an immune response to immunogenic or antigenic stimuli, such as exposure to one or more immunogens or antigens or ensembles of antigens or allergens. Immune cells include, but are not limited to, lymphocytes, T-cells, B-cells, plasma cells, memory cells, dendritic cells, monocytes, macrophages, antigen presenting cells (APCs), neutrophils, basophils, eosinophils, granulocytes, mast cells or any other cells considered to be part of the immune system of an organism.

As used herein, the term “immune response” or “immunological response” includes T cell (cellular) mediated and/or B cell (humoral) mediated immune responses, or both cellular and humoral responses. In particular, the term “immune response” may include an IgE-mediated immune response (e.g., an allergic immune response). Exemplary immune responses include T cell responses, such as Th2 responses resulting in cytokine production and/or cellular cytotoxicity. In addition, the term “immune response” includes responses that are indirectly affected by T cell activation, e.g., antibody production (humoral responses) and activation of cytokine responsive cells, e.g., eosinophils or macrophages. Immune cells involved in immune responses include lymphocytes, such as T cells (CD4+, CD8+, Th1 and Th2 cells, memory T cells, regulatory T cells) and B cells; antigen presenting cells (e.g., professional antigen presenting cells such as dendritic cells, macrophages, B lymphocytes, Langerhans cells, and non-professional antigen presenting cells such as keratinocytes, endothelial cells, astrocytes, fibroblasts, and oligodendrocytes); natural killer (NK) cells; and myeloid cells, such as macrophages, eosinophils, mast cells, basophils, and granulocytes. A particular immune response includes the production of immunoglobulin (Ig) isotype antibodies or decreasing IgE antibodies.

As used herein, the term “major histocompatibility complex (MHC)” refers to MHC Class I (and/or HLA Class I) and MHC Class II (and/or HLA Class II) genes and the proteins encoded thereby. Molecules of the MHC bind small peptides and present them on the surface of cells for recognition by T-cell receptor-bearing T-cells. MHCs are both polygenic (there are several MHC/HLA Class I and MHC/HLA Class II genes) and polymorphic (there are multiple alleles of each gene). The terms MHC-I, MHC-II, MHC-1 and MHC-II are variously used herein to indicate these classes of molecules. Included are both classical and nonclassical MHC molecules. An MHC molecule is made up of multiple chains (alpha and beta chains) which associate to form a molecule. The MHC molecule contains a cleft which forms a binding site for peptides. Peptides bound in the cleft may then be presented to T-cell receptors. The term “MHC binding region” refers to the cleft region of the MHC molecule where peptide binding occurs. As used herein, the term “haplotype” refers to the HLA alleles found on one chromosome and the proteins encoded thereby. Haplotype may also refer to the allele present at any one locus within the MHC. Each class of MHC is represented by several loci: e.g., HLA-A (Human Leukocyte Antigen-A), HLA-B, HLA-C, HLA-E, HLA-F, HLA-G, HLA-H, HLA-J, HLA-K, HLA-L, HLA-P and HLA-V for class I and HLA-DRA, HLA-DRB1-9, HLA-, HLA-DQA1, HLA-DQB1, HLA-DPA1, HLA-DPB1, HLA-DMA, HLA-DMB, HLA-DOA, and HLA-DOB for class II. Mutations and other variations of WIC and HLA molecules are included within the scope of the present disclosure. The terms “HLA allele” and “MHC allele” are used interchangeably herein. HLA alleles are listed on the world wide web at hla.alleles.org/nomenclature/naming.html.

As used herein, the term “biological sample,” or “patient sample” refers to a sample obtained from an organism or components (e.g., cells, or bodily fluids) of an organism. The sample can be of any biological tissue or fluid. The sample may be a non-clinical sample or a clinical sample, which is a sample derived from a patient. Such samples include but are not limited to, whole blood, serum, plasma, sputum, blood cells (e.g., peripheral blood mononuclear cells (PBMC), T-cells, B-cells, basophils, etc.), immune cells, tissue samples, biopsy samples, urine, tears, peritoneal fluid, pleural fluid, breast duct fluid, breast exudate, breast milk, saliva, semen, mucous, lymph, cytosol, ascites, amniotic fluid, bladder washes, bronchioalveolar lavage, or cells therefrom. Patient samples may be fresh or frozen, and may be treated, e.g., with heparin, EDTA, citrate or any other suitable treatment known in the art.

As used herein, “treat”, “treating” and “treatment” mean the treatment of a disease in a subject, e.g., in a human. This includes: (a) inhibiting the disease, i.e., arresting its development; and (b) relieving the disease, i.e., causing regression of the disease state. Contemplated methods may reduce or eliminate allergic symptoms including, for example: eczema, asthma, atopic dermatitis, bronchospasm, cough, rhinorrhea, angioedema, gastric hypermotility, urticaria (hives), pruritis, fatigue, bradycardia, allergic rhinitis, allergic conjunctivitis, and/or hypotension.

As used herein, the terms “subject” and “patient” refer to an organism to be treated by the methods and compositions described herein. Such organisms preferably include, but are not limited to, mammals, e.g., human, a companion animal (e.g., dog, cat, or rabbit), or a livestock animal (for example, cow, sheep, pig, goat, horse, donkey, and mule, buffalo, oxen, or camel)). In certain embodiments, a subject is a pediatric subject (e.g., less than or equal to 18 years old).

Throughout the description, where compositions are described as having, including, or comprising specific components, or where processes and methods are described as having, including, or comprising specific steps, it is contemplated that, additionally, there are compositions of the present disclosure that consist essentially of, or consist of, the recited components, and that there are processes and methods according to the present disclosure that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present disclosure, whether explicit or implicit herein. For example, where reference is made to a particular compound, that compound can be used in various embodiments of compositions of the present disclosure and/or in methods of the present disclosure, unless otherwise understood from the context. In other words, within this application, embodiments have been described and depicted in a way that enables a clear and concise application to be written and drawn, but it is intended and will be appreciated that embodiments may be variously combined or separated without parting from the present teachings and disclosure. For example, it will be appreciated that all features described and depicted herein can be applicable to all aspects of the disclosure described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

It should be understood that the order of steps or order for performing certain actions is immaterial so long as the present disclosure remains operable. Moreover, two or more steps or actions may be conducted simultaneously.

The use of any and all examples, or exemplary language herein, for example, “such as” or “including,” is intended merely to illustrate better the present disclosure and does not pose a limitation on the scope of the disclosure unless claimed. No language in the specification should be construed as indicating any non-claimed element as essential to the practice of the present disclosure.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure. Where the use of the term “about” is before a quantitative value, the present disclosure also includes the specific quantitative value itself, unless specifically stated otherwise.

### II. METHODS OF IDENTIFYING CROSS-REACTIVE ALLERGENIC PROTEINS

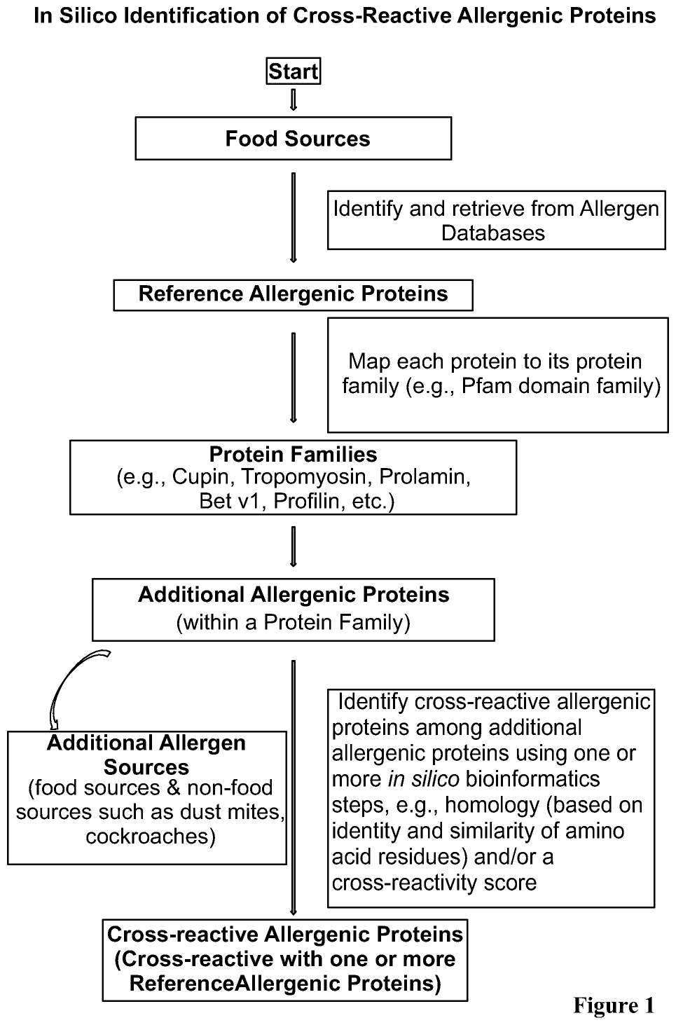

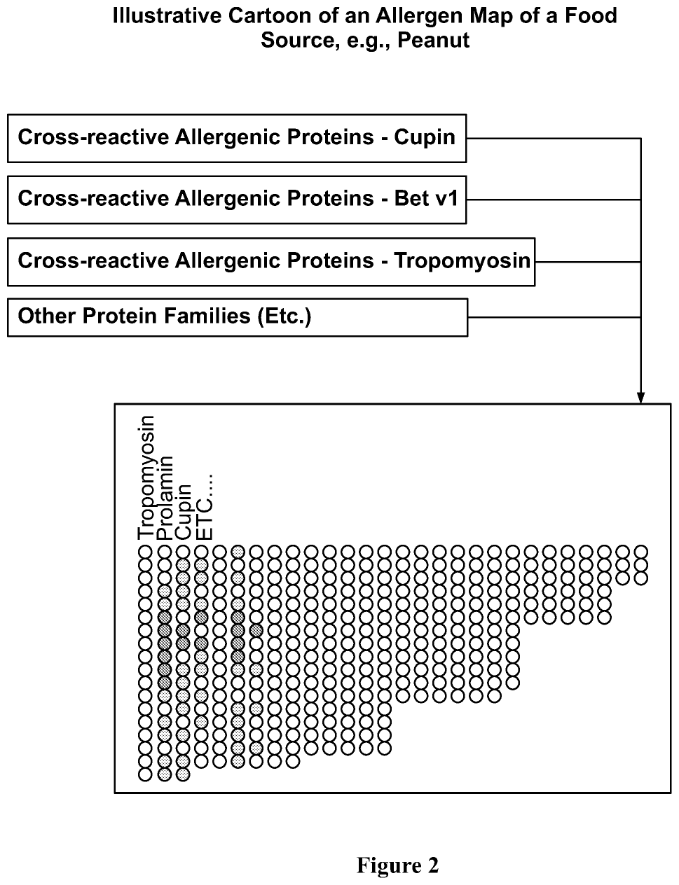

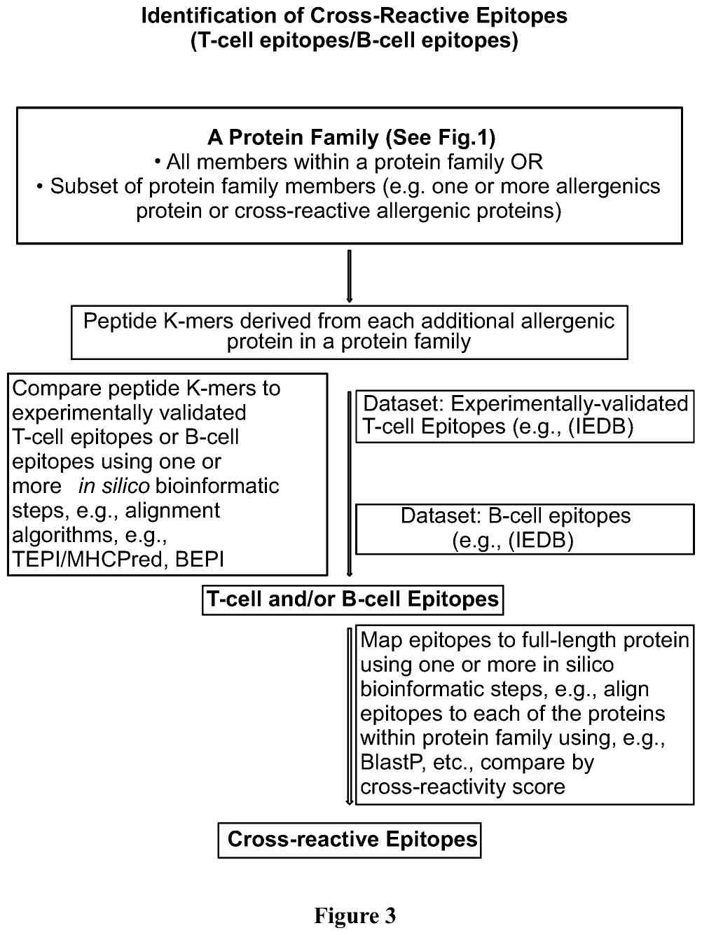

FIG. 1 provides as an illustration of a work flow of an exemplary for identification of cross-reactive allergenic proteins of the present disclosure; FIG. 2 provides an illustrative cartoon of an exemplary allergen map of a food source containing identified allergenic proteins and cross-reactive allergenic proteins in the food source that can be prepared using the methods of the present disclosure; and FIG. 3 provides an illustration of work flow of an exemplary method for identification of cross-reactive epitopes (T-cell and B-cells) which can be mapped to the identified allergenic proteins present and cross-reactive allergenic proteins present in a food source. Thus, a specific allergenic protein can be correlated with other allergenic proteins with a protein family to provide cross-reactive allergenic proteins and cross-reactive epitopes. Further details on the methods for identification of cross-reactive allergenic proteins and MHC-binding T-cell epitopes follow.

### Identified or Reference Allergenic Proteins

Allergens originate from many different sources and can stimulate human immune system to produce immunoglobulin E (IgE) antibodies and/or are responsible for eliciting symptoms of allergy in sensitized individuals. Currently over one thousand such molecules have been identified and registered by the World Health Organization and International Union of Immunological Societies (WHO/IUIS). Allergy databases are useful resources for obtaining information on all known and putative allergenic proteins in food sources and there exist several databases that collect and curate information of all known allergens (e.g., meduniwien.ac.at/allfam, allergenonline.org, and allergen.org available on the world wide web, AllerBase, FAARP 2019, and the like).

One of skill in the art will recognize that a variety of standard methods and software tools are available for manipulation, extraction, querying and analysis of data stored in databases. By using the standardized database designs these tools can be readily used individually or in combinations.

In some embodiments, an allergen database such as the AllFam database, is screened using one or more bioinformatics steps to identify allergenic proteins present in one or more food sources. Each allergenic protein present in a food source can be identified and its protein sequence retrieved on a food source-by-food source basis or in batches or as individual protein sequences. In some embodiments, the allergen databases are screened manually. As another non-limiting example, all food sources listed in Table 1 were identified by screening allergen databases in a batch for information on allergens present in food source and information, including associated GenBank or SWISS-PROT ID identifiers or numbers, retrieved via FTP transfer and stored locally. These identifiers are used to screen the GenBank, SWISS-PROT databases and other protein databases to retrieve all amino acid sequences of the identified allergenic proteins. As a yet another example, FAARP database or Genbank can be screening using one or more bioinformatics tools (e.g., using FASTA algorithm to identify allergenic proteins in the database. Exemplary parameters can be: BLOSUM 50 matrix, gap penalty=12 gap, gap extension penalty=2, Z=2,000 and z=1.). As can be seen from Example 1, identified allergenic proteins of allergenic foods of Table 1 include the exemplary identified allergenic proteins are listed in Table 2. The present disclosure specifically provides that the methods disclosed herein identify allergenic proteins present in food sources provided as identified allergenic proteins in Table 1.

### Identified Protein Families and Additional Allergenic Proteins

The present disclosure further provides, for example, that each of the identified allergenic proteins present in an allergenic food (such as those in Table 1) can be mapped to its protein family. Information on the protein families to which these identified allergenic proteins belong are obtained from the curated and annotated databases provided above and those known and/or published in the art (e.g., Genbank, Swiss-prot, NCBI, AllFam, and the like). Thus, each identified allergenic protein can be mapped to a protein family to which it belongs (each an identified protein family) using one or more in silico bioinformatics methods. Protein families (or superfamilies, as applicable) may be based on Pfam domains of each allergenic protein (also referred to as protein domain family herein) identifying additional allergenic proteins. Amino acid sequences of all identified allergenic protein members of each such identified protein family as well as the additional allergenic proteins present in each of the identified protein family may be, for example, retrieved in a format that is conducive for bioinformatics manipulation and analyses in one or more bioinformatics steps to be performed in the methods disclosed here. For example, the amino acid sequences of all proteins in each of the identified proteins families to which the identified allergenic proteins belong are retrieved in FASTA format. As a non-limiting example, all identified allergenic proteins present in the exemplary food sources listed in Table 1 are provided in Table 2 (see also Example 1). One of skill in the art will appreciate that additional allergenic proteins may be, for example, present in other food sources, and other allergens such as inhalants, pollens, and the like that are not food sources. Thus, the methods of the present disclosure are useful in mapping identified allergenic proteins of Table 2 to additional allergenic proteins present in other non-food allergens and other food sources (See Table 8).

### Identification of Cross-Reactive Allergenic Proteins, in Silico Bioinformatics

To further evaluate the potential of the additional allergenic proteins within each protein family to be cross-reactive with one or more of the identified allergenic proteins also present in each of the identified protein family, each identified allergenic protein may be, for example, mapped to each of the additional allergenic proteins within its identified protein family as described herein. Typically, one or more full-length identified allergenic proteins in foods (such as those listed in Table 2) are mapped to full-length additional allergenic proteins within a protein family (such as those provided in Table 3). One of skill will recognize that isoforms may also be useful in the identification of cross-reactive allergenic proteins and can be included in the analyses.

In some embodiments, contemplated methods include mapping each identified allergenic protein in a family with each of the additional allergenic proteins within the same family. The mapping includes performing one or more in silico bioinformatics steps to determine the homology of one or more full-length identified allergenic protein to each full-length additional allergenic protein within a protein family (full-length homology), determining a cross-reactive score with respect to each additional allergenic protein and correlating one or more of the identified allergenic proteins with one or more of the additional allergenic proteins.

Each additional allergenic protein within a protein family may be, for example, correlated with one or more identified allergenic protein based on its homology and cross-reactivity score. Homology is based on pairwise identity and similarity of a pair of full-length protein sequences (an additional allergenic protein and an identified allergenic protein (“reference protein”). In some embodiments, the full-length homology of each of the allergenic protein sequences within a protein family (which includes one or more identified allergenic proteins within each protein family) with respect to each of the other allergenic proteins within the protein family are determined based on pairwise identity and similarity between the pair of full-length protein sequences. A homology score may be, for example, provided based on identity and similarity of the amino acids between each pair of protein sequences.

Contemplated in silico bioinformatics steps include the use of various bioinformatics tools, methods, algorithms etc. that compare sequences (e.g., pairwise), create multiple sequence alignments, etc. Methods and algorithms for alignment of sequences and for comparison of the sequences are well known in the art. Optimal alignment of sequences for comparison can be conducted, e.g., by the local homology algorithm of Smith and Waterman, Adv. Appl. Math. 2:482 (1981), by the homology alignment algorithm of Needleman and Wunsch, J. Mol. Biol. 48:443 (1970), by the search for similarity method of Pearson and Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al. eds. 1995 supplement)). Default parameters may be used or alternative parameters may be used. Examples of algorithms that are suitable for determining percent sequence identity and sequence similarity are the BLAST and BLAST 2.0, FASTDB, or ALIGN, CLUSTALW, CLUSTAL-OMEGA algorithms, which are publicly available (e.g., NCBI: National Center for Biotechnology Information). Those skilled in the art can determine appropriate parameters for aligning sequences. For example, the BLASTN program (for nucleotide sequences) can use as defaults a wordlength (W) of 11, an expectation (E) of 10, M=5, N=−4, and a comparison of both strands. Comparison of amino acid sequences using BLASTP can use as defaults a wordlength (W) of 3, an expectation (E) of 10, and the BLOSUM62 scoring matrix (see Henikoff and Henikoff, 1989, Proc Natl Acad Sci USA. 89:10915-9). In case of the FASTA algorithm, the intent is to identify local alignments between a pair of sequences to find portions of two proteins that may describe the core areas of shared sequence and this local alignment is consistent with how epitopes tend to localized as small portions (clusters) within the larger intact protein sequence. FASTA as a tool has been used to evaluate sequences and a conservative metric of minimum 35% shared identity, plus a minimum of 80 amino acid overlap length has become the criteria to establish significant shared sequence between an unknown protein allergen and a known allergen. In some embodiments, sequence similarity and Identity are calculated by any method known in the art, for example, FASTA and other tools available at the SIAS server (available on the world wide web at imed.med.ucm.es/Tools/sias.html).

In connection with amino acid sequences, “sequence identity” refers to sequences which have the stated value when assessed using, for example, Clustal-OMEGA, an alignment engine for aligning profile hidden Markov models (HMIs) to each other instead of the conventional dynamic programing and profile alignment. Pairwise alignments may be, for example, performed using the k-tuple method, Sampling method: mBed to speed, Matrix: Gonnet PAM 250 matrix; gap open penalty: 6 bits, gap extension: 1 bit. mmultiple sequence alignment is produced using the HHalign package, which aligns two profile hidden Markov models (HMM). The guide tree may be, for example, next constructed using the UPGMA method (see Daugelaite et al. (2013) International Scholarly Research Notices, vol. 2013, Article ID 615630, and ebi.ac.uk/seqdb/confluence/display/THD/Help+-+Clustal+Omega+FAQ #Help-ClustalOmegaFAQ-top available on the world wide web).

A full-length homology of at least 70% over the full length of the pair of allergenic protein sequences is an exemplary acceptable minimum homology in the context of the present disclosure. In various embodiments, the minimum homology acceptable is at least: 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, or 98% homologous to an identified allergenic protein (a reference protein) present in any of the food sources (such as those listed in Table 1) or another additional allergenic protein (also a reference protein).

In some embodiments, the methods for identification of cross-reactive allergenic proteins include one or more steps to determination a cross-reactivity score for each allergenic protein within a protein family with respect to an identified allergenic protein to predict cross-reactivity between two allergenic proteins (e.g., an additional allergenic protein and an identified allergenic protein, or one additional allergenic protein and another additional allergenic protein) and using a minimal acceptable cross-reactivity score for prediction of cross-reactivity between each pair of allergenic protein sequences.

Cross-reactivity score (an “A-RISC score”) may be calculated for each allergenic protein within a protein family of interest (e.g., Cupin, Tropomyosin, Bet v 1, Prolamin, Profilin, Lipid Transfer Protein, EF Hand, etc.) to predict cross-reactivity of an allergenic protein to another allergenic protein using an Allergens'-Relative Identity, Similarity and Cross-Reactivity (A-RISC) indexing algorithm as described in Chruszcz et al. (2018) PLoS One 13(11):e0208276 and as further described in Example 2. This cross-reactivity scoring method is based on the identity and similarity of each allergenic protein (for example, of amino acid sequence residues) with respect to a reference protein, which in the context of this disclosure is typically an identified allergenic protein or in some cases another additional allergenic protein within the same protein family or another identified allergenic food source (each a “reference protein”). The methods may, for example, include one or more bioinformatics steps to determine the cross-reactivity score for each allergenic protein within a protein family. Pairwise amino acid sequence comparison and/or multiple sequence alignment may be, for example, performed to determine the identity and similarity scores for each amino acid residue in the protein sequence for each allergenic protein as described above. Sequence similarities and identities may be, for example, calculated with SIAS (Immunomedicine Group: Tools >>SIAS (ucm.es)) using default parameters. The SIAS webpage divides the amino acids into the following similarity groups: aromatic, aliphatic, positively charged, negatively charged, small with hydroxyl group and neutral polar. A cross-reactivity scores may be, for example, categorized into 4 levels. A cross-reactivity scores may be, for example, categorized as low (A-RISC=≤0.25), medium-low (A-RISC=≥0.25), medium-high (A-RISC=≥0.5) and high (A-RISC=1) for likelihood of cross-reactivity between each additional allergenic protein and its reference protein (an identified allergenic protein or another additional allergenic protein in the same protein family).

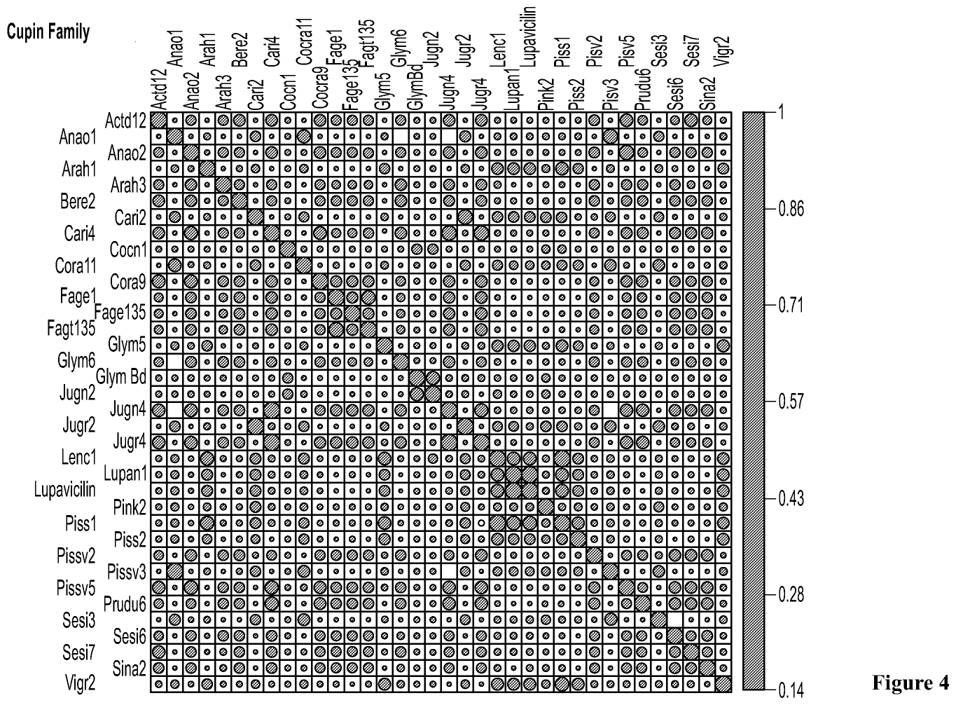

FIGS. 4-7 provide exemplary result for the mapping of each additional allergenic protein in an indicated protein family with respect to other additional allergenic proteins within the same protein family. The 2-dimensional matrix indicates the degree of cross-reactivity between each protein pair within a protein family. Size of the dots in each matrix reflects the predicted cross-reactivity of each pair of protein sequences.

Each allergenic protein having a cross-reactivity score of ≥0.5 to 1 with respect to its reference protein using the methods disclosed herein may be, for example, identified as having cross-reactivity with its reference protein (e.g., an identified allergenic protein or another allergenic protein, as applicable) and may be, for example, identified as a cross-reactive allergenic protein. Accordingly, in some embodiments, the cross-reactive allergenic proteins identified using the methods of the present disclosure have a cross-reactivity score of ≥0.5-1. In some embodiments, the identified cross-reactive allergenic proteins will have a cross-reactivity score of ≥0.5. In some embodiments, the identified cross-reactive allergenic proteins may be stratified by their cross-reactivity score, for example, a cross-reactivity score of ≥0.5, ≥0.6, ≥0.65, ≥0.7, ≥0.75, ≥0.8, ≥0.85, ≥0.9, ≥0.95, ≥0.96, ≥0.97, ≥0.98, ≥0.99 or about 1. Such stratification may be desirable for selection of various identified cross-reactive allergenic proteins, for example, for using in therapeutics, for use in screening of immune response to cross-reactive allergenic proteins, for development of assays and tests for diagnosing, treating and monitoring a subject and/or progression (or remission or tolerization) of their allergies.

Examples of allergenic proteins in Cupin protein family that are cross-reactive with respect to other allergenic proteins within the Cupin protein family are provided in Table 10. Similarly, a non-exhaustive list of allergenic proteins in Tropomyosin and Bet v1 protein families with respect to other allergenic proteins within their respective protein families are provided in Tables 11 and 12. One of skill will appreciate an identified allergenic protein which serves as a reference protein to a cross-reactive allergenic protein is itself a cross-reactive allergenic protein with respect to that cross-reactive allergenic protein. By way of example, Ber e2, a Cupin family additional allergenic protein is cross-reactive to the identified allergenic protein ara h3 and by the same token, ara h3 the identified allergenic protein present in peanuts is cross-reactive to Ber e2 which is the reference protein with respect to ara h3.

Accordingly, the additional allergenic proteins having a cross-reactivity score of ≥0.5 with respect to an identified allergenic protein (e.g., such as those present in Table 2) or another additional allergenic protein within its protein family are identified as cross-reactive allergenic proteins with respect to the identified allergenic protein or the another additional allergenic protein within the same protein family. In certain embodiments, the identified cross-reactive allergenic proteins are at least 70% homologous to or have a cross-reactivity score of ≥0.5 with respect to an identified allergenic protein listed in Table 2. In certain embodiments, the identified cross-reactive allergenic proteins are at least 80% homologous to or have a cross-reactivity score of ≥0.75 with respect to an allergenic protein listed in Table 2.

These identified cross-reactive allergenic proteins with a cross-reactivity score of ≥0.5 and homology of at least 70% may be, for example, co-recognized with an identified allergenic protein and may be, for example, predicted to induce immune response in an individual, a biological sample or a cell. More stringently cutoffs may be used as desired.

Thus, the present disclosure provides that allergenic proteins in various food sources (including those used in multiple allergen oral immunotherapy) may be identified and mapped to additional allergenic proteins in a protein family using in or more in silico bioinformatics steps, which may include, for example, determining the homology of, and cross-reactivity score for, each additional allergenic protein as disclosed herein and correlating it with respect to its reference protein, e.g., an identified allergenic proteins to identify cross-reactive allergenic proteins. Cross-reactive allergenic proteins identified by the methods of the present disclosure may be, for example, further tested in vitro using bioassays described herein.

One of skill in the art will appreciate that the methods disclosed herein are not limited to identification of cross-reactivity to only identified allergenic proteins of Table 2 and that the methods disclosed herein can be used to identify cross-reactive allergenic proteins to allergenic proteins present in any allergenic food source which can serve as reference proteins for identification of cross-reactive allergenic proteins. One of skill in the art will also appreciate that identified cross-reactive allergenic proteins are also present in non-food allergens and thus subjects having an allergy or food-intolerance to a food allergen may be based on cross-reactivity to one or more allergenic proteins in the food may have another allergy which can be to another food that is not listed in Table 1 or it can be to a non-food allergen (such dust mites, cat allergens, and the like) and cross-reactive allergenic proteins contained therein.

Identification of cross-reactive allergenic proteins using the methods disclosed here reveal the potential for individuals consuming food sources that include any one or more identified cross-reactive allergenic proteins to become allergic to such food sources. Thus, one of skill in the art will recognize that the utility of the methods disclosed herein to identify and map additional cross-reactive allergenic proteins. In the context of the present disclosure, a food allergenic protein thus may be, for example, a plant allergenic protein or an animal allergenic protein. For example, the food allergenic protein may be one or more allergenic protein from: a legume (e.g., peanut, soybean, green pea, lentils, mung beans); a tree (e.g., pine, birch, alder, oak, hazel, hornbeam, willow, poplar, plantanus, tilia, olea); a tree nut (e.g., pecan, hazelnut, almond, pistachio, brazil nut, English walnut, European chestnut, cashew); a drupe (e.g., coconut); a cereal (e.g., wheat, corn, barley, oat); a grass (e.g., ryegrass, timothy-grass); a weed (e.g., ragweed, plantoago, nettle, Atemesia, vulgaris, Chenopodium album, sorrel); a fruit (e.g., tomato, apple, banana, apricot, rice, avocado, apricot, plum, cherry, each, pear, strawberry, kiwi, pineapple); a vegetable (e.g., celery, potato; eggplant); an allergen from a seed (e.g., sesame, buckwheat, mustard); a flower (e.g., lupin); an allergen from a fish (e.g., cod, salmon, carp); a crustacean (e.g., shrimp, prawns, crab, lobster, crayfish); a mollusk (e.g., mussels, scallops, oysters, clams, abalone); a cephalopod (e.g., squid, octopus, nautilus); poultry (e.g., chicken, duck), a dairy product (e.g., egg, milk, cheese); etc. Allergenic proteins present in any of these food sources or others known in the art may be obtained and cross-reactive allergenic proteins may be identified using the methods disclosed herein. Further, the methods disclosed herein can be used for identification of cross-reactive allergenic proteins to allergenic proteins from non-food sources, such as inhalants, animals, dust mites, cats etc.

### III. EPITOPES

Adaptive and humoral immune responses to an allergenic antigens are dependent on the binding of T-cell epitopes and/or B-cell epitopes to WIC molecules via presentation of the MHC-bound epitope complexes at the cell surface of antigen presenting cells, and recognition of those complexes by effector or T-reg cells, an interaction that can be evaluated in WIC or HLA binding assays and in in-vitro T-cell assay and/or by B-cells. A number of WIC-binding motif-based tools that permit scanning of protein sequences for potential T-cell epitopes are known in the art. The methods of the present disclosure can be used, for example, to analyze, identify and provide cross-reactive epitopes comprising B-cell epitopes and/or peptides that bind to one or more members of an WIC or HLA superfamily (e.g., T-cell epitopes) from a variety of sources. The identified cross-reactive epitopes may, for example, immune response in a subject, a biological sample or a cell upon exposure to such epitopes or proteins comprising such epitopes.

The present disclosure provides that cross-reactive epitopes may be, for example, shared by or common to one or more identified allergenic proteins in a food source as well as in additional allergenic proteins that are cross-reactive with such one or more identified allergenic proteins. In some embodiments, the cross-reactive epitopes may be conserved in the entire protein family containing allergenic proteins. As used herein, “shared by” or “common to” with respect to cross-reactive epitopes refers to epitopes peptides sequences that are either identical between the one or more identified allergenic proteins and one or more of cross-reactive allergenic proteins. are nearly identical (e.g., conserved), or have sufficient homology as to permit at least weak binding or co-recognition of the one or more identified allergenic proteins and one or more of cross-reactive allergenic proteins.

The present disclosure is not limited to use of full-length sequences of allergenic proteins; shorter isoforms of such allergenic proteins and partial sequences of allergenic proteins are also useful for the identification of cross-reactive epitopes, e.g., 30%, 40%, 50%, 60%. 70%, 80%, 90% or 95% of the full-length sequences. Indeed, the methods of the present disclosure may be, for example, applied to sequences in an entire protein family containing one or more allergenic proteins or the methods may, for example, be applied to certain subset of allergenic proteins, which include one or more identified allergenic proteins present in a food source and one or more additional allergenic protein or one or more cross-reactive allergenic proteins identified by the methods of the present disclosure.

Cross-reactivity can be between allergenic proteins or epitopes present in one or more food sources or cross-reactivity between one or more allergenic proteins or epitopes present in a food source and one or more cross-reactive allergenic proteins or epitopes present in a non-food source. Non-limiting examples of sources of allergenic protein sequences that can be a source for identification of cross-reactive epitopes are provided in Table 1 (food sources) and Table 8.

In some embodiments, the source of cross-reactive epitopes is one or more of identified protein families (for example, to which one or more identified allergenic proteins belong). Exemplary identified protein families include: Tropomyosin, EF hand family (Beta-parvalbumin), ATP:guanido phosphotransferase (Arginine Kinase), Triosephosphate isomerase, Fructose bisphosphate aldolase class I, Alpha/beta casein, Lipocalin, Kappa-casein, Transferrin, C-type lysozyme/alpha-lactalbumin family, Serum albumin, Enolase, Serpin serine protease inhibitor, Kazal-type serine protease inhibitor, Collagen, Lipoprotein, Prolamin superfamily, Cupin, Profilin, Bet v 1 family, Oleosin, Isoflavone reductase family, Plant defensin, 60S acidic ribosomal protein, Heat shock protein Hsp70, Fe/Mn superoxide dismutase, Papain-like cysteine protease, Thaumatin-like protein, Unclassified, GILT family, Hevein-like and class I/II chitinase, Zinc finger, C3HC4 type (RING finger), SCAI family, Peroxidase, Serpin serine protease inhibitor, Thionin, Thioredoxin, Transcription elongation factor 1, Lipid transfer protein, and Triosephosphate isomerase protein families. Exemplary identified allergenic proteins include the allergenic proteins listed in Table 2. In some embodiments, the source of cross-reactive epitopes is one or more cross-reactive allergenic proteins selected from those listed in Table 4. In some embodiments, the source of cross-reactive epitopes is one or more of allergenic proteins present in the Cupin protein family (such as those listed in Table 5). In some embodiments, the source of cross-reactive epitopes is one or more cross-reactive allergenic proteins within the Cupin protein family. In some embodiments, the source of MHC-binding T-cell epitopes and B-cell epitopes are Ber_e_2, Car_i_2, Fag_e_1, Fag_e_13S, Fag_t_13S_globulin, Jug_n_4, Lup_a_vicilin, Lup_an_1, Pin_k_2, Pis_s_1, Pis_s_2, Pis_v_2, Ses_i_3, Ses_i_6, Ses_i_7, Sin_a_2, Vig_r_2, Car_i_4, Gly_m_5, Gly_m_6, Jug_r_4, Len_c_1, and/or Pru_du_6.

### Cross-Reactive Epitopes

The Immune Epitope Database (Zhang et al. 2008, Nucleic Acids Res. 36:W513-W518) provides a registry of all currently known epitope sequences. Several T-cell epitope prediction tools for the identification and selection of T-cell epitopes from such allergenic and cross-reactive allergenic proteins for use in the context of the present disclosure are known in the art (De Groot and Moise. Current Opinion in Drug Discovery & Development 2007, 10(3)) and a majority of allergenic proteins have been cloned and sequenced (see allergen.org available on the world wide web, a register of validated data maintained by the Allergen Nomenclature Sub-committee of the World Health Organization and International Union of Immunological Societies (TUTS)). Validated T-cell epitope mapping tools include EpiMatrix (EpiVax Inc.), SYFPEITHI (Univ. of Tubingen; syfpeithi.de/home.htm available on the world wide web), MHC Thread (Hebrew University), MHCPhred (Edward Jenner Inst. For Vaccine Research, jenner.ac.uk/MHCPred available on the world wide web), EpiJen (Edward Jenner Inst. For Vaccine Research), NetMHC (Inst. Of Medical Microbiology And Immunology), NetCTL (Inst. Of Medical Microbiology and Immunology), MHC-I/MHC-II binding (IEDB), nHLAPred (Inst. Of Microbial Technology), SVMHC (Univ of Tubingen, bs.informatik.uni-tuebingen.de/SVMHC available on the world wide web), and Bimas (NIH). These algorithms allow prediction within a protein of one or more nonapeptide sequences which will fit into the groove of an MHC II molecule.

IEDB, AntiJen, and B-cell oriented databases such as BciPep, Epitome, and SDAP are known B-cell epitope databases useful in the context of the present disclosure. Various B-cell epitope mapping tools for the prediction of linear B-cell epitopes from protein sequence including amino acid scales and HMIs, DiscoTope, ElliPro, Paratome, and PIGS are available from IEDB. Other useful B-cell epitope mapping tools are known in the art (see Potocnakova et al. (2016) J. Immunol Res. Volume 2016, Article ID 6760830).

Accordingly, the present disclosure further provides in silico methods for identifying one or more epitopes that are cross-reactive, e.g., are shared by or common to one or more identified allergenic proteins and one or more cross-reactive allergenic proteins (or shared by or common to at least two proteins within a protein family).

The in silico bioinformatic methods of the present disclosure may include, for example, performing one or more bioinformatics steps to map or link the experimentally-validated T-cell epitopes and/or B-cell epitopes to all the proteins present within an identified protein family (which includes one or more identified allergenic proteins and all additional allergenic proteins within the protein family) in an unsupervised manner to eliminate selection bias at the protein family and epitope level.

In some cases, it may be desirable to identify T-cell and/or B-cell epitopes only on one or more cross-reactive allergenic proteins (e.g., additional allergenic proteins that are cross-reactive (having a cross-reactivity score of ≥0.5, and/or having a homology score of ≥70%) with one or more allergenic proteins (e.g., an identified allergenic protein from a food source) within a protein family). Accordingly, in some embodiments, the in silico methods of the present disclosure include performing one or more bioinformatics steps to map or link the experimentally-validated T-cell epitopes and/or B-cell epitopes to one or more (or all) identified cross-reactive allergenic proteins in allergenic foods within each identified protein family (such as those in Tables 4-7 (Tropomyosin, Prolamin, EF Hand, Profilin, etc.) to which one or more identified allergenic proteins belong.

To identify T-cell and/or B-cell epitopes in these cases, in some embodiments, the full-length proteins within each identified protein family e.g., Cupin protein family, that includes all allergenic proteins, or a subset of allergenic proteins within a protein family as described above may be chosen as a dataset. Typically, however the entire dataset containing all protein family members are analyzed unsupervised to avoid bias. Experimentally validated T-cell epitopes, and/or B-cell epitopes from a source of experimentally-validated epitopes (such as IEDB) are retrieved and stored locally. A plurality of T-cell epitopes and/or a plurality of B-cell epitopes are probed with a plurality of peptides generated from the allergenic proteins using one or more bioinformatics steps that would include use of bioinformatics tools, algorithms etc.

Contemplated methods may, for example, include one or more in silico bioinformatics steps, wherein peptides (K-mers) are generated from each allergenic protein within a protein family (e.g., Cupin, Tropomyosin, Prolamin, Lipid transfer protein, Bet v 1, EF hand, and the like, which include one or more identified allergenic protein sequences listed in Table 2). Algorithms for generating overlapping peptides are known in the art (e.g., PepSequencer from JPT Innovative Tools). Peptides (K-mers), in the context of the present disclosure, are generally 5-200 amino acids long (5 mer-200-mer) with a sliding window of 1-5 amino acids. In some embodiments, peptide K-mers are 8-150 amino acids long (8 mer-150-mer) with a sliding window of 1-5 amino acids. In some embodiments, the peptides are overlapping peptides of length ranging from 9-mer to 200-mer with a sliding window of 1-5 amino acids. In other embodiments, the peptide K-mers are 20-mers with a sliding window of 1-5 amino acid. In at least one embodiment, the peptide K-mer is a 14 mer with a sliding window of 1-5 amino acids. In at least one embodiment, the peptide K-mer is a 15 mer with a sliding window of 1-5 amino acids (See Example 4).

The peptide K-mers derived from an identified protein family (or in some cases, the identified cross-reactive allergenic proteins and one or more identified allergenic proteins) may, for example, be used to probe the experimentally-validated T-cell epitopes and/or B-cell epitopes (typically contained in separate datasets) to identify linear T-cell epitopes and/or linear B-cell epitopes, and the resulting “hits” may be, for example, collected for further analysis and/or use (e.g., for testing for MHC-binding (in silico and/or in vitro), testing for induction of immune response (e.g., induction of cross-reactive IgE, IgG, cytokine release, activation of immune cells, degranulation, etc.) using various cell biology techniques and biochemical assays or immunoassays, development of peptide vaccines, assaying a blood sample or immune cells for presence of cross-reactive antibodies such as IgE, IgG, IgM, etc., MHC-II-binding assays etc. In some embodiments, peptides are used to probe T-cell epitopes. In other embodiments, peptides are used to probe B-cell epitopes.

Example 4 further describes methods for determining or mapping experimentally-validated T-cell epitopes on all the allergenic proteins present in the exemplary Cupin protein family using the methods of the present disclosure and providing cross-reactive T-cell epitopes that are shared by or common to two or more proteins within the identified Cupin protein family (containing one or more identified allergenic proteins and additional allergenic proteins).

### Identification of Cross-Reactive Peptides Comprising B-Cell Epitopes

As described above for T-cell epitopes, provided herein are, for example, methods for identifying B-cell epitopes on cross-reactive allergenic proteins. In some embodiments, the B-cell epitopes are present on only a single cross-reactive allergenic protein within a protein family. In some embodiments, the B-cell epitopes are present on two or more cross-reactive allergenic proteins, typically within the same protein family (cross-reactive B-cell epitopes). The peptide K-mers of each allergenic protein within a protein family may be, for example, screened against the set of experimentally-validated B-cell epitopes by applying an alignment algorithm, such as BLAST, BLATP, TBLASTN, FASTA and others known in the art using the B-cell epitope prediction tools described above. Peptides sequences having at least 70% identity over a 70% of the peptide sequence may be, for example, collected and stored in an in silico library. In some embodiments peptide sequences having at least 80% identity over 70% of a peptide sequence are collected and stored in an in silico library. Cross-reactivity scores may be, for example, calculated as described below (such as that used for MHC-II binding T-cell epitopes below and in Example 5) to identify cross-reactive B-cell epitopes and locations of each residue can be identified by aligning the identified B-cell epitopes along the full length of each allergenic protein within the protein family.

Example 6 provides a non-limiting exemplary embodiment of an in silico method for determining or mapping experimentally-validated B-cell epitopes on all the proteins present in the Cupin protein family and provides exemplary cross-reactive B-cell epitopes that are shared by or common to proteins within the identified Cupin protein family (containing one or more identified allergenic proteins and additional allergenic proteins). Thus, in another aspect, the present disclosure provides in silico methods for identification of cross-reactive B-cell epitopes. Exemplary cross-reactive epitopes present shared by or common to Cupin protein include those listed in Tables 13-23. One of skill in the art will appreciate that the B-cell epitopes on any cross-reactive allergenic protein that is not shared by or common to two or more allergenic proteins in a protein family are non-cross-reactive B-cell epitopes and may be used for identification of specific allergenic proteins or peptides thereof.

Cross-reactive T-cell epitopes and B-cell epitopes described herein include, for example, those for Cupin family proteins: Ber_e_2, Car_i_2, Fag_e_1, Fag_e_13S, Fag_t_13S_globulin, Jug_n_4, Lup_a_vicilin, Lup_an_1, Pin_k_2, Pis_s_1, Pis_s_2, Pis_v_2, Ses_i_3, Ses_i_6, Ses_i_7, Sin_a_2, Vig_r_2, Cor_a_11, Pis_v_3, and Pis_v_5.

### Identification of MHC-II Binding Epitopes

The methods of the present disclosure further include, for example, performing one or more bioinformatics steps to assess the ability or the binding potential of identified T-cell epitope peptides and identified B-cell epitope peptides to bind MHC-II molecules.

In some embodiments, identified T-cell epitopes and B-cell epitopes are further tested for their binding affinity to MHC or HLA class II molecules. This can be performed in a number of different ways. For example, the identified T-cell epitopes above (either predicted or in vitro-validated) can be tested for MHC or HLA binding using one or more bioinformatics steps. As stated above, several MHC-II-binding prediction tools are known in the art. For instance, the Immune Epitope Database (IEDB) at available on the world wide web at IEDB.org hosts a collection of experimentally validated MHC-II binding epitopes. T-cell epitopes (predicted or selected based on stimulation index from in vitro T-cell proliferation studies) and B-cell epitopes may be, for example, probed using IEDB's MHC-II binding tool against the IEDB dataset containing MHC-II binding T-cell epitopes and/or identified B-cell epitopes using one or more bioinformatics steps. Additional bioinformatic tools include NN_MLP (PrintC-MMC_II-NN), NetMHC II (version 2.0), NetMHC II Pan (version 1.0) accessible from internet servers (see, e.g., cbs.dtu.dk/services/NetMHC available on the world wide web).

In some embodiments, alignment algorithms such as BLASTP or FASTA may be used for screening against the IEDB MHC-II binding epitopes. An experimentally-validated MEW (HLA) epitope having a minimal acceptable homology based on pairwise identity of ≥70% over 80% of the peptide may be, for example, a “hit” or a “match” in the context of the present disclosure.

Contemplated epitopes may be selected, for example, on the basis of a binding affinity of ≤500 nM Kd to ≤1 nM Kd. In some embodiments, IEDB MHC-II binding epitopes are screened with binding affinity parameter set at ≤500 nM Kd. In some embodiments, IEDB MHC-II binding epitopes can be screening with high binding affinity from Kd≤1 nM Kd, ≤5 nM Kd, ≤10 nM, ≤20 nM Kd, ≤25 nM Kd, ≤50 nM Kd, ≤75 nM Kd, ≤100 nM Kd, ≤150 nM Kd, ≤200 nM Kd, ≤250 nM Kd, ≤300 nM Kd, ≤350 nM Kd, ≤400 nM Kd, ≤450 nM Kd, or ≤500 nM Kd. In some embodiments, IEDB MHC-II binding epitopes are screened with binding affinity parameter set at ≤200 nM Kd to screen for medium to high affinity binders. In some embodiments, IEDB WIC-II binding epitopes are screened with binding affinity parameter set at ≤100 nM Kd to select for medium-high to high affinity binders. In some embodiments, IEDB WIC-II binding epitopes are screened with binding affinity parameter set at ≤50 nM Kd to select for high affinity binders. Accordingly, the resulting “hit” peptides comprising WIC class II binding T-cell epitopes may, for example, have a binding affinity ranging from ≤500 nM Kd to ≤1 nM Kd. In some embodiments, the resulting hit peptides comprising T-cell epitopes have a binding affinity of ≤500 nM Kd. In some embodiments, the resulting hit peptides comprising T-cell epitopes have a binding affinity of ≤100 nM Kd. In some embodiments, the resulting hit peptides comprising T-cell epitopes have a binding affinity of ≤50 nM Kd.

Input peptide datasets for screening for WIC-II binding epitopes can be, for example (a) T-cell epitopes predicted from use of T-cell epitope prediction tools such as Tepitool (TEPI), or (b) B-cell epitopes predicted from use of B-cell epitope prediction tools, available at IEDB.org performed using overlapping linear K-mer peptides derived from all proteins in a protein family generated as described above in an unsupervised manner to eliminate the potential for bias in selection of WIC-II-binding epitopes peptide. Resulting WIC-II-binding T-cell epitopes and/or WIC-binding B-cell epitopes hits may be, for example, evaluated for coverage and homology. Allergenic peptides comprising WIC-II-binding T-cell epitope peptides and WIC-binding B-cell epitopes may, for example, have homology to the MHC-II-binding T-cell epitopes or MHC-II-binding B-cell epitopes of IEDB.org of at least 70% identity with 80% coverage of the allergenic peptides in the context of the present disclosure. In some embodiments, homology of at least 80% with 70% coverage of the allergenic peptides is selected.

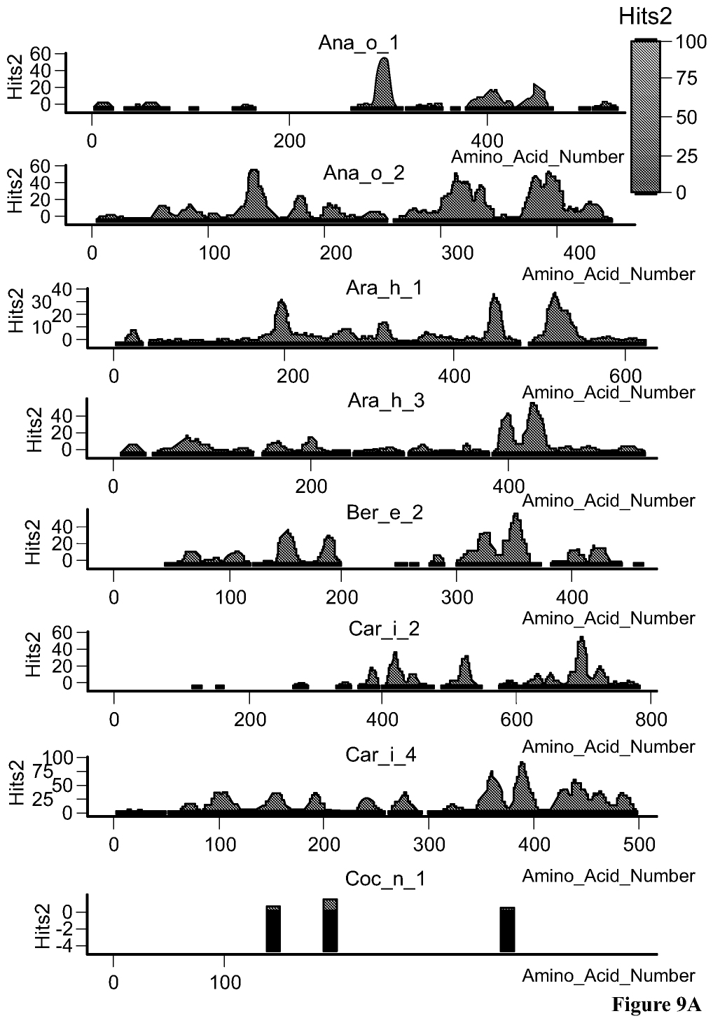

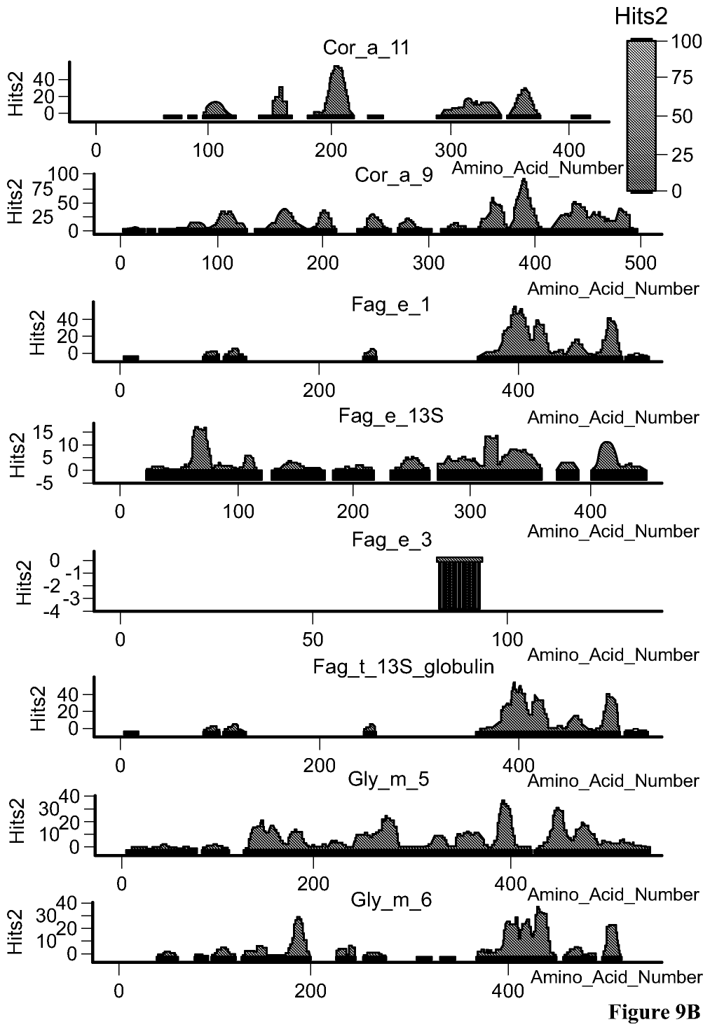

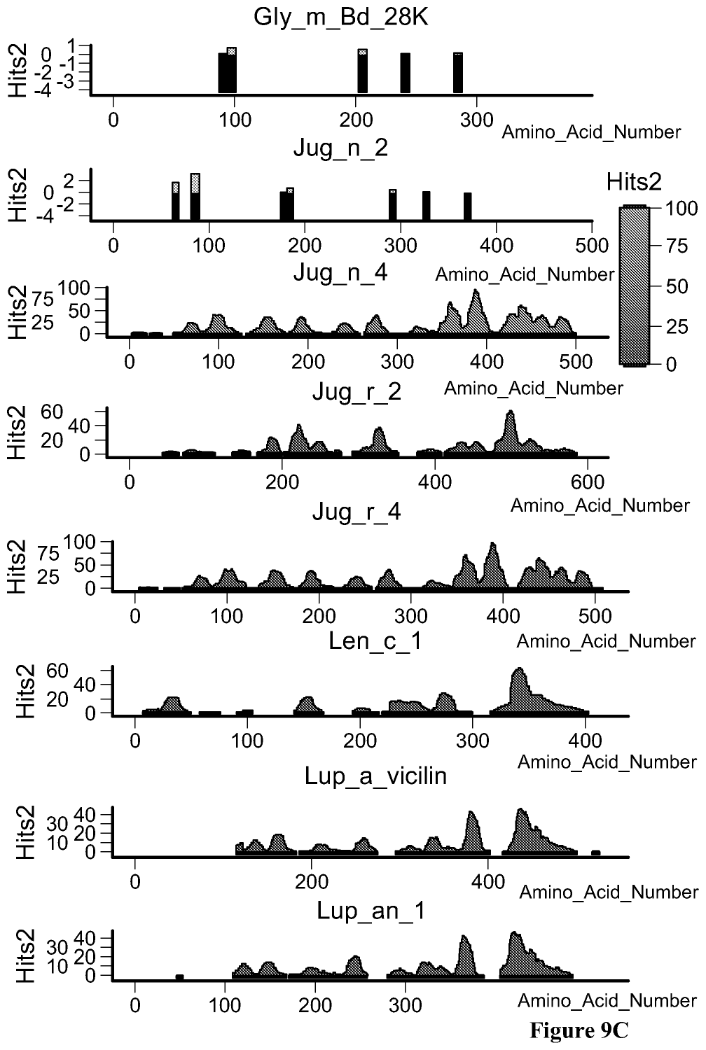

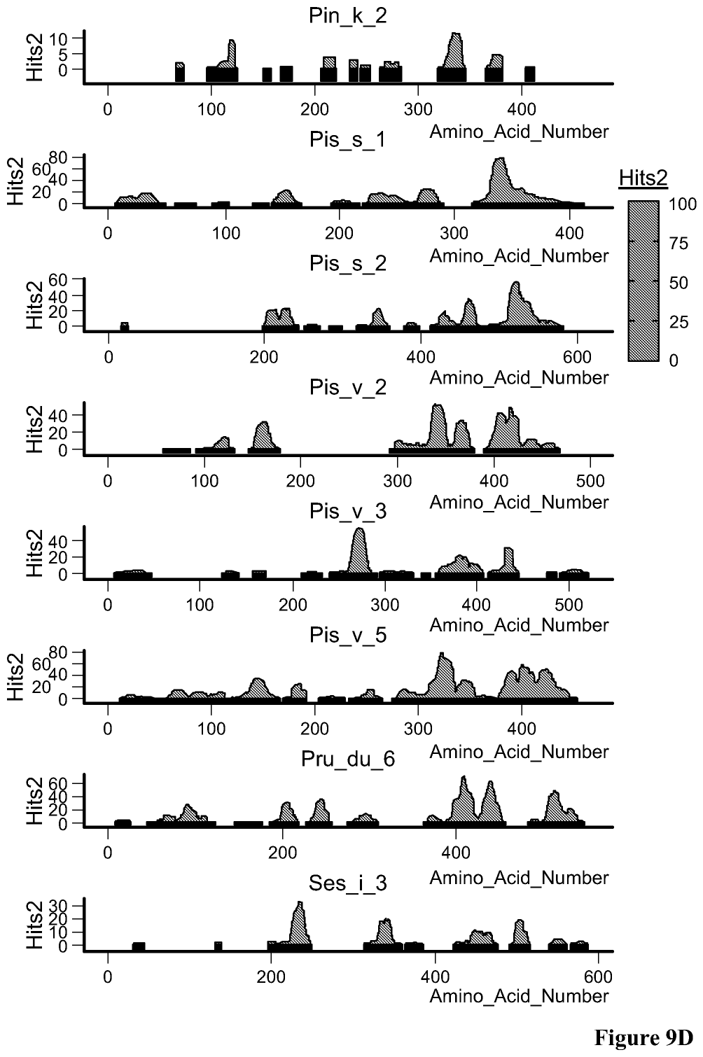

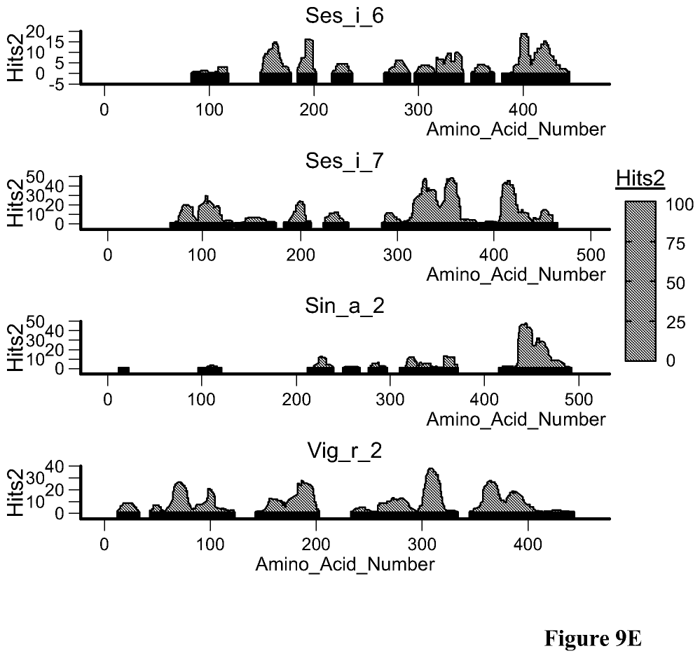

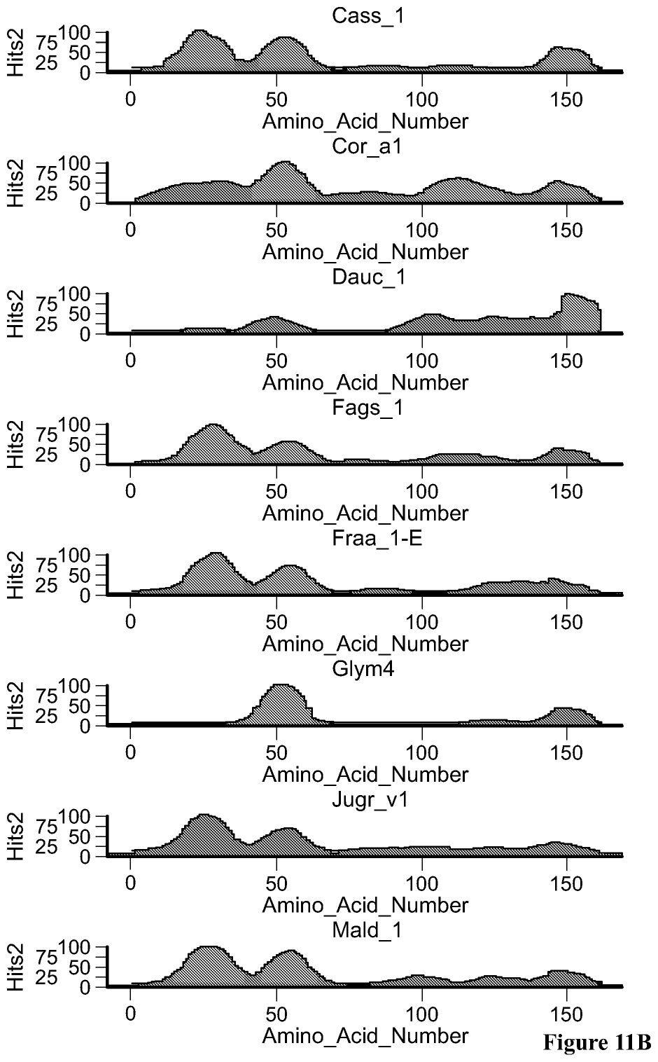

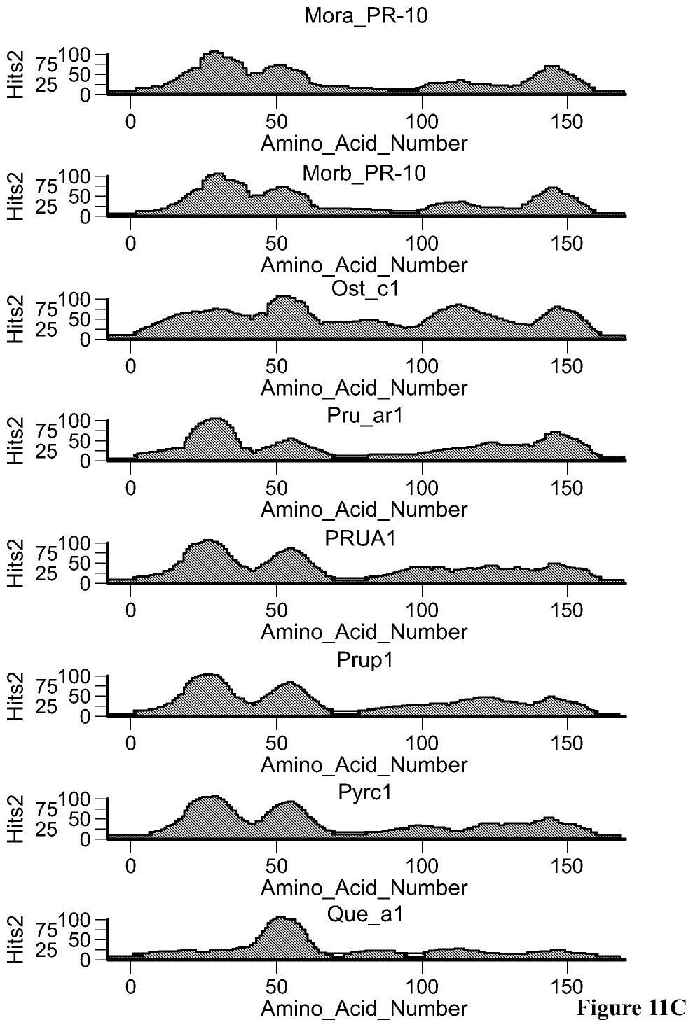

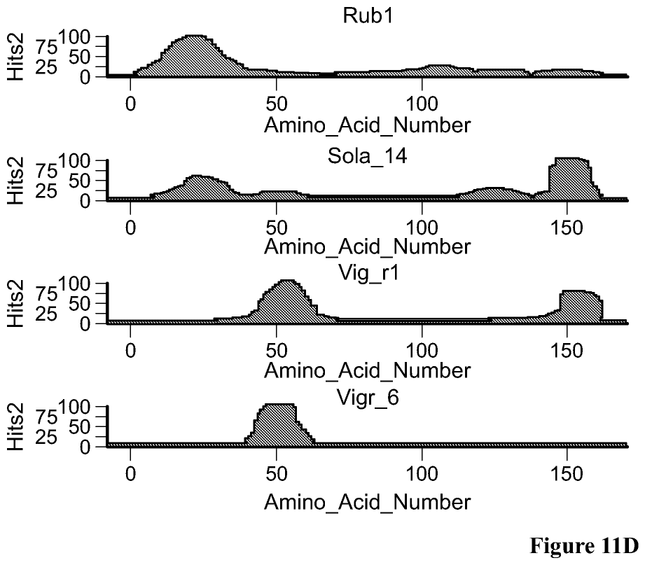

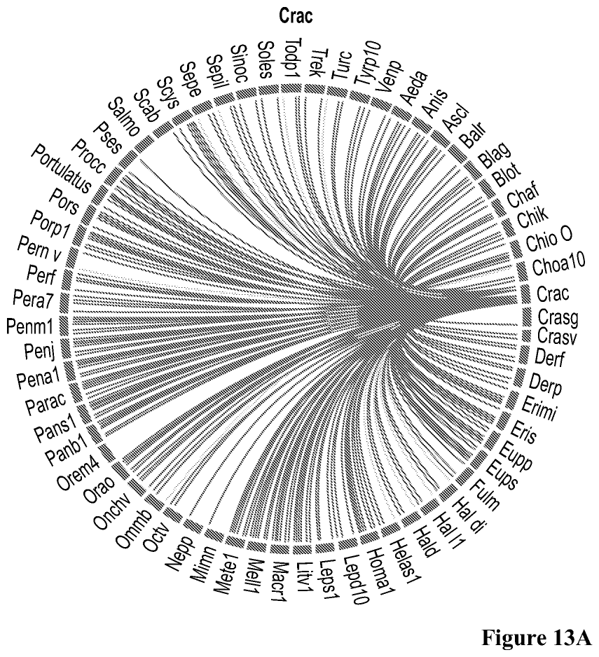

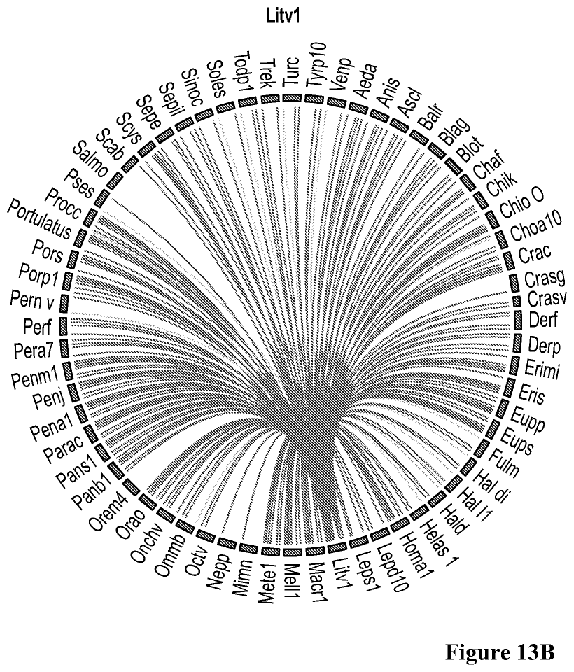

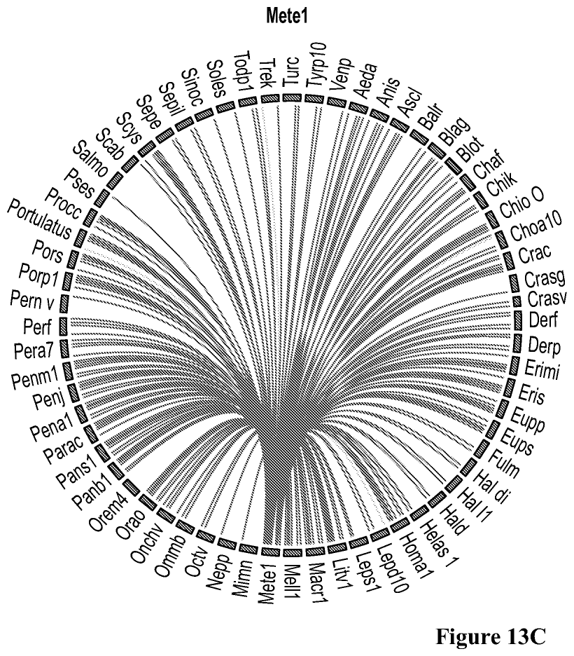

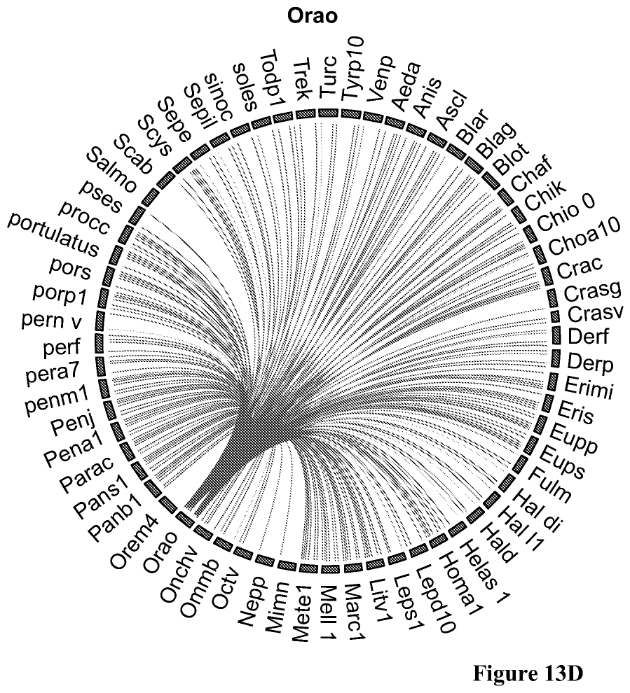

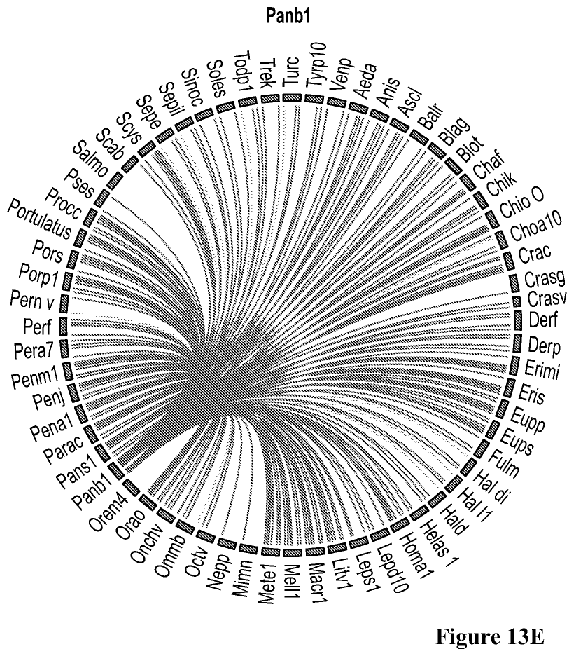

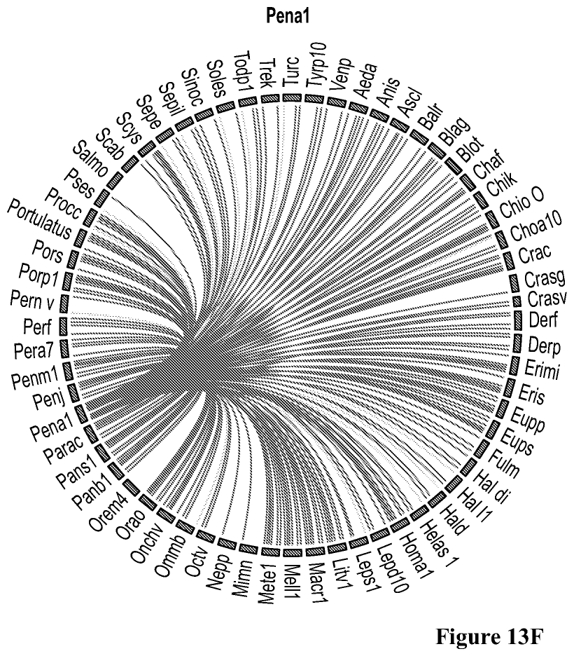

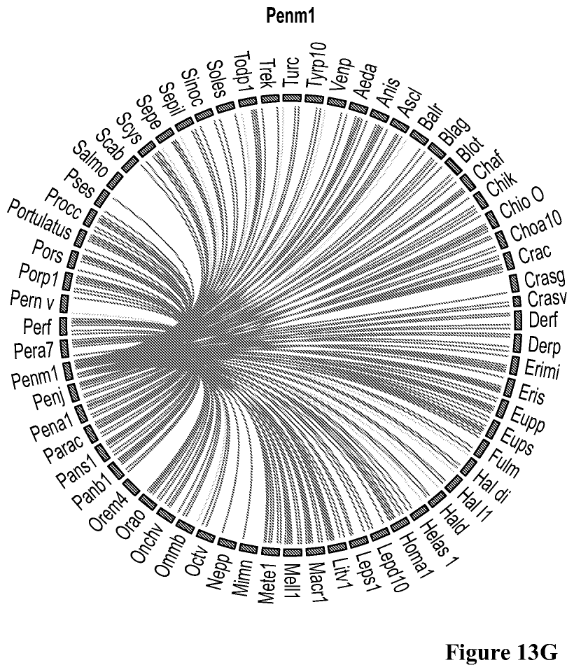

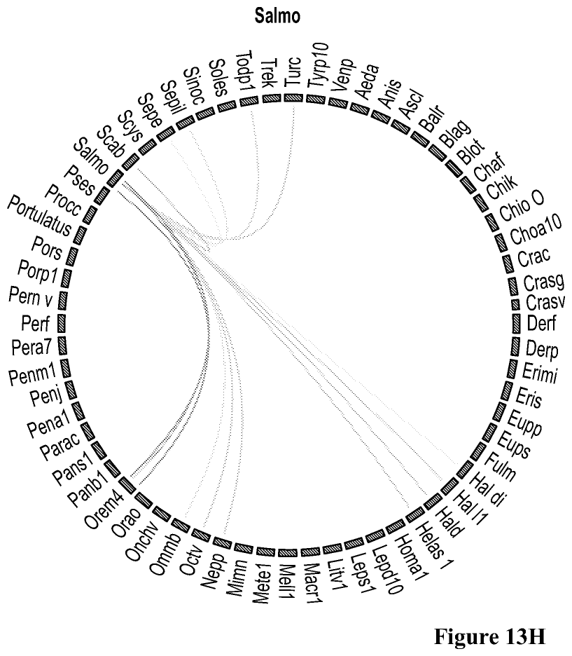

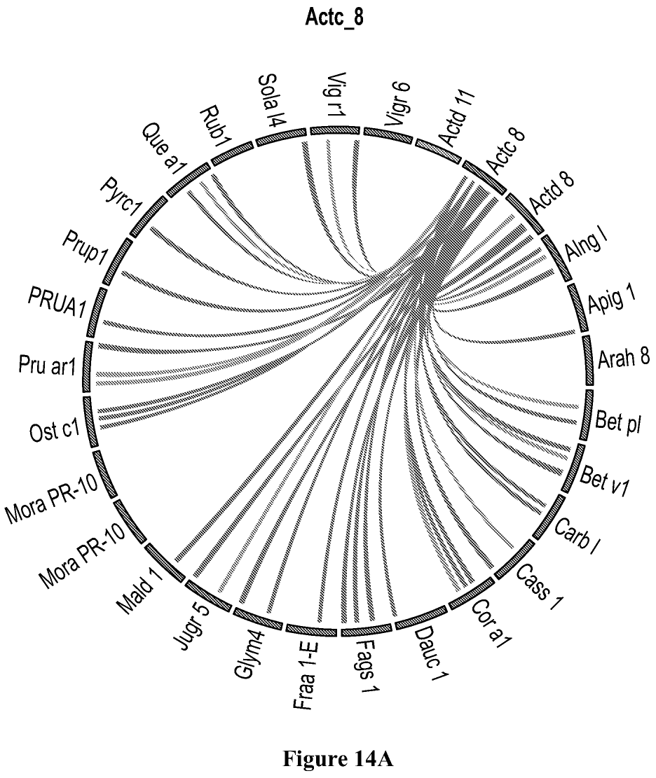

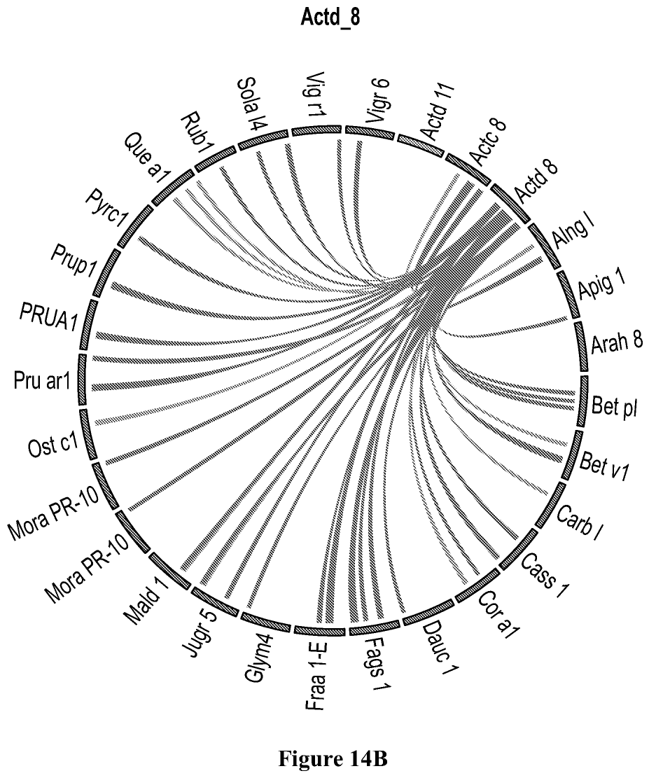

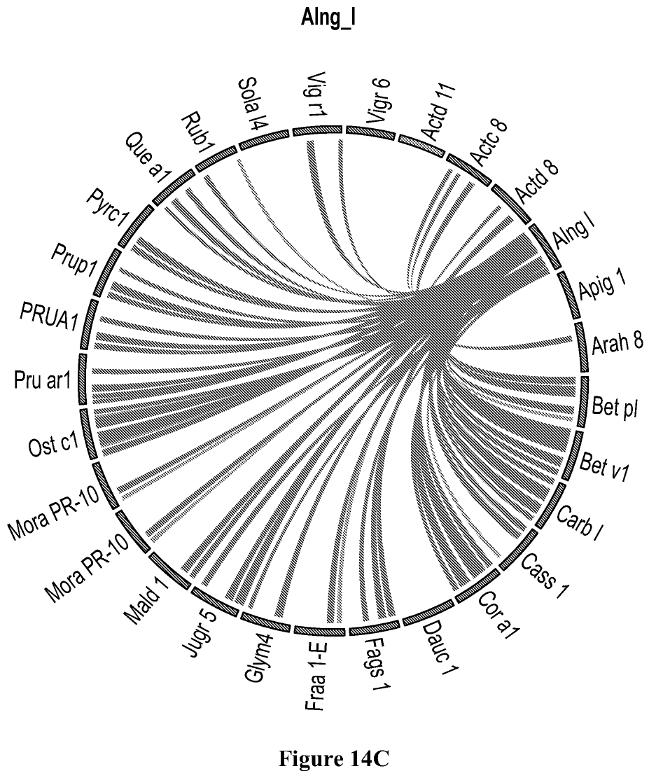

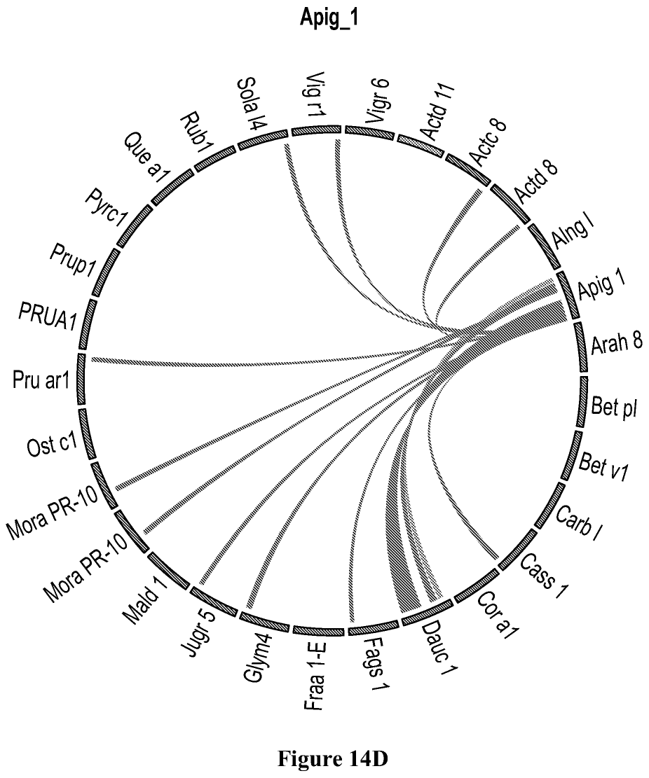

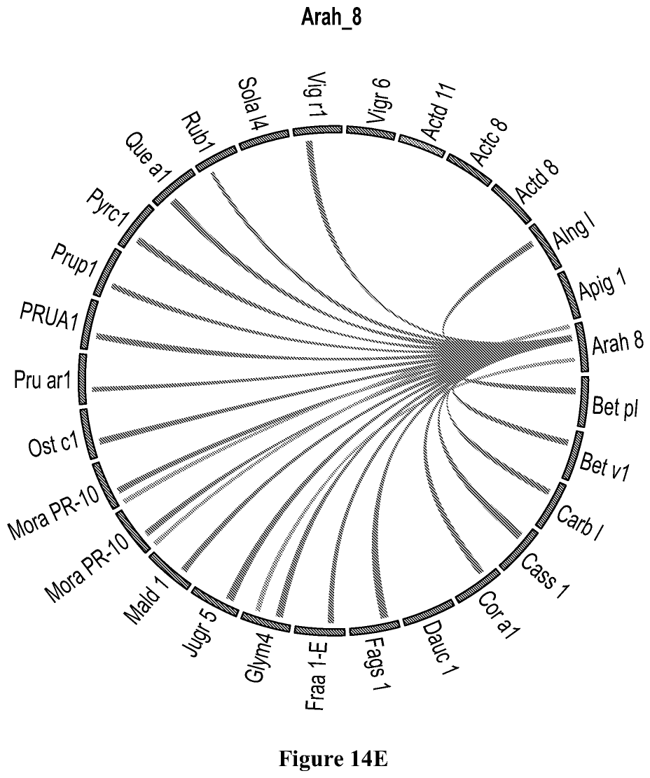

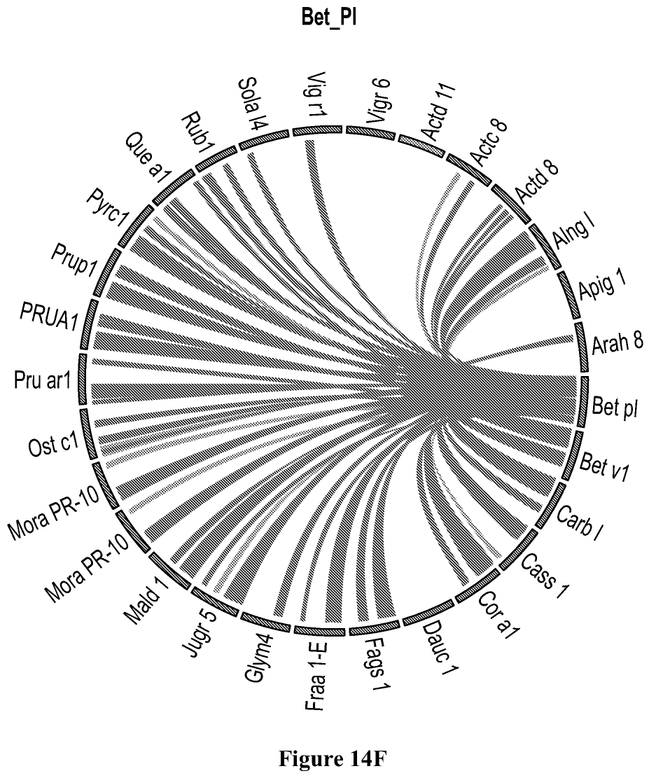

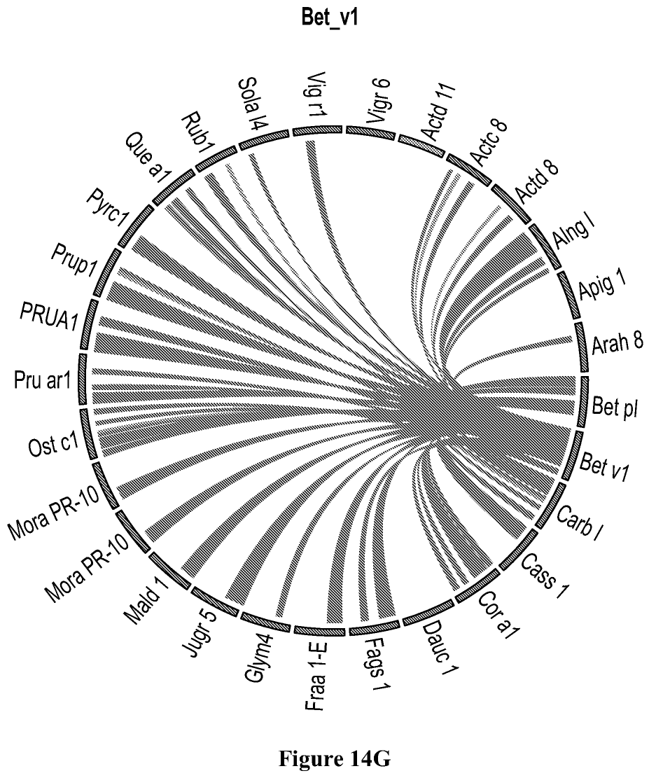

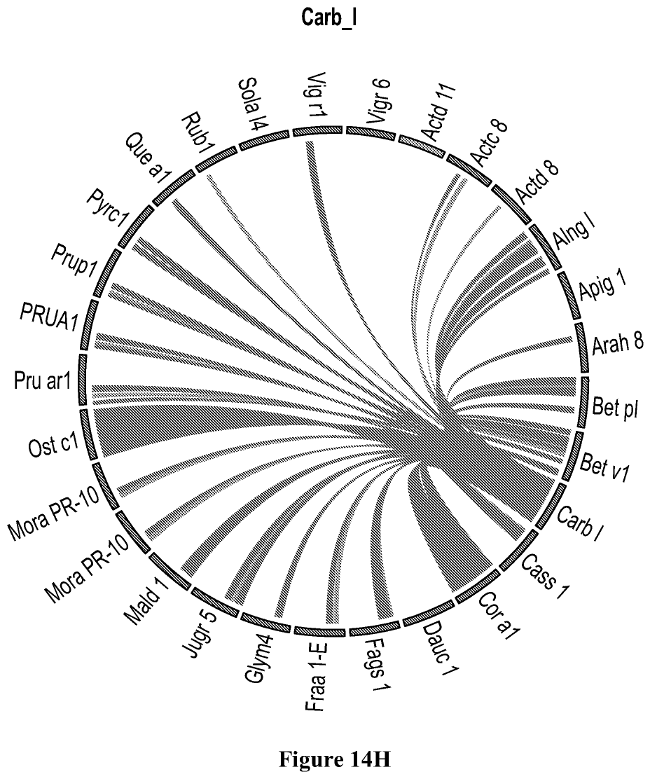

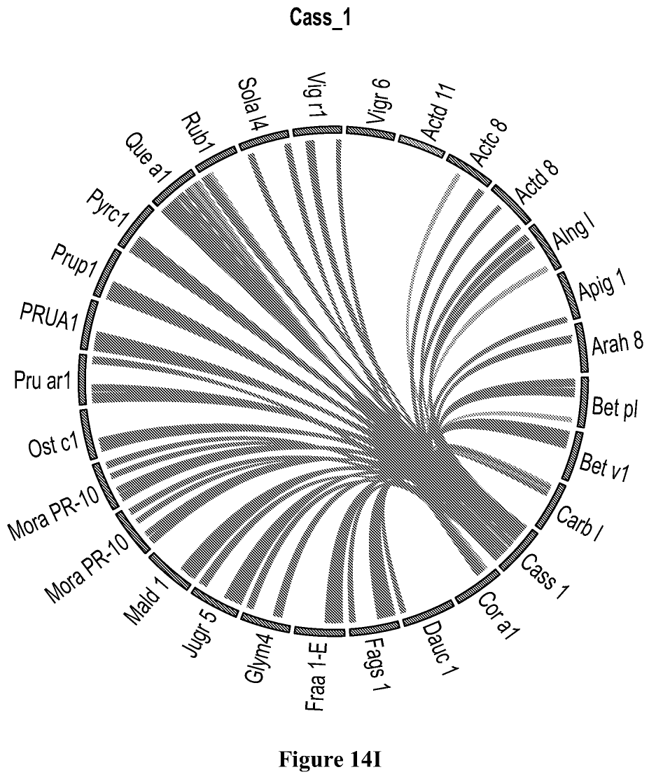

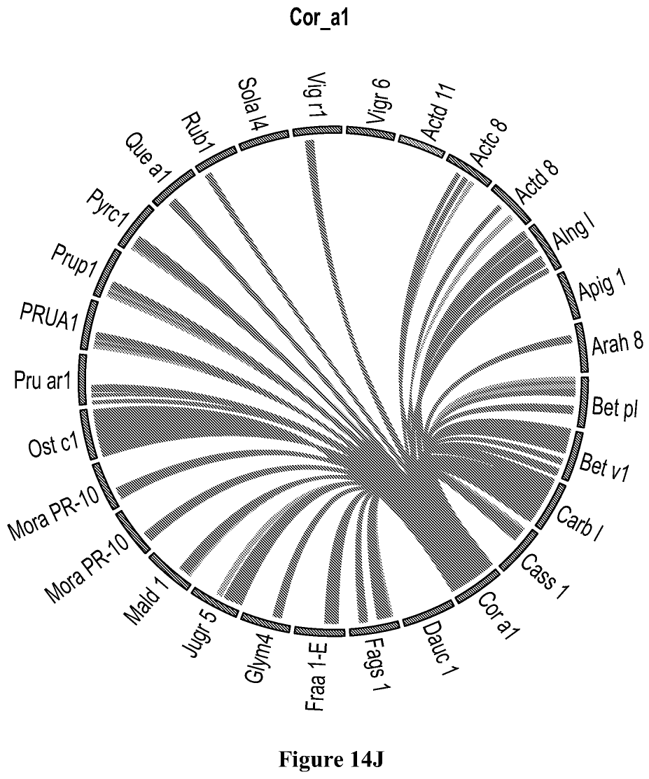

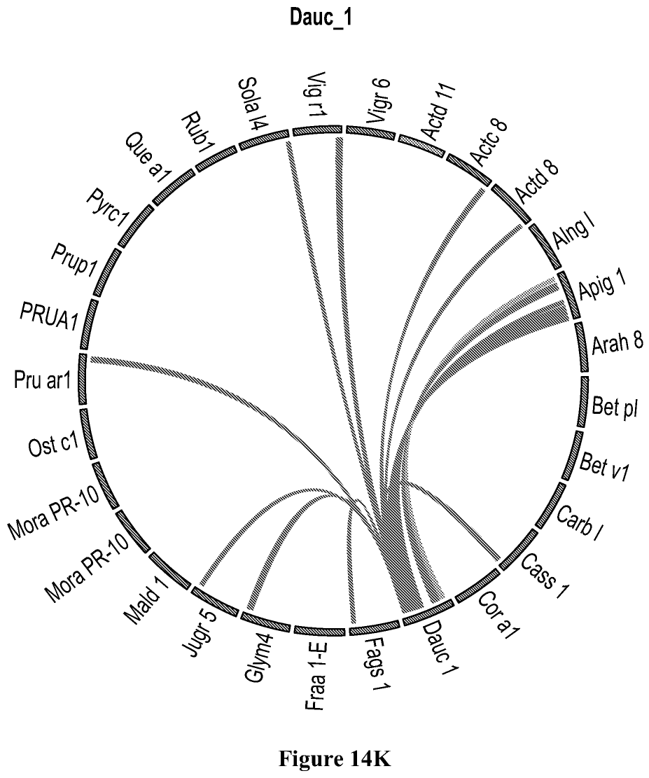

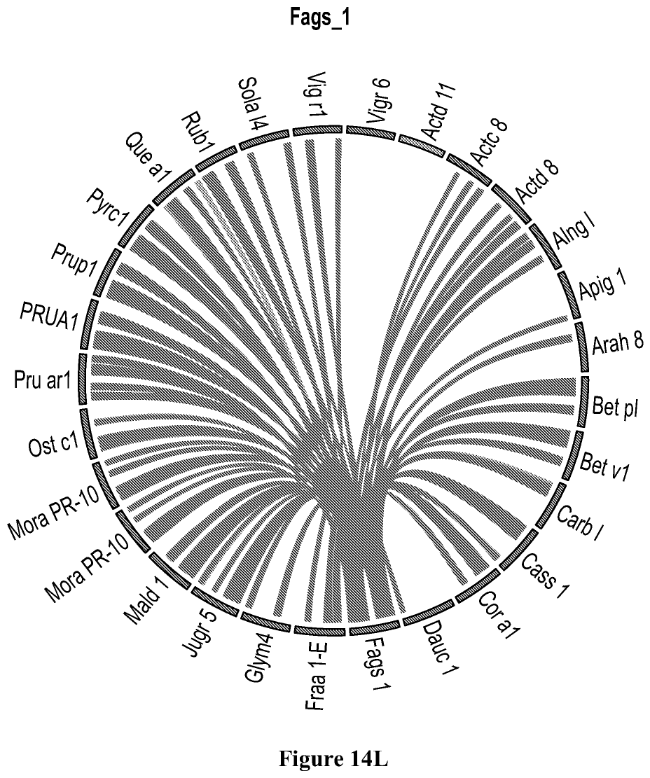

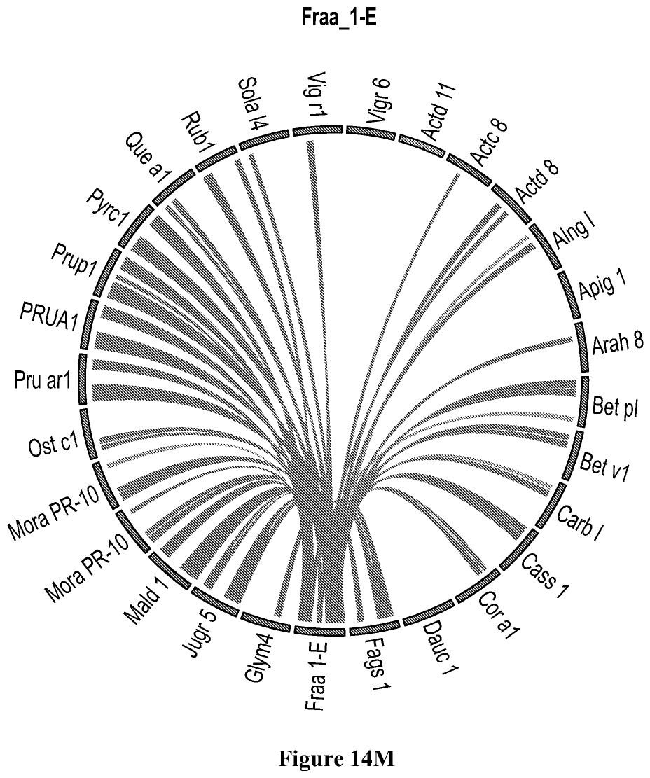

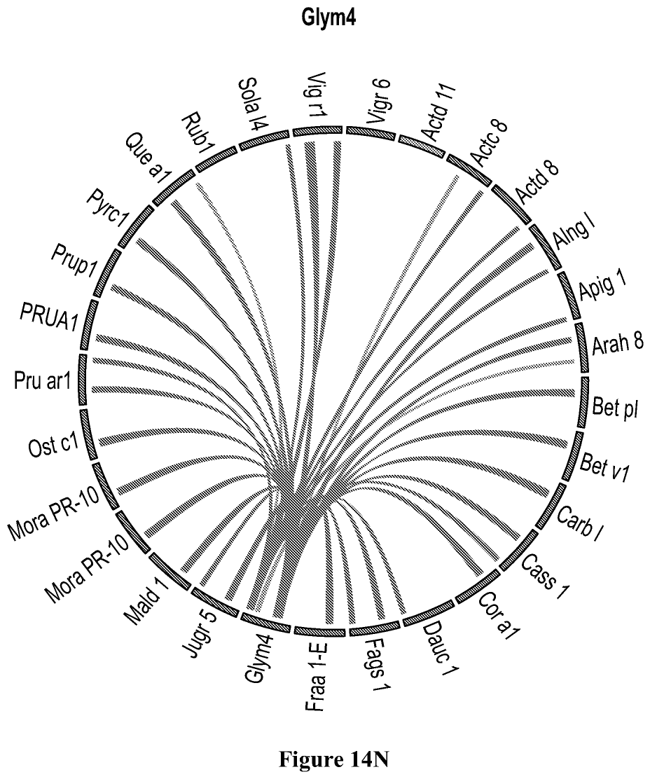

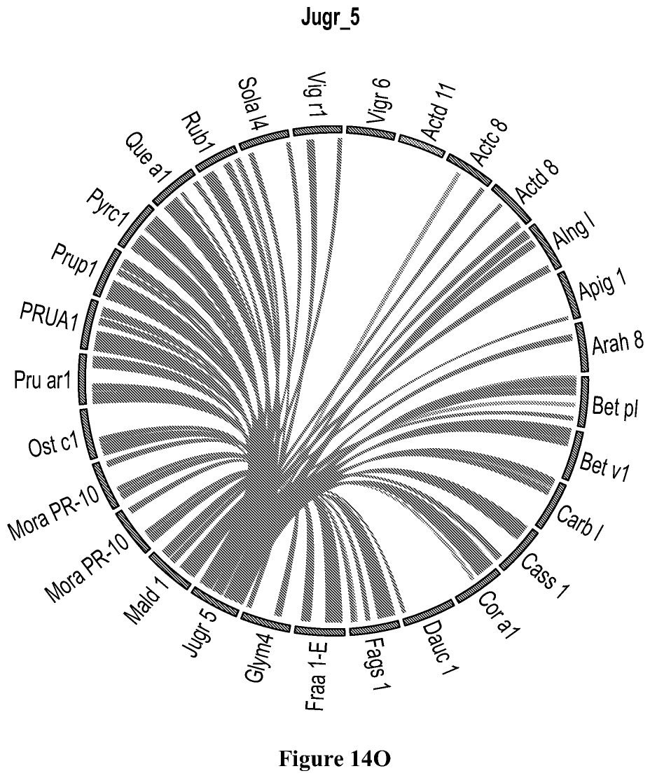

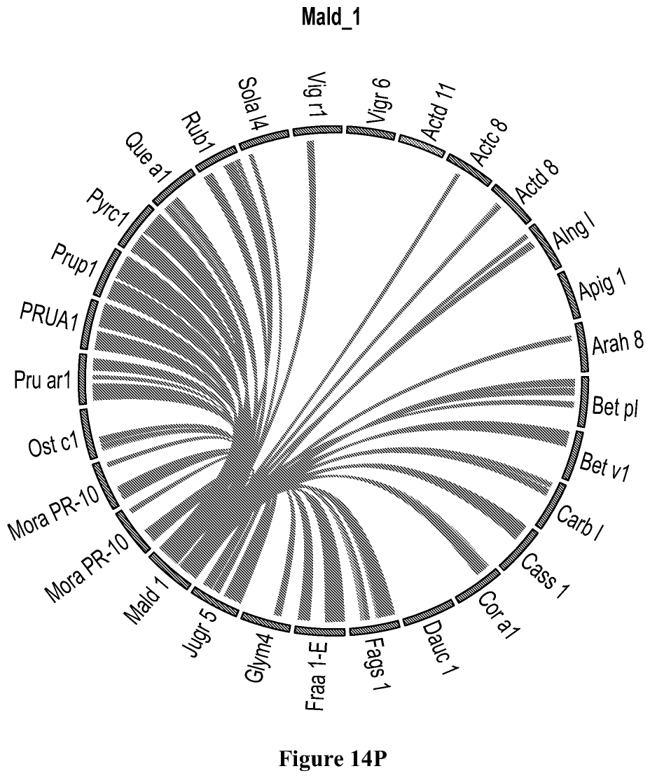

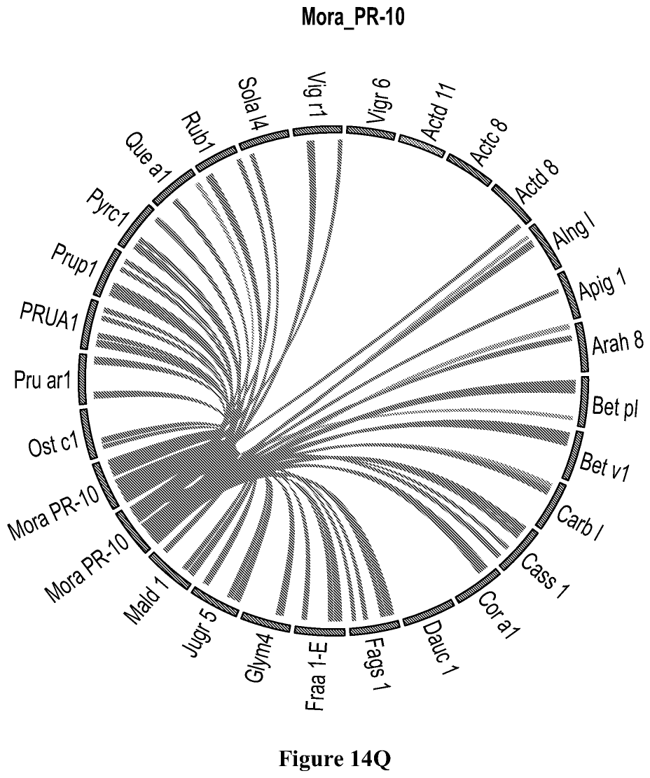

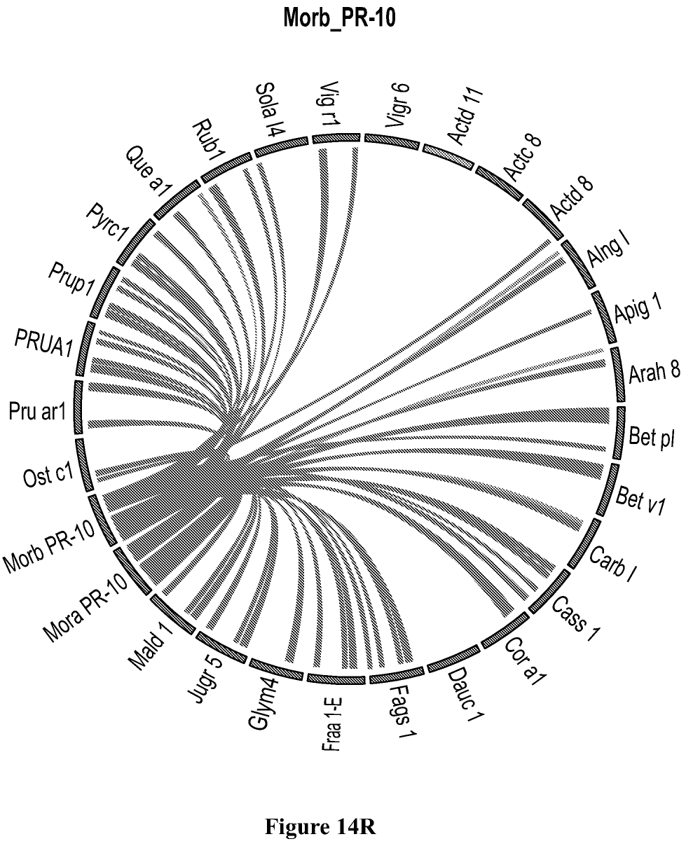

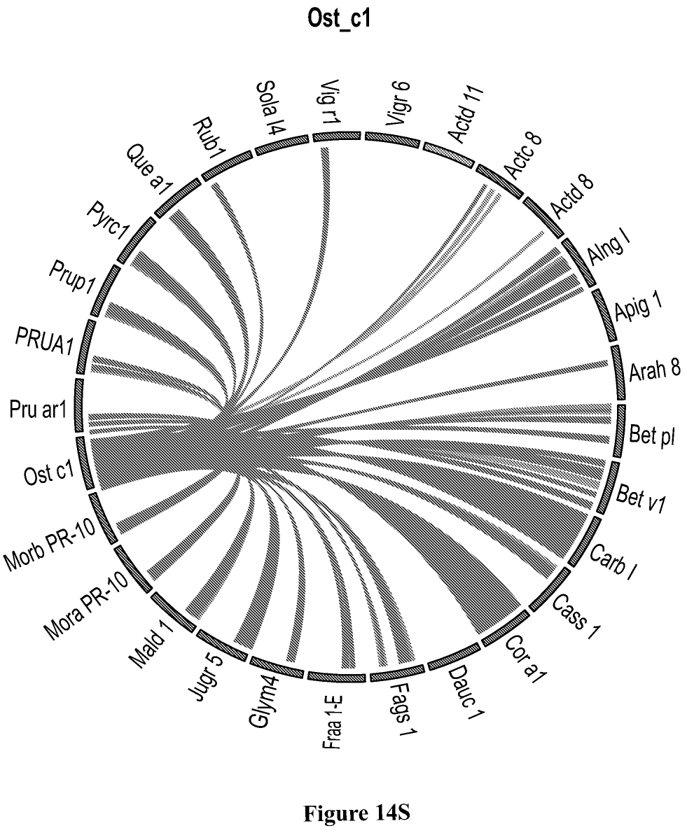

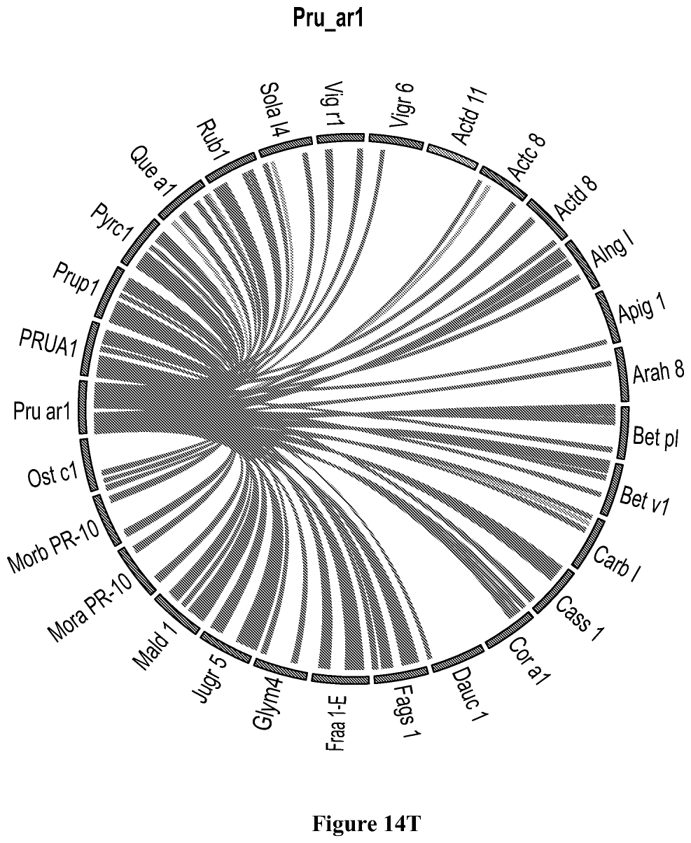

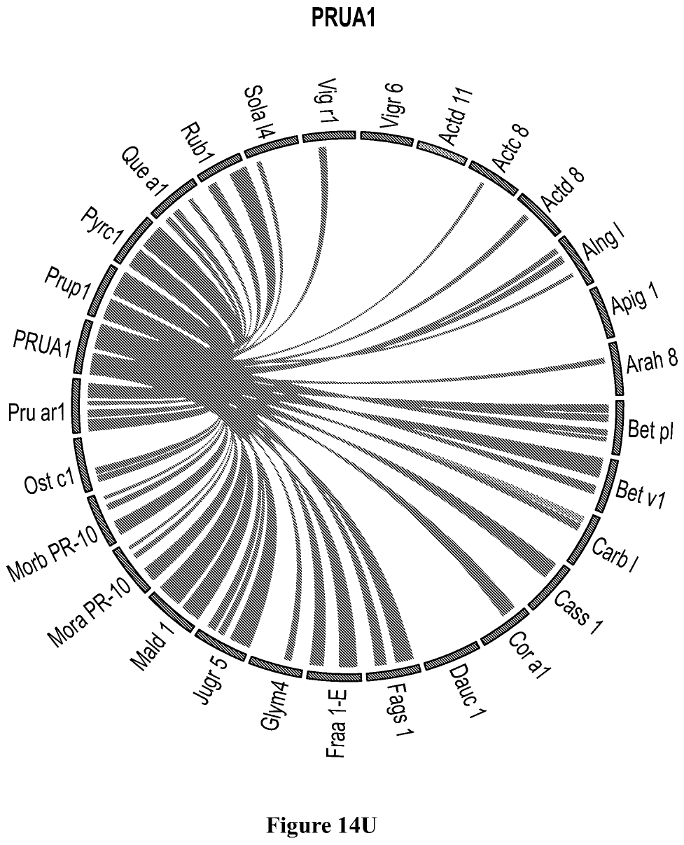

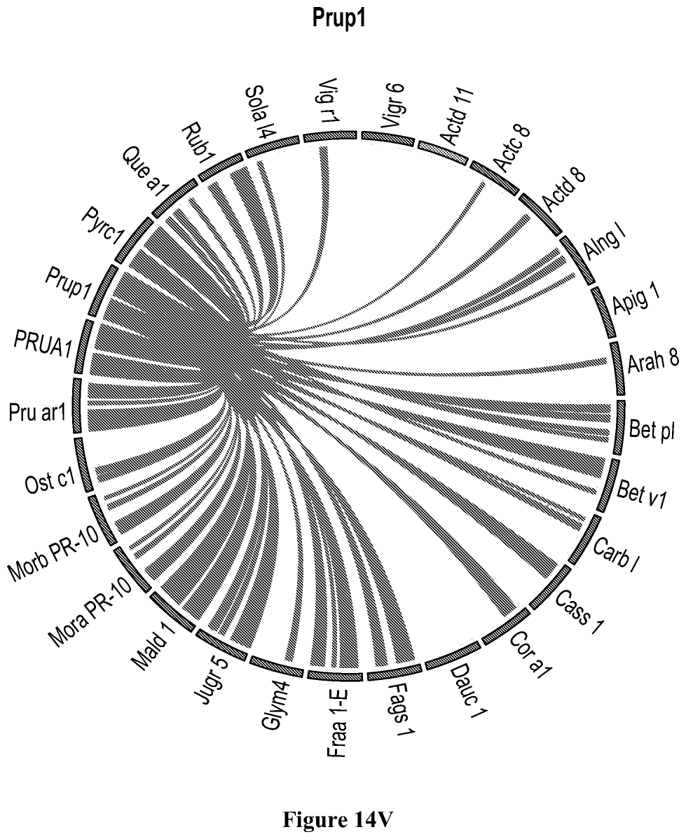

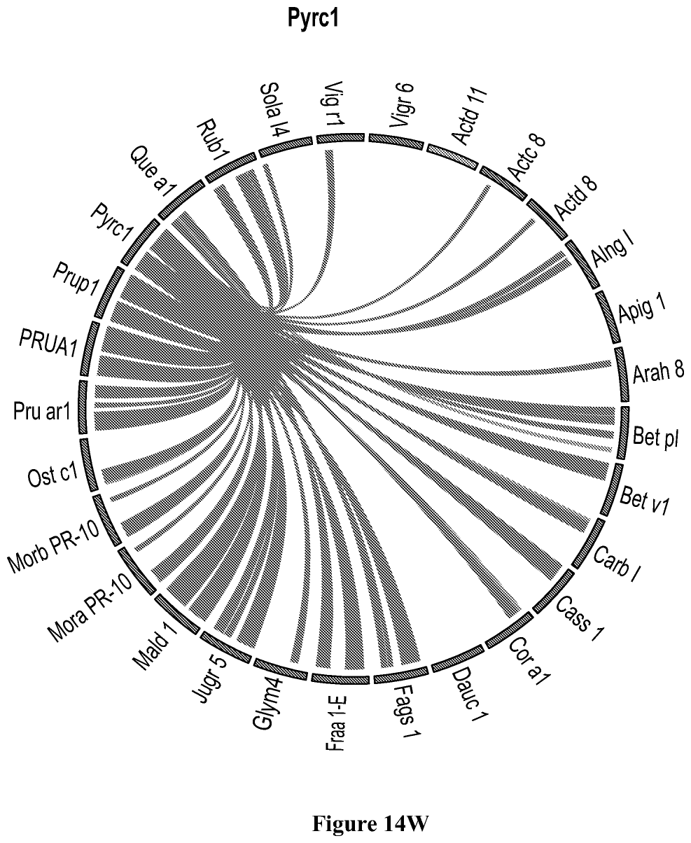

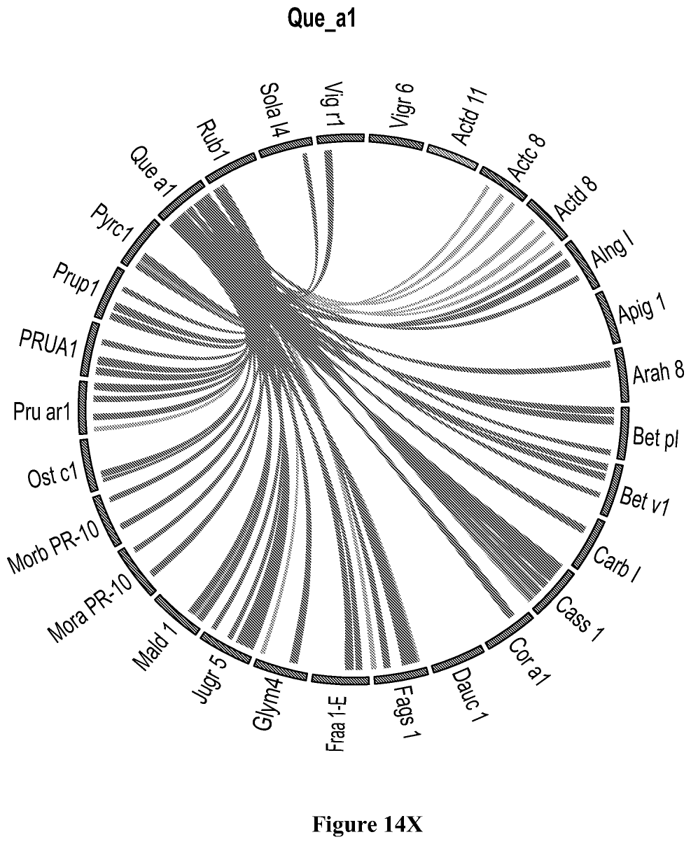

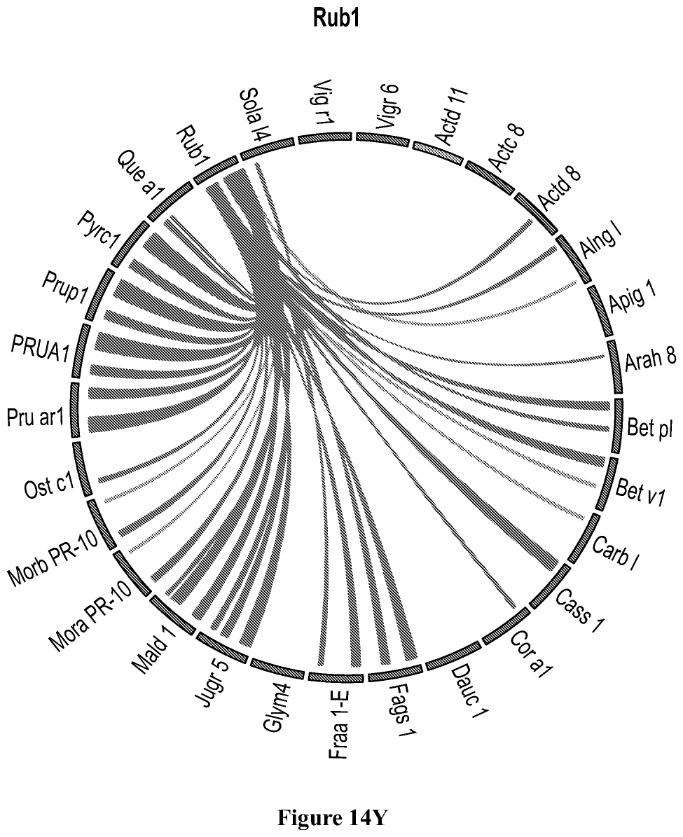

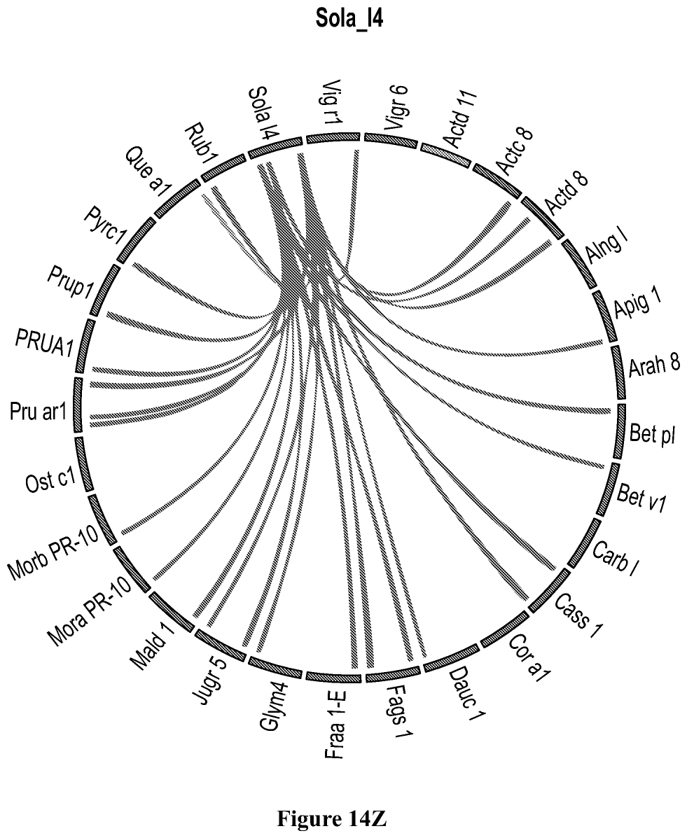

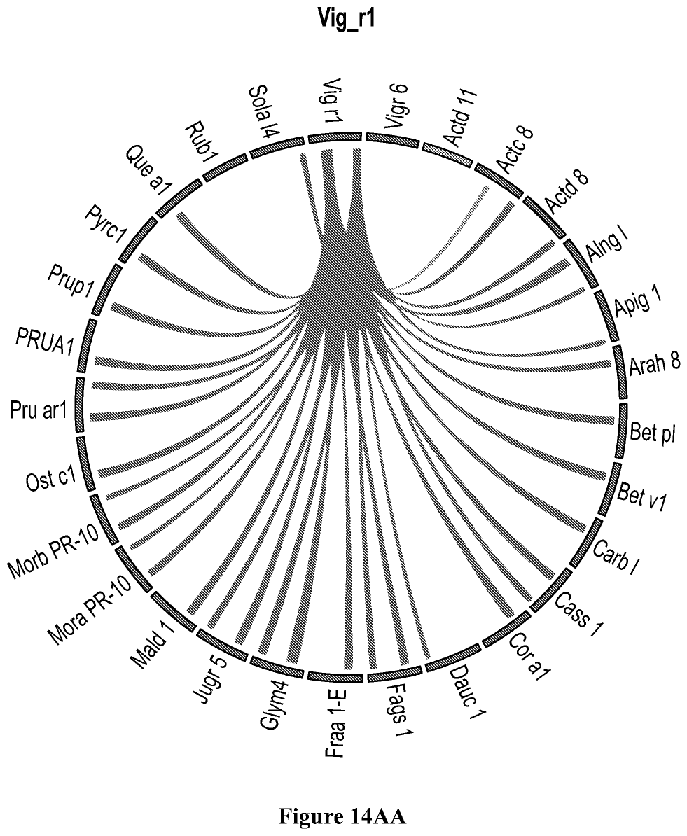

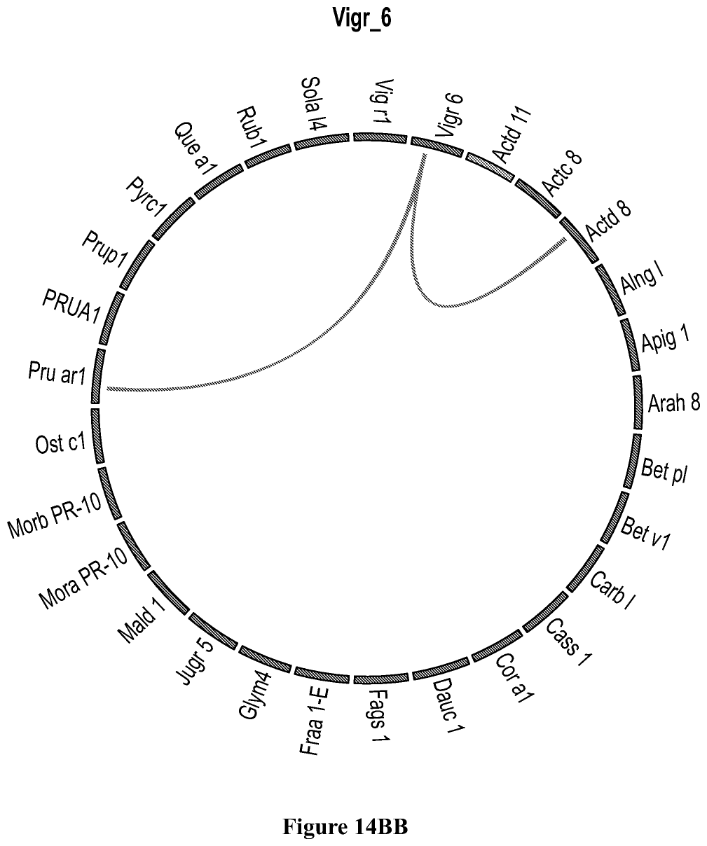

The resulting WIC-binding T-cell epitopes and MHC-binding B-cell epitopes may be, for example, mapped back to each individual allergenic full-length protein within a protein family to determination the location of each of the WIC-binding T-cell epitopes and the WIC-binding B-cell epitopes, and to identify cross-reactive MHC-binding epitopes shared between the two or more proteins within the protein family. Any identified MHC-binding epitope having a hit on two or more proteins within the protein family may be, for example, identified as cross-reactive epitopes. Alignment algorithms useful for the purpose include BLAST, BLASTP, FASTA, MegAlign etc. Epitope Hit graphs showing the frequency with which the MCH-binding epitopes align along the full-length amino acid sequence of a protein can be generated. Example 5 and FIGS. 9-11 provide further description of this process and the identified MHC-II-binding T-cell epitopes from an exemplary protein family, the Cupin protein family. Each cross-reactive MHC-binding epitope between proteins can be visualized as Circos plots (See exemplary FIGS. 11-14).

A cross-reactivity score may, for example, be applied to each identified MHC-binding epitope to allow rank ordering of the MHC-binding T-cell epitopes. The cross-reactivity score may, for example, be calculated as:

$$Cross - reactivity score = ( \frac{{\sum }_{i = 1}^{l} h}{l} )$$

where, l is length of peptide (≤15) or number of amino acids in a cross-reactive site (≤15 amino acids). h=number of times the given amino acid in the cross-reactive sites matches with high-confidence Kmers, given that these Kmers must also be matching with at least one other allergen.

Contemplated cross-reactive sites include regions (length ≤15 amino acids) in any allergenic protein sequence that match (using BLAST) with high-confidence Kmer(s) in such a way that the same given Kmer also matches with another allergen too. Cross-reactive sites may be characterized by the number of redundant high-confidence Kmers that match (using BLAST) a given region of a protein sequence. Again, these Kmers may, for example, be matching with at least two allergens (using BLAST). It is contemplated that if a given amino acid is covered by large number of Kmer sequences then h of that amino acid must be large. Accordingly, in certain embodiments, the cross-reactive score of a given cross-reactive site is the sum of h-scores of its amino acids divided by length of the site, i.e., mean of h-scores.

In another aspect, the present disclosure provides, for example, that the identified cross-reactive allergenic epitopes can be further ranked ordered and correlated with cross-reactive allergenic proteins identified the method described above and in Examples 1-3 based on cross-reactivity of full-length proteins. Accordingly, in some embodiments, methods for identification of cross-reactive epitopes further include screening the allergenic peptides comprising MHC-II-binding T-cell epitopes of IEDB.org having homology of at least 70% identity and 80% coverage of the peptides against the full-length cross-reactive allergenic proteins (e.g., the additional allergenic proteins having a cross-reactivity score of ≥0.5 and/or homology of at least 70% with respect to at least one identified allergenic protein).

In another aspect, the present disclosure provides, for example, that the cross-reactive WIC-binding T-cell epitopes identified by the methods disclosed herein bind at least one WIC molecule (haplotype or variants thereof). Information on HLA binding can be obtained for each of the experimentally validated T-cells from IEDB. Exemplary MHC-binding T-cell epitopes of Tables 13-23 bind 1 or more, 10 or more, 25 or more, 50 or more, 75 or more or 100 or more HLA molecules. In some embodiments, the present disclosure provides that the cross-reactive WIC-binding T-cell epitopes bind 1 to 150 WIC molecules and their variants. It is contemplated that immunotherapies, such as vaccines or T-cell epitope therapy, that include epitopes having degenerate HLA-binding may be more likely to succeed. (See, Example 5 and Tables 13-23).

Identified T-cell epitopes above can also be further tested in vitro for their binding affinity to MCH Class II molecules. For example, soluble HLA class II molecules may, for example, be obtained by lysis of cells homozygous for a given class II WIC molecule. The latter can be purified by affinity chromatography. Soluble Class II molecules may, for example, be incubated with a biotin-labeled reference peptide produced according to its strong binding affinity for that class II molecule. Peptides to be assessed for class II molecule binding may, for example, be incubated at different concentrations and their capacity to displace the reference peptide from its class II binding calculated by addition of neutravidin. See, for example, Texier et al. (2000) J. Immunology 164, 3177-3184.

In order to determine optimal T cell epitopes by, for example, fine mapping techniques, a cross-reactive epitope peptide having T-cell stimulating activity and thus comprising at least one T-cell epitope as determined by T-cell biology techniques can be modified by addition or deletion of amino acid residues at either amino- or carboxy-terminus of the peptide and tested to determine a change in T-cell reactivity. If two or more peptides share an area of overlap in a native cross-reactive allergenic protein as determined by T-cell biology techniques, additional peptides can be produced combining all or parts of such peptides and these peptides can be tested by a similar method. Following these methods, cross-reactive peptides may, for example, be selected and produced recombinantly or synthetically.

In one aspect, the present disclosure provides cross-reactive MHC-binding T-cell epitopes, for example those present in Cupin protein family. Exemplary MHC-binding cross-reactive T-cell epitopes are presented in Tables 13-23.

### Identification of Cross-Reactive Peptides Comprising B-Cell Epitopes and T-Cell Epitopes

In some embodiments, provided herein are allergenic peptides comprising overlapping or adjacent MHC-binding T-cell epitopes and B-cell epitopes. In some embodiments, provided herein are cross-reactive allergenic peptides comprising overlapping or adjacent MHC-binding T-cell epitopes and B-cell epitopes. Such peptides comprising overlapping or adjacent MHC-binding T-cell epitopes and B-cell epitopes can be identified by comparing identified T-cell epitopes (e.g., the MHC-II-binding T-cell epitopes of Tables 13-18, 20, 22) with identified B-cell epitopes on the proteins within a protein family (e.g., B-cell epitopes of Tables 19, 21, and 23).

### Determination of Surface Amino Acids for Determination of Discontinuous Epitopes

In one aspect, contemplated methods include identification of discontinuous epitopes that may be present on cross-reactive allergenic proteins. Information from identified epitope peptides that include MHC-II-binding T-cell epitopes and/or B-cell epitopes may be supplemented with information regarding discontinuous epitopes. This may be performed by obtaining multiple structural representatives or models of one or more cross-reactive allergenic proteins. Hidden Markov Model profiles may, for example, be generated using the program HMMer3. The HMM may, for example, be used to detect homologous structures in PDB with a minimum of e-value of 1×10⁻³. The HMM is used to align the structures' sequences to a reference sequence (e.g., an identified allergenic protein such as ara h 1 obtained from PDB (DOI: 10.2210/pdb3S7I/pdb)). An expected minimum threshold of 30% identity between at least one member of the cross-reactive allergenic protein or an epitope thereof (MHC-II-binding T-cell epitope or B-cell epitope) may, for example, be set to assert sufficient homology. In certain embodiments, no more than 10% insertions/deletions in the local alignment region are permitted.

Surface exposure of amino acids from the cross-reactive allergenic protein sequence and the identified allergenic protein may, for example, be predicted using the method described by Holbrook et al. (Protein Eng. 1990 August; 3(8):659-65. doi: 10.1093/protein/3.8.659). A comparison of the predicted surface amino acid residues with linear peptides including MHC-II-binding T-cell epitopes may, for example, aid in the selection of peptides including MHC-II-binding T-cell epitopes.

Predictions for surface exposure and surface antigenicity may also be performed using NetsurfP 2.0 (BioRxiv. 2018; 311209) and Bepipred2.0 (Jespersen et al. Nucleic Acids Res. 2017; 45:W24-9) software (for example, using default parameters), with the primary sequence of any of the identified allergenic proteins or any of the identified protein family members (such as Cupin, Tropomyosin, Prolamin, Lipid transfer protein, Bet v 1, EF hand, and the like) as an input, which assign either a value of 1 or 0 to each residue. The resulting prediction values may then be averaged over 15-residue epitopes, converted to a percent value, and plotted. The uniqueness and the cross-reactivity of each 15-residue epitope of each of the allergenic proteins among all epitopes in each of the protein families may then be determined by performing a BlastP2.0 search with the parameters defined in the heuristic string method by Berglund et al. (Protein Sci. 2008; 17:606-13.). Many matches may be obtained for each epitope, and the match with greatest homology with the MHC-T-cell binding epitope may be extracted, the corresponding homology converted to a percent value, and plotted as “maximum homology.”

### IV. PREPARATION OF ALLERMAP OF FOOD SOURCES

An allergenic protein identified to be present in a food source may, for example, be correlated with other additional allergenic proteins within a protein family by providing a map of identified additional proteins to its protein family, then mapping the full-length homology with each additional allergenic protein with the protein family to identify cross-reactive allergenic proteins, and then mapping homology of the cross-reactive allergenic proteins at the MHC-binding epitope (e.g., T-cell) or B-cell epitope.

Provided herein are methods for mapping allergenic proteins and cross-reactive allergenic proteins for a food sources to create an allergenic food map (an “AllerMap”) for a food source. Allergenic proteins present in a food and cross-reactive allergenic proteins (predicted and/or validated) that are not present in that food may, for example, be determined by the methods disclosed herein. Allergenic proteins having at least 70% homology and/or ≥0.5 A-RISC cross-reactivity score with respect to its identified allergenic protein (reference proteins) may, for example, be included in an AllerMap. In some embodiments, T-cell epitopes and/or B-cell epitopes in each of the identified allergenic proteins and cross-reactive allergenic proteins are included in an AllerMap.

An AllerMap, in some embodiments, is prepared as an array. FIG. 3 provides an exemplary AllerMap. In FIG. 3, each column of circles represents a protein family and each circle represent a member protein of that protein family. All protein families containing at least one known or potential allergenic protein would be represented in a column. Closed dark circles in each protein family represent known allergenic proteins for a food source for which the AllerMap is created (primary food source). Closed gray circles in each protein family column represent any protein across any protein family or food source that has at least 70% homology and/or ≥0.5 cross-reactivity score with respect to a known allergen in the primary food source. In this manner, the potential for cross-reactivity for each allergenic food source could be captured and depicted for easy, convenient, and visual reference for food allergies for parents of children, subjects, allergists/physicians and healthcare professionals and other having a need to know. An AllerMap is thus contemplated to be helpful in the design and preparation of multiple allergen oral immunotherapy.

### V. SYSTEMS, MEDIA AND METHODS FOR IDENTIFICATION OF CROSS-REACTIVE ALLERGENIC PROTEINS AND EPITOPES THEREOF, DATA TRANSMISSION AND STORAGE

Contemplated methods disclosed herein include computer-implemented methods. Exemplary embodiments are provided below.

Provided herein are, for example, computer-implemented methods for identification of cross-reactive allergenic proteins and/or preparing a mixed allergen oral immunotherapy, comprising: (a) mapping one or more identified allergenic proteins present in an multiple allergen oral immunotherapy to full length homology of other allergenic proteins within a protein domain family; (b) identifying cross-reactive allergenic proteins with the identified allergenic proteins by pairwise identity and similarity between the sequences; (c) correlating one or more identified allergenic proteins with other allergenic proteins in the same protein domain family; and/or (d) selecting two or more identified allergenic proteins as a component in an multiple allergen oral immunotherapy if there is at least 70% homology (identity and similarity) with two or more identified cross-reactive allergenic proteins.

Provided herein are, in some embodiments, computer-implemented methods for identification of an allergenic protein that is cross-reactive with an identified allergenic protein (e.g., to which a subject may have an allergy or intolerance, or that which may be present in Table 2). Such methods may, for example, comprise: (a) receiving, in a computer processing system, a data input comprising a first dataset further comprising a plurality of full-length identified allergenic protein sequences (e.g., those present in Table 2 or those selected to be present in a multiple allergen oral immunotherapy) and a second dataset comprising a plurality of full-length additional allergenic protein sequences in a protein family (e.g., the Cupin protein family, see Table 5); (b) applying an algorithm to compare each of the plurality of full-length additional allergenic protein sequences present in the first dataset to one or more of the plurality of full-length identified allergenic protein sequences present in the second dataset, and determining homology (based on pairwise identity and similarity) of each of the plurality of full-length additional allergenic protein sequences in first dataset to each of the plurality of full-length identified allergenic proteins sequences in the second dataset; (c) applying an algorithm for determining a cross-reactivity score for each of the plurality of the full-length additional allergenic protein sequences in the second dataset with respect to each of the full-length identified allergenic proteins (or reference proteins) sequences in the first dataset to predict cross-reactivity of each of the plurality of full-length additional allergenic protein sequences in the first dataset to the reference proteins; and (d) identifying one or more full-length additional allergenic protein sequences from the second dataset as cross-reactive with one or more of the full-length identified allergenic proteins in the first dataset, wherein the identified full-length cross-reactive allergenic protein is has at least 70% homology to a reference protein and/or a cross-reactivity score of ≥0.5 with respect to a reference protein.

Provided herein, in some embodiments, are computer-implemented methods for identification of cross-reactive epitopes shared by one or more identified allergenic proteins present in a food source (for example, one or more food sources that may be present in a multiple allergen immunotherapeutic composition) and one or more allergenic proteins within a protein family to which the one or more identified allergenic proteins belong, comprising:
(a) receiving, in a computer processing system, a data input comprising a first dataset comprising a plurality of full-length allergenic protein sequences present in an identified protein family, wherein the plurality of full-length allergenic protein sequences comprise the one or more identified allergenic protein present in the food source, a second dataset comprising a plurality of overlapping peptide K-mers derived from the plurality of full-length additional allergenic protein sequences from the first dataset; a third dataset comprising a plurality of validated T-cell epitopes; a fourth dataset comprising a plurality of validated MHC-binding T-cell epitopes (e.g., MHC-II-binding epitopes); and/or a fifth dataset comprising a plurality of validated B-cell epitopes;
(b) applying an algorithm to map the:
(i) plurality of validated T-cell epitopes in the third dataset on to the overlapping peptide K-mers of the second dataset to identify the presence of one or more validated T-cell epitopes in the second dataset;
(ii) plurality of validated MHC-binding T-cell epitopes in the fourth dataset on to the overlapping peptide K-mers of the second dataset to identify the presence of one or more validated MHC-binding T-cell epitopes in the second dataset; and/or
(iii) plurality of validated B-cell epitopes in the fifth dataset on to the overlapping peptide K-mers of the second dataset to identify the presence of B-cell epitopes in the second dataset;
(c) applying an algorithm to the identified validated T-cell epitopes, MHC-binding T-cell epitopes, and/or the B-cell epitopes in (b) to each of the plurality of full-length allergenic proteins in the first dataset to identify cross-reactive epitopes; and optionally,
(d) applying an algorithm to compare each of the plurality of full-length allergenic protein sequences present in the first dataset to each of the other full-length allergenic protein sequences present in the first dataset, and determining homology (based on pairwise identity and similarity) of each of the plurality of full-length allergenic protein sequences in first dataset to each of the other full-length allergenic protein sequences in the first dataset; wherein each of the plurality of full-length allergenic proteins is a reference protein with respect to the other full-length allergenic proteins in the first dataset;
(e) applying an algorithm for determining a cross-reactivity score for each of the plurality of full-length allergenic protein sequences in the first dataset with respect to each of the other full-length allergenic protein sequences (each a reference protein) in the first dataset to identify cross-reactivity of each of the plurality of full-length allergenic protein sequences in the first dataset with respect to a reference protein; and/or
(f) identifying one or more full-length allergenic protein sequences from the first dataset as cross-reactive with one or more full-length allergenic sequences in the first dataset, wherein the identified full-length cross-reactive allergenic protein has at least 70% homology to a reference protein and/or a cross-reactivity score of ≥0.5 with respect to a reference protein.

Provided herein are, for example, non-transitory computer-readable media storing thereon executable instructions, that when executed by a computer or a device, causes the computer or a device to execute a method for identification of cross-reactive allergenic protein sequences that are cross-reactive to one or more identified allergenic proteins present in a multiple allergen oral immunotherapy, the method comprising: (a) receiving, in a computer processing system, a data input comprising a first dataset comprising a plurality of identified allergenic proteins sequences (e.g., those present in Table 2 or those selected to be present in a multiple allergen oral immunotherapy) and a second dataset comprising a plurality of additional allergenic protein sequences; (b) applying an algorithm to compare each of the plurality of additional allergenic protein sequences present in the second dataset to one or more of the plurality of identified allergenic protein sequences present in the first dataset, and determining homology (based on pairwise identity and similarity) of each of the plurality of additional allergenic protein sequences in second dataset to each of the plurality of identified allergenic proteins sequences in the first dataset; (c) applying an algorithm for determining a cross-reactivity score for each of the plurality of additional allergenic protein sequences in the second dataset with respect to each identified allergenic protein (or reference protein) sequences in the first dataset (e.g., the identified allergenic proteins present in a multiple allergen oral immunotherapy) to predict cross-reactivity of each of the plurality of additional allergenic protein sequences in the second dataset to a reference protein; and/or (d) identifying one or more additional allergenic protein sequences from the second dataset as cross-reactive with one or more identified allergenic sequences in the first dataset, wherein the identified cross-reactive allergenic protein is at least 70% homologous to a reference protein and has a cross-reactivity score of ≥0.5 with respect to a reference protein, wherein the cross-reactive allergenic protein from the second dataset is not present in the first dataset.

Provided herein are, for example, non-transitory computer-readable storage media encoded with a computer program including instructions executable by a digital processing device to create an application, the application comprising: (a) a software module configured to obtain a data input comprising a first dataset comprising a plurality of identified allergenic proteins sequences and a second dataset comprising a plurality of additional allergenic protein sequences; (b) a software module configured to apply an algorithm to compare each of the plurality of additional allergenic protein sequences present in the second dataset to one or more of the plurality of identified allergenic protein sequences present in the first dataset, and determining homology (based pairwise identity and similarity) of each of the plurality of additional allergenic protein sequences in second dataset to each of the plurality of identified allergenic proteins sequences in the first dataset; (c) a software module configured to apply an algorithm for determining a cross-reactivity score for each of the plurality of additional allergenic protein sequences in the second dataset with respect to each identified allergenic protein (or reference protein) sequence in the first dataset to predict cross-reactivity of each of the plurality of additional allergenic protein sequences in the second dataset to the reference protein; and (d) a software module for identifying one or more full-length additional allergenic protein sequences from the second dataset as cross-reactive with one or more full-length identified allergenic proteins (or reference proteins) in the first dataset, wherein the identified cross-reactive allergenic protein is at least 70% homologous to a reference protein and/or has a cross-reactivity score of ≥0.5 with respect to a reference protein, wherein the cross-reactive allergenic protein from the second dataset is not present in the first dataset.

Provided herein are, for example, computer-implemented systems, that are configured to implement the methods of the disclosure. The system can include a digital processing device that is programmed to implement the methods described herein. In some embodiments, provided herein is a computer-implemented system comprising: (a) a digital processing device comprising an operating system configured to perform executable instructions, and a memory device; and (b) a computer program including instructions executable by a digital processing device to create an application comprising: (i) a software module configured to receive a software module configured to obtain a data input comprising a first dataset comprising a plurality of full-length identified allergenic protein sequences and a second dataset comprising a plurality of full-length additional allergenic protein sequences; (ii) a software module configured to apply an algorithm to compare each of the plurality of full-length additional allergenic protein sequences present in the second dataset to one or more of the plurality of full-length identified allergenic protein sequences present in the first dataset, and determining homology (based on pairwise identity and similarity) of each of the plurality of full-length additional allergenic protein sequences in second dataset to each of the plurality of full-length identified allergenic proteins sequences in the first dataset; (iii) a software module configured to apply an algorithm for determining a cross-reactivity score for each of the plurality of full-length additional allergenic protein sequences in the first dataset with respect to each of the full-length identified allergenic protein (or reference protein) sequences in the second dataset to predict cross-reactivity of each of the plurality of full-length additional allergenic protein sequences in the first dataset to the full-length reference proteins; and/or (iv) a software module for correlating one or more additional allergenic protein sequences from the second dataset as cross-reactive with one or more full-length identified allergenic protein sequences in the second dataset, wherein the full-length identified cross-reactive allergenic protein is at least 70% homologous to a full-length reference protein and/or has a cross-reactivity score of ≥0.5 with respect to a full-length reference protein, wherein the full-length cross-reactive allergenic protein from the first dataset is not present in the second dataset.

In some embodiments, algorithms applied include alignment algorithms such as FASTA, BLAST, BLASTP, TBLASTN and others disclosed herein and known in the art.

In some embodiments, an algorithm applied includes a A-RISC score calculation.

In some embodiments, a computer-mediated method, non-transitory computer-readable media, non-transitory computer-readable storage media, and/or computer-mediated system of the present disclosure includes one or more bioinformatics steps to generate an in silico library comprising a plurality of peptides derived a plurality of full-length additional allergenic protein sequences (for example, from a second dataset). Algorithms for generating overlapping peptides are known in the art (e.g., PepSequencer from JPT Innovative Tools). In some embodiments, contemplated methods further comprise obtaining a dataset comprising overlapping peptides derived from one or more allergenic protein sequences (for example, from a plurality of full-length additional allergenic protein sequences in a dataset, for example, a second dataset). Such may, for example, be overlapping peptides (Kmers), for example, from 5 amino acids to 100 amino acids in length. In some embodiments, contemplated peptides are 5-30 mers and have a sliding window of 1-5 amino acids. In some embodiments, contemplated peptides are overlapping 15-mer peptides with a sliding window of 1-5 amino acids.

In some embodiments, a computer-mediated method, non-transitory computer-readable media, non-transitory computer-readable storage media, and/or computer-mediated system of the present disclosure further includes obtaining a dataset containing a plurality of T-cell epitopes present in one or more allergens (for example, one or more food allergens). T-cell epitopes are available from databases such as IEDB, MHCBN (available on the world wide web at crdd.osdd.net/raghava/mhcbn/). An alignment algorithm may, for example, be applied to each of a plurality of peptides to determine homology (based on pair-wise identity and similarity) and generate an in silico T-cell epitope library containing a plurality of peptides containing T-cell epitopes identified to be present on cross-reactive allergenic proteins. Homology may, for example, be determined by application of an algorithm such as BLAST, BLASTP, FASTA as described above or any other algorithm known in the art for aligning and/or identification of short peptide sequences.

In some embodiments, a computer-mediated method, non-transitory computer-readable media, non-transitory computer-readable storage media, and/or computer-mediated system of the present disclosure further includes determining the potential for binding to MHC-II molecules by a peptides containing a T-cell epitopes (for example, T-cell epitopes identified using method disclosed herein). Accordingly, contemplated methods further include obtaining a dataset comprising a plurality of experimentally validated MHC-II-binding T-cell epitopes. An alignment algorithm may, for example, be applied to each of the plurality of peptides containing T-cell epitopes to identify a plurality of peptides containing MHC-II-biding T-cell epitopes. Binding affinity parameter ranges can be, for example, 1 Kd to ≤500 Kd. In some embodiments, a binding parameter is set at ≤500 Kd. In some embodiments, a binding affinity parameter is set at ≤100 Kd.

In some embodiments, a computer-mediated method, non-transitory computer-readable media, non-transitory computer-readable storage media, and/or computer-mediated system of the present disclosure includes applying an alignment algorithm to an overlapping peptide and an MHC-II-biding T-cell epitope to identify the potential for MHC-II binding by the overlapping peptides.

In some embodiments, overlapping peptides have homology with MHC-II-binding T-cell epitopes (for example, have at least 70% identity and 80% coverage over the length of the peptide). Homology can be determined by application of algorithms such as BlastP and/or FASTA as described above. In some embodiments, peptides having homology (based on 70% identity (exact match) and 80% coverage)) with MHC-II-binding epitopes having a binding affinity of 1 Kd to ≤500 Kd and are selected and stored locally in an in silico MHC-II-binding epitope library. In some embodiments, peptides having homology (based on 70% identity (exact match) and 80% coverage)) with MHC-II-binding epitopes have a binding affinity of 1 Kd to ≤100 Kd and are selected and stored locally in an in silico MHC-II-binding epitope library.

In some embodiments, a computer-mediated method, non-transitory computer-readable media, non-transitory computer-readable storage media, and/or computer-mediated system of the present disclosure includes correlating one or more specific allergenic proteins with other allergenic proteins within a protein family to identify cross-reactive peptides containing specific T-cell epitopes and/or specific B-cell epitopes. Such correlation includes mapping each of the plurality of identified allergenic proteins to its protein family, mapping the full-length homology and cross-reactivity scores of all allergenic proteins present within each protein family to identify cross-reactive allergenic proteins within each protein family, and then mapping T-cell epitopes or B-cell epitopes or both on each cross-reactive allergenic protein with a protein family. Correlation is performed by applying one or more algorithms, such as Blastp, FASTA and/or others known in the art to determine pair-wise identity and similarity between a pair of allergenic sequences (specific allergenic protein and other allergenic proteins) with a protein family thereby identifying cross-reactive proteins. Each specific allergenic protein within a protein family is thus mapped to other allergenic proteins with each protein family for identification of cross-reactive peptides containing specific T-cell epitopes and/or specific B-cell epitopes.

In some embodiments of a computer-mediated method, non-transitory computer-readable media, non-transitory computer-readable storage media, and/or computer-mediated system of the present disclosure, peptides containing MHC-II-binding T-cell epitopes in an in silico MHC-II-binding T-cell epitope library are screened in in vitro T-cell binding assays and or IgE binding assays using the methods known in the art to select desirable peptides containing the T-cell epitopes.

### Digital Processing Device

In some embodiments, systems, media and methods described herein include a digital processing device or a use of the same. In further embodiments, a digital processing device includes one or more of a hardware central processing unit (CPU) or a processor that carry out a devices' function. Non-limiting examples of processors include microprocessors, digital signal processors, graphics processing units, and the like. In further embodiments, a digital processing device comprises an operating system configured to perform executable instructions. A digital processing device may also include processors such as microprocessors, digital signal processors, graphics processing units, and data acquisition units. A digital processing device may also be optionally connected to a: computer network, the intranet, and/or the internet such that it accesses world wide web. In further embodiments, a processor is optionally connected to a cloud computing infrastructure, a memory device and/or a data storage device.

In accordance with the description herein, suitable processors include, by way of non-limiting examples, server computers, desktop computers, laptop computers, notebook computers, sub-notebook computers, netbook computers, netpad computers, set-top computers, handheld computers, mobile smartphones, tablet computers, and the like. Those of skill in the art will recognize that many smartphones, television, video players, and digital music players with optional computer network connectivity are suitable for use in the system described herein.

In other embodiments, a digital processor device includes an operating system configured to perform executable instructions. An operating system is for example, software, including programs and data, which manages a device's hardware and provides services for execution of applications. Those of skill in the art will recognize that suitable server operating systems include by way of non-limiting examples, FreeBSD, OpenBSD, NetBSD®, Linux, Apple®, Mac OS X Server®, Oracle®, Solaris®, Windows Server®, VMware, and Novell Netware®. Those of skill in the art will recognize that suitable personal computer operating systems include, by way of non-limiting examples, Microsoft®, Windows®, Apple®, Mac OS X®, UNIX®, and Unix-like operating systems, such as GNU/Linux®. In some embodiments, a device includes a storage and/or memory device.

In some embodiments, a digital processing device includes a display to send visual information to a user. In some embodiments, a display is a cathode ray tube (CRT), a liquid crystal display (LCD), a thin film transistor liquid crystal display (TFT-LCD), organic light-emitting diode (OLED), a passive-matrix OLED (PMOLED), active-matrix OLED (AMOLED) display, a plasma display, a video projector or a combination thereof.

In some embodiments, a digital processor device includes an input device to receive information from a user. In some embodiments, a user is a subject having two or more food allergies or two or more food-intolerances such as peanuts, cashews and like, medical professional, researcher, analyst, or a combination thereof. In some embodiments, a medical professional is an allergist, clinician, doctor, nurse, physician's assistant, pharmacist, medical consultant, or other hospital or medical professional. In some embodiments, an input device is a keyboard. In some embodiments, an input device is a pointing device, by way of non-limiting examples, a mouse, trackball, trackpad, or stylus. In other embodiments, an input device is a touch screen or multi-touch screen.

### Non-transitory Computer-Readable Storage Media

In some embodiments, contemplated systems, media, and methods disclosed herein include one or more non-transitory computer-readable storage medium encoded with a program including instructions executable by an operating system of an optionally networked digital processing device. In further embodiments, a computer-readable storage medium is a tangible component of a digital processing device. In further embodiments, a computer-readable medium is optionally removable from a digital processing device. In some embodiments, a computer-readable storage medium includes CD-ROMS, DVDs, DVD-ROM, flash memory drives, magnetic tape drives, magnetic disk drives, optical disc drives, solid-state drives, and cloud computing-based systems such as azure, AWS, and the like. In further embodiments, programs and instructions are permanently, substantially permanently, semi-permanently, or non-transitorily encoded on the media.

### Computer Programs

In some embodiments, systems, media, and methods disclosed herein include at least one computer program, or use of the same. A computer program includes a sequence of instructions, executable in a digital processing device's computer, (such as a CPU or a microprocessor) written to perform a specified task. Computer-readable instructions may be implemented as a computer, program module, such as function, object, application, Application Programming Interfaces (APIs), data structure, and the like, that performs a particular task or implements particular abstract data types. Those of skill in the art will recognize that a computer program may be written in various versions of various languages. In various embodiments, a computer program includes one or more software modules. In various embodiments, a computer program includes, in part or in whole, one or more web applications, one or mobile applications, one or more standalone applications, one or more web browser plugins, extensions, add-ins, or combinations thereof.

### Web Applications

In some embodiments, a computer program includes a web application. One of skill in the art will understand that a web application, in various embodiments, utilizes one or more software frameworks and one or more database systems. In some embodiments, a web application is created upon a software framework such as JAVA, Microsoft.NET or Ruby on Rails (RoR). In some embodiments, a web application may utilize one or more database systems such as Microsoft® SQL Server, mySQL®, noSQL, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or extensible Markup Language (XML).

### Mobile Application

In some embodiments, a computer program includes a mobile application provided to a mobile digital processing device. In some embodiments, an application is provided to a mobile digital processing device via a computer network. In other embodiments, a mobile application is provided to a mobile digital processing device at the time it is manufactured.

A mobile application may be, for example, created by methods known to those of skill in the art using hardware, languages, and development environments known to the art. Those of skill in the art will recognize that mobile applications are written in several languages, such as C, C++, C #, Objective-C, Java™, Javascript, Pascal, Object Pascal, Python™, Ruby, R, VB.NET, WML, and XHTML/HTML with or without CSS, or combinations thereof. Suitable mobile application development environments are available, such as AirplaySDK, alcheMo, Appcelerator®, Celsius, Bedrock, Flash Lite, .NET Compact Framework, Rhomobile, and WorkLight Mobile Platform, Lazarus, MobiFlex, MoSync, and Phonegap, mobile software developer kits such as iPhone and iPad (iOS) SDK, Android™ SDK, BlackBerry® SDK, BREW SDK, Palm® OS SDK, Symbian SDK, webOS SDK, and Windows® Mobile SDK.

### Software Modules

In some embodiments, the systems, media, and methods disclosed herein include software, server, and/or database modules, or use of the same. Software modules may be created by techniques known to those of skill in the art using machines, software, and languages known to the art. The software modules disclosed herein may be implemented in a multitude of ways. In various embodiments, a software module comprises a file, a section of code, a programming object, a programming structure, or combinations thereof. In further embodiments, a software module comprises a plurality of files, a plurality of sections of code, a plurality of programming objects, a plurality of programming structures, or combinations thereof. In various embodiments, one or more software modules comprise, by way of non-limiting examples, a web application, a mobile application, and a standalone application.

### Databases

In some embodiments, the systems, media, and methods disclosed herein include one or more databases, data sources, or use of the same. Those of skill in the art will recognize that many databases are suitable for storage and retrieval of data. In various embodiments, suitable databases include, by way of non-limiting examples, relational databases, non-relational databases, object-oriented databases, object databases, entity-relationship model databases, associative databases, and XML databases. In some embodiments, a database is internet-based, web-based, cloud computing-based or on one or more local computer storage devices.

In some embodiments, databases or data sources are selected from genomic databases, allergen databases, clinical trial databases, scientific databases, or a combination thereof. In some embodiments, the one or more databases or sources comprise publicly available databases, proprietary databases, or a combination thereof.

### Data Transmission

Any appropriate method can be used to communicate information pertaining to data generated by the compositions and methods of the present disclosure to another person. In some embodiments, the systems, media and methods disclosed herein further comprise one or more transmission devices comprising an output means for transmitting one or more data, results, outputs, information, reports and images. In some embodiments, the output means takes any form which transmits the data, results, requests, and/or information and comprises a monitor, printed format, printer, computer, processor, memory location, or a combination thereof. In some embodiments, the transmission device comprises one or more processors, computers, and/or computer systems for transmitting information.

In some embodiments, transmission comprises tangible transmission media and/or carrier-wave transmission media. In some embodiments, tangible transmission media include coaxial cables; copper wire and fiber optics, including the wires that comprise a bus within a computer system. In some embodiments, carrier-wave transmission media takes the form of electric or electromagnetic signals, or acoustic or light waves such as those generated during radio frequency (RF) and infrared (IR) data communications.

The output can be presented to a user with the aid of a user interface, such a graphical user interface.

### VI. CROSS-REACTIVE ALLERGENIC PROTEINS AND CROSS-REACTIVE EPITOPES

In one aspect, provided herein are cross-reactive allergenic proteins and cross-reactive epitopes (for example, T-cell epitopes, MHC-binding binding epitopes, and B-cell epitopes) identified (or identifiable) by methods of the present disclosure.

In another aspect, provided herein are T-cell epitopes present on cross-reactive allergenic proteins Ber_e_2, Car_i_2, Fag_e_1, Fag_e_13S, Fag_t_13S_globulin, Jug_n_4, Lup_a_vicilin, Lup_an_1, Pin_k_2, Pis_s_1, Pis_s_2, Pis_v_2, Ses_i_3, Ses_i_6, Ses_i_7, Sin_a_2, Vig_r_2, Car_i_4, Gly_m_5, Gly_m_6, Jug_r_4, Len_c_1 and Pru_du_6 belonging to the Cupin protein family (see Tables 13-18). In some embodiments, the T-cell epitopes are cross-reactive T-cell epitopes.

In another aspect, provided herein are B-cell epitopes present on cross-reactive allergenic proteins Ber_e_2, Car_i_2, Fag_e_1, Fag_e_13S, Fag_t_13S_globulin, Jug_n_4, Lup_a_vicilin, Lup_an_1, Pin_k_2, Pis_s_1, Pis_s_2, Pis_v_2, Ses_i_3, Ses_i_6, Ses_i_7, Sin_a_2, Vig_r_2, Car_i_4, Gly_m_5, Gly_m_6, Jug_r_4, Len_c_1 and Pru_du_6 belonging to the Cupin protein family (see Table 19). In some embodiments, the B-cell epitopes are cross-reactive B-cell epitopes.

One of skill in the art will appreciate that polynucleotides and oligonucleotides (including DNA, cDNA, mRNA and the like) that can be translated to provide the cross-reactive allergenic proteins and the cross-reactive epitopes of the present disclosure are within the scope of the disclosure. Sequences for polynucleotides and oligonucleotides corresponding to the cross-reactive B-cell and T-cell epitopes including WIC-binding T-cell epitopes are readily ascertainable from Genbank/Swiss Protein using Genbank/Swiss_prot ID and allergen ID and/or by reverse translation. It is understood that all polynucleotides and oligonucleotides encoding the cross-reactive allergenic proteins and cross-reactive epitopes are within the scope of the present disclosure. It is also understood that cross-reactive epitopes of the present disclosure include all epitopes, including T-cell epitopes and B-cell epitopes, that are present on allergenic proteins and variants thereof, for example, determined or determinable to be cross-reactive using the methods of the present disclosure.

Cross-reactive allergenic proteins and cross-reactive epitope peptides may, for example, be useful in the treatment, prevention and management of not only IgE-mediated diseases, disorders and conditions, including but not limited to, allergies, food-intolerance, inflammatory diseases, autoimmune diseases, general immune health and the like, but also any disease that is sensitive to or involves the cross-reactive allergenic proteins and/or cross-reactive epitopes of the present disclosure. Cross-reactive allergenic proteins and the cross-reactive epitopes of the present disclosure may, for example, also be useful in preparation of compositions and kits comprising cross-reactive allergenic proteins and/or cross-reactive epitope peptides. Cross-reactive allergenic proteins and cross-reactive epitope peptides of the present disclosure may, for example, also be useful in the preparation of vaccines, T-cell peptide therapeutics, and other immunotherapeutics, binding partners such as antibodies (and/or fragments thereof), tests, assays, diagnostics, reagents, kits, and single allergen and multiple allergen immunotherapeutic compositions, etc.

In some embodiments, provided herein are cross-reactive allergenic proteins and compositions comprising them. Non-limiting exemplary cross-reactive allergenic proteins include identified allergenic proteins of Table 2, the cross-reactive allergenic proteins present in identified protein families of Table 3, and cross-reactive allergenic proteins present in Tables 4-7 and 10-12. In some embodiments, one or more cross-reactive allergenic proteins are selected from Cupin protein family (Tables 5 and 10). In some embodiments, compositions comprise three or more cross-reactive allergenic proteins from Cupin protein family (Tables 5 and 10). In some embodiments, cross-reactive allergenic proteins are selected from Table 2. In some embodiments, compositions comprise one or more cross-reactive allergenic proteins selected from Tables 4-7 and 10-12. In some embodiments, compositions comprise one or more cross-reactive allergenic protein selected from the group consisting of Ber_e_2, Car_i_2, Fag_e_1, Fag_e_13S, Fag_t_13S_globulin, Jug_n_4, Lup_a_vicilin, Lup_an_1, Pin_k_2, Pis_s_1, Pis_s_2, Pis_v_2, Ses_i_3, Ses_i_6, Ses_i_7, Sin_a_2, Vig_r_2, Car_i_4, Gly_m_5, Gly_m_6, Jug_r_4, Len_c_1 and Pru_du_6, or any combination thereof. In some embodiments, compositions comprise at least two cross-reactive allergenic proteins selected from the group consisting of Ber_e_2, Car_i_2, Fag_e_1, Fag_e_13S, Fag_t_13S_globulin, Jug_n_4, Lup_a_vicilin, Lup_an_1, Pin_k_2, Pis_s_1, Pis_s_2, Pis_v_2, Ses_i_3, Ses_i_6, Ses_i_7, Sin_a_2, Vig_r_2, Car_i_4, Gly_m_5, Gly_m_6, Jug_r_4, Len_c_1 and Pru_du_6.

In some embodiments, compositions comprise one or more whole (complete) food sources (for example a whole food flour or powder, or protein extracts from a whole food source), for example, food sources provided in Table 8.

In some embodiments, cross-reactive allergenic proteins and cross-reactive epitope peptides of the present disclosure are used to prepare compositions, kits, vaccines and T-cell peptide therapies as described in detail below and also to make tests and assays, such as diagnostic assays, cross-reactive IgE assays, etc. Such cross-reactive allergenic proteins may be present in a composition as isolated or extracted proteins or as recombinant, or synthesized full-length proteins. In some embodiments, compositions are single allergen immunotherapeutic compositions. In some embodiments, compositions are multiple allergen immunotherapeutic compositions. In some embodiments, single allergen immunotherapeutic compositions comprise a cross-reactive allergenic protein (e.g., an identified allergenic protein, or an allergenic protein from any of the identified protein families such as Cupin protein family). In some embodiments, a multiple allergen immunotherapeutic composition comprises two or more allergenic proteins. Each of the two or more allergenic proteins may, for example, be from a single protein family or from 2 different protein families. Two or more allergenic proteins can be identified allergenic proteins from a single food source or they can be from different food sources. Allergenic proteins may, for example, be identified allergenic proteins (e.g., from Table 2), cross-reactive allergenic proteins from the same protein family, or cross-reactive proteins from a different protein family. In some multiple allergen immunotherapeutic compositions, no allergenic protein listed in Table 2 is included. Allergenic proteins may be full-length proteins or peptides or fragments thereof. A single allergen immunotherapeutic composition may comprise one or more cross-reactive epitope peptides from a single allergenic protein.

In some embodiments, provided herein are cross-reactive epitope peptides and compositions comprising the same. In some embodiments, cross-reactive epitope peptides are MHC-binding T-cell epitopes. In some embodiments, cross-reactive epitopes peptides are MHC-II-binding T-cell epitope peptides located in cross-reactive allergenic proteins in the Cupin protein family. Examples of MHC-II-binding T-cell epitope peptides include but are not limited to the MHC-II-binding T-cell epitope peptides provided in Tables 13-18. In some embodiments, cross-reactive epitopes peptides are B-cell epitope peptides located in cross-reactive allergenic proteins in the Cupin protein family. Examples of B-cell epitope peptides include but are not limited to the MHC-II-binding T-cell epitope peptides provided in Table 19. In some embodiments, one or more cross-reactive epitope peptides are derived from Ber_e_2, Car_i_2, Fag_e_1, Fag_e_13S, Fag_t_13S_globulin, Jug_n_4, Lup_a_vicilin, Lup_an_1, Pin_k_2, Pis_s_1, Pis_s_2, Pis_v_2, Ses_i_3, Ses_i_6, Ses_i_7, Sin_a_2, Vig_r_2, Car_i_4, Gly_m_5, Gly_m_6, Jug_r_4, Len_c_1 and Pru_du_6, or any combination thereof. In some embodiments, a composition comprises at least 4 cross-reactive epitope peptides, for example, derived from Ber_e_2, Car_i_2, Fag_e_1, Fag_e_13S, Fag_t_13S_globulin, Jug_n_4, Lup_a_vicilin, Lup_an_1, Pin_k_2, Pis_s_1, Pis_s_2, Pis_v_2, Ses_i_3, Ses_i_6, Ses_i_7, Sin_a_2, Vig_r_2, Car_i_4, Gly_m_5, Gly_m_6, Jug_r_4, Len_c_1 and/or Pru_du_6.

In some embodiments, provided herein are compositions comprising one or more cross-reactive epitope peptides. Contemplated ross-reactive epitope peptides may be synthetic, recombinant or natural. In some embodiments, the present disclosure provides one or more recombinant or synthetic cross-reactive epitopes.

In some embodiments, a cross-reactive epitope peptide is from about 5 to about 200 amino acids in length, about 6 to about 150 amino acids in length, about 7 to about 100 amino acids in length, about 8 to about 50 amino acids in length, or about 8 to about 35 amino acids in length. In some embodiments, a cross-reactive epitope peptide is from about 5 to 25 amino acids in length. In some embodiments, a cross-reactive epitope peptide is from about 8 to about 20 amino acids in length. In some embodiments, a cross-reactive epitope peptide is 20 amino acids in length. In some embodiments, a cross-reactive epitope peptide is 14 amino acids in length. In some embodiments, a cross-reactive epitope peptide is 15 amino acids in length. In some embodiments, a cross-reactive epitope peptide is a T-cell epitope, e.g., a MHC-II-binding T-cell epitope.

The disclosure also provides polynucleotides or oligonucleotides encoding cross-reactive allergenic proteins and cross-reactive epitope peptides, for example, those disclosed herein.

In some embodiments, provided herein are isolated cross-reactive epitope peptides comprising one or more cross-reactive epitopes that bind to one or more members of an MHC or HLA binding region. In some embodiments, an MHC is MHC class II or HLA class II. In some embodiments, cross-reactive epitope peptides which bind to MHC binding regions may be intracellularly located. In some embodiments, cross-reactive epitope peptide that bind to MHC are partially extracellularly located or partially intracellularly located. In some embodiments, a cross-reactive epitope peptide that binds to MHC may be located extracellularly. In some embodiments, MHC binding peptides are T-cell peptides. In some embodiments, T-cell epitopes are linear epitopes. In some embodiments, MHC-II-binding T-cell epitopes are linear epitopes.

In some embodiments, a cross-reactive epitope peptide that binds to an WIC is from about 5 to about 200 amino acids in length. In some embodiments, a cross-reactive epitope peptide is from about 6 to about 150 amino acids in length. In some embodiments, a cross-reactive epitope peptide that binds to an MHC is from about 5 to about 25 amino acids in length. In some embodiments, a cross-reactive epitope peptide that binds to an MHC is from about 8 to about 20 amino acids in length. In some embodiments, a peptide that binds an MHC is 8, 9, 10, 11, 12, 13, 14, 14, 15, 16, 17, 18, 19 or 20 amino acids in length. In some embodiments, a peptides is 15 amino acids in length. In some embodiments, a cross-reactive epitope peptide is 14 amino acids in length.

In some embodiments, several cross-reactive epitope peptides comprising MHC-binding T-cell epitopes (such as 2, 3, 4, 6, or 50 peptides) are attached as contiguous peptide molecule. In some embodiments, several peptides may be linked as polypeptides. In some embodiments, an MHC is a human WIC (HLA). In some embodiments, an WIC is a mouse WIC. In some embodiments, a cross-reactive epitope peptide has at least 70% identity with at least 80% coverage over the length of the peptide. In some embodiments, a cross-reactive epitope peptides has at least 80% identity with at least 70% coverage over the length of the peptide. In some embodiments, a cross-reactive epitope peptide that binds to an WIC is shared by or common to 2 or more, 3 or more, 4 or more, 5 or more, 6 or more, 7 or more, 8 or more, 9 or more, or 10 or more allergenic proteins within a protein family. Non-limiting exemplary cross-reactive MHC-binding T-cell peptides are provided in Tables 13-18, 20, and 22. Non-limiting exemplary cross-reactive B-cell peptides are provided in Tables 19, 21, and 23.

In some embodiments, a cross-reactive WIC-binding T-cell epitope on an allergenic protein is cross-reactive with 1 or more identified allergenic proteins within the protein family. In some embodiments, a cross-reactive MHC-binding T-cell epitope on an allergenic protein is cross-reactive with 2 or more, 3 or more, 4 or more, 5 or more, 6 or more, 7 or more, 8 or more, 9 or more, or 10 or more other allergenic proteins within a protein family. In some embodiments, a cross-reactive MHC-binding epitope on a cross-reactive protein is cross-reactive with 2 or more, 3 or more, 4 or more, 5 or more, 6 or more, 7 or more, 8 or more, 9 or more, or 10 or more cross-reactive allergenic proteins within a protein family. In some embodiments, a cross-reactive WIC-binding T-cell epitope on a cross-reactive allergenic protein is cross-reactive with all allergenic proteins within a protein family.

In some embodiments, a cross-reactive T-cell epitope peptide binds to one or more MHC binding regions with a binding affinity for at least one WIC binding region of ≤500 nM Kd, ≤400 nM Kd, ≤300 Kd, ≤200 nM Kd, ≤150 nM Kd, ≤100 nM Kd, ≤75 nM Kd, ≤50 nM Kd, ≤25 nM Kd, ≤20 nM Kd, ≤15 nM Kd, ≤10 nM Kd, ≤5 nM Kd, or ≤1 nM Kd. In some embodiments, a cross-reactive epitope peptide that binds to one or more WIC binding regions has a binding affinity for at least one WIC-II of ≤100 nM Kd. Exemplary cross-reactive peptides that bind one or more WIC-binding regions are provided in Tables 13-18 (Cupin MHC-binding T-cell epitopes). In some embodiments, a cross-reactive epitope peptide that binds to one or more WIC binding regions has a binding affinity for at least one WIC-II of ≤50 nM Kd. In some embodiments, a cross-reactive epitope peptide that binds to one or more WIC binding regions is a weak affinity binder, a medium affinity binder or a high affinity binder. In some embodiments, a binding affinity is determined by application of MHC binding predictive tools and algorithms such as TEPI on the server available on the world wide web at IEDB.org.

Further, information on the type of WIC molecules (e.g., WIC-II DR, DQ, etc. and variants thereof) that contemplated experimentally-validated T-cells and/or B-cells bind can be obtained and analyzed as described in Example 5. The present disclosure provides compositions, kits, vaccines, antibodies and diagnostic assays that are matched to subjects having a particular HLA allele or haplotype. In some embodiments, peptides of the present disclosure comprise one or more epitopes that bind with a strong affinity to from 1 to 150, 1 to 100, 1 to 80, 1 to 75, 10 50, 1 to 30, 1 to 25, 1 to 20, 1 to 15, 1 to 10, 1 to 5, 1 to 2, 2 or 1 HLA alleles and/or their variants. In some embodiments, peptides of the present disclosure comprise one or more epitopes that bind with a medium affinity to from 1 to 150, 1 to 100, 1 to 80, 1 to 75, 10 50, 1 to 30, 1 to 25, 1 to 20, 1 to 15, 1 to 10, 1 to 5, 1 to 2, 2 or 1 HLA allele or haplotypes. In some embodiments, peptides of the present disclosure comprise one or more epitopes that bind with a weak affinity to from 1 to 150, 1 to 100, 1 to 80, 1 to 75, 10 50, 1 to 30, 1 to 25, 1 to 20, 1 to 15, 1 to 10, 1 to 5, 1 to 2, 2 or 1 HLA haplotypes and/or their variants.

Disclosed herein, for example, are cross-reactive WIC-II binding T-cell epitopes present in cross-reactive allergenic proteins that are able to bind large number of MHC molecules (from 1 to over 150 MHC variants) (See Tables 13-18, 20, and 22).

To obtain degenerate cross-reactive MHC-II-binding T-cell epitope peptides, methods of the present disclosure can be used to analyze the allergenic proteins within Tables 4-7. Tables 13-18, 20, and 22 provide a summary of disclosed cross-reactive MHC-II-binding T-cell epitopes. Binding affinity for epitopes in Tables 13-18, 20, and 22 is at least ≤100 Kd. It is expected that MHC degeneracy observed with disclosed cross-reactive MHC-II-binding T-cell epitope peptides as well as the medium-high and high affinity binding of MHC molecules by disclosed cross-reactive epitopes may, for example, be advantageous for use in diagnostics and therapeutics such as vaccines and T-cell peptide therapy, multiple allergen immunotherapy, etc. Further, one of skill in the art will appreciate that the compositions comprising the cross-reactive allergenic proteins and cross-reactive epitopes of the present disclosure may, for example, be matched to a subject having a particular MHC haplotype and/or variant, wherein the match is determined by the binding affinity of a particular cross-reactive T-cell epitope or epitopes to the HLA allele of the subject.

In some embodiments, a cross-reactive MHC-binding T-cell epitope peptide also includes a B-cell epitope. In some embodiments, a B-cell epitope is also cross-reactive with one or more allergenic proteins. Peptide sequences containing both a B-cell epitope and a MHC-binding T-cell epitope may overlap or be adjacent to each other. Cross-reactive epitope peptides sequences containing both the MHC-binding T-cell epitope and a B-cell epitope within a defined area of overlap are readily ascertainable by mapping (e.g., by alignment algorithm such as FASTA, BLAST, MegAlign) identified epitopes within a source protein, for example, a cross-reactive allergenic Cupin protein as described in Example 5. In some embodiments, a B-cell epitope and a MHC-binding T-cell epitope overlap or are adjacent. The overlap of sequences may be within about 1 to about 20 amino acids, about 2 to about 20 amino acids, about 3 to about 20 amino acids, about 1 to about 10 amino acids, about 2 to about 5 amino acids, about 3 to about 7 amino acids, or about 2 to about 8 amino acids.

In some embodiments, cross-reactive peptides are B-cell epitope peptides. In other embodiments, cross-reactive peptides are MHC-binding peptides.

The present disclosure provides, for example, a nucleic acid encoding one or more of the cross-reactive epitope peptides or cross-reactive allergenic proteins described herein (for example an identified allergenic protein of Table 2 and/or a cross-reactive allergenic protein in any of the identified protein families of Table 3 (for example, Cupin protein family)).

Cross-reactive epitopes and peptides comprising such cross-reactive epitopes may be further derivatized, for example, to include a hapten. Haptens or small low molecular weight molecules may be attached to cross-reactive epitope peptides or polypeptides comprising such cross-reactive epitopes. Haptens include but are not limited to fluorophores, biotin, and phosphate group linked to an amino acid (e.g., phosphothreonine, phosphotyrosine, etc.).

In some embodiments, a polypeptide includes a flanking sequence extending beyond the region comprising a cross-reactive T-cell epitope peptide and/or a cross-reactive B-cell epitope peptide. Such a flanking sequence may be used in assuring a synthetic version of the peptide is displayed in such a way as to represent a topological arrangement in its native state. Flanking sequences may be included to allow multiple peptides to be arranged together as epitopes that occur adjacent to each other in a native protein. A flanking sequence may be used to facilitate expression as a fusion polypeptide, for example an immunoglobulin Fc region fusion polypeptide to ensure secretion. In such embodiments where flanking regions are included the flanking regions may comprise, for example, from 1-20, from 1-50, from 10-20, 20-30 or 40-50 amino acids. Flanking sequences may be included on either or both of the N terminal end or the C terminal end of a cross-reactive epitope peptide. The location of each cross-reactive epitope peptide in a native protein may be determined by referring to the Genbank/SWISS-PROT coordinates. In some embodiments, synthetic cross-reactive epitope peptides of the present disclosure comprise the entire proteins of which the cross-reactive epitope peptides identified by the specific SEQ ID NO is a part.

Also contemplated are polynucleotide and/or polypeptide sequences that have a high degree of homology to the cross-reactive epitope peptides (or polynucleotides and oligonucleotides encoding them) disclosed herein. One of skill in the art will appreciate that sequences provided herein can be altered, for example by substituting one or more amino acids in the sequences with a different sequence or in a flanking region. Such mutated or variant sequences are within the scope of the present disclosure. The substitutions may be conservative or non-conservative. Accordingly, in some embodiments, the present disclosure provides proteins, polypeptide and peptide sequences that share at least 70%, 71%, 72%, 73%, 74%, 75%, 75%, 76%, 77%, 78%, 79%, 80%, 81%, 82%, 83%, 84%, 85%, 86%,87%,88%, 89%, 90%, 91%, 92%, 93%, 94%, 95%, 96%, 97%, 98%, or 99% identity with a cross-reactive epitope (or a polypeptide or a polynucleotide comprising the cross-reactive epitope) set forth in Tables 13-23. In some embodiments, the variant sequences have about 1, 2, 3, 4, 5, 6, 7, 8, 9, or 10 amino acid substitutions, or a range of substitutions form about 1 to about 10 substitutions, for example, from about 1 to about 5 substitutions, about 2 to about 4 substitutions, about 3 to about 6 substitutions, about 4 to about 9 substitutions, etc.

In some embodiments, compositions of the present disclosure include one or more excipients as further described below.

### VII. VACCINES; T-CELL PEPTIDE THERAPY

In some embodiments, provided herein are vaccines comprising one or more of cross-reactive epitopes disclosed herein. In some embodiments, a vaccine is matched to a subject with a particular MHC haplotype. In some embodiments, a vaccine comprises one or more of the peptides or polypeptides disclosed herein and an adjuvant. In some embodiments, a vaccine comprises a recombinant or synthetic allergenic protein or a cross-reactive epitope peptide disclosed herein (for example, a cross-reactive epitope peptide that binds to at least one matched MHC/HLA). In some embodiments, a vaccine comprises one or more cross-reactive T-cell epitope peptides. In some embodiments, a vaccine comprises one or more B-cell epitope peptides and/or MHC-binding epitopes.

Contemplated vaccines may include a single peptide or protein or any combination of peptides or proteins. In some embodiments, a vaccine comprises a full-length cross-reactive allergenic protein or a polynucleotide or nucleic acid encoding the full-length cross-reactive allergenic protein. In some embodiments, a vaccine comprises two or more cross-reactive allergenic proteins.

Contemplated vaccines may be formulated for oral, parenteral, nasal, and/or topical administration to individuals in need thereof. Vaccines may be, for example, protein or peptide vaccines, or nucleotide vaccines (e.g., DNA vaccines, mRNA vaccines, etc.). Contemplated vaccines comprise one or more cross-reactive allergenic proteins and/or one or more cross-reactive epitope peptides of the present disclosure. Vaccines and compositions comprising the vaccines may, for example, be able to elicit an immune response in a subject, a biological sample or a cell (e.g., manifested by the presence (or absence where applicable) of an immune marker such as cross-reactive IgE and/or IgG antibody expression, cytokine release, T-cell anergy, etc.). Contemplated vaccines may, for example, provide immunogenic and/or immunotherapeutic compositions that may, for example, be able to induce tolerance in a subject, a biological sample or a cell (e.g., manifested by the presence (or absence where applicable) of an immune marker such as cross-reactive IgE and/or IgG antibody expression, cytokine release, T-cell anergy, etc.).

In some embodiments, a contemplated multiple allergen composition or vaccine may include at least 2, 3, 4, 5, 6, 8, 10, 15, 20, 25, 30, 40, 50, 60, 70, 80, 90, 100, 200, 300, 400, or 500 distinct cross-reactive allergenic proteins, peptides, or polynucleotides encoding the same. In some embodiments, a contemplated multiple allergen composition or vaccine may include two or more distinct cross-reactive allergenic proteins, peptides, or polynucleotides encoding the same. In some embodiments, contemplated multiple allergen composition or vaccine may include at least 6 distinct cross-reactive allergenic proteins, peptides, or polynucleotides encoding the same. In some embodiments, a composition or vaccine may include allergenic proteins selected from the group consisting of a shrimp allergen, a cod allergen, a salmon allergen, a hen's egg allergen, a cow's milk allergen, a peanut allergen, a sesame allergen, a soy allergen, a pecan allergen, a cashew allergen, a hazelnut allergen, a walnut allergen, a pistachio allergen, an almond allergen, and a wheat allergen. In some embodiments, a composition or vaccine may include identified allergens listed in Table 2. In some embodiments, a composition or vaccine may include proteins selected from the protein families provided in Table 3 or Table 4. In some embodiments, a composition or vaccine may include proteins selected from the Cupin family (Table 5, Table 10). In some embodiments, a composition or vaccine may include one or more of: Ber_e_2, Car_i_2, Fag_e_1, Fag_e_13S, Fag_t_13S_globulin, Jug_n_4, Lup_a_vicilin, Lup_an_1, Pin_k_2, Pis_s_1, Pis_s_2, Pis_v_2, Ses_i_3, Ses_i_6, Ses_i_7, Sin_a_2, Vig_r_2, Car_i_4, Gly_m_5, Gly_m_6, Jug_r_4, Len_c_1 and Pru_du_6.

In some embodiments, a contemplated multiple allergen composition or vaccine may include at least 2, 3, 4, 5, 6, 8, 10, 15, 20, 25, 30, 40, 50, 60, 70, 80, 90, 100, 200, 300, 400, 500, 600, 700, 800, 900, 1000, 2000, 3000, 4000, 5000, 6000, 7000, 8000, 9000, or 10,000 cross-reactive epitope peptides. In some embodiments, a contemplated multiple allergen composition or vaccine may include at most 2, 3, 4, 5, 6, 8, 10, 15, 20, 25, 30, 40, 50, 60, 70, 80, 90, 100, 200, 300, 400, 500, 600, 700, 800, 900, 1000, 2000, 3000, 4000, 5000, 6000, 7000, 8000, 9000 or 10,000 cross-reactive epitope peptides. In some embodiments, a composition or vaccine may include at least 4 distinct cross-reactive epitopes derived from a single allergenic protein or a single allergenic source (e.g., allergenic food source).

In some embodiments, a contemplated multiple allergen composition or vaccine may include at least 1, 2, 3, 4, 5, 6 7, 8, 9, 10, 12, 15, 20, 25, 30, 40, 50, 60, 70, 80, 90, or 100 or more cross-reactive epitope peptides from a single cross-reactive allergenic protein or an identified or reference allergenic protein. In some embodiments, a contemplated multiple allergen composition or vaccine may include at least 1, 2, 3, 4, 5, 6 7, 8, 9, 10, 12, 15, 20, 25, 30, 40, 50, 60, 70, 80, 90, or 100 or more cross-reactive epitope peptides from 2 or more distinct identified or reference allergenic proteins. In some embodiments, a contemplated multiple allergen composition or vaccine may include at least 1, 2, 3, 4, 5, 6 7, 8, 9, 10, 12, 15, 20, 25, 30, 40, 50, 60, 70, 80, 90, or 100 peptides from at least 6 distinct identified or reference allergenic proteins. In some embodiments, a contemplated multiple allergen composition or vaccine may include at least 1, 2, 3, 4, 5, 6 7, 8, 9, 10, 12, 15, 20, 25, 30, 40, 50, 60, 70, 80, 90, or 100 or more cross-reactive epitope peptides from 2 or more, 3 or more, 4 or more, 5 or more, 6 or more, 7 or more, 8 or more, 9 or more, 10 or more, 11 or more, 12 or more, 13 or more, 14 or more, or 15 or more of the allergenic proteins of Table 2. In some embodiments, a contemplated multiple allergen composition or vaccine may include at least 1, 2, 3, 4, 5, 6 7, 8, 9, 10, 12, 15, 20, 25, 30, 40, 50, 60, 70, 80, 90, or 100 or more cross-reactive epitope peptides from one or more food sources selected from the group consisting of shrimp, cod, salmon, hen's egg, cow's milk, peanut, sesame, soy, pecan, cashew, hazelnut, walnut, pistachio, almond, and wheat. In some embodiments, a contemplated multiple allergen composition or vaccine may include at least 1, 2, 3, 4, 5, 6 7, 8, 9, 10, 12, 15, 20, 25, 30, 40, 50, 60, 70, 80, 90, or 100 or more cross-reactive epitope peptides from one or more allergens selected from the group consisting of a shrimp allergen, a cod allergen, a salmon allergen, a hen's egg allergen, a cow's milk allergen, a peanut allergen, a sesame allergen, a soy allergen, a pecan allergen, a cashew allergen, a hazelnut allergen, a walnut allergen, a pistachio allergen, an almond allergen, and a wheat allergen.

In some embodiments, a contemplated multiple allergen composition or vaccine may include at least 1, 2, 3, 4, 5, 6 7, 8, 9, 10, 12, 15, 20, 25, 30, 40, 50, 60, 70, 80, 90, or 100 or more cross-reactive epitope peptides from 2 or more, 3 or more, 4 or more, 5 or more, 6 or more, 7 or more, 8 or more, 9 or more, 10 or more, 11 or more, 12 or more, 13 or more, 14 or more, or 15 or more distinct allergenic proteins. The allergenic proteins may, for example, be selected from one or more protein families (such as those listed in Table 3, Table 4). In some embodiments, a composition or vaccine comprises one or more Cupin protein family allergenic proteins or peptides. In some embodiments, a composition or vaccine may include at least 1, 2, 3, 4, 5, 6 7, 8, 9, 10, 12, 15, 20, 25, 30, 40, 50, 60, 70, 80, 90, or 100 or more cross-reactive epitope peptides from 2 or more, 3 or more, 4 or more, 5 or more, 6 or more, 7 or more, 8 or more, 9 or more, 10 or more, 11 or more, 12 or more, 13 or more, 14 or more, or 15 or more distinct cross-reactive Cupin allergenic proteins (e.g., those listed in Table 5 or 10). In some embodiments, a composition or vaccine comprises one or more cross-reactive epitope peptides derived from: Ber_e_2, Car_i_2, Fag_e_1, Fag_e_13S, Fag_t_13S_globulin, Jug_n_4, Lup_a_vicilin, Lup_an_1, Pin_k_2, Pis_s_1, Pis_s_2, Pis_v_2, Ses_i_3, Ses_i_6, Ses_i_7, Sin_a_2, Vig_r_2, Car_i_4, Gly_m_5, Gly_m_6, Jug_r_4, Len_c_1 or Pru_du_6, or any combination thereof. In some embodiments, a composition or vaccine includes 4 or more cross-reactive epitopes each a Cupin family protein (for example, from two or more, three or more, or up to 37 Cupin family proteins).

In some embodiments, the present disclosure provides a vector comprising a polynucleotide and/or an oligonucleotide that encodes a cross-reactive allergenic protein (for example, a cross-reactive allergenic protein disclosed herein) and/or a cross-reactive epitope peptide (for example, a cross-reactive epitope peptide disclosed herein). In some embodiments, the present disclosure provides a cell (for example, a host cell) comprising a contemplated vector.

### VIII. PREPARATION OF PROTEINS, PEPTIDES AND VACCINES

Cross-reactive allergenic proteins and/or cross-reactive epitope peptides may be prepared using any suitable method provided herein or known in the art.

Chemical peptide synthesis methods are well described and peptides can be ordered from companies such as Applied Biosystems, and others. Peptide synthesis can be performed as either solid phase peptide synthesis (SPPS) or solution phase peptide synthesis. The best known SPSS methods are t-Boc and F-moc solid phase chemistry. During peptide synthesis several protecting groups may, for example, be used. For example, hydroxyl and carboxyl functionalities may, for example, be protected by t-butyl group; lysine and tryptophan may, for example, be protected by t-Boc group; asparagine, glutamine cysteine and histidine may, for example, be protected by trityl group; and arginine may, for example, be protected by pbf group. In some embodiments, such protecting groups are left on the peptide after synthesis.

Peptides may, for example, be linked together to form longer peptides (for example to include two or more cross-reactive T-cell epitopes) using chemoselective coupling of unprotected peptide fragments (as described by Kent et al. (1992) Int. J. Pept. Protein Res. 40:180-193 and reviewed in Tam et al. (2001) BioPolymers 60:194-205) which provides the potential to achieve protein synthesis beyond the scope of SPPS. Many proteins and peptides of length 100-300 have been synthesized by this method.

Alternatively, cross-reactive allergenic proteins and/or cross-reactive epitope peptides of the present disclosure may be synthesized using traditional biochemical purification methods and/or by recombinant technology, using nucleic acid molecules which encode cross-reactive allergenic proteins and/or cross-reactive epitope peptides of the present disclosure. Such nucleic acid molecules (e.g., DNA molecules) maybe readily prepared using an automated synthesizer (e.g., an automated DNA synthesizer) utilizing the well-known codon-amino acid relationship of the genetic code.

Amino acid sequences of cross-reactive allergenic proteins and/or cross-reactive epitope peptides of the present disclosure may be reverse translated into corresponding nucleic acid sequences or nucleic acid sequences may be obtained from Genbank/SwissProt and other databases. Nucleic acid sequences may be optimized by applying codon optimization techniques known in the art for particular expression systems to be used. Polynucleotide sequences encoding cross-reactive allergenic proteins and/or cross-reactive epitope peptides may be cloned into an appropriate expression vector and expressed in a host cell. Suitable host cells include bacteria (e.g., E. coli), fungal cells (e.g., S. cerevisiae), insect cells (e.g., Sf9, Sf21, Hi5 cell lines), animal cells (e.g., CHO, HEK293, HeLA), and plant cells (e.g., N. tabacum, A. thaliana), and the like.

Methods well known to one of skill in the art may be used to construct cloning vectors containing nucleic acid sequence encoding allergenic proteins of the present disclosure, and other transcriptional and translational control elements. Exemplary techniques are described in Sambrook et al. (1989) Molecular Cloning, a Laboratory Manual, Cold Spring Harbor Press, Plainview N.Y.; Ausubel et al. (1989) Current Protocols in Molecular Biology, John Wiley & Sons, New York, N.Y.; and Green et al. (1997) Genome Analysis, A Laboratory Manual, Cold Spring Harbor Press, Plainview N.Y. In some cases, sequences generated by methods of the present disclosure may be synthetically manufactured prior to cloning into expression vectors. Polynucleotides may be provided in the form of expression cassettes which include control sequences operably linked to the inserted sequences. Expression cassettes in turn are typically provided within vectors (e.g., plasmids, or recombinant viral vectors) which may, for example, be suitable for use as reagents for nucleic acid immunization. Such an expression cassette may be administered to a host subject. In some embodiments, a polynucleotide is prepared and administered using a genetic vector. Suitable vectors may be any vectors which are capable of carrying a sufficient amount of genetic information and allowing expression of a protein, polypeptide, and/or peptide of the present disclosure.

The present disclosure provides expression vectors comprising disclosed polynucleotide sequences. The present disclosure also provides a vector for use in preventing or treating an allergy (e.g., a food allergy) or a food-intolerance comprising four or more polynucleotide sequences which each encode a different peptide, polypeptide, and/or protein described herein. A contemplated vector may, for example, comprise 4, 5, 6, 7, 8 or more polynucleotide sequences which encode different peptides, polypeptides, and/or proteins of the present disclosure.

Vectors, e.g., plasmid vectors will typically have appropriate initiators, promotors, enhancers, and other elements such as polyadenylation signals which may be necessary and which are positioned in the correct orientation in order to allow for expression of a protein, polypeptide, a peptide of the present disclosure. Proteins, polypeptides, and peptides of the present disclosure may be provided by delivering such a vector to a cell and allowing expression to occur from the vector. In some embodiments, a polynucleotide of the present disclosure is operably linked to a control sequence which is capable of providing for expression of a coding sequence by a host cell.

Cross-reactive allergenic proteins and/or cross-reactive epitope peptides may, for example, be prepared using a variety of synthetic or enzymatic schemes known in the art. Where short peptides are desired, such peptides may, for example, be prepared using automated peptide synthesis in solution or in solid support in accordance with conventional techniques. Various automated synthesizers are commercially available and may, for example, be used in accordance with known protocols and manufacturers' instructions. See, for example, Stewart and Young, Solid Phase Peptide Synthesis, 2d. ed., Pierce Chemical Co., (1984); Tam et al., J. Am. Chem. Soc., 105:6442, (1983); Merrifield, Science, 232: 341-347, (1986); Barany and Merrifield, The Peptides, Gross and Meienhofer, eds, Academic Press, New York, 1-284, (1979); Fields, (1997) Solid-Phase Peptide Synthesis. Academic Press, San Diego.); Andersson et al., Large-scale synthesis of peptides. Biopolymers (Pept. Sci.), 55, 227-250 (2000); Burgess et al., DiSSiMiL: Diverse Small Size Mini-Libraries applied to simple and rapid epitope mapping of a monoclonal antibody. J. Pept. Res., 57, 68-76, (2001); and Peptides for the New Millennium, Fields, J. P. Tam & G. Barany (Eds.), Kluwer Academic Publisher, Dordrecht.

Cross-reactive allergenic proteins and/or cross-reactive epitope peptides may, for example, also be modified, and such modification may be carried out on a synthesizer with minor modifications. For example, an amide could be added at the C-terminus of a peptide; an acetyl group could be added to the N-terminus of a peptide; and/or biotin, stearate, and other modifications could also be added to the N-terminus of a peptide.

Cross-reactive allergenic proteins and/or cross-reactive epitope peptides may, for example, be expressed in bacteria, such as lactococcus and lactobacillus, or in virus or virus-like particles, for example, for use as vaccines.

In some embodiments, cross-reactive allergenic proteins and/or cross-reactive epitope peptides are incorporated into other carriers known in the art. For example, cross-reactive allergenic proteins and/or cross-reactive epitope peptides may be conjugated or otherwise attached to a carrier protein, such as keyhole limpet hemocyanin (KLH), bovine serum albumin, ovalbumin, thyroglobulin, etc. In other embodiments, cross-reactive allergenic proteins and/or cross-reactive epitope peptides may be fused to an Fc region of an immunoglobulin, for example, for delivery to a mucosal site bearing corresponding receptors.

Cross-reactive allergenic proteins and/or cross-reactive epitope peptides of the present disclosure may be administered as part of a single allergen therapy or multiple allergen therapy. Cross-reactive allergenic proteins and/or cross-reactive epitope peptides may be administered as a primary therapy or as adjuvant therapy to another therapy. Cross-reactive allergenic proteins and/or cross-reactive epitope peptides may be administered to treat a target or primary food allergy or food intolerance or a cross-reactive epitope-sensitive condition.

Vaccines of the present disclosure may be prepared, for example, by combining one or more cross-reactive peptides with a pharmaceutically acceptable carrier. Methods for preparation of such compositions are further described below.

### IX. BINDING PARTNERS; ANTIBODIES

In one aspect, the present disclosure relates to the identification of antigen binding proteins or binding partners that recognize and bind to one or more cross-reactive allergenic proteins and/or cross-reactive epitopes of the present disclosure. A binding partner may be any agent that recognizes and binds to one or more cross-reactive allergenic proteins and/or cross-reactive epitopes of the present disclosure. Examples of binding partners include, but are not limited to, antibodies, B-cell receptor (BCR) expressing cells (e.g., B-cells, host cells expressing BCRs), T-cell receptor (TCR) expressing cells (e.g., T-cells, host cells expressing TCRs, etc.), MHC-expressing cells (e.g., MHC-I/HLA-I- or MHC-II/HLA-II-expressing cells) such as monocytes, antigen presenting cells (APC) such as macrophages, dendritic cells, B-cells, non-professional APCs, etc., and the like. In some embodiments, a binding partner is a B-cell receptor expressing cell such as a B-cell or a host cell expressing a B-cell receptor. In other embodiments, a binding partner is an MHC-expressing cell such as an antigen presenting cell (e.g. monocytes, macrophages, dendritic cells, non-professional APCs known in the art, and the like). In some embodiments, a binding partner is an antibody.

It is contemplated that cross-reactive allergenic proteins and/or cross-reactive epitope peptides (e.g., B-cell epitopes, MHC-binding epitopes, and/or T-cell epitopes) which are recognized and specifically bound by binding partners may, for example, be identified by in silico methods described herein. Accordingly, provided herein in some embodiments are assays or methods for identifying antibodies that bind (e.g., specifically bind) to one or more cross-reactive allergenic proteins and/or peptides (e.g., including a B-cell epitope, T-cell epitope, and/or MHC-binding epitope). In some embodiments, contemplated methods identify antibodies (e.g., specific antibodies) that bind to B-cell epitope peptides.

In some embodiments, provided herein are methods of identifying one or more antibodies (e.g., specific IgE or specific IgG antibodies) that, for example, are useful for the detection of allergenic food antigens and/or cross-reactive non-food allergens. Contemplated methods comprise: correlating one or more allergenic proteins with other allergenic proteins in the same protein family to identify cross-reactive B-cell epitope peptides; and identifying IgE or IgG antibodies (e.g., specific IgE or specific IgG antibodies) that bind to identified cross-reactive B-cell epitope peptides. One or more allergenic proteins may, for example, be correlated with other allergenic proteins in the same protein family to identify cross-reactive specific B-cell epitope peptides by providing a map of identified proteins in the same protein family, mapping full-length homology within a protein family to identify cross-reactive proteins, and/or mapping homology of cross-reactive proteins at the T-cell or B-cell epitope level. In some embodiments, a map of pairwise identity and similarity between an allergenic protein and other allergenic proteins within the same protein family is generated to identify cross-reactive allergenic proteins. The present disclosure provides in silico methods for determining MHC binding potential of T-cells. Exemplary MHC-II-binding T-cell epitopes (Tables 13-18, 20, and 22) and B-cell epitopes (Tables 19, 21, and 23) are provided to which one or more binding partners may bind, for example, specific antibodies such as IgE and/or IgG.

In some embodiments, a binding partner (e.g., an IgE or IgG antibody, e.g., a specific IgE or IgG antibody) may recognize and bind to one or more allergenic proteins present in a protein family selected from the group consisting of: Tropomyosin, EF hand family (Beta-parvalbumin), ATP:guanido phosphotransferase (Arginine Kinase), Triosephosphate isomerase, Fructose bisphosphate aldolase class I, Alpha/beta casein, Lipocalin, Kappa-casein, Transferrin, C-type lysozyme/alpha-lactalbumin family, Serum albumin, Enolase, Serpin serine protease inhibitor, Kazal-type serine protease inhibitor, Collagen, Lipoprotein, Prolamin superfamily, Cupin, Profilin, Bet v 1 family, Oleosin, Isoflavone reductase family, Plant defensin, 60S acidic ribosomal protein, Heat shock protein Hsp70, Fe/Mn superoxide dismutase, Papain-like cysteine protease, Thaumatin-like protein, Unclassified, GILT family, Hevein-like and class I/II chitinase, Zinc finger, C3HC4 type (RING finger), SCAI family, Peroxidase, Serpin serine protease inhibitor, Thionin, Thioredoxin, Transcription elongation factor 1, Lipid transfer protein, and Triosephosphate isomerase protein family, or any combination thereof (Table 3).

In some embodiments, a binding partner (e.g., an IgE or IgG antibody, e.g., a specific IgE or IgG antibody) may recognize and bind to one or more allergenic proteins selected from the group consisting of a shrimp allergen, a cod allergen, a salmon allergen, a hen's egg allergen, a cow's milk allergen, a peanut allergen, a sesame allergen, a soy allergen, a pecan allergen, a cashew allergen, a hazelnut allergen, a walnut allergen, a pistachio allergen, an almond allergen, and a wheat allergen. In some embodiments, a binding partner (e.g., an IgE or IgG antibody, e.g., a specific IgE or IgG antibody) may recognize and bind one or more specific allergenic proteins provided in Table 2.

In some embodiments, a binding partner (e.g., an IgE or IgG antibody, e.g., a specific IgE or IgG antibody) may recognize and bind to one or more cross-reactive allergenic proteins set forth in Tables 4-7 and/or 10-12. In some embodiments, a binding partner (e.g., an IgE or IgG antibody, e.g., a specific IgE or IgG antibody) may recognize and bind to one or more Cupin protein selected from the group consisting of: Pru du 6, Jug n 2, Jug n 4, Ana o 1, Ana o 2, Jug r 2, Jug r 4, Cor a 11, Cor a 9, Ara h 1, Ara h 3, Car i 2, Car i 4, Pis v 2, Pis v 3, Pis v 5, Ses i 3, Ses i 6, Ses i 7, Gly m 5, Gly m 6, Gly m Bd, Ber e 2, Coc n 1, Fag e 13s globulin, Fag e 3, Fag e legumin, Pis s 1, Pis s 2, Act d 12, Pin k 2, Len c 1, Vig r 2, Lup an 1, Fag t 13S globulin, Lup a vicilin, and Sin a 2. In some embodiments, a binding partner (e.g., an IgE or IgG antibody, e.g., a specific IgE or IgG antibody) may recognize and bind to one or more cross-reactive allergenic proteins or peptides set forth in Tables 4-7 and 10-23.

In some embodiments, a binding partner (e.g., an IgE or IgG antibody, e.g., a specific IgE or IgG antibody) may recognize and bind to a single cross-reactive allergenic protein. In some embodiments, a binding partner (e.g., an IgE or IgG antibody, e.g., a specific IgE or IgG antibody) may recognize and bind to two or more cross-reactive allergenic proteins.

In some embodiments, a binding partner (e.g., an IgE or IgG antibody, e.g., a specific IgE or IgG antibody) may recognize and bind to one or more of: a cross-reactive B-cell epitope peptides, an MHC-binding epitope peptides, and/or a T-cell epitope peptide. In some embodiments, a binding partner may recognize and bind one or more cross-reactive B-cell epitope peptides derived from a cross-reactive allergenic protein. In some embodiments, a binding partner may recognize and bind a B-cell epitope on a cross-reactive allergenic protein set forth in Tables 19, 21, and 23. In some embodiments, a binding partner may recognize and bind one or more cross-reactive T-cell epitope peptides derived a cross-reactive allergenic protein. In some embodiments, a binding partner may recognize and bind one or more cross-reactive MHC-binding epitope peptides derived from a cross-reactive allergenic protein. In some embodiments, a binding partners may recognize and bind an MHC-II binding T-cell epitope on a cross-reactive allergenic protein set forth in Tables 13-18, 20, and 22. In some embodiments, a binding partner (e.g., an IgE or IgG antibody, e.g., a specific IgE or IgG antibody) may recognize and bind to 2 or more, 3 or more, 4 or more, 5 or more, 6 or more, 7 or more, or 8 or more of such cross-reactive epitopes.

In some embodiments, a binding partner may recognize and bind two or more B-cell epitope peptides, MHC-binding epitope peptides, and/or T-cell peptides derived from two or more cross-reactive allergenic proteins. In some embodiments, a binding partner may recognize and bind a peptide comprising a B-cell epitope and/or an MHC-II-binding T-cell epitope set forth in Tables 13-23. In some embodiments, a binding partner (e.g., an IgE or IgG antibody, e.g., a specific IgE or IgG antibody) may recognize and bind to one or more peptides comprising cross-reactive B-cell epitopes, MHC-binding epitopes, and/or T-cell epitopes derived from one or more Cupin proteins selected from the group consisting of: Pru du 6, Jug n 2, Jug n 4, Ana o 1, Ana o 2, Jug r 2, Jug r 4, Cor a 11, Cor a 9, Ara h 1, Ara h 3, Car i 2, Car i 4, Pis v 2, Pis v 3, Pis v 5, Ses i 3, Ses i 6, Ses i 7, Gly m 5, Gly m 6, Gly m Bd, Ber e 2, Coc n 1, Fag e 13s globulin, Fag e 3, Fag e legumin, Pis s 1, Pis s 2, Act d 12, Pink 2, Len c 1, Vig r 2, Lup an 1, Fag t 13S globulin, Lup a vicilin, and Sin a 2.

In some embodiments, a binding partner (e.g., an IgE or IgG antibody, e.g., a specific IgE or IgG antibody) may recognize and bind to a food allergenic antigen. In some embodiments, a binding partner may recognize and bind to a non-food allergenic antigen, for example, a cross-reactive allergenic protein and/or a cross-reactive epitope peptides, for example, those disclosed in Table 8, and/or are disclosed herein.

In one aspect, the present disclosure provides antibodies (e.g., IgE or IgG antibodies, e.g., specific IgE or IgG antibodies) that bind a particular cross-reactive allergenic protein and/or a peptide comprising a cross-reactive epitope (e.g., a B-cell epitope, MHC-binding epitope, and/or T-cell epitope). In some embodiments, an antibody binds an allergenic protein (such as an allergenic protein present in foods, for example, those provided in Table 1 and/or Table 8) or a peptide comprising a cross-reactive B-cell epitope, MHC-binding epitope, and/or a T-cell epitope derived from an allergenic protein. In some embodiments, an antibody binds to a peptide comprising a cross-reactive B-cell epitope, an MHC-binding epitopes, and/or T-cell epitope present on a particular or specific cross-reactive non-food allergenic protein (for example, those provided in Table 8 as well as those that may be identified by the methods provided herein).

In some embodiments, provided herein are antibodies that bind (e.g., specifically bind) one or more cross-reactive allergenic proteins and/or peptides comprising a cross-reactive B-cell epitope, MHC-binding peptide, and/or T-cell epitope derived from a cross-reactive allergenic protein. In some embodiments, an antibody binds (e.g., specifically binds) an identified or reference allergenic protein. In some embodiments, an antibody binds (e.g., specifically binds) a cross-reactive allergenic protein. In some embodiments, an antibody binds (e.g., specifically binds) a cross-reactive epitope on an allergenic protein provided in Table 2. In some embodiments, an antibody binds (e.g., specifically binds) one or more allergenic proteins selected from the group consisting of a shrimp allergen, a cod allergen, a salmon allergen, a hen's egg allergen, a cow's milk allergen, a peanut allergen, a sesame allergen, a soy allergen, a pecan allergen, a cashew allergen, a hazelnut allergen, a walnut allergen, a pistachio allergen, an almond allergen, and a wheat allergen, or a cross-reactive B-cell epitope, MHC-binding epitope, and/or T-cell epitope derived from any of the foregoing.

In some embodiments, an antibody binds (e.g., specifically binds) a cross-reactive allergenic protein listed in Table 3. In some embodiments, an antibody binds (e.g., specifically binds) a cross-reactive epitope on a Cupin protein (for example, listed in Table 5 or Table 10). In some embodiments, an antibody binds (e.g., specifically binds) one or more cross-reactive allergenic proteins present in a protein in tropomyosin, prolamin, lipid transfer protein, profilin, EF-Hand family, or Bet v 1 protein families (Table 4). In some embodiments, an antibody binds (e.g., specifically binds) a cross-reactive epitope and/or cross-reactive allergenic protein that may be identified by methods disclosed herein. In some embodiments, an antibody binds (e.g., specifically binds) to one or more cross-reactive B-cell epitopes provided in Tables 19, 21, and 23, and/or cross-reactive MHC-binding T-cell epitopes provided in Tables 13-18, 20, and 22.

In some embodiments, an antibody binds (e.g., specifically binds) a cross-reactive allergenic protein at a non-cross-reactive B-cell epitope, MHC-binding epitope, and/or T-cell epitopes derived from a cross-reactive allergenic proteins (for example, an epitope that is located on a cross-reactive allergenic protein, identified allergenic protein, or reference allergenic protein, but is not shared by or common to two or more allergenic proteins). For example, a contemplated antibody recognizes the cross-reactive allergenic protein Ana_o_2 but no other allergenic protein within the Cupin protein family.

In some embodiments, an antibody binds (e.g., specifically binds) B-cell epitopes, MHC-binding epitopes, and/or T-cell epitopes on one or more allergenic Cupin proteins (for example, listed in Table 5 or Table 10). In some embodiments, an antibody binds (e.g., specifically binds) B-cell epitopes, MHC-binding epitopes, and/or T-cell epitopes on one or more Cupin proteins selected from the group consisting of: Pru du 6, Jug n 2, Jug n 4, Ana o 1, Ana o 2, Jug r 2, Jug r 4, Cor a 11, Cor a 9, Ara h 1, Ara h 3, Car i 2, Car i 4, Pis v 2, Pis v 3, Pis v 5, Ses i 3, Ses i 6, Ses i 7, Gly m 5, Gly m 6, Gly m Bd, Ber e 2, Coc n 1, Fag e 13s globulin, Fag e 3, Fag e legumin, Pis s 1, Pis s 2, Act d 12, Pin k 2, Len c 1, Vig r 2, Lup an 1, Fag t 13S globulin, Lup a vicilin, and Sin a 2. In some embodiments, an antibody binds (e.g., specifically binds) bind B-cell epitopes, MHC-binding epitopes, and/or T-cell epitopes on one or more cross-reactive allergenic protein listed in Table 10. In some embodiments, B-cell epitopes, MHC-binding epitopes, and/or T-cell epitopes bound antibodies of the present disclosure are cross-reactive epitopes.

Contemplated antibodies can be of various isotypes, including, but not limited to: IgG (e.g., IgG1, IgG2, IgG2a, IgG2b, IgG2c, IgG3, IgG4); IgM; IgA1; IgA2; IgAsec; IgD; and IgE. In some embodiments, an antibody is an IgG isotype. In other embodiments, an antibody is an IgE isotype. Antibodies can be full-length (e.g., a full-length IgG1, IgG2, IgG3, or IgG4 antibody) or can include only a fragment or an analog thereof. The terms ‘full-length antibody’, ‘intact antibody’, and ‘whole antibody’ are used interchangeably to refer to an antibody in its substantially intact ‘native’ form, not antibody fragments as defined above. The terms particularly refer to an antibody with heavy chains that each comprise a variable domain and an Fc region.

A contemplated antibody may be an isolated polyclonal antibody, which has been raised against cross-reactive allergenic proteins and/or epitope peptides (e.g., B-cell epitope peptides and/or T-cell epitope peptides) of the present disclosure. Polyclonal antibodies can be obtained from any mammalian species of convenience: an antibody can, e.g., be isolated from a rabbit, mouse, rat, cat, dog, horse, cow, camel, llama, or even a human being.

A contemplated antibody may be a monoclonal antibody which binds (e.g., specifically binds) a cross-reactive allergenic protein or peptide of the present of the present disclosure. An antibody may be, for example, a chimeric, a humanized or a human antibody. In some embodiments, a humanized antibody is a human immunoglobulin (recipient antibody) in which HVR residues of the recipient antibody are replaced by residues from an immunoglobulin HVR of a non-human species (donor antibody) such as mouse, rat, rabbit, or nonhuman primate. In some embodiments, framework (FR) residues of a human recipient antibody are replaced by corresponding non-human residues. Furthermore, humanized antibodies may comprise residues that are not found in a recipient antibody or in a donor antibody. The term ‘monoclonal antibody’ refers to an antibody obtained from a population of substantially homogeneous antibodies, in that the individual antibodies comprising the population are identical except for possible mutations, such as naturally occurring mutations, that may be present in minor amounts. Thus, the modifier ‘monoclonal’ indicates the character of the antibody as not being a mixture of discrete antibodies. In contrast to polyclonal antibody preparations, which typically include different antibodies directed against different determinants (epitopes), each monoclonal antibody of a monoclonal antibody preparation is directed against the same single determinant on an antigen.

A contemplated antibody may also be presented in the form of a “derivative”, wherein one or more of the amino acids of an antibody, a fragment, or an analogue are chemically modified, e.g., by alkylation, PEGylation, acylation, ester formation or amide formation or the like, e.g., for linking an antibody to a second molecule. This includes, but is not limited to, PEGylated antibodies, cysteine-PEGylated antibodies, and variants thereof.

In addition to methods of identifying binding partners for cross-reactive allergenic proteins and/or epitope peptides derived from cross-reactive allergenic proteins, the present disclosure also provides antibodies that bind one or more cross-reactive allergenic proteins or epitope peptides derived from cross-reactive allergenic proteins. Such specific antibodies may, for example, be useful as immunotherapeutics, and in assays for identifying the presence of allergic diseases, the presence of specific foods or cross-reactive non-food allergens in subjects, and for potency testing of immunotherapeutic compositions.

Traditionally, antibodies have been developed to neutralize only single allergens. However, the present disclosure provides, for example, methods, compositions and kits that include antibodies that neutralize particular cross-reactive allergenic proteins. In some embodiments, antibodies neutralize at least two cross-reactive allergenic proteins. In some embodiments, antibodies neutralize a single cross-reactive allergenic protein.

In some embodiments, contemplated antibodies have an affinity (Kd) of less than 10⁻⁶, 10⁻⁷, 10⁻⁸, 10⁻⁹, 10⁻¹⁰, 10⁻¹¹, or 10⁻¹². In some embodiments, an antibody binds to an antigen with a Kd of at least 3 μM, 2 μM, 1 μM, 900 nM, 800 nM, 700 nM, 600 nM, 500 nM, 400 nM, 300 nM, 200 nM, 100 nM, 50 nM, 1 nM, 900 μM, 800 μM, 700 μM, 600 μM, 500 μM, 400 μM, 300 μM, 200 μM, 100 μM, 50 μM, 1 μM, 900 fM, 800 fM, 700 fM, 600 fM, 500 fM, 400 fM, 300 fM, 200 fM, 100 fM, 50 fM, or 1 fM. In some cases antibodies may have a Kd of at most 3 μM, 2 μM, 1 μM, 900 nM, 800 nM, 700 nM, 600 nM, 500 nM, 400 nM, 300 nM, 200 nM, 100 nM, 50 nM, 1 nM, 900 μM, 800 μM, 700 μM, 600 μM, 500 μM, 400 μM, 300 μM, 200 μM, 100 μM, 50 μM, 1 μM, 900 fM, 800 fM, 700 fM, 600 fM, 500 fM, 400 fM, 300 fM, 200 fM, 100 fM, 50 fM, or 1 fM as measured by any suitable biochemical assay, including but not limited to surface plasmon resonance analysis using, for example, a BIACORE system and software. In some embodiments, a binding affinity is ≤500 nM Kd. In some embodiments, a binding affinity is ≤100 nM Kd. In some embodiments, a binding affinity is ≤50 nM Kd. In some embodiments, a binding affinity is ≤1 nM Kd.

In one aspect, the present disclosure provides methods for preparation of antibodies that bind to an epitope on a cross-reactive allergenic protein. In some embodiments, provided herein are methods for preparation of antibodies that bind (e.g., specifically bind) to cross-reactive epitopes on two or more cross-reactive allergenic proteins. In some embodiments, antibodies may be neutralizing antibodies, for example, for use as passive immunotherapeutics. In some embodiments, antibodies may be linked to food allergenic peptides to create multiple allergen immunotherapeutics. In some embodiments, may be used as diagnostic reagents, either alone or in combination with various tags including, but not limited to, fluorescent markers. In some embodiments, an antibody is a monoclonal antibody or a polyclonal antibody. In some embodiments, a monoclonal antibody prepared using the methods of the present disclosure is a murine antibody, a bovine antibody, a chimeric antibody, a humanized antibody, or a human antibody.

In a contemplated method, individual cross-reactive allergenic proteins and/or peptides may, for example, be selected and expressed as cell surface epitopes, for example, by selecting peptides which comprise transmembrane helices in regions flanking epitopes of interest and introducing them into continuous cell lines using a retroviral vector transfection method, such that the polypeptide epitopes are displayed on the surface of the mammalian cells and anchored by the flanking transmembrane domains. Further, if an underlying cell line used is syngeneic with the intended host to be immunized, an immune response may, for example, be directed primarily to allergenic cross-reactive peptides of interest, thereby simplifying the process of selecting a high affinity antibody directed to a cross-reactive allergenic peptide of interest.

Contemplated antibodies may be raised for example, in any species of interest, for example, in mice (most commonly the species used to prepare hybridomas), guinea pigs, goats, chickens and others. Such antibodies may, for example, then be harvested for experimental or therapeutic use without the need to further produce hybridomas. A cell line established for expression of a protein may be a preexisting continuous line as is the case for Balb/c mice in which the 3T3 line is available (ATCC reference) or may be a primary line e.g., of fibroblasts established from the species, or individual, intended for immunization.

Monoclonal antibodies may be, for example, prepared by methods known in the art, including production of hybridomas, use of humanized mice, combinatorial display techniques, and the like. See, e.g., of Kohler and Milstein, Nature, 256:495 (1975), Wood et al., WO 91/00906, Kucherlapati et al., WO 91/10741; Lonberg et al., WO 92/03918; Kay et al., WO 92/03917; N. Lonberg et al., Nature, 368:856-859 [1994]; L. L. Green et al., Nature Genet., 7:13-21 [1994]; S. L. Morrison et al., Proc. Nat. Acad. Sci. USA, 81:6851-6855 [1994]; Bruggeman et al., Immunol., 7:33-40 [1993]; Tuaillon et al., Proc. Nat. Acad. Sci. USA, 90:3720-3724 [1993]; and Bruggeman et al. Eur. J. Immunol., 21:1323-1326 [1991]); Sastry et al., Proc. Nat. Acad. Sci. USA, 86:5728 [1989]; Huse et al., Science, 246:1275 [1989]; and Orlandi et al., Proc. Nat. Acad. Sci. USA, 86:3833 [1989]); U.S. Pat. No. 5,223,409; WO 92/18619; WO 91/17271; WO 92/20791; WO 92/15679; WO 93/01288; WO 92/01047; WO 92/09690; WO 90/02809; Fuchs et al., Biol. Technology, 9:1370-1372 [1991]; Hay et al., Hum. Antibod. Hybridomas, 3:81-85 [1992]; Huse et al., Science, 46:1275-1281 [1989]; Hawkins et al., J. Mol. Biol., 226:889-896 [1992]; Clackson et al., Nature, 352:624-628 [1991]; Gram et al., Proc. Nat. Acad. Sci. USA, 89:3576-3580 [1992]; Garrad et al., Bio/Technolog, 2:1373-1377 [1991]; Hoogenboom et al., Nuc. Acid Res., 19:4133-4137 [1991]; and Barbas et al., Proc. Nat. Acad. Sci. USA, 88:7978 [1991].

Chimeric and humanized antibodies and fragments thereof such as scFvs may be prepared as described in e.g., Robinson et al., PCT/US86/02269; European Patent Application 184,187; European Patent Application 171,496; European Patent Application 173,494; WO 86/01533; U.S. Pat. No. 4,816,567; European Patent Application 125,023; Better et al., Science, 240:1041-1043 [1988]; Liu et al., Proc. Nat. Acad. Sci. USA, 84:3439-3443 [1987]; Liu et al., J. Immunol., 139:3521-3526 [1987]; Sun et al., Proc. Nat. Acad. Sci. USA, 84:214-218 [1987]; Nishimura et al., Canc. Res., 47:999-1005 [1987]; Wood et al., Nature, 314:446-449 [1985]; and Shaw et al., J. Natl. Cancer Inst., 80:1553-1559 [1988]), U.S. Pat. No. 5,225,539; Jones et al., Nature, 321:552-525 [1986]; Verhoeyan et al., Science, 239:1534 [1988]; and Beidler et al., J. Immunol., 141:4053 [1988]).

In some embodiments, contemplated methods involve the steps of obtaining a plurality of candidate antibody producing cells, contacting the plurality of candidate antibody-producing cells with cross-reactive allergenic proteins, polypeptides or peptides comprising B-cell epitopes and producing an antibody that binds (for example, specifically binds) to a target cross-reactive allergenic protein, polypeptide or peptide comprising a B-cell epitope, thereby identifying an antibody that binds (for example, specifically binds) to a target cross-reactive allergenic protein, polypeptide, peptide comprising a B-cell epitope.

In some embodiments, antibodies that bind an allergenic antigen can be prepared by contacting one or more specific allergenic antigens (for example immobilized on a substrate) with a biological sample (for example, peripheral blood mononuclear cells (PBMCs) or isolated B-cells) and identifying allergen-positive B-cells. Allergen-positive B-cells can be identified and isolated. Immunoglobulin genes can then be amplified from allergen-positive B cells using methods known in the art, for example, by performing RT-PCR protocols on a single B-cell using kits commercially available from Invitrogen/Life Sciences Technologies, CA, USA. Amplification products of a PCR process can be then sequenced to determine binding epitopes and variable and light chain sequences of antibodies. Paired immunoglobulin heavy and light chains can be selected after sequencing, and DNA encoding selected heavy and light chains may be purified with a commercially available PCR Purification Kit such as the QIAquick 96 PCT Purification from Qiagen. Host cells may be transformed with heavy and light chains for example, NEB® 5-alpha competent E. coli (New England Biolabs) may be transformed with heavy and light chain ligation products and grown followed by selection of transformed cells. Clones are then selected and screened for vector insertion of amplified immunoglobulin heavy and light chains. Vectors harboring insertions are then amplified and purified and plasmid DNA is then sequenced to determine similarity to previous sequences. Isolated plasmid DNA from selected heavy and light chains may be transfected into host cells (for example, HEK293T) using commercially available DNA transfection kits (for example from SignaGen, Rockville, Md.) according to manufacturer's protocols. Antibodies may then be purified from transfected cells according to standard techniques. Allergen specificity can be then validated by ELISA assay (ImmunoCAP) for purified recombinant antibodies according to manufacturer's protocols.

In some embodiments, yeast surface display using a DNA library can be performed as described previously (Chao, G. et al. (2006) Nat. Protoc. 1(2):755-68) or through used of substrate immobilized antigens to confirm specificity of antibodies.

Antibodies produced by and/or used in methods of the present disclosure may, for example, have binding affinities of about 1 fm to ≤about 3 μM. In some embodiments, antibodies produced by and/or used in the methods of the present disclosure have a binding affinity of less than 100 nM, less than 10 nM, or less than 2 nM, as measured by a surface-plasmon resonance assay e.g., BIACORE.

Antibodies that bind cross-reactive allergenic proteins and epitopes present on cross-reactive allergenic proteins may, for example, be used to prepare compositions and kits as described in further detail below.

### X. PHARMACEUTICAL COMPOSITIONS

The disclosure also provides compositions comprising one or more active ingredients (such as cross-reactive allergenic proteins and/or peptides, and/or polynucleotides encoding such cross-reactive allergenic proteins and/or peptides, and/or antibodies that recognize and bind any of the foregoing). In some embodiments, such compositions are pharmaceutically acceptable compositions.

In some embodiments, the present disclosure provides single allergen or multiple allergen immunotherapeutic compositions comprising a plurality (e.g., 2, 3, 4, 5, 6, 7, 8, 9, 10 or more, including, e.g., up to about 5000) of distinct cross-reactive epitope peptides (or polypeptides comprising the cross-reactive peptides) from one or more allergenic proteins (e.g., identified allergenic proteins and/or cross-reactive allergenic proteins).

Provided herein in various embodiments are methods for preparing a vaccine comprising cross-reactive allergenic protein or cross-reactive epitope peptides. Contemplated methods may include identifying a cross-reactive epitope from an identified allergenic protein or a cross-reactive allergenic protein as described herein, synthesizing a cross-reactive epitope peptide or a cross-reactive protein, and creating a single or multiple allergen immunotherapeutic composition that includes a cross-reactive epitope peptide or cross-reactive allergenic protein in a pharmaceutically acceptable carrier. In some embodiments, methods include adsorbing a cross-reactive protein or cross-reactive peptide on to an adjuvant suitable for administration to subjects, for example, aluminum hydroxide (alum).

In some embodiments, methods may include identifying 4 or more cross-reactive epitope peptides; synthesizing the 4 or more cross-reactive epitope peptides; and creating a single or multiple allergen immunotherapeutic composition that includes the 4 or more cross-reactive epitope peptides in a pharmaceutically acceptable carrier. In some embodiments, methods include identifying at least two distinct allergenic proteins, each comprising at least 4 cross-reactive epitopes; and synthesizing the at least two distinct allergenic proteins to create a multiple allergen immunotherapeutic composition. In some embodiments, methods include identifying 6 or more distinct allergenic proteins, each comprising at least 4 cross-reactive epitopes; and synthesizing the at least 6 distinct allergenic proteins to create a multiple allergen immunotherapeutic composition. In some embodiments, methods include adsorbing a cross-reactive protein or cross-reactive peptide on to an adjuvant suitable for administration to subjects, for example, aluminum hydroxide (alum).

In some embodiments, methods may include identifying at least 6 distinct identified allergenic proteins or at least 6 distinct cross-reactive allergenic proteins; synthesizing the at least 6 distinct identified allergenic proteins or at least 6 distinct cross-reactive allergenic proteins; and creating a multiple allergen immunotherapeutic composition that includes the at least 6 distinct identified allergenic proteins or at least 6 distinct cross-reactive allergenic proteins in a pharmaceutically acceptable carrier. In some embodiments, methods include adsorbing a cross-reactive protein or cross-reactive peptide on to an adjuvant suitable for administration to subjects, for example, aluminum hydroxide (alum).

In some embodiments, methods may include identifying one or more cross-reactive epitopes from one or more identified allergenic proteins and/or one or more cross-reactive allergenic proteins as described above, synthesizing peptides comprising the one or more cross-reactive epitopes (or polypeptides comprising the one or more cross-reactive epitopes), and creating a multiple allergen immunotherapeutic composition that includes the one or more cross-reactive epitope peptides (or polypeptides comprising the one or more cross-reactive epitopes) in a pharmaceutically acceptable carrier. Methods may include identifying four or more cross-reactive epitopes from one or more identified allergenic proteins and/or one or more cross-reactive allergenic proteins as described above, synthesizing peptides comprising the four or more cross-reactive epitopes (or polypeptides comprising the four or more cross-reactive epitopes), and creating a multiple allergen immunotherapeutic composition that includes the four or more cross-reactive epitope peptides (or polypeptides comprising the four or more cross-reactive epitopes) in a pharmaceutically acceptable carrier. In some embodiments, the methods include identifying one or more (or four or more) cross-reactive epitopes from at least 6 distinct identified allergenic proteins and/or one or more cross-reactive allergenic proteins as described above to prepare multiple allergen immunotherapeutic compositions as described above.

Contemplated methods for creating single and multiple allergen immunotherapeutic compositions include, for example, determining and mapping cross-reactive epitopes (e.g., B-cell epitopes, T-cell epitopes, and/or MHC-binding T-cell epitopes) located in each of one or more allergenic proteins (e.g., one or more identified allergenic proteins such as those present in a multiple allergen oral immunotherapeutic composition) to other allergenic proteins within a protein family as described herein to identify cross-reactive epitopes, and selecting one or more identified cross-reactive T-cell epitopes. In some embodiments, homology of at least 70% identity over at least 80% coverage over a peptide is a criterion used for identification and/or mapping of a cross-reactive epitopes on an allergenic protein. In some embodiments, homology of at least 80% identity over at least 70% coverage over a peptide is a criterion used for identification and/or mapping of a cross-reactive epitope on an allergenic protein.

In some embodiments, provided herein are methods for preparing a multiple allergen oral immunotherapeutic composition that, for example, is capable of treating an allergy to one or more cross-reactive allergenic proteins, for example, an allergy to one or more cross-reactive allergenic proteins that are not present in the multiple allergen oral immunotherapeutic composition. Contemplated methods may, for example, include: (a) mapping one or more identified allergenic proteins present in a multiple allergen oral immunotherapy to full length homology of other allergenic proteins within a protein domain family; (b) identifying cross-reactive allergenic proteins with the identified allergenic proteins as described herein (e.g., based on homology by pairwise identity and similarity and/or or cross-reactivity prediction between the pair of sequences); (c) correlating one or more identified allergenic proteins with other allergenic proteins in the same protein domain family; and/or (d) selecting two or more identified allergenic proteins as a component in the multiple allergen oral immunotherapy, for example, if there is at least 70% homology or an A-RISC cross-reactivity score of ≥0.5 with two or more identified cross-reactive allergenic proteins. In some embodiments, methods for preparing a multiple allergen oral immunotherapeutic composition comprises a computer-implemented method for performing steps (a)-(d) as described herein. In some embodiments, the methods further include determining and mapping cross-reactive epitopes on the two or more identified allergenic proteins and two or more identified cross-reactive allergenic proteins. For example, in an embodiment, two or more identified allergenic proteins in a food source are mapped as described above to each of the additional allergenic proteins within a protein family (such as an identified protein family) to which these two or more identified allergenic proteins belong, comparing each additional allergenic protein and an identified allergenic protein (reference protein) to determine homology by pairwise identity and similarity, determining a cross-reactivity score for each of the additional allergenic protein within the protein family with respect to a reference protein (an identified allergenic protein or another additional allergenic proteins within the same protein family) and identifying as cross-reactive allergenic proteins those additional allergenic proteins that have a cross-reactivity score of ≥0.5-1 with respect to a reference protein. In some embodiments, mapping comprises i) providing a dataset of full-length sequences of identified allergenic proteins; ii) inputting the dataset into a computer readable medium; iii) searching the dataset of sequences using an alignment algorithm such as a BLAST, BLATP, TBLASTN, FASTA, etc., to identify other allergenic proteins having a minimal acceptable homology based on identity and similarity of at least 70% or an A-RSC cross-reactivity score of ≥0.5 with respect to the identified allergenic proteins to identify cross-reactive allergenic proteins.

In some embodiments, the methods comprise mapping the allergenic proteins present in any of the protein families provided in Table 3. In some embodiments, the methods comprise the mapping one or more of the identified allergenic protein selected from the group consisting of a shrimp allergen, a cod allergen, a salmon allergen, a hen's egg allergen, a cow's milk allergen, a peanut allergen, a sesame allergen, a soy allergen, a pecan allergen, a cashew allergen, a hazelnut allergen, a walnut allergen, a pistachio allergen, an almond allergen, and a wheat allergen. In some embodiments, the methods comprise the mapping the identified allergenic proteins set forth in Table 2. In some embodiments, the methods further include identifying specific antibodies that bind to identified cross-reactive epitopes of the present disclosure. In some embodiments, the methods further include identifying specific antibodies that bind to one or more of the cross-reactive epitopes present in allergenic proteins present in Table 3.

In some embodiments, the methods comprise preparing a multiple allergen oral immunotherapeutic composition comprising three or more allergenic proteins or cross-reactive epitopes present on three or more allergenic proteins selected from any of the identified protein families listed in Table 3. In some embodiments, the three or more allergenic proteins included in a multiple allergen oral immunotherapeutic composition are selected from the Cupin protein family, the tropomyosin protein family, the prolamin protein family, the lipid transfer protein family, the EF hand protein family, and the Bet v 1 protein family.

Accordingly, in some embodiments the methods comprise preparing a multiple allergen oral immunotherapeutic composition comprising three or more Cupin family proteins, three or more Tropomyosin family proteins, three or more Prolamin family proteins, three or more Lipid transfer protein family proteins, three or more EF hand family proteins, or three or more Bet v 1 family proteins, and a pharmaceutically acceptable excipient. In some embodiments, the methods comprise preparing a multiple allergen oral immunotherapeutic composition comprising three or more Cupin family proteins selected from Table 5 or Table 10.

In some embodiments, the methods comprise preparing a multiple allergen oral immunotherapeutic composition comprising three or more Cupin family proteins selected from Ber_e_2, Car_i_2, Fag_e_1, Fag_e_13S, Fag_t_13S_globulin, Jug_n_4, Lup_a_vicilin, Lup_an_1, Pin_k_2, Pis_s_1, Pis_s_2, Pis_v_2, Ses_i_3, Ses_i_6, Ses_i_7, Sin_a_2, Vig_r_2, Car_i_4, Gly_m_5, Gly_m_6, Jug_r_4, Len_c_1, and Pru_du_6. In some embodiments, the methods further include identifying specific antibodies that bind to one or more of cross-reactive epitopes present in allergenic proteins in the Cupin, Tropomyosin, Prolamin, Lipid transfer protein, EF hand, or Bet v 1 protein families. In some embodiments, the methods further include identifying specific antibodies that bind to one or more of the cross-reactive B-cell epitopes provided in Tables 19, 21, and 23, and/or the cross-reactive WIC-binding T-cell epitopes provided in Tables 13-18, 20, and 22.

In an aspect, the present disclosure provides methods for preparing pharmaceutically acceptable compositions comprising a specific antibody that recognizes and specifically binds one or more epitopes present in a cross-reactive allergenic protein, comprising: (a) identifying a specific antibody that binds to B-cell epitope peptides (or polypeptides or proteins comprising such B-cell epitope peptides) by the methods of the present disclosure, (b) preparing the antibody recombinantly or by chemical synthesis, and (c) creating an immunotherapeutic composition that includes the specific antibody in a pharmaceutically acceptable carrier.

In some embodiments, provided herein are methods for preparation of pharmaceutically acceptable compositions comprising two or more specific antibodies that recognize two or more cross-reactive allergenic protein or epitopes thereof, comprising: (a) identifying a specific antibody that binds to B-cell epitope peptides (or polypeptides or proteins comprising such B-cell epitope peptides) by the methods of the present disclosure; (b) preparing the two or more specific antibodies recombinantly or by chemical synthesis; and (c) creating a multiple allergen immunotherapeutic composition that includes the two or more specific antibodies in a pharmaceutically acceptable carrier. In some embodiments, each of the two or more antibodies bind different cross-reactive allergenic proteins. In other embodiments, the two or more antibodies bind the same cross-reactive allergenic proteins. In some embodiments, the two or more antibodies bind the same cross-reactive allergenic proteins at different epitopes.

In some embodiments, methods for preparation of antibodies that recognize and bind to B-cell cell epitopes include (a) correlating one or more specific allergenic proteins with other allergenic proteins in the same protein family to identify cross-reactive specific B cell epitope peptides; (b) identifying the specific IgE or IgG antibodies that bind to the identified cross-reactive B cell epitope peptides; and (c) preparing the one or more specific IgE or IgG antibodies recombinantly or by chemical synthesis. In some embodiments, correlation comprises: (a) mapping each identified allergenic protein in a food to its protein family or superfamily (e.g., based on protein domains), (b) determining and mapping homology of each of the identified allergenic protein to the full-length additional allergenic proteins within the protein family in order to identify cross-reactive allergenic proteins within the protein family, and (c) determining and mapping the identified T-cell and/or B-cell epitopes on cross-reactive allergenic proteins and the identified allergenic proteins in each protein family. In some embodiments, the correlation further comprises: generating a map of the pairwise identity and similarity between the specific allergenic protein and the other allergenic proteins in the same patent family to identify cross-reactive proteins.

In some embodiments, provided herein are the methods for preparation of an immunotherapeutic composition that is capable of treating an allergic disease to one or more cross-reactive allergenic proteins that are not present in the multiple oral immunotherapeutic composition, comprising: (a) correlating one or more specific allergenic proteins with other allergenic proteins in the same protein family to identify cross-reactive specific B cell epitope peptides; (b) identifying the specific IgE or IgG antibodies that bind to the identified cross-reactive B cell epitope peptides; (c) preparing the one or more specific IgE or IgG antibodies recombinantly or by chemical synthesis; (d) creating an immunotherapeutic compositions by combining the one or more specific IgE or IgG antibodies in a pharmaceutically acceptable carrier. One of skill in the art will appreciate that the immunotherapeutic compositions prepared by this embodiment of the method includes the one or more specific antibodies that are capable of binding cross-reactive B-cell epitopes present in the one or more specific allergenic proteins as well as in other allergenic proteins, and thus the immunotherapeutic composition comprising one or more specific antibodies directed to one or more specific allergenic protein is also capable of treating a subject for an allergic disease directed to the other allergenic proteins that can be recognized and bound by the one or more specific antibodies present in the immunotherapeutic composition. In some embodiments, the immunotherapeutic composition prepared by this method is a single allergen immunotherapeutic composition. In some embodiments, the immunotherapeutic composition prepared by this method is a multiple allergen immunotherapeutic composition. In at least one embodiment, the multiple allergen immunotherapeutic composition is a multiple allergen oral immunotherapeutic composition. In some embodiments, the one or more specific allergenic proteins is selected from the group of identified allergenic proteins set forth in Table 2.

In some embodiments, provided herein are methods for preparing a multiple allergen oral immunotherapy composition that is capable of treating an allergy to one or more cross-reactive allergenic proteins that are not present in the multiple allergen oral immunotherapy, comprising: (a) mapping one or more identified allergenic proteins present in the multiple allergen oral immunotherapy to full length homology of other allergenic proteins within a protein family; (b) identifying cross-reactive allergenic proteins with the identified allergenic proteins by pairwise identity and similarity between the sequences and/or (c) correlating one or more identified allergenic proteins with other allergenic proteins in the same protein domain family; and (d) selecting two or more identified allergenic proteins as a component in the multiple allergen oral immunotherapy if there is a greater than 70% homology (identity and similarity) with two or more identified cross-reactive allergenic proteins or if each of the two or more cross-reactive allergenic proteins has an A-RISC cross-reactivity score of ≥0.5 with respect to at least one of the one or more identified allergenic proteins. Mapping generally comprises i) providing a dataset of full-length sequences of identified allergenic proteins; ii) inputting the dataset into a computer readable medium; iii) searching the dataset of sequences using an alignment algorithm selected from the group consisting of GAP, BESTFIT, FASTA, TFASTA, BLAST, BLASTP, TBLASTN, FASTDB, ALIGN, CLUSTALW, and CLUSTAL-Omega, including any version thereof.

In some embodiments, pharmaceutically acceptable compositions further include a pharmaceutically acceptable excipient.

Techniques for formulating and administration can also be found in Remington's Pharmaceutical Sciences, Mack Publishing Co., Easton, Pa, latest edition. Cross-reactive allergenic proteins and cross-reactive epitopes may be prepared as described above and in Examples below using chemical and recombinant technology. General methods for preparation of compositions comprising cross-reactive allergenic proteins, polynucleotides encoding cross-reactive allergenic proteins, peptides comprising cross-reactive epitopes, and/or antibodies that recognize and bind them are known in the art.

Pharmaceutically acceptable compositions may be single allergen immunotherapeutic compositions (for example, in that a composition includes a single allergenic protein or epitopes from a single allergenic protein, wherein such epitopes may be present only on the single allergenic protein or may be cross-reactive with other cross-reactive allergenic proteins) or multiple allergen immunotherapeutic compositions. Multiple allergen immunotherapeutic compositions of the present disclosure may include two or more allergenic proteins, one or more epitopes from two or more allergenic proteins, or two or more antibodies each binding to a specific allergenic protein or an antibody that binds to two or more allergenic proteins. One of skill in the art will appreciate that various combination are possible for the preparation of multiple allergen immunotherapeutic compositions of the present disclosure. Pharmaceutically acceptable compositions may be formulated for administration by any suitable route of administration, for example, orally, parenterally, nasally or topically. Accordingly, provided herein in some embodiments are multiple allergen oral immunotherapeutic compositions, multiple allergen parenteral immunotherapeutic compositions, multiple allergen nasal immunotherapeutic compositions, and multiple allergen topical immunotherapeutic compositions.

When compositions of the present disclosure contain two or more active ingredients (e.g., cross-reactive allergenic proteins, cross-reactive epitopes, antibodies, etc.), each active ingredient, may be present in equal amounts or they may be present in different amounts on a w/w basis of the final composition. In some embodiments, compositions may comprise two or more active ingredients wherein each of two or more active ingredients may be held at a concentration insufficient to elicit an immune response by itself, but when combined in a multiple allergen immunotherapeutic composition, the sum of the two or more active ingredient is sufficient to elicit an immune response. In some embodiments, where multiple allergen compositions comprise two or more whole (complete) food sources (e.g., from Table 8), each may be present in the composition in equal amounts or in differing amounts. In some embodiments, each of the two or more whole food sources may be held at a concentration insufficient to elicit an immune response by itself, but when combined in a multiple allergen immunotherapeutic composition, the sum of the two or more individual food sources is sufficient to elicit an immune response. One of skill in the art will appreciate that presence of a second or third (or more) cross-reactive allergenic protein or cross-reactive epitope peptide may, in certain embodiments, boost the ability of the first (or as may be the case, primary) allergenic protein or epitope peptide in inducing an immune response.

In some embodiments, compositions comprise two or more cross-reactive T-cell epitopes, wherein cross-reactive T-cell epitopes from a first cross-reactive allergenic protein (e.g., an identified allergenic protein of Table 2) synergize with cross-reactive T-cell epitopes from an other (second or third or fourth and so on) cross-reactive allergenic protein to boost the effect of the cross-reactive T-cell epitopes from the first cross-reactive allergenic protein (and vice versa). In some embodiments, compositions comprise two or more polypeptides or cross-reactive allergenic proteins comprising one or more cross-reactive epitopes.

Pharmaceutically acceptable excipients are known in the art and are compatible with other ingredients in the formulation and are not toxic to the subjects to whom they are administered. Such pharmaceutically acceptable excipients include pharmaceutically acceptable carriers which can be liquid carriers, finely divided solid carriers, or both. Suitable pharmaceutically acceptable carriers include, but are not limited to, sterile water, saline, glucose, dextrose, alcohols, buffered solutions, and other organic and inorganic solvents. Pharmaceutically acceptable excipients include agents such as diluents, stabilizers (e.g., sugars and amino acids), preservatives (such as ethyl-p-hydroxybenzoate), bulking agents (such as glycine, mannitol, etc.), suspending agents (such as methyl cellulose, tragacanth, sodium alginate) wetting agents (such as lecithin, polyoxyethylene stearate, polyoxyehtylene sorbitan mono-oleate, etc.), emulsifying agents such as surfactants (such as non-ionic surfactants, polymeric surfactants, polyoxyethylene sorbitan ester surfactants, polysorbates such as Polysorbate 80, Tween 20, poloxamers), pH buffering agents, additives that enhance viscosity, granulating and disintegrating agents (e.g., starch, alginic acids), binding agents (e.g., starch, gelatin, HPMC, CMC, acacia etc.), lubricating agents (e.g., magnesium stearate, stearic acid, talc, etc.), antibacterial and antifungal agents (such as phenol, sorbic acid, chlorobutanol, etc.), odor masking agents, flavoring agents, coloring agents, and any other excipient conventionally added to pharmaceutical formulations, and any combinations thereof. In some embodiments, excipients are plant derived. In other embodiments, excipients are not human or animal derived.

Non-limiting examples of surfactants include TWEEN20 (polysorbate 20), TWEEN40 (polysorbate 40), TWEEN60 (polysorbate 60), TWEEN80 (polysorbate 80), TRITON X-100, IGEPAL CA-630, Nonidet P-40, PLURONIC F-68, PLURONIC F-88, and PLURONIC F-127 (poloxamers), and Brij 35 (polyoxyethylene alkyl ether) available from various commercial manufacturers.

In some cases, a pharmaceutically acceptable composition further comprises a physiologically acceptable adjuvant. An adjuvant typically provides for increased immunogenicity. An adjuvant can be one that provides for slow release of the active ingredients (example, a liposome), or it can be an adjuvant that is immunogenic in its own right and which may, for example, thereby function synergistically with a cross-reactive protein or peptide. For example, an adjuvant can be a known adjuvant or other substance that is capable of promoting the uptake of the active ingredients, recruiting immune cells to the site of administration, or facilitating the immune activation of responding lymphoid cells. Adjuvants include, but are not limited to, immunomodulatory molecules (e.g., cytokines such as hIL-12), CpG motifs (synthetic oligonucleotides containing immunomodulatory CpG motifs), virosomes, oil and water emulsions, mineral salts (e.g., aluminum hydroxide, calcium or aluminum phosphate gels), glucan, dextran sulfate, iron oxide, sodium alginate, bacto-adjuvant, synthetic polymers such as poly amino acids and co-polymers of amino acids, saponin, ISCOMS, paraffin oil, muramyl dipeptide, Immundaptin (C3d tandem array), and inert particles such as gold particles. For example, polysorbate 80 may be used in the preparation of pharmaceutically acceptable immunotherapeutic compositions comprising vaccines.

In some embodiments, compositions of the present disclosure are provided as lyophilized forms and may be resolubilized at the time of administration. Such lyophilized forms are stable for handling, transportation, and long-term storage. Lyophilized forms may include excipients such as bulking agents, stabilization agents, and surfactants to maintain the stability of the cross-reactive allergenic proteins, epitope peptides thereof, vaccines, and/or antibodies of the present disclosure. A lyophilized form may be reconstituted at the time of use or it may be included neat or with additional excipients described herein in a solid dosage form such as tablet, caplet, or capsule.

In some embodiments, pharmaceutically acceptable compositions may comprise a pharmaceutically acceptable carrier. Such compositions may be formulated as liquid formulations. In some embodiments, liquid formulations may be provided as hydroalcoholic compositions and/or aqueous compositions.

In some embodiments, active ingredients may be encapsulated, adsorbed to or associated with particulate carriers, such as lipid particles, polymethyl methacrylate polymer particles, PLG particles derived from poly(lactides) and poly(lactide-co-glycolides) (See Jeffry et al. (1993) Pharm. Res. 10:362-368). Other particulate systems and polymers may also be used, e.g., polymers such as polylysine, polyarginine. Polyornithine spermine, spermidine, as well as conjugates thereof.

In some embodiments, pharmaceutically acceptable compositions may be formulated as lipid microparticles or lipid nanoparticles, etc. Such microparticles and/or nanoparticles may comprise lipid vesicles, lipid microspheres, nanosphere, etc. In various embodiments, lipid microparticles and lipid nanoparticles may be any of solid lipid nanoparticles (SLN, made up of solid lipids with a photon correlation spectroscopy mean diameter of approximately between 50 and 1000 nm and SLM have an equivalent composition to SLN, but with a larger particle size (>1000 nm)), nanostructured lipid carriers, lipid-drug conjugate nanoparticles, Lipid nanocapsules. Methods for preparation of such lipid microparticles and lipid nanoparticles are known in the art and further described in Battaglia and Ugazio available on the world wide web at doi.org/10.1155/2019/2834941 and U.S. Pat. No. 6,287,591B1.

Lipid nano- and microparticles can be orally administered in the form of aqueous dispersions or, alternatively, after transformation into a solid dosage form, such as tablets, pellets, capsules, or powders in cachets. Aqueous particulate dispersions can be used as a granulation fluid for the production of tablets. Alternatively, they can be transformed into a powder (e.g., by spray-drying or freeze-drying) and added to a tableting powder mixture or used to fill hard gelatin capsules.

Lipid microparticles and lipid nanoparticles can be used, for example, for parenteral applications: from intra-articular to intramuscular, subcutaneous, and intravenous administration. Because of their small size, lipid nanoparticles can be injected intravenously and used to target drugs to specific organs. In some embodiments, “stealth” lipid nanoparticles are prepared by coating their surface with polyethylene glycol (PEG) to avoid clearance from circulation by liver and spleen.

Lipid microparticles and nanoparticles of the present disclosure are also useful for topical administration. Cutaneous drug delivery of lipid particles may, for example, provide the ability to protect chemically labile ingredients against chemical decomposition, the ability to modulate drug release, and the ability to form adhesive lipid films on the skin, providing a possible occlusive effect.

In some embodiments, pharmaceutically acceptable compositions are emulsions, such as oil-in-water emulsions, water-in-oil emulsions, water-in-oil-in water emulsions, oil-in-water-in-oil emulsion, etc. Emulsions may, for example, include at least three core ingredients: an oil component; an aqueous component; and a surfactant component. In general, oil-in-water emulsions are formed by combining at least one oil (e.g., an oil component), an aqueous component such as water, and at least one surfactant which has been subjected to the methods described herein. Although spontaneous emulsification is possible, mixing will normally involve mechanical aid, such as the use of a homogenizer and/or a microfluidizer. In an embodiment, an emulsification process mixes components first by using a homogenizer and then by using a microfluidizer. Where a component includes more than one compound (e.g., two different oils in the oil component, two different surfactants in the surfactant component, etc.), these may be combined in various orders prior to emulsification, e.g., a first surfactant and an oil may be pre-mixed, to be combined with a mixture of a second surfactant and an aqueous component.

Provided herein in one embodiment are methods for preparation of pharmaceutically acceptable compositions comprising the active ingredients of the present disclosure including: (a) providing a surfactant, such as polysorbate 80, and (b) combining the purified surfactant (e.g., polysorbate 80) with an oil component to provide an emulsion. In addition to the purified surfactant and the oil component, the emulsion will include an aqueous component (and, as described in more detail below, possibly further components as well). Aqueous material may be added as a third component in step (b), or may be combined with the surfactant and/or the oil prior to step (b). Thus, the surfactant (e.g., polysorbate 80) which is provided in step can be an aqueous solution of the surfactant (e.g., polysorbate 80), but may also be non-aqueous surfactant (e.g., a surfactant which has not been diluted with water (or any other aqueous material) prior to being filtered). The oil-in-water emulsion thus obtained is then combined with a two or more cross-reactive allergenic proteins to create a pharmaceutically acceptable composition. Although it is possible to administer oil in water emulsion adjuvants on their own to subjects (e.g., to provide an adjuvant effect for an antigen that has been separately administered to a subject), it is more usual to admix the adjuvant with an antigen prior to administration, to form an immunogenic composition, e.g., a vaccine. Mixing of emulsion and antigen may take place extemporaneously, at the time of use, or can take place during vaccine manufacture, prior to filling. The methods of the present disclosure can be applied in both situations.

Compositions can be used when preparing mixed vaccines or when preparing kits including active ingredients (antigens or antibodies) and adjuvant ready for mixing. Where mixing takes place during manufacture then the volumes of bulk active ingredients and emulsion that are mixed will typically be greater than 1 liter, e.g., >5 liters, >10 liters, >20 liters, >50 liters, >100 liters, >250 liters, etc. Where mixing takes place at the point of use then the volumes that are mixed will typically be smaller than 1 milliliter, e.g., <0.6 ml, <0.5 ml, <0.4 ml, <0.3 ml, <0.2 ml, etc. In both cases it is usual for substantially equal volumes of emulsion and antigen solution to be mixed substantially 1:1 (e.g., between 1.1:1 and 1:1.1, preferably between 1.05:1 and 1:1.05, and more preferably between 1.025:1 and 1:1.025). In some embodiments, however, an excess of emulsion or an excess of antigen may be used. Where an excess volume of one component is used, the excess will generally be at least 1.5:1, e.g., >2:1, >2.5:1, >3:1, >4:1, >5:1, etc.

In some embodiments, 3 cross-reactive allergenic proteins are combined as active ingredients with emulsions of the present disclosure to create a multiple allergen immunotherapeutic composition that, for example, is suitable for oral, parenteral, nasal, and/or topical administration.

In some embodiments, an oil-in-water emulsion obtained is combined with one or more epitope peptides (such as cross-reactive T-cell epitope peptides, B-cell epitope peptides, and/or MHC-binding epitope peptides) to create a pharmaceutically acceptable composition. In some embodiments, an oil-in-water emulsion is combined with 4 or more cross-reactive T-cell epitope peptides, B-cell epitope peptides, or MHC-binding epitope peptides, or a combination thereof, of a single cross-reactive allergenic protein to create an allergen immunotherapeutic composition or vaccine. In some embodiments, an oil-in-water emulsion is combined with 4 or more cross-reactive T-cell epitope peptides, B-cell epitope peptides, or MHC-binding epitope peptides, or a combination thereof, selected from the group set forth in Tables 13-23 to create an allergen immunotherapeutic composition or vaccine.

In some embodiments, an oil-in-water emulsion is combined with one or more antibodies that recognize and bind epitope peptides (cross-reactive or non-cross-reactive) of allergenic proteins to create a pharmaceutically acceptable composition. In some embodiments, an oil-in-water emulsion is combined with a single antibody that recognizes and binds to a cross-reactive B-cell epitope peptide and/or MHC-binding epitope peptide set forth in Tables 13-23.

An aqueous component may comprise an aqueous solution which may be plain water (e.g., w.f.i.) or can include further components, e.g., solute(s). For instance, it may include salts to form a suitable buffer, e.g., citrate or phosphate salts, such as sodium salts. Typical buffers include, but are not limited to: a phosphate buffer, a Tris buffer, a borate buffer, a succinate buffer, a histidine buffer, and a citrate buffer. Buffers will typically be included in the 5-50 mM range, depending on the particular buffer being used.

In some embodiments, a total amount of lipids (oil(s)) (% by volume) in a final emulsion is between about 1-30%, e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29% about 30%. In some embodiments, a total amount of oil(s) (% by volume) in a final emulsion is between about 1-20%, between about 5-15%, between about 8-12%, or about 10%.

In some embodiments, amounts of surfactants range from about 0.01% to about 2%, about 0.5% to about 2% or about 1% on a w/w/basis of a final emulsion. In some embodiments, a surfactant is polysorbate 80, commercially available from many manufacturers at a pharmaceutical grade (e.g., the ultra-pure “Polysorbate (HX2)” product from NOF Corporation). Polysorbate 80 may be present in a final composition in amounts ranging from 0.01% to about 2%. In some embodiments, multiple surfactants may be used. The HLB value of the surfactants may be in the range of 1-10 (e.g., SPAN 85, with an HLB of 1.8). Surfactants which can be used in addition to polysorbate 80 include, but are not limited to: the polyoxyethylene sorbitan esters surfactants (commonly referred to as the Tweens), such as polysorbate 20; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy (oxy-1,2-ethanediyl) groups, with octoxynol-(TRITON™ X-100, or t-octylphenoxypolyethoxyethanol) being of particular interest; (octylphenoxy)polyethoxyethanol (IGEPAL CA-630/NP-40); phospholipids such as phosphatidylcholine (lecithin); polyoxyethylene fatty ethers derived from lauryl, cetyl, stearyl and oleyl alcohols (known as Brij surfactants), such as triethyleneglycol monolauryl ether (Brij 30); polyoxyethylene-9-lauryl ether; and sorbitan esters (commonly known as the SPANs), such as sorbitan trioleate (SPAN® 85) and sorbitan monolaurate.

The term lipid as used herein includes, for example, triglycerides (e.g., tristearin), partial glycerides, fatty acids (e.g., stearic acid), steroids (e.g., cholesterol), and waxes (e.g., cetyl palmitate). Lipids in an emulsion's oil components will typically be biodegradable, metabolizable, and biocompatible. An oil component in an emulsion may include a single oil, or may comprise a combination of oils, e.g., comprising a fatty acid such as stearic acid and at least one further oil for example a triglyceride such as tristearin. Other lipids will be apparent to one of skill in the art.

In some embodiments, excipients do not include any human or animal products.

Compositions disclosed herein may comprise comprise a suitable concentration of each protein, polypeptide, peptide, polynucleotide, or binding agent (e.g., antibody) to be effective. Exemplary concentrations of each cross-reactive allergenic protein or peptide include those in the range of about 0.01 to 500 nmol/mL. In some embodiments, a range will be about 0.1 to 300 nmol/mL, about 1 to 200 nmol/mL, about 10 to 150 nmol/mL or about 25 to 100 nmol/mL. Where compositions are multiple allergen immunotherapeutic composition, each cross-reactive allergenic protein in the composition (or cross-reactive peptide) may be present in equal amounts or differing amounts. Where more than two active ingredients are present, two or more may be in the same amount while the remaining active ingredients may be in differing amounts.

In some embodiments, a concentration of an antibody in a composition will range from 0.01 μg/mL to 500 mg/mL. In some embodiments, a concentration of an antibody will range from 1 μg/mL to 400 mg/mL, from about 10 μg/mL to about 300 mg/mL, from about 10 mg/mL to about 300 mg/mL, or from about 1 mg/mL to about 500 mg/mL. Active ingredients in compositions or formulations may, for example, have a purity of greater than 95%, 96%, 97%, 98%, or 99%.

In some embodiments, multiple allergen immunotherapeutic compositions comprising two or more whole (complete) food sources selected from Table 8 comprise each food source in equal proportions. In some exemplary compositions, the two or more whole food sources are provided as a whole food flour or powder. In some embodiments, the composition comprises proteins extracted from each whole food source. In some embodiments, a multiple allergen composition comprising two or more whole food source includes 0.1% to 50% total protein and a pharmaceutically acceptable excipient. In some embodiments, two or more whole food sources are present in ratios ranging about 1:1. Compositions comprising two or more whole food sources may be formulated as a tablet, caplet or a capsule. In some embodiments, the compositions may be in liquid dosage forms. Dosage forms can be, for example, from 0.1 mg to 2 gm per dosage unit. In some embodiments, each dosage unit comprises about 0.1 mg, 0.5 mg, 1 mg, 5 mg, 10 mg, 25 mg, 50 mg, 100 mg, 200 mg, 500 mg, 1000 mg, 1500 mg or 2000 mg per dosage unit. Pharmaceutically acceptable excipients are known in the art and described above. In some embodiments, excipients do not include any human or animal products.

In some embodiments, provided herein are methods for preparation of multiple allergen compositions comprising two or more whole food sources (for example, food sources provided in Table 8). In some embodiments, multiple allergen compositions are formulated as orally administered solid dosage forms, such as tablets, caplets and/or capsules. In some embodiments, a fill includes a binding agent to enhance flowability such as those provided above and an odor masking agent and/or a flavoring agent. Equal amounts of the food sources are included in some embodiments. Excipients suitable for inclusion in a multiple allergen oral composition can be any of the excipients known in the art.

In some embodiments, pharmaceutically acceptable compositions of the present disclosure, when administered to a subject, biological sample, or cell, are capable of eliciting an immune response (such as IgE and/or IgG production, T-cell activation, basophil activation, etc.) in the subject, biological sample, or cell. Pharmaceutically acceptable compositions of the present disclosure comprising, for example, DNA, peptides, cross-reactive allergenic proteins, B-cell epitope peptides, MHC-II-binding epitope peptides, and/or T-cell epitope peptides, may, for example, be useful as vaccine immunotherapeutic compositions for prophylactic or therapeutic treatment of one or more food allergies or food-intolerances or symptoms thereof or other epitope-sensitive conditions. Pharmaceutically acceptable compositions of the present disclosure comprising, for example, binding partners of DNA, peptides, cross-reactive allergenic proteins, B-cell epitope peptides, MHC-II-binding epitope peptides, and/or T-cell epitope peptides, may, for example, be useful as immunotherapeutic compositions for prophylactic or therapeutic treatment of one or more food allergies or food-intolerances or symptoms thereof or other epitope-sensitive conditions.

Compositions of the present disclosure (for example, comprising antibodies or other binding partners) may also, for example, be useful in assays for detection of specific allergenic antigens (food antigens and/or cross-reactive non-food antigens) and for identification of candidate subjects that would benefit from administration of a pharmaceutically acceptable composition of the present disclosure. Compositions of the present disclosure (for example, comprising allergenic proteins or peptides comprising epitopes of cross-reactive allergenic proteins) may also, for example, be useful in assays for detection of antibodies in a subject, a biological sample, or a cell and/or in assays for testing the potency of pharmaceutically acceptable compositions comprising cross-reactive proteins and/or cross-reactive peptides.

In some embodiments, compositions (e.g., oral, parenteral, nasal, topical etc.) disclosed herein, when administered to a subject, are capable of treating an allergic disease due to a cross-reactive allergenic protein and/or an identified allergenic protein that is not present in the composition.

### XI. DETECTION; DIAGNOSTICS

Cross-reactive allergenic proteins and/or cross-reactive epitopes identified using methods provided herein may, for example, induce immune responses, for example, mediated by cross-reactive IgE, IgG, T-cells (such as CD4+ cells, T-regs), and/or or other cells. Such cross-reactive allergenic proteins or cross-reactive epitope peptides capable of inducing an immune response may also be referred to as “immunologically cross-reactive allergenic proteins” or “immunologically cross-reactive allergenic peptides.” Antibodies that are cross-reactive with immunologically cross-reactive allergenic proteins or cross-reactive epitope peptides can be readily detected using methods provided herein. In one aspect, the present disclosure provides methods for detection of such cross-reactive antibodies, for example, IgE and/or IgG antibodies (such as IgG1, IgG2, IgG3, and/or IgG4). Contemplated methods may, for example, be useful for confirming the presence of a suspected allergy, for example, based on a subject's clinical history. Contemplated methods may also, for example, be useful for monitoring the efficacy of an allergy therapy such as with a multiple allergen oral immunotherapy. Contemplated methods may also, for example, be useful for determining the extent or severity of an allergy, or the potential (or risk) for developing a cross-reactive sensitization or an allergy mediated by one or more cross-reactive allergenic proteins.

The disclosure provides, for example, methods for the detection of an immune response (e.g., by the detection of the presence of cross-reactive antibodies such as IgE and IgG) in a subject, a biological sample, or a cell. The disclosure also provides, for example, methods for the multiplexed detection of an immune response (e.g., by the detection of the presence of cross-reactive antibodies such as IgE and IgG, and/or by the detection of T-cell or basophil activation or response) in a subject, a biological sample, or a cell.

In some embodiments, cross-reactive proteins or epitope peptides of the present disclosure are coupled to a solid support or substrate (for example, via a linker) to form one or more protein-solid support complexes and/or epitope peptide-solid support complexes.

In some embodiments, cross-reactive allergenic proteins (including identified allergenic proteins) or cross-reactive epitope peptides of the present disclosure are immobilized to a solid support substrate. In some embodiments, 2 or more, 3 or more, 4 or more, 5 or more, 6 or more, 7 or more, 8 or more, 25 or more, 50 or more, 100 or more, 1000 or more, or at least 5000 distinct allergenic proteins or peptides are immobilized to a solid support substrate. In some embodiments, at least 6 distinct identified allergenic proteins (such as those in Table 2) are immobilized to a solid support or substrate.

In some embodiments, 1 or more, 2 or more, 3 or more, 4 or more, 5 or more, 6 or more, 7 or more, 8 or more, 25 or more, or 50 or more, 100 or more, or 1000 or more distinct cross-reactive allergenic proteins (for examples, proteins in protein families listed in Tables 3-4 or proteins listed in Tables 4-7 and 10-12). In some embodiments, one or more cross-reactive allergenic proteins are selected from: Ber_e_2, Car_i_2, Fag_e_1, Fag_e_13S, Fag_t_13S_globulin, Jug_n_4, Lup_a_vicilin, Lup_an_1, Pin_k_2, Pis_s_1, Pis_s_2, Pis_v_2, Ses_i_3, Ses_i_6, Ses_i_7, Sin_a_2, Vig_r_2, Car_i_4, Gly_m_5, Gly_m_6, Jug_r_4, or Len_c_1, or Pru_du_6.

In some embodiments, cross-reactive epitope peptides of the present disclosure are immobilized or coupled to a solid support or substrate. In some embodiments, cross-reactive MHC-II binding epitope peptides derived from identified allergenic proteins or cross-reactive allergenic proteins are immobilized or coupled to a solid support or substrate. Such cross-reactive epitope peptides (for example, peptides comprising MHC-II-binding T-cell epitope peptides or B-cell epitope peptides) may, for example, be derived from at least 2 distinct allergenic proteins, at least 3 distinct allergenic proteins, at least 4 distinct allergenic proteins, at least 5 distinct allergenic proteins, at least 6 distinct allergenic proteins, or more, for example, at least 50 distinct allergenic proteins, at least 100 distinct allergenic proteins, at least 500 distinct allergenic proteins, at least 1000 distinct allergenic proteins, or at least 5000 distinct allergenic proteins, etc. Non-limiting examples of cross-reactive peptides that may, for example, be immobilized on a solid support or substrate include cross-reactive epitope peptides derived from cross-reactive allergenic proteins in Tables 4-7 and 10-12. In some embodiments, cross-reactive MHC-II-binding T-cell peptides are selected from the group of cross-reactive MHC-II binding T-cell peptides provided in Tables 13-18, 20, and 22. In some embodiments, the -reactive MHC-II binding T-cell peptides are derived from one or more of: Ber_e_2, Car_i_2, Fag_e_1, Fag_e_13S, Fag_t_13S_globulin, Jug_n_4, Lup_a_vicilin, Lup_an_1, Pin_k_2, Pis_s_1, Pis_s_2, Pis_v_2, Ses_i_3, Ses_i_6, Ses_i_7, Sin_a_2, Vig_r_2, Car_i_4, Gly_m_5, Gly_m_6, Jug_r_4, or Len_c_1, or Pru_du_6. In some embodiments, cross-reactive MHC-II-binding T-cell peptides are selected from the group of cross-reactive B-cell epitope peptides and/or MHC-binding epitope peptides set forth in Tables 13-23.

Provided herein are methods for detecting the presence of specific food antigens and cross-reactive non-food antigens in a subject, a biological sample, or a cell. Methods include, for example, providing one or more specific antibodies (e.g., IgG, IgE, IgM, IgA, IgD, etc.) to a cross-reactive B-cell epitope peptide and/or MHC-binding epitope peptide, contacting a biological sample that is suspected to (may) have an allergenic protein under conditions sufficient to permit binding of the antibodies, and detecting binding of the antibodies, to identify the specific food antigen and/or specific cross-reactive non-food antigen. A food antigen can be any food antigen. Exemplary food antigens to which antibodies may, for example, bind include those provided in the present disclosure, including but not limited to, food antigens from the food sources listed in Table 1 and Table 8. Exemplary cross-reactive non-food antigens to which antibodies may, for example, bind include those present in the non-food sources provided in Table 8. In some embodiments, antibodies bind to exemplary Cupin cross-reactive epitope peptides set forth in Tables 13-19. In some embodiments, antibodies bind to exemplary Tropomyosin and Bet v 1 cross-reactive epitope peptides set forth in Tables 20-23.

### Substrates/Supports

Identified allergenic proteins, cross-reactive allergenic proteins (including immunologically cross-reactive proteins), cross-reactive epitope peptides, and/or antibodies of the present disclosure can be immobilized onto a solid support or substrate. Substrates include any suitable rigid or semi-rigid substrate, such as membranes, filters, chips (such a microchips), slides (such as microscope slides), wafers fibers, magnetic or non-magnetic beads, gels, tubing, plates (such as microtiter plates), polymers, microparticles, and capillaries. Substrates may have any of a variety of surface forms, such as wells, trenches, pins, channels, and pores, to which proteins or peptides bind. In some embodiments, substrates are optically transparent. In some embodiments, a substrate is a poly-L-lysine coated microscope slide. In some embodiments, a substrate is a multi-well microtiter plate. In some embodiments, a substrate is a bead.

### Microarrays

In some embodiments, identified allergenic proteins and/or one or more immunologically cross-reactive allergenic proteins are coupled to a solid substrate that is an array, such as a microarray. In some embodiments, antibodies capable of detecting particular or specific food antigens or cross-reactive non-food antigens are couple or immobilized on to a solid support or substrate, such as those described above. In some embodiments, antibodies capable of detecting particular or specific food antigens or cross-reactive non-food antigens are couple or immobilized on a solid support or substrate in an array such as a microarray. Such microarray technologies may, for example, allow for the evaluation of large numbers of biological samples simultaneously in a high-throughput manner. Microarrays can be fabricated using a variety of technologies, including printing with fine-pointed pins, photolithography using pre-made masks, photolithography using dynamic micromirror devices, ink-jet printing, microcontact printing, or electrochemistry on microelectrode arrays. In standard microarrays, probe molecules are attached via surface engineering to a solid surface of supporting materials, which include glass, silicon, plastic, hydrogels, agaroses, nitrocellulose and nylon.

Several techniques are well-known in the art for attaching polynucleotides to a solid substrate such as a glass slide. One method is to incorporate modified bases or analogs that contain a moiety that is capable of attachment to a solid substrate, such as an amine group, a derivative of an amine group, or another group with a positive charge, into amplified polynucleotides. An amplified product is then contacted with a solid substrate, such as a glass slide, which may be coated with an aldehyde or another reactive group which can form a covalent link with the reactive group that is on the amplified product and become covalently attached to the glass slide. Microarrays comprising amplified products can be fabricated using a Biodot (BioDot, Inc., Irvine, Calif) spotting apparatus and aldehyde-coated glass slides (CEL Associates, Houston, Tex.). Amplification products can be spotted onto aldehyde-coated slides, and processed according to published procedures (Schena et al. (1995) Proc. Natl. Acad. Sci. U.S.A. 93:10614-10619). Arrays can also be printed by robotics onto glass, nylon (Ramsay, G., Nature Biotechnol. (1998), 16:40-44), polypropylene (Matson, et al. (1995) Anal Biochem. 224(1):110-6), and silicone slides (Marshall and Hodgson, Nature Biotechnol. (1998) 16:27-31). Other approaches to array assembly include fine micropipetting within electric fields (Marshall, and Hodgson, Nature Biotechnol. (1998) 16:27-31), and spotting polynucleotides directly onto positively coated plates. Methods such as those using amino propyl silicon surface chemistry are also known in the art, as disclosed on the world wide web at cmgm.stanford.edu/pbrown/.

Commercial antibody array printing services are available for the preparation of custom antibody arrays, for example, membrane-based arrays using a chemiluminescent imager (CCD camera or x-ray film/dark room) as a detection system, glass slide-based arrays using laser microarray scanner for detection, and bead array using a flow cytometer for detection (RayBiotech, USA; Full Moon Biosystems, USA; and Kinexus, Canada).

Assays of the present disclosure may be implemented in a multiplex format. Multiplex methods are provided employing 2, 3, 4, 5, 6, 8, 10, 15, 20, 25, 50, 100, 200, 500, 1000 or more different cross-reactive allergenic proteins (or peptides thereof) which can be used simultaneously to assay for cross-reactive antibodies, for example, IgE or IgG, for example, IgG4 antibodies. Multiplex methods are also provided employing 2, 3, 4, 5, 6, 8, 10, 15, 20, 25, 50, 100, 200, 500, 1000 or more different antibodies recognizing and binding cross-reactive allergenic proteins (or epitope peptides thereof) which can be used simultaneously to assay for the presence of cross-reactive proteins (food allergens and/or cross-reactive non-food allergens). In some embodiments, six or more antibodies (e.g., IgE or IgG) are bound to a solid support or substrate, wherein the six or more antibodies bind to B-cell epitopes and/or MHC-binding epitopes in cross-reactive allergenic proteins present in any of the identified protein families set forth in Tables 3-4. In some embodiments, six or more antibodies (e.g., IgE or IgG) are bound to a solid support or substrate, wherein the six or more antibodies bind to B-cell epitopes set forth in Tables 19, 21 and 23. In some embodiments, six or more antibodies (e.g., IgE or IgG) are bound to a solid support or substrate, wherein the six or more antibodies bind to MHC-binding epitopes selected set forth in Tables 13-18, 20, and 22.

In some embodiments, four or more epitopes are bound to a solid support or substrate, wherein the four or more epitope peptides (B-cell epitope peptides, T-cell epitope peptides, and/or MHC-binding epitope peptides) are selected from the group set forth in Tables 13-23.

Where different substrates are included in a multiplex assay as part of a capture probe conjugate, the different substrates can be encoded so that they can be distinguished. Any encoding scheme can be used; for example, an encoding scheme can employ one or more different fluorophores, which can be fluorescent semiconductor nanocrystals. High density spectral coding schemes can be used.

The present disclosure provides solid support or substrates described above including particles, microparticles or beads, e.g., magnetic beads, that can be used, for example, for microarray-based assays. Particles or beads can be prepared from a variety of different polymers, including but not limited to polystyrene, cross-linked polystyrene, polyacrylic acid, polylactic acid, polyglycolic acid, poly(lactide coglycolide), polyanhydrides, poly(methyl methacrylate), poly(ethylene-co-vinyl acetate), polysiloxanes, polymeric silica, latexes, dextran polymers and epoxies. Materials have a variety of different properties with regard to swelling and porosity, which are well understood in the art. Preferably, beads are in the size range of approximately 10 nanometers to 1 millimeter, preferably 100 nanometers to 10 micrometers, and can be manipulated using normal solution techniques when suspended in a solution. The terms “particle,” “nanoparticle,” “bead,” “microbead,” “nanobead,” “sphere,” “microparticle,” and “microsphere” are used interchangeably herein, unless indicated otherwise.

Suitable chemical compositions for magnetic particles may be ferromagnetic materials and include rare earth containing materials such as, e.g., iron-cobalt, iron-platinum, samarium-cobalt, neodynium-iron-boride, and the like. Other magnetic materials, e.g., superparamagnetic materials such as iron oxides (Fe3O4) may be used as well. Preferred magnetic materials include iron-cobalt as such material is generally easier to magnetize, has a stronger magnetization (about 1.7 Tesla) and is less susceptible to corrosion.

### Detection

Antibodies in a test sample or allergen or epitope peptides in a test sample may be detected using any convenient detection method. Immunocomplexes of an IgE from a test sample (such as a biological sample) and epitope peptides of an allergenic protein, or immunocomplexes of allergenic proteins or epitope peptides from a test sample and antibodies that recognize and bind the allergenic proteins or epitope peptides may be detected using a variety of techniques. For instance, many such methods (which may be, for example, qualitative or semi-quantitative) rely on the use of antibodies. For instance, detection may entail contacting a sample with an antibody of the present disclosure and detecting binding of material in the sample to the antibody. Such assays can, e.g., be in the form of agglutination assays or immunoblots (dot blot analysis, quantitative dot blot, Western blot) of any format. To facilitate detection an antibody may be labelled with a radioactive isotope, a component of a ligand/receptor pair, a luminescent or fluorescent label, an enzyme, etc.

A sample (for example, a biological sample, for example, obtained from a subject, an allergen extract, an immunotherapy composition comprising an allergen whole food source, an allergenic protein peptide of the present disclosure) may, for example, be incubated with one or more identified allergenic proteins and/or immunologically cross-reactive allergenic proteins (for example, using a microarray) under conditions that allow immunocomplexes to form. As another example, a biological sample which may have specific food allergens or cross-reactive non-food allergens may, for example, be incubated with antibodies (for example using a microarray) under conditions that allow immunocomplexes to form. After an incubation period, the solid support may, for example, be washed to remove excess biological sample that has not bound to immobilized allergenic proteins or peptides thereof, or antibodies, and immunocomplexes may be detected, for example, using a labeling agent such as an incorporated fluorophore.

Labeling agents can be any suitable labeling agent known in the art, including radioisotopes, fluorophores, and biotin, etc. In some embodiments, the labeling agent is a fluorophore, such as fluorescein isothocyanate, rhodamine, phycoerythrin, phycocyanin, allophycocyanin, o-pthaldehyde, florescamine or fluorescence-emitting metals such as ¹⁵²Eu or other lanthanides. These metals may, for example, be attached to antibodies (or peptides and polypeptides) using metal chelators, In some embodiments, the labeling agent is chemiluminescent compound. The presence of chemiluminescence-tagged antibody or antigen peptide may, for example, be determined by detecting luminescence that arises during the course of chemical reaction. Example of bioluminescent compounds include luciferin, luciferase, and aequorin. In some embodiments, the labeling agent may be a radio label such as ¹²⁵I, ³⁵S and ¹⁴C.

Contemplated methods may use a second antibody that may, for example, be specific for epitopes characteristic of a particular human immunoglobulin type, for example, IgM, IgG1, IgG2, IgG3, IgG4, IgE and the like, which may permit identification of the isotype or isotypes of antibodies in a sample. A second antibody may be specific for an idiotype of a detection antibody (e.g., an antibody disclosed herein) or an antibody present in a biological sample.

One of skill in the art will be able to determine operative and optimal conditions for each determination by routine experimentation. Further, steps of washing stirring, shaking, filtering and the like may be added to the assays as is customary or necessary for the particular situation.

In some embodiments, a bioassay used is an enzyme-linked immunosorbent assay (ELISA), wherein an enzyme is used as a detectable label bound either to an antibody or an antigen. When exposed to its substrate, the enzyme reacts to produce a chemical moiety which can be detected, for example, by spectrophotometer, fluorometric or visual means. Enzymes useful in the context of the present disclosure of are known in the art. In some embodiments, fluorescence microscopy can be used to detected the presence of bound IgE (or IgG) etc., for example by examining a scanned image to determine the intensity of signal from a cross-reactive antibody.

In some embodiments employing particles, readout devices may not be necessary. For example, particles on microarray spots can be viewed directly with naked eyes if the spots are larger than 0.03 millimeters in diameter. Alternatively, assay results with any spot sizes, from 0.01 millimeters to 5 millimeters in diameter, can be photographed with an ordinary camera or viewed under an appropriate magnification microscope.

Binding partners of the present disclosure (e.g., specific antibodies, epitope-specific T-cells, etc.) may, for example, be useful in the detection of an immune response to one or more cross-reactive allergens in a subject, a biological sample, or a cell.

In some embodiments, antigens (cross-reactive allergenic proteins (including identified allergenic proteins and cross-reactive epitope peptides)) of the present disclosure are not immobilized on a solid support or substrate, but can be used in a solution-based assay for detection of antibodies. In some embodiments, antibodies of the present disclosure are not immobilized on a solid or substrate, but can be used in a solution-based assay for detection of an allergenic antigen. Such assays are well-known in the art and comprise contacting an antigen (or antibody) with a test sample to allow formation of an antigen-antibody complex. Detect of a label provides a measure for the presence of antibodies (or antigen, as applicable) in a test sample.

In some embodiments, the present disclosure provides methods for detecting the presence of a complex of an allergenic antigen and an antibody in a fluid sample. In some embodiments, an allergenic antigen may be a cross-reactive allergenic protein (for example an identified allergenic protein from Table 2, or a cross-reactive protein, e.g., from Tables 4-7 or 10-12) or a cross-reactive MHC-II-binding epitope peptide (e.g., from Tables 13-18, 20, or 22) that binds an antibody that is present in a test sample. A test sample may be a biological sample or may contain a biological sample (for example, diluted or undiluted, obtained from a subject).

In an exemplary method, a complex which may be immobilized to a solid support or substrate is detected by contacting the complex with a detectably labelled binding partner that specifically binds to the antibodies in the complex or that specifically binds to the antigen in the complex. The binding partner thus binds to the complex allowing for its detection. The complex with the detectable binding partner may be precipitated and detection of the label may be used as a measure of the peptide-antibody complex. For example, a second antibody such as a fluorescently-labeled antibody or enzymatically-labeled antibody may be used. In some embodiments, the solid support or substrate is a multi-well microtiter plate, or microarray slide or bead.

Contemplated methods may be used in a point of care type method on fresh biological samples, or on refrigerated, frozen, or otherwise preserved biological samples.

The disclosure provides, for example, methods for detecting at least two target antibodies in a biological sample comprising: contacting a first solid support comprising a first cross-reactive allergenic protein immobilized to the solid support or substrate with a biological sample; contacting a second solid support comprising a second cross-reactive allergenic protein immobilized to the solid support or substrate with the biological sample; and detecting the presence or absence of the two target antibodies.

The disclosure provides, for example, methods for detecting T-cells that bind to one or more cross-reactive epitopes of the present disclosure. In some embodiments, methods include contacting one or more cross-reactive allergenic proteins or one or more cross-reactive epitope peptides immobilized on a solid support or substrate with a biological sample that may contain T-cells (e.g., from peripheral blood mononuclear cells (PBMCs) or isolated T-cells) and detecting the binding of any T-cells with the one or more cross-reactive allergenic proteins or one or more cross-reactive epitope peptides using methods known in the art. Allergen-positive T-cells can be identified and isolated. Immunoglobulin genes can be amplified from the allergen-positive T cells using methods known in the art, for example, by performing RT-PCR protocols on single T-cells using kits commercially available from Invitrogen/Life Sciences Technologies, CA, USA. Amplification products of the PCR process can be then sequenced to determine the binding epitopes and the variable and light chain sequences of the antibodies.

### Diagnostic Methods

In one aspect, the present disclosure provides methods, compositions and kits for the detection and diagnosis in subjects of one or more allergies or food-intolerance. In some embodiments, one or more allergies or food-intolerance are unidentified or suspected allergies to cross-reactive allergenic proteins. Subjects may have one or more existing allergies and may be sensitized to one or more cross-reactive allergens or at risk of becoming sensitized to one or more cross-reactive allergenic proteins. For example, a subject may have an existing single food allergy to a food source listed in Table 2 (e.g., a peanut allergen ara h1) and may have either become sensitized to one or more allergenic proteins that are cross-reactive with ara h1 or be at risk of becoming sensitized to one or more allergenic proteins that are cross-reactive with ara h1. The methods provided herein may, for example, be useful for assessing a biological sample obtained from such an individual to determine the presence of an immune response to such one or more cross-reactive allergenic proteins, e.g., the presence of cross-reactive antibodies (IgE, IgG4, etc.), and/or the presence or activation of a cellular response in T-cells, basophils, mast cells, granulocytes etc. In some embodiments, it may be desirable to assess cytokine release and/or degranulation in such subjects.

The disclosure also provides allergenic profiles of subjects and methods of preparing such allergenic profiles. Such profiles and/or assays may be used to determine foods to which a subject is allergic or tolerized (or at risk of becoming tolerized/desensitized) to improve treatment of a subject and/or a subjects' quality of life. Methods provided herein may be used, for example, for the preparation of allergenic profiles or AllerMaps for each food source for each individual. Such personalized allergenicity maps may, for example, be useful for tracking and monitoring existing allergies and for preventing development of suspected or unidentified allergies to cross-reactive allergenic proteins. Accordingly, the disclosure also provides methods for the determination of serological or allergenic profiles of subjects including providing a biological sample comprising cross-reactive antibodies, performing assays on the biological sample to confirm the presence of cross-reactive B-cell and/or WIC-binding T-cell epitope antigens in the biological sample of the subject, and using such information to generate a profile of the subject.

In some embodiments, disclosed methods are used to monitor efficacy of a therapy (e.g., a multiple allergen oral immunotherapy) in subjects. Differences in IgE and/or IgG binding to proteins (or peptides thereof) before, during, and/or after multiple allergen oral immunotherapy treatment may, for example, be monitored. By analyzing changes in patterns of IgE and/or IgG (e.g., IgG4) binding, efficacy of a treatment may, for example, be determined. Such methods may, for example, be used to rapidly screen large numbers of populations to determine the effects of multiple allergens on a population.

In some embodiments, provided herein are methods of detecting development of and/or achievement of tolerance to a multiple allergen oral immunotherapy in a subject being administered the multiple allergen oral immunotherapy, or of monitoring the efficacy of multiple allergen oral immunotherapy in a subject being administered the multiple allergen oral immunotherapy. Contemplated methods comprise: (a) obtaining a biological sample from the subject (for example, before, after, or during one or more intervals of administration); (b) contacting the biological sample with one or more identified allergenic proteins present in the multiple allergen oral immunotherapy and/or one or more immunologically cross-reactive proteins; (c) measuring immune response/markers in the biological sample to the identified allergenic proteins present in the multiple allergen oral immunotherapy and/or one or more immunologically cross-reactive proteins; and/or (d) comparing the measured immune response/markers with a previous measurement, to assess the efficacy of the multiple allergen oral immunotherapy. Immune response/markers that may be measured include, but are not limited to, the presence or binding of cross-reactive IgE and/or IgG (e.g., IgG4), T-cell proliferation (Th1/Th2 shift), activation of basophils, mast cells, cytokine release (differences in non-terminally differentiated T-cell produced cytokines (IFNγ, IL-10) and terminally differentiated Th2-produced cytokines (IL-4, IL-5, IL9, IL-13, IL-15, and IL-17)), degranulation (histamine, Serotonin), etc. In some embodiments, methods comprise measuring the presence or amount (e.g., concentration) of IgE or IgG (e.g., IgG4), binding of IgE or IgG (e.g., IgG4), and/or a T-cell response (as further described in Example 10) to one or more identified allergenic proteins and/or one or more immunologically cross-reactive proteins; and comparing the presence or amount (e.g., concentration) of IgE or IgG (e.g., IgG4), binding of IgE or IgG (e.g., IgG4), or T-cell response with a previous measurement of the presence or amount (e.g., concentration) of IgE or IgG (e.g., IgG4), binding of IgE or IgG (e.g., IgG4), or T-cell response to assess the efficacy of the multiple allergen oral immunotherapy.

Any appropriate immune response may be measured and used to indicate tolerance. For example, Th2 cell proliferation may be reduced, e.g., as determined using an assay described in the Examples. The magnitude of Th2 cell proliferation reduction may vary, and in certain instances may range from 1.2× to 10×, such as 2× to 4×. Specific IgG4 levels may be increased and the magnitude of IgG4 level increase may vary, and in certain instances may range from 1.2× to 100×, such as 2× to 6×. Specific IgE levels may be reduced and the magnitude of IgE level reduction may vary, and in certain instances may range from 1.1× to 7×, such as 2× to 6×.

Provided herein are methods for assessing the presence or absence of cross-reactive IgE antibodies that bind to one or more allergenic proteins, or T-cells recognizing T-cell epitopes on one or more allergenic proteins (e.g., cross-reactive allergenic proteins described herein). In some embodiments, one or more allergenic proteins are present in a multiple allergen oral therapeutic composition administered to a subject. Contemplated methods include, for example, contacting a biological sample obtained from a subject with one or more identified allergenic proteins (for example, that are included in a multiple allergen oral therapeutic composition administered to a subject). Binding of IgE antibodies and/or T-cells to one or more identified allergenic proteins may, for example, be measured as described in the Examples or by methods known in the art. In some embodiments, methods disclosed herein are used to determine whether one or more identified allergenic proteins that bind to IgE antibodies and/or T-cells present in a biological sample are cross-reactive to any additional allergenic proteins. In some embodiments, a method does not include testing the additional allergenic proteins in vitro or ex vivo for binding to IgE antibodies and/or T-cells and/or the additional allergenic proteins have not previously been tested in vitro or ex vivo for binding to IgE antibodies and/or T-cells. One or more identified allergenic proteins that may, for example, be used in such methods include those from any protein family listed in Table 3. In some embodiments, the one or more identified allergenic proteins are selected from the group consisting of a shrimp allergen, a cod allergen, a salmon allergen, a hen's egg allergen, a cow's milk allergen, a peanut allergen, a sesame allergen, a soy allergen, a pecan allergen, a cashew allergen, a hazelnut allergen, a walnut allergen, a pistachio allergen, an almond allergen, and a wheat allergen. In some embodiments, the one or more identified allergenic proteins are selected from Table 2. Additional or untested allergenic proteins or peptides can be any cross-reactive allergenic protein or cross-reactive epitope peptide. In some embodiments, additional or untested allergenic proteins (which may or may not be cross-reactive) are from a family selected listed Table 3, or are from the Cupin family (Tables 5 and 10). In some embodiments, the identified allergenic proteins and the additional or untested allergenic proteins are from the same protein family. In some embodiments, the identified allergenic proteins and the additional or untested allergenic proteins are from different protein family.

In some embodiments, one or more identified allergenic proteins are immobilized on a substrate or a solid support. Substrates and solid supports suitable in the context of the present disclosure are described above. In some embodiments, the substrate is a bead. In some embodiments, one or more identified allergenic proteins are present in an array.

Contemplated methods include obtaining or testing a biological sample from a subject at any time or interval prescribed by a health professional (e.g., an allergist or a physician). For example, a biological sample may be obtained from a subject before treatment, during treatment, and/or post-treatment. By way of illustration, a biological sample may be obtained at baseline (for example, prior to initiating a therapy, e.g., a multiple allergen oral therapy) and then again during treatment at one or more intervals, for example, daily, every other day, weekly, biweekly, semi-monthly, monthly, upon completion of treatment or achieving tolerization or desensitization, etc.

Biological samples can be any suitable biological sample. In some embodiments, a biological sample is selected from the group consisting of whole blood, serum, plasma, sputum, blood cells (e.g., peripheral blood mononuclear cells (PBMC), T-cells, B-cells, basophils, etc.), immune cells, tissue samples, biopsy samples, urine, tears, peritoneal fluid, pleural fluid, breast duct fluid, breast exudate, breast milk, breast fluids, saliva, semen, mucous, lymph, cytosol, ascites, amniotic fluid, bladder washes, and bronchioalveolar lavage or cells therefrom. Patient samples may be fresh or frozen, and may be treated, e.g., with heparin, EDTA, citrate or any other suitable treatment known in the art. In some embodiments, a biological sample is whole blood, serum, plasma, or peripheral blood mononuclear cells (PBMC).

### XII. KITS

In another aspect, the disclosure provides kits for the detection and/or characterization of allergy. In some embodiments, the kits contain one or more identified allergenic proteins, one or more immunologically cross-reactive allergenic proteins, and/or one or more cross-reactive epitope peptides that binds to antibodies such as IgE, IgG (e.g., IgG4), IgA, IgM and IgD, or to T-cells or to MHC-II molecules. In some embodiments, kits contain detection reagents and buffers. In some embodiments, kits contain reagents specific for detection of cross-reactive IgE and/or IgG antibodies. In some embodiments, kits contain IgE-specific and/or IgG-specific (e.g., IgG4-specific) labeling reagents. In some embodiments, kits contain all of the components necessary to perform a detection or a diagnostic assay, including all controls, labeling agents, buffers (including binding buffers, wash buffers, and detection buffers), and instructions for use. In some embodiments, any necessary software for identification of cross-reactive allergenic proteins and identified allergenic proteins, and for analysis and presentation of results are included.

In certain embodiments, provided herein are kits for biological cross-reactivity IgE and/or IgG assays comprising six or more distinct proteins, each bound to a solid support. Solid supports suitable for use in kits include substrates described above or those known in the art. In some embodiments, a solid support is a bead, an array or a microplate. In some embodiments, six or more distinct proteins are selected from the group consisting of identified allergenic proteins listed in Table 2. In some embodiments, six or more distinct protein are selected from the group consisting of allergenic proteins listed in Table 3. In some embodiments, the six or more distinct proteins are selected from those listed in Tables 2, 4-7 and 10-12. In some embodiments, kits further contain a IgE-specific and/or IgG-specific (e.g., IgG4-specific) labeling reagent, packaged together with allergenic proteins and instructions for use.

In some embodiments, provided herein are detection kits comprising six or more specific IgE or IgG antibodies each bound to a solid support or substrate, wherein the six or more specific IgE or IgG antibodies are antibodies to B-cell epitopes selected from the group of B-cell epitopes set forth in Tables 19, 21, and 23. Solid support or substrates that may be included in the kits are described above. In some embodiments, each solid support is a bead. In some embodiments, the kit further comprises an IgE or IgG specific labeling reagent and instructions for use. In some embodiments, the labeling agents may be packaged together with the specific antibodies and instruction for use. In some embodiments, the kit further comprises one or more of a binding buffer, a wash buffer and a detection buffer.

Kits, compositions, and methods disclosed herein may, for example, be useful in potency testing for multiple allergen oral immunotherapeutic compositions.

In some embodiments, kits contain a immunotherapeutic composition, for example, an immunotherapeutic composition disclosed herein. In some embodiments, kits comprise one or more of an identified allergenic protein, a cross-reactive allergenic protein, and/or a cross-reactive epitope. In some embodiments, kits comprise one or more antibodies, e.g., antibodies disclosed herein. Identified allergenic proteins, cross-reactive allergenic proteins, cross-reactive epitopes, and/or antibodies may be provided in a solution or in lyophilized form. In some embodiments, kits comprise vaccines, antibodies, polypeptides, and/or polynucleotides, vectors, oligonucleotides/primers, host cells, and the like. In some embodiments, kits comprise four or more cross-reactive epitope peptides from a cross-reactive allergenic protein in a pharmaceutically acceptable carrier, and instructions for use. In some embodiments, kits comprise four or more cross-reactive epitope peptides from an identified allergenic protein in a pharmaceutically acceptable carrier, and instructions for use. In some embodiments, kits comprise a single allergen immunotherapeutic composition. In some embodiments, kits comprise a multiple allergen immunotherapeutic composition, for example an oral, parenteral, nasal, or topical immunotherapeutic composition.

### XIII. TREATMENT, PREVENTION, MONITORING

The present disclosure provides, for example, methods for parallel tolerization in subjects to not only allergies to known food allergenic proteins and but also to cross-reactive allergenic proteins that are present in additional food sources and non-food allergens. In one aspect, the present disclosure provides methods for tolerization in subjects with allergies, food intolerances, epitope-sensitive conditions, disorders or diseases, and any other conditions that would benefit from the administration of the compositions of the present disclosure. In certain embodiments, contemplated methods improve immune health, immune-mediated health, immune balance, gut health, and/or quality of life. In certain embodiments, contemplated methods treat and/or prevent allergies (e.g., food allergies), food intolerances, B-cell epitope-sensitive, MEW-binding epitope-sensitive, and T-cell epitope-sensitive conditions, disorders or diseases, inflammatory disorders, and/or autoimmune disorders.

In an exemplary method, a subject having an existing allergic disease (e.g., allergy or food-intolerance) to one or more allergenic proteins (for example, from Table 2) is administered a multiple allergen immunotherapeutic composition (e.g., orally, nasally, parenterally, topically etc.) comprising one or more allergenic proteins (for example, from Table 2). In certain embodiments, an allergenic protein also boosts the resistance of a subject to an other allergen, or tolerizes or treats an allergy to an other allergen in a subject (known or undiagnosed, unidentified, or suspected). In some embodiments, the other allergen is not included in the multiple allergen immunotherapeutic composition. It is contemplated that multiple allergen immunotherapeutic compositions comprising at least one allergenic protein (or cross-reactive T-cell epitope present in the allergenic protein) to which the subject has a primary allergy or food-intolerance, when administered to a subject, may also, for example, treat an additional known, unidentified, undiagnosed or suspected allergy to one or more cross-reactive allergenic proteins.

In another exemplary method, a subject having an existing allergic disease (e.g., allergy or food-intolerance) to one or more identified allergenic proteins (for example, from Table 2) is administered a multiple allergen immunotherapeutic composition (e.g., oral, nasal, parenteral, topical etc.) comprising one or more cross-reactive allergenic proteins (e.g., Cupin proteins from Tables 5 or 10) to which the subject has no known or diagnosed allergy but which are cross-reactive to the one or more identified allergenic protein to which the subject is known to be allergic. In some embodiments, the one or more identified allergenic proteins are not included in the multiple allergen immunotherapeutic composition. It is contemplated that multiple allergen immunotherapeutic compositions comprising one or more cross-reactive allergenic proteins (or cross-reactive T-cell epitope present in the allergenic protein), when administered to a subject, may also, for example, treat the known or primary allergy or food-intolerance in the subject mediated by the one or more identified allergenic proteins.

In another exemplary method, a subject may be administered a multiple allergen oral immunotherapeutic composition comprising not only one or more identified allergenic proteins to which the subjects are allergic or have food-intolerance, but also one or more cross-reactive allergenic proteins. It is contemplated that the inclusion of the one or more cross-reactive allergenic proteins in the multiple allergen compositions may, for example, boost the resistance of the subjects to the one or more identified allergenic proteins to which the subject is allergic or has food intolerance or other cross-reactive T-cell epitope-sensitive conditions, disorders and disease. It is also contemplated that, in certain embodiments, through use of cross-reactive allergenic proteins, subjects may be administered lower doses of one of more identified allergenic proteins to which the subject is allergic. Such lower or suboptimal doses of primary allergenic proteins when combined with cross-reactive allergenic proteins may, for example, function to elicit an immune response in the subject.

In another exemplary method, it is contemplated that if a subject has an undiagnosed, unidentified, or a suspected allergy to a cross-reactive allergenic protein in addition to an existing allergy to an identified allergenic protein, then administration of a multiple allergen immunotherapeutic composition comprising either one or more identified allergenic proteins or one or more cross-reactive allergenic proteins or comprising both can induce an immune response and/or parallel tolerization to both allergens in the subject.

In another exemplary method, it is contemplated that if a subject has a food allergy or food-intolerance and is administered a multiple allergen immunotherapeutic composition comprising an identified allergenic proteins or cross-reactive allergenic protein present in a food, in certain embodiments, depending on the allergenic protein selected for inclusion in the immunotherapeutic composition, such immunotherapeutic composition may, for example, induce an immune response against a cross-reactive allergenic protein present in a non-food source. For example, a multiple allergen oral immunotherapeutic composition comprising one or more identified allergenic proteins from Table 2 that belong to the tropomyosin protein family and are present in a food (e.g., tropomyosin family proteins present in shrimp) may, for example, induce an immune response against a cross-reactive allergenic protein in the tropomyosin family present in cockroaches and/or dust mites. Additionally, for example, a multiple allergen oral immunotherapeutic composition comprising one or more identified allergenic proteins from Table 2 that belongs to the Bet v 1 protein family are present in food (e.g., Bet v 1 family proteins present in peanut or walnut) may, for example, induce an immune response against a cross-reactive allergenic protein in the bet v 1 family present in white oak tree pollen or birch pollen. It is contemplated that such methods have parallel tolerizing effects on primary allergens as well as cross-reactive allergens.

Subjects can be, for example, administered such multiple allergen compositions orally, parenterally, nasally or topically.

Accordingly, in one aspect the present disclosure provides methods for treatment of subject that are multi-allergic or have the potential to become multi-allergic. In some embodiments, subjects have two or more allergies or food-intolerances. Such two or more diseases may be directed to identified allergenic proteins or to cross-reactive allergenic proteins or to both. In another aspect, the present disclosure provides methods for the treatment of subjects that have undiagnosed, suspected, or unidentified allergies or food-intolerances. In some embodiments, subjects have an existing allergy and may have another allergy directed to a cross-reactive allergenic protein that is undiagnosed, suspected, or unidentified. As can be appreciated from the exemplary methods provided above, identification of identified allergenic proteins, cross-reactive allergenic proteins, and cross-reactive T-cell epitopes by the methods of the present disclosure are believed to provide advantages for methods, compositions, and kits for the treatment, prevention, monitoring, and management of allergies and food-intolerances, autoimmune diseases, as well as any cross-reactive T-cell epitope-sensitive disorders, diseases or conditions.

In some embodiments, subjects are humans. Subject may be infants, juveniles or adults. In some embodiments, subject may be pregnant adults or in utero or unborn children. As such, in certain embodiments, a subject is an infant younger than 1 year of age, whereas in other embodiments the subject is older, e.g., 1 year old or more, 5 years old or more, etc., and including adults. In some embodiments, subjects are non-human mammals.

In some embodiments, subjects in need thereof may be administered a composition of the present disclosure for treatment, prevention, and/or monitoring of allergies, food-intolerances, or any diseases or epitope-sensitive conditions.

In some embodiments, subjects have a single food allergy or single food-intolerance. In some embodiments, subjects have two or more allergies or food-intolerances or subjects are multi-allergic or multi-food-intolerant. In some embodiments, multi-allergic subjects having a single food allergy or single food-intolerance (primary allergy) may have or be at risk of developing another allergy or another food-intolerance, which may as yet be undiagnosed, unidentified, or suspected. In some embodiments, based on a subject's clinical/medical history (or food diaries that may be maintained by a subject or subject's family or caregivers), any of a physician, allergist, other health care professional, subject, parent, and/or caregiver may request the subject be screened for another allergy. Such “another allergy” or “another food-intolerance” may be directed to one or more allergenic proteins having cross-reactivity with the subjects' one or more primary allergens (e.g., one or more identified allergenic proteins in a food source which a subject has an existing allergy). Such another allergy may also be interchangeably referred herein to as a “cross-reactive allergy.” One or more cross-reactive allergenic proteins disclosed herein or one or more cross-reactive allergenic proteins identified or identifiable by the methods of the present disclosure may be cross-reactive with one or more primary allergens of the subjects' existing allergies or food-intolerances and pose a threat of sensitizing such subjects and/or developing an allergy to such cross-reactive allergenic proteins. Subjects may have or develop cross-reactive IgE antibodies or cross-reactive T-cells that recognize cross-reactive epitopes on primary allergenic proteins as well as any cross-reactive allergenic proteins that may be present in a subject. It is contemplated that, in certain embodiments, if such cross-reactive IgE antibodies or cross-reactive T-cells are present, such subjects may benefit from and are candidates for treatment with a multiple allergen immunotherapy. Such subjects may, for example, be administered an effective amount of a multiple allergen oral composition, for example, a composition disclosed herein.

Subjects having two or more allergic diseases (e.g. allergies) may be administered a multiple allergen oral immunotherapeutic composition comprising a shrimp allergen, a cod allergen, a salmon allergen, a hen's egg allergen, a cow's milk allergen, a peanut allergen, a sesame allergen, a soy allergen, a pecan allergen, a cashew allergen, a hazelnut allergen, a walnut allergen, a pistachio allergen, an almond allergen, and a wheat allergen. In some embodiments, the subjects may be administered a multiple allergen oral immunotherapeutic composition comprising one or more cross-reactive allergenic proteins from protein families provided in Table 3 or Table 4. In some embodiments, the subjects may be administered a multiple allergen oral immunotherapeutic composition comprising one or more cross-reactive allergenic proteins provided in Tables 4-7 and 10-12. In some embodiments, the subjects may be administered a multiple allergen oral immunotherapeutic composition comprising one or more Cupin family proteins or cross-reactive MHC-binding T-cell epitopes from Tables 13-19 that are derived from one or more Cupin family proteins. In some embodiments, the subjects are administered a multiple allergen oral composition comprising one or more the Cupin family proteins from Tables 5 or 10. In some embodiments, the subjects are administered a multiple allergen oral composition comprising one or more of the cross-reactive allergenic proteins Ber_e_2, Car_i_2, Fag_e_1, Fag_e_13S, Fag_t_13S_globulin, Jug_n_4, Lup_a_vicilin, Lup_an_1, Pin_k_2, Pis_s_1, Pis_s_2, Pis_v_2, Ses_i_3, Ses_i_6, Ses_i_7, Sin_a_2, Vig_r_2, Car_i_4, Gly_m_5, Gly_m_6, Jug_r_4, Len_c_1 or Pru_du_6. In some embodiments, the subjects are administered a multiple allergen oral composition comprising one or more cross-reactive epitope peptides present on cross-reactive allergenic proteins Ber_e_2, Car_i_2, Fag_e_1, Fag_e_13S, Fag_t_13S_globulin, Jug_n_4, Lup_a_vicilin, Lup_an_1, Pin_k_2, Pis_s_1, Pis_s_2, Pis_v_2, Ses_i_3, Ses_i_6, Ses_i_7, Sin_a_2, Vig_r_2, Car_i_4, Gly_m_5, Gly_m_6, Jug_r_4, Len_c_1 or Pru_du_6. In some embodiments, the subjects are administered a multiple allergen oral composition comprising four or more cross-reactive epitope peptides present on cross-reactive allergenic proteins Ber_e_2, Car_i_2, Fag_e_1, Fag_e_13S, Fag_t_13S_globulin, Jug_n_4, Lup_a_vicilin, Lup_an_1, Pin_k_2, Pis_s_1, Pis_s_2, Pis_v_2, Ses_i_3, Ses_i_6, Ses_i_7, Sin_a_2, Vig_r_2, Car_i_4, Gly_m_5, Gly_m_6, Jug_r_4, Len_c_1 or Pru_du_6. In some embodiments, the subjects are administered a multiple allergen oral composition comprising four or more cross-reactive epitope peptides per protein present on two or more of cross-reactive allergenic proteins selected from the group consisting of Ber_e_2, Car_i_2, Fag_e_1, Fag_e_13S, Fag_t_13S_globulin, Jug_n_4, Lup_a_vicilin, Lup_an_1, Pin_k_2, Pis_s_1, Pis_s_2, Pis_v_2, Ses_i_3, Ses_i_6, Ses_i_7, Sin_a_2, Vig_r_2, Car_i_4, Gly_m_5, Gly_m_6, Jug_r_4, Len_c_1 and Pru_du_6.

In some embodiments, the multiple allergen immunotherapeutic compositions comprise one or more allergenic proteins or peptides that are cross-reactive with one or more cross-reactive allergenic proteins present in identified protein families of Table 3. In some embodiments, the multiple allergen immunotherapeutic compositions comprise one or more allergenic proteins or peptides that are cross-reactive with one or more cross-reactive allergenic proteins provided in Tables 4-7 or 10-12. In some embodiments, the multiple allergen immunotherapeutic compositions comprise one or more allergenic proteins or peptides that are cross-reactive with one or more cross-reactive allergenic proteins provided in Tables 4-7 or 10-12. In some embodiments, the multiple allergen immunotherapeutic compositions comprise one or more allergenic proteins or peptides that are cross-reactive with one or more cross-reactive allergenic MHC-II binding peptides provided in Tables 13-18, 20, and 22.

Non-limiting examples of other allergies and other food-intolerances include allergies and food-tolerances directed to food sources of Table 8 or disclosed herein. In some embodiments, a subject's other allergy is not an allergy to an allergenic protein included in a multiple allergen composition administered to the subject.

Subjects in need of treatment, prevention and/or monitoring may, for example, be first identified as having an existing allergy to a primary (first) allergen (food source or an identified allergenic protein). This can be performed, for example, by screening a subject's medical or clinical history. Then, subjects' undiagnosed or unidentified allergy (or food-intolerance) or subjects' risk of developing another allergy or food-intolerance to another food source containing one or more cross-reactive allergenic protein may be determined. Presence of another allergy or food-intolerance may, for example, be confirmed by testing either by skin prick test or by a bioassay performed on a biological sample obtained from the subject. The potential for developing another (additional) food allergy or food-intolerance may, for example, be determined by obtaining or having obtained a biological sample from the subjects and assessing the biological sample using the methods of the present disclosure. For example, a biological sample can be assessed for one or more immune markers or immune response upon exposure of the biological sample to an identified allergenic protein and one or more cross-reactive allergenic proteins of the present disclosure as described herein or by other methods known in the art. Immune response or immune markers that may be assessed in a biological sample obtained from a subject include: presence of cross-reactive antibodies (e.g., IgE, IgG such as IgG4, IgM, IgD, and IgA), Th1/Th2 profile, for example presence of Th2 cells indicating allergic profile, activation of other immune cells such as basophils, granulocytes, eosinophils and mast cells, release of cytokines indicative of Th2 (e.g., IL-4, IL-5, IL9, 11-13, IL-17 etc.) and of Th1 or undifferentiated T-cells such as TNFγ and IL-10, and/or degranulation (e.g., histamine and/or serotonin).

Also provided herein are methods for testing if a subject is a candidate for a multiple allergen oral immunotherapy, which includes assessing the subject for an unidentified allergy (or unidentified food-intolerance) by detecting the presence of IgE antibodies or T-cells in a biological sample from the subject that binds with one or more cross-reactive allergenic proteins (for example, cross-reactive allergenic proteins disclosed herein). If, for example, binding of IgE antibodies and/or T-cells to one or more cross-reactive allergenic proteins is detected, then that subject may be a candidate for administration of a multiple allergen oral immunotherapy. In some embodiments, the methods further comprise identifying a subject as having an allergy or food-intolerance to a single food having one or more identified allergenic proteins.

Administration of an oral immunotherapeutic composition comprising at least two or more identified allergenic proteins or peptides thereof to a patient having an allergy or at risk of having an allergy to one or more of the two of more identified allergenic proteins may, for example, boost resistance in the patient to the protein compared to treatment with one identified allergenic protein alone. Administration of an oral immunotherapeutic composition comprising at least two or more cross-reactive allergenic proteins or peptides thereof to a patient having an allergy or at risk of having an allergy to one or more of the two of more cross-reactive allergenic protein may, for example, boost resistance in the patient to the protein compared to treatment with one cross-reactive allergenic protein alone. In some embodiments, subjects are administered an oral immunotherapeutic composition comprising three or more isolated cross-reactive allergenic proteins.

In some embodiments, patients are administered two or more Cupin family proteins. In some embodiments, patients are administered three or more Cupin family proteins.

### Methods for Induction of Tolerization (Cross-Tolerization or Parallel Tolerization)

The present disclosure provides, for example, methods of administering a multiple allergen immunotherapeutic composition (for example, oral composition) to a subject to induce tolerization to multiple allergens which are cross-reactive allergens, for example, to induce tolerization to multiple allergens including a primary allergen (for example an identified allergenic protein) and one or more additional allergens that are cross-reactive with the primary allergen (e.g., the identified allergenic protein). Typically, multi-allergic subjects will have two or more existing allergies. Some subjects may be intolerant to two or more allergens or identified allergenic proteins. As a non-limiting example, two or more allergens could be from Table 1 or Table 2. However, some subjects may have one existing allergy that can be ascertained by review of their medical/clinical history and food diaries and may also have one or more unidentified or undiagnosed allergies or food-intolerances. The present disclosure provides that such unidentified and/or undiagnosed allergies or intolerances may be to one or more allergens that are cross-reactive to one or more of their primary allergens. Such subjects may show symptoms of poor immune health conditions, poor gut health, food-intolerance and/or allergies. Accordingly, provided herein are methods for treatment and monitoring of such multi-allergic subjects (e.g., subjects having 2 or more allergies) and of subjects having 2 or more food-intolerances. Further, some subjects in addition to having one or more existing allergy or food-intolerance may have become or may be at risk of becoming sensitized to additional allergenic proteins that are cross-reactive with one or more of their primary allergens.

The present disclosure provides, for example, methods for prevention, monitoring and treatment of subjects having or at risk of having an allergy or intolerance to one or more cross-reactive allergenic proteins that is cross-reactive with one or more of their primary allergenic proteins. Such subjects may, for example, be administered a multiple allergen oral immunotherapeutic composition of the present disclosure. By way of illustration only, a subject having known allergies to peanuts, cashews, and walnuts and having a potential or risk for developing an allergy or food intolerance to buckwheat (based on cross-reactivity of one or more allergenic proteins present in buckwheat to one or more of the allergenic proteins present in peanuts, cashews, and/or walnuts), can be treated with a multiple allergen oral immunotherapeutic composition containing one or more of the allergenic proteins present in peanuts, cashews, and walnuts (but not one or more cross-reactive allergenic proteins present in buckwheat) to induce parallel desensitization in the subject to not only peanuts, cashews, and walnuts, but also to buckwheat. Desensitization can be detected, for example, by the presence of cross-reactive IgEs in a biological sample obtained from the subject and assessed using any of the methods of the present disclosure. In this example, it is contemplated that the subject's resistance to buckwheat and its cross-reactive allergenic proteins may, for example, be increased as an effect of the one or more identified allergenic proteins of peanuts, cashews, and walnuts present in the multiple allergen oral immunotherapeutic composition. Further, inclusion of multiple allergenic proteins in a multiple allergen oral composition has an added benefit of increasing the diversity of allergenic epitopes to which subjects are exposed.

It is contemplated that in methods for promoting parallel tolerization to different allergens a subject may, for example, achieve tolerance to each allergen at different rates, e.g., tolerance to each allergen may be achieved within different periods of time even if treatment for each allergen is initiated at the same time using the same allergenic immunotherapy.

In some embodiments, subjects have an allergy or food intolerance to one or more foods listed in Table 1. In some embodiments, subjects have an allergy or intolerance to one or more identified allergenic proteins listed in Table 2.

Treatment with multiple allergen oral immunotherapy may, for example, manifest in the form of a modulation of a surrogate immune marker of a disease condition. For example, Th2 cell proliferation may be reduced, e.g., as determined using the assay described in the Examples below. The magnitude of Th2 cell proliferation reduction may vary, and in certain instances may range from 1.2× to 10×, such as 2× to 4×. Shift in Th1/Th2 balance may be reversed, as determined by using a Th1/Th2 panel bioassay such as that commercially available from Thermo Fisher Scientific, USA. Th1 and Th2 cytokines release may be determined. Any of IL-4, IL-5, IL9, 11-13, IL-17 and/or other cytokine indicators of Th2 cells as well as TNFγ, IL-10 and other indicators of non-terminally differentiated T-cells or Th1 cells may be determined using the methods disclosed herein and otherwise known in the art. Levels of exemplary cytokines IL-4, IL-5, IL9, 11-13 and/or IL-17 may be reduced and TNFγ and IL-10 may be increased as determined by using a Th1/Th2 panel bioassay.

In some embodiments, the presence or absence of cross-reactive antibodies may also be determined. For example, specific and cross-reactive IgG4 levels may be increased, e.g., as determined using a bioassay of the present disclosure. The magnitude of IgG4 level increase may vary, and in certain instances may range from 1.2× to 100×, such as 2× to 6×. As another example, specific and cross-reactive IgE levels may be reduced. The magnitude of IgE level reduction may vary, and in certain instances may range from 1.1× to 7×, such as 2× to 6×.

As another non-limiting example, activation of basophils (indicating IgE-mediated immune response) may be determined as further described in Example 10. A decrease in the activation of basophils would be indicative of tolerance.

Allergy symptoms that may be ameliorated by the methods disclosed herein include, but are not limited to: eczema, asthma, atopic dermatitis, bronchospasm, cough, rhinorrhea, angioedema, gastric hypermotility, urticaria (hives), pruritis, fatigue, bradycardia, and/or hypotension. The magnitude of the symptom reduction may vary, where in some instances the magnitude is 2-fold or greater, e.g., 5-fold or greater, including 10-fold or greater, e.g., as compared to a suitable control. In some instances, treatment of an allergy results the subject being cured of the allergy, such that the subject no longer suffers from the allergy.

In some embodiments, subjects with one or more allergies (for example, a single food allergy or two more food allergies of which at least one may be undiagnosed, or at least one may be unidentified as to its allergen) are administered a multiple allergen oral immunotherapy.

### Methods for Improving Gut Health

Provided herein in some embodiments are methods of increasing gut health of subjects having an intolerance to one or more food allergens. Such methods may, for example, result in the subject having an enhanced maintenance of healthy microbiota, or improving the resilience of microbiota, for instance, by reducing the numbers or colonization of pathogenic bacteria or viruses, or by maintaining and improving the intestinal integrity and barrier function. Gut health or gut balance may be characterized by inflammation, for example, when T cell proliferation is decreased (e.g., as described in greater detail below in the Examples), or there is a shift in Th1/Th2 balance. The magnitude of gut health enhancement may vary, where in some instances the magnitude is 2-fold or greater, e.g., 5-fold or greater, including 10-fold or greater, e.g., as compared to a suitable control.

### Methods for Improving Immune Health of Subjects

Provided herein, in some embodiments, are methods of increasing immune health or immune balance of a subject. In some embodiments, disclosed methods result in a subject having an improved immune response to a given challenge. In some embodiments, disclosed methods result in subjects having improved immune response to an existing allergy (or food-intolerance) as well as a suspected or hitherto undiagnosed or unidentified allergy (or food-intolerance) to a cross-reactive allergenic protein (e.g., an additional allergenic protein that is cross-reactive with the allergenic protein of the existing allergy). Immune health or immune balance may be characterized by inflammation, decrease of cross-reactive IgE, increase of cross-reactive IgG4 in plasma, or decrease of Th2 response, degranulation, and/or activation of basophils, granulocytes, and/or mast cells (e.g., as described in greater detail below in the Examples). The magnitude of immune health enhancement may vary, where in some instances the magnitude is 2-fold or greater, e.g., 5-fold or greater, including 10-fold or greater, e.g., as compared to a suitable control.

### Methods of Decreasing the Potential of the Subject for Developing an Immune Mediated Condition

Provided herein are methods of decreasing the potential of or a risk for the subject for developing an immune mediated condition, such as an immune-mediated inflammatory disease condition. Potential of or risk for developing an immune-mediated condition due to sensitization to cross-reactive allergenic proteins may exist in subjects having an existing food allergy or existing food-intolerance or are sensitized to allergens or antigens that are also implicated in an immune-mediated condition or are cross-reactive with an antigen implicated in an immune-mediated condition. For example, to determine risk reduction, if 100 different subjects were administered the composition, 20% or more of the subjects would show a decrease in their immune markers, e.g., IgE, as compared to the control group. The magnitude of the decrease in potential may vary, where in some instances the magnitude is 2-fold or greater, e.g., 5-fold or greater, including 10-fold or greater, e.g., as compared to a suitable control. Accordingly, provided herein are methods of preventing subjects from developing an immune mediated condition. As such, the methods of the present disclosure include administering a multiple allergen oral immunotherapeutic composition of the present disclosure to a subject that is not known to have or does not have an immune mediated condition. While the subject may not have or may not be known to have the immune mediated condition, the subject may be one that is suspected to be or known to be at risk of developing the immune mediated condition. In some embodiments, the subjects may have one or more food allergies or food-intolerances. In some embodiments, the subjects have at least one single food allergy. Subjects having a single food allergy, may be one that is suspected to be or known to be at risk for developing immune mediated condition.

### Methods for Improving Quality of Life

Provided herein are methods for improving quality of life (QoL) of a subject with two or more allergies or food-intolerances. The methods include administering to the subject a multiple allergen immunotherapeutic composition. Subjects who suffer from allergies and/or food-intolerances have a reduced QoL. Subjects administered with multiple allergen immunotherapeutic compositions, for example, orally, are likely to have an improvement in QoL. In some embodiments, subjects are informed that they are being administered to multiple allergen oral immunotherapeutic compositions to treat their allergies or food-intolerances (not a placebo). Knowledge of such treatment improves subjects' QoL.

Before, during or after an multiple allergen oral immunotherapy, a subject may undergo a QoL assessment. Even absent allergic reactions, subject having allergies suffer impaired QoL, including from the fear and anxiety of accidental ingestion of allergens, fear of allergic reactions, financial burden of allergies, social restrictions, etc. Subjects and their families and caregivers devote a significant time for food selections, for example, vetting and validating them by reading food labels, inquiring of ingredients in unlabeled foods, or by food avoidance in environments with potential for food cross-contamination. Anxiety and depression are often observed in subjects with allergies and food intolerance and in their families and caregivers.

Methods for measuring QoL, such as disease-related QoL, before, during, or after multiple allergen oral immunotherapy are developed and validated for the measurement of QoL in subjects with subjects having or at risk of having one or more food allergies and/or food-intolerances. Common methods for assessing QoL employ questionnaire designed to assess one or more domains, across one or more issues each of the burden imposed by food allergy or food-intolerance (for example for a single food allergy, for two or more food allergies, etc.) in qualitative and quantitative terms. Exemplary questionnaires are generally: (1) valid, (2) reproducible, (3) responsive, and (4) interpretable. QoL questionnaires are generally age-appropriate and validated. QoL questionnaires are selected from: Food allergy quality of life-parental burden questionnaire, Food Allergy Impact Scale, Food Allergy Independent Measure, Food Allergy Independent Measure child form, Food Allergy Independent Measure teen form, Food Allergy Independent Measure adult form, Food Allergy Independent Measure parent form, Food Allergy Parent Questionnaire, Child Health Questionnaire Parental form, Food Allergy Self-Efficacy Scale for parents, Pediatric Allergic Disease QoL Questionnaire, Food Allergy QoL Questionnaire-parent form, Food Allergy QoL questionnaire child form, Food Allergy QoL questionnaire-teenager form, Food Allergy QoL Assessment Tool for Adolescents, You and Your Food Allergy Questionnaire, the Food Allergy QoL-adult form, the 36-item short form health survey, a EuroQoL EQ-5D, such as EuroQoL EQ-5D Youth, 5-Level EQ-5D, the Hospital anxiety and Depression Scale, the Allergy to Peanuts Impacting Emotions and Life survey, and the Pediatric QoL Inventory. Such QoL questionnaires may be proxy reported or self-reported.

Improvement in QoL is a change between at least 2 points in time. One of skill in the art will understand that improvement is with respect to a particular instrument (such as a particular QoL questionnaire or other QoL assessment). As a non-limiting example, if a subject's QoL is assessed by a QoL questionnaire, then an improvement is an improvement in the score of the same QoL questionnaire. In some embodiments, the improvement is clinically significant, such as equal to or greater than the questionnaire's minimal clinical important difference (MCID). In some embodiments, the improvement is a change in a domain score, or a total score, of a QoL questionnaire between one time point such as baseline measurement, to a second time point such a time point during or after multiple allergen oral immunotherapy. Each domain of QoL questionnaire is measured on a scale from 1 to 7, with 7 being the worst quality of life.

Improvement in a subject's QoL may persist (including continuing to improve) for a period of time after completion of multiple allergen oral immunotherapy. The improvement may persist for about at least 1 month, 2 months, 3 months, 6 months, 9 months, 12 months, 18 months, 24 months, 36 months, 48 months, or 60 months. In some embodiments, the subjects' QoL is improved for any of about at least 1 month, 2 months, 3 months, 6 months, 9 months, 12 months, 18 months, 24 months, 36 months, 48 months, or 60 months while the subjects continue to be administered a multiple allergen oral immunotherapy composition according to a multiple allergen oral immunotherapy schedule. In some embodiments, the subject's QoL is improved for any of about at least 1 month, 2 months, 3 months, 6 months, 9 months, 12 months, 18 months, 24 months, 36 months, 48 months, or 60 months after the subject has achieved a peak desensitization to at least one allergen in the multiple allergen oral immunotherapy or to at least one cross-reactive allergen (such as by measurement by oral food challenge or by assessing a biological sample obtained from the subject for immune markers such as presence IgE).

The appropriate dosage of the allergenic proteins (e.g., identified allergenic proteins, cross-reactive allergenic proteins) and/or cross-reactive epitope peptides of the present disclosure may depend on a variety of factors. Such factors may include, but are in no way limited to, a patient's physical characteristics (e.g., age, weight, sex), whether the cross-reactive epitope is being used as single agent, multiple agent (e.g., multiple epitopes from a single allergenic protein and/or from two or more distinct cross-reactive allergenic proteins) or adjuvant therapy, the type of MHC restriction of the subject, the progression (or pathological state) of the food allergy or food-intolerance or other epitope-sensitive conditions, and other factors that may be recognized by one skilled in the art. In general, a cross-reactive epitope or combination of cross-reactive epitopes may be administered to a subject in an amount of from about 50 μg to about 5 mg. In some embodiments, dosage may be in an amount of from about 50 μg to about 500 μg.

The vaccines may be administered by any suitable parenteral, oral, nasal, and/or topical methods, including, but not limited to, systemic injections (e.g., subcutaneous, epicutaneous, intradermal, intramuscular, intravenous, etc.), mucosal administration (e.g., nasal, ocular, oral, vaginal, anal, etc.), topical administration (e.g., by a patch or a tape delivery), or by any other pharmacologically appropriate method. In some embodiments, the vaccines are administered by gene delivery methods known in the art. The vaccine may be administered for delivery at particular time intervals or in a single administration. For example, for a subcutaneous administration, exemplary dose of 50 μg of one or more cross-reactive peptides adsorbed on alum may be administered via inject on 3 occasions at 2-week intervals. The number of injections can vary depending on multiple factors.

### T-Cell Therapy

In another aspect, the present disclosure relates to antigen-specific T-cells, for example, regulatory T-cells (T-regs) or suppressor T-cells. In some embodiments, subjects are administered the cross-reactive allergenic proteins and cross-reactive MHC-II binding epitopes of the present disclosure to induce the presence of T-regs in vaccinated individuals or individuals administered T-cell epitope therapy. In some embodiments, subjects are administered a multiple allergen oral immunotherapy (comprising vaccines or across-reactive MHC-II binding T-cell peptides) and T-regs are collected from the subjects and clones are propagated. The antigen-specific T-regs that are obtained from the subjects can be used for immunotherapy, for example by amplification of the T-reg clones, for the treatment, prevention of allergy and other autoimmune diseases. Allogenic and autogenic cells may be used.

### Routes of Administration

The administration route employed in a given method may vary, e.g., depending on the nature of the allergen immunotherapeutic composition. As reviewed above, physiologically acceptable compositions that include a multiple allergen composition may be formulated for delivery to a subject using a variety of different administration routes, such as but not limited to: oral, parenteral, nasal, and topical administration. As such, aspects of the methods may include orally, parenterally, nasally and topically, etc., administering a multiple allergen composition or physiologically acceptable composition that includes multiple allergens disclosed herein to a subject.

### Dosing Schedule

A dosing schedule may vary as desired or prescribed by a clinician or other health professionals, and may depend on a number of different factors, e.g., purpose of the administration, age of the subject, condition of the subject, nature of the physiologically acceptable composition, etc.

Subjects may, for example, be administered multiple allergen oral immunotherapy by regular exposure to increasing doses of multiple allergen oral immunotherapeutic compositions. Protocols for multiple allergen oral immunotherapy may, for example, involve updosing (also sometimes called a build-up phase) and/or a maintenance phase. A multiple allergen oral immunotherapy can further include an optional initial escalation phase. An initial escalation phase may, for example, involve exposure to small doses of allergens over the course of several hours (e.g. or more) to two days. These small doses may, for example, be increased until the subject reaches a goal dose or a highest tolerated dose (HTD) for the initial escalation phase. A subject then usually begins an updosing phase. Additional multiple allergen oral immunotherapy may, for example, include a maintenance phase involving continued administration of multiple allergen oral immunotherapeutic compositions for a period of time. One goal of the multiple allergen oral immunotherapy is establishing a desensitized state, wherein a subject being treated is less likely to suffer a severe or life-threatening allergic reaction upon accidental exposure to the primary allergens and/or the cross-reactive allergens, for example, establishing parallel tolerization to multiple allergens (those present in the multiple allergen oral immunotherapeutic composition as well as other allergens that are cross-reactive with allergens present in multiple allergen oral immunotherapeutic compositions is induced in the subject). In some embodiments, desensitization or tolerance is clinical tolerance.

In certain embodiments, a multiple allergen composition or physiologically acceptable composition that includes the same is administered to a subject on an hourly basis, on an every few hours basis (e.g., every 2, 3, 4, 6, 8, 12 hours), on a daily basis, on a weekly basis, on a bi-weekly basis, on a monthly basis, on a bimonthly basis, on a quarterly basis, on a semi-annual basis, on an annual basis, etc., for a treatment period of time. The treatment period of time may also vary, for example, in some instances the treatment period of time is 1 day or longer, 1 week or longer, 2 weeks or longer, 1 month or longer, 3 months or longer, 6 months or longer, 1 year or longer, 2 years or longer, 3 years or longer, 5 years or longer, 10 years or longer, etc., up to the life of the subject.

### XIV. EXEMPLARY EMBODIMENTS

Embodiment 1. A method of identifying additional allergenic proteins that are cross-reactive with one or more allergenic proteins present in a multiple allergen oral immunotherapy comprising:
(a) performing one or more in silico bioinformatics steps to identify additional allergenic proteins that are homologous to one or more identified allergenic proteins (reference protein(s)) present in the multiple allergen oral immunotherapy, wherein the homology is based on full-length proteins;
(b) determining a cross-reactivity score of each of the identified additional allergenic proteins within a protein family with respect to a reference protein; wherein the reference protein is an allergenic protein within the protein family and present in the multiple allergen oral immunotherapy; and
(c) identifying one or more additional allergenic proteins as a cross-reactive allergenic protein based on homology and/or the cross-reactivity score of each additional allergenic protein with respect to the reference protein.
Embodiment 2. The method of embodiment 1, wherein the protein family is selected from the group consisting of: Tropomyosin, EF hand family (Beta-parvalbumin), ATP:guanido phosphotransferase (Arginine Kinase), Triosephosphate isomerase, Fructose bisphosphate aldolase class I, Alpha/beta casein, Lipocalin, Kappa-casein, Transferrin, C-type lysozyme/alpha-lactalbumin family, Serum albumin, Enolase, Serpin serine protease inhibitor, Kazal-type serine protease inhibitor, Collagen, Lipoprotein, Prolamin superfamily, Cupin, Profilin, Bet v 1 family, Oleosin, Isoflavone reductase family, Plant defensin, 60S acidic ribosomal protein, Heat shock protein Hsp70, Fe/Mn superoxide dismutase, Papain-like cysteine protease, Thaumatin-like protein, Unclassified, GILT family, Hevein-like and class I/II chitinase, Zinc finger, C3HC4 type (RING finger), SCAI family, Peroxidase, Serpin serine protease inhibitor, Thionin, Thioredoxin, Transcription elongation factor 1, Lipid transfer protein, and Triosephosphate isomerase.
Embodiment 3. The method of embodiments 1 or 2, the method further comprising mapping one or more cross-reactive epitopes such as a B-cell epitope, a T-cell epitope or an MHC-binding epitope on each protein within the protein family member.
Embodiment 4. The method of embodiment 3, wherein cross-reactive epitopes of embodiment 3 are correlated with the one or more identified allergenic proteins present in the multiple allergen oral therapeutic and/or one or more cross-reactive allergenic protein.
Embodiment 5. The method of embodiments 1-4, the method further comprising mapping one or more cross-reactive epitopes such as a B-cell epitope, a T-cell epitope or an MHC-binding epitope on a subset of proteins with the protein family comprising one or more identified allergenic protein and/or one or more cross-reactive allergenic protein.
Embodiment 6. The method of embodiments 3-5, one or more cross-reactive epitopes binds to one or more, 2 or more, 3 or more, 4 or more, 5 or more, 6 or more 7 or more, 8 or more 9 or more, or 10 or more MHC class II molecules.
Embodiment 7. The method of any of preceding embodiments, wherein the multiple allergen oral immunotherapy comprises one or more food sources selected from the group consisting of shrimp, cod, salmon, hen's egg, cow's milk, peanut, sesame, soy, pecan, cashew, hazelnut, walnut, pistachio, almond, and wheat.
Embodiment 8. The method of any of preceding embodiments, wherein the multiple allergen oral immunotherapy comprises shrimp, cod, salmon, hen's egg, cow's milk, peanut, sesame, soy, pecan, cashew, hazelnut, walnut, pistachio, almond, and wheat.
Embodiment 9. The method of any of preceding embodiments, wherein the one or more the identified allergenic protein is selected from the group consisting of a shrimp allergen, a cod allergen, a salmon allergen, a hen's egg allergen, a cow's milk allergen, a peanut allergen, a sesame allergen, a soy allergen, a pecan allergen, a cashew allergen, a hazelnut allergen, a walnut allergen, a pistachio allergen, an almond allergen, and a wheat allergen.
Embodiment 10. The method of any of preceding embodiments, wherein the one or more of the identified allergenic proteins present in the multiple allergen oral immunotherapy is selected from the group consisting of identified allergens listed in Table 2.
Embodiment 11. The method of any of the preceding embodiments, wherein the additional allergenic proteins are one or more of the additional allergenic proteins provided in: Table 4.
Embodiments 12. The method of any of the preceding embodiments, wherein the identified cross-reactive allergenic proteins have a cross-reactivity score of ≥0.5 with respect to the reference protein and/or have ≥70% Homology to the reference protein.
Embodiments 13. The method of any of the preceding embodiments, wherein the identified cross-reactive allergenic proteins in the Cupin protein family are provided in: Tables 5 or 10.
Embodiments 14. The method of any of the preceding embodiments, wherein the identified cross-reactive epitopes are provided in Tables 13-23.
Embodiment 15. The method of embodiment 14, wherein the at least one or more MHC class II molecule to which a cross-reactive epitope of Embodiment 14 binds is provided in Tables 13-23.
Embodiment 16. The method of any of the preceding embodiments, wherein the method further comprises determining homology and/or a cross-reactivity score of each full-length additional allergenic protein with respect to a full-length reference protein that is not one or more of the identified allergenic proteins present in the multiple allergen oral immunotherapy, wherein cross-reactivity score of >0.5 and/or a homology of at least 70% with respect to the reference protein will identify additional cross-reactive allergenic proteins.
Embodiments 17. The method of any of the preceding embodiments, wherein the method further comprises generating an allergenic map (AllerMap) of a food source, wherein generating the AllerMap comprises mapping all identified allergenic proteins present in a food source and cross-reactive allergenic proteins that are not present in the food source.
Embodiment 18. The method of any of the preceding embodiments, wherein the one or more bioinformatics steps comprises applying one or more algorithms to determine the homology of one or more of the full-length additional allergenic proteins to one or more of the identified allergenic proteins in a multiple allergen oral immunotherapy based on pairwise identity and similarity.
Embodiment 19. The method of any of the preceding embodiments, wherein the one or more bioinformatics steps comprises applying one or more algorithms to determine the cross-reactivity score of one or more of the additional allergenic proteins with respect to the one or more of the identified allergenic proteins within the protein family.
Embodiment 20. The method of any of the preceding embodiments, wherein the method further comprises one or more bioinformatic steps for obtaining a structural model of identified allergenic proteins present in the multiple allergen oral immunotherapy and identifying surface exposed amino acids on one or more of the cross-reactive allergenic proteins or one or more of cross-reactive epitope.
Embodiment 21. A computer-implemented method for identification of additional allergenic proteins that are cross-reactive with one or more identified allergenic proteins present in multiple allergen oral immunotherapy, comprising:
(a) receiving, in a computer processing system, a data input comprising a first dataset comprising a plurality of identified allergenic protein sequences present in a multiple allergen oral immunotherapy and a second dataset comprising a plurality of additional allergenic protein sequences within the protein family; and
(b) performing one or more bioinformatic steps comprising:
(i) applying one or more algorithms to compare each of the plurality of additional allergenic protein sequences present in second dataset to one or more of the plurality of identified allergenic protein sequences present in the first dataset and determining homology based on pairwise identity and similarity of each of the plurality of additional allergenic protein sequences in second dataset and each of the plurality of identified allergenic protein sequences in the first dataset; and
(ii) applying one or more algorithms for determining a cross-reactivity score for each of the plurality of additional allergenic protein sequences in the second dataset and/or the dataset with respect to a reference protein sequence to identify cross-reactivity of each of the plurality of additional allergenic protein sequences in the second dataset to the reference protein; wherein the reference protein sequence is an identified allergenic protein sequences present in multiple allergen oral immunotherapy; and
(c) identifying one or more additional allergenic protein sequences from the second dataset as cross-reactive with one or more identified allergenic sequences in the first dataset if the one or more additional allergenic protein has a cross-reactivity score of ≥0.5 and/or at least 70% homology to the reference protein.
Embodiment 22. A non-transitory computer-readable medium storing thereon executable instructions, that when executed by a computer, causes the computer to execute a method for identification of allergenic protein sequences that are cross-reactive to one or more allergenic proteins present in multiple allergen oral immunotherapy, the method comprising:
(a) receiving, in a computer processing system, a data input comprising a first dataset identified allergenic protein sequences present in a multiple allergen oral immunotherapy and a second dataset comprising a plurality of additional allergenic protein sequences within a protein family;
(b) applying an algorithm to compare each of the plurality of additional allergenic protein sequences present in second dataset to one or more of the plurality of identified allergenic protein sequences present in the first dataset and determining homology based on pairwise identity and similarity of each of the plurality of additional allergenic protein sequences in second dataset and each of the plurality of identified allergenic protein sequences in the first dataset;
(c) applying an algorithm for determining a cross-reactivity score for each of the plurality of additional allergenic protein sequences in the second dataset and/or the dataset with respect to an reference protein sequence to identify cross-reactivity of each of the plurality of additional allergenic protein sequences in the second dataset to the reference protein; wherein the reference protein sequence is an identified allergenic protein sequences present in multiple allergen oral immunotherapy; and
(d) identifying one or more additional allergenic protein sequences from the second dataset as cross-reactive with one or more identified allergenic sequences in the first dataset, wherein the identified cross-reactive additional allergenic protein and has a cross-reactivity score of ≥0.5 and/or at least 70% homology to a reference protein.
Embodiment 23. A non-transitory computer-readable storage medium encoded with a computer program including instructions executable by a digital processing device to create an application, the application comprising:
(a) a software module configured to obtain data input comprising a first dataset comprising a plurality of identified allergenic protein sequences present in a multiple allergen oral immunotherapy and a second dataset comprising a plurality of additional allergenic protein sequences within a protein family;
(b) a software module configured to apply an algorithm to compare each of the plurality of additional allergenic protein sequences present in second dataset with one or more of the plurality of identified allergenic protein sequences present in the first dataset and determining homology based on pairwise identity and similarity of each of the plurality of additional allergenic protein sequences in second dataset and each of the plurality of identified allergenic protein sequences in the first dataset;
(c) a software module configured to apply an algorithm for determining a cross-reactivity score for each of the plurality of additional allergenic protein sequences in the second dataset and/or the dataset with respect to a reference protein sequence to identify cross-reactivity of each of the plurality of additional allergenic protein sequences in the second dataset to the reference protein; wherein the reference protein sequence is an identified allergenic protein sequences present in multiple allergen oral immunotherapy; and
(d) a software module for identifying one or more of the plurality of additional allergenic protein sequences from the second dataset as cross-reactive with one or more of the plurality of identified allergenic sequences in the first dataset, wherein the identified cross-reactive additional allergenic protein has a cross-reactivity score of ≥0.5 and/or at least 70% homology to a reference protein.
Embodiment 24. A computer-implemented system, comprising:
(a) a digital processing device comprising an operating system configured to perform executable instructions, and a memory device;
(b) a computer program including instructions executable by a digital processing device to create an application comprising;
(i) a software module configured to obtain data input comprising a first dataset comprising a plurality of identified allergenic protein sequences present in a multiple allergen oral immunotherapy and a second dataset comprising a plurality of additional allergenic protein sequences within a protein family;
(ii) a software module configured to apply an algorithm to compare each of the plurality of additional allergenic protein sequences present in second dataset with one or more of the plurality of identified allergenic protein sequences present in the first dataset and determining homology based on pairwise identity and similarity of each of the plurality of additional allergenic protein sequences in second dataset and each of the plurality of identified allergenic protein sequences in the first dataset;
(iii) a software module configured to apply an algorithm for determining a cross-reactivity score for each of the plurality of additional allergenic protein sequences in the second dataset and/or the dataset with respect to a reference protein sequence to identify cross-reactivity of each of the plurality of additional allergenic protein sequences in the second dataset to the reference protein; wherein reference protein sequence is an identified allergenic protein sequences present in multiple allergen oral immunotherapy; and
(iv) a software module configured for identifying one or more additional allergenic protein sequences from the second dataset having a cross-reactivity score of >0.5 and/or at least 70% homology to an reference protein as a cross-reactive allergenic protein, wherein the reference protein is an identified allergenic protein present in the multiple allergen oral immunotherapy and the identified cross-reactive allergenic protein sequence from the first dataset is not present in the multiple allergen oral therapy.
Embodiment 25. The method of any one of embodiments 21-24, wherein the method comprises generating an allergenic map of all allergenic proteins identified to be present in a food sources and all cross-reactive allergenic proteins to at least one of the identified allergenic proteins present in the food source.
Embodiment 26. The method of any one of embodiments 21-25, wherein method further includes generating an in silico library comprising a plurality of peptides derived from the plurality of additional allergenic protein sequences in the second Dataset.
Embodiment 27. The method of embodiment 26, wherein the peptides are overlapping 5-200 mers with a sliding window of 1-5 amino acids.
Embodiment 28. The method of embodiment 26 or 27, wherein the peptides are overlapping 8 mer-15-mer peptides with a sliding window of 1-5 amino acids.
Embodiment 29. The method of any one of embodiments 26-28, wherein the method further comprises obtaining a third dataset comprising a plurality of T-cell epitope peptides present in food allergens and applying an alignment algorithm to each of the plurality of peptides in the in silico library with the plurality of T-cell epitope peptides present in third dataset to determine homology (based on pair-wise identity and similarity) and generate an in silico T-cell epitope peptide library comprising a plurality of T-cell epitope peptides identified as present on cross-reactive allergenic proteins.
Embodiment 30. The method of embodiment 29, wherein the method further comprises obtaining a fourth dataset comprising a plurality of MHC-II-binding T-cell epitopes and applying an alignment algorithm to each of plurality of the identified T-cell epitope peptides in the T-cell epitope library to identify a plurality of peptides comprising MHC-II-binding T-cell epitopes based on homology (based on 70% identity and 80% coverage of the peptides).
Embodiment 31. The method of any one of embodiments 5-30, wherein the binding affinity (IC50) of one or more of the plurality of peptides comprising an MHC-II binding T-cell epitope ranges from 500 nM Kd to 1 nM Kd.
Embodiment 32. The method of any one of embodiment 5-31, wherein the binding affinity (IC50) of one or more of the plurality of peptides comprising MHC-II binding T-cell epitope peptides is ≤100 nM Kd.
Embodiment 33. The multiple allergen oral composition of embodiment 1 further comprising one or more cross-reactive allergenic proteins.
Embodiment 34. A method for identification of identifying cross-reactive allergenic proteins that are cross-reactive with one or more identified allergenic protein present in a food source comprising:
(a) performing one or more in silico bioinformatics steps to identify additional allergenic proteins that are homologous to the one or more identified allergenic proteins within a protein family; and/or
(b) determining a cross-reactivity score of each of the additional allergenic proteins within the protein family with respect to each of the identified allergenic proteins; and
(c) identifying one or more additional allergenic proteins as a cross-reactive allergenic protein based on minimal acceptable homology and/or a minimal acceptable cross-reactivity score of each additional allergenic protein with respect to the reference protein.
Embodiment 35. The multiple allergen composition of embodiment 34, wherein the homology is based on pairwise identity and similarity comparison of full-length proteins and the minimal acceptable homology is at least 70%.
Embodiment 36. The multiple allergen composition of embodiment 34, wherein the minimal acceptable cross-reactivity score is ≥0.5.
Embodiment 37. The multiple allergen composition of any one of embodiments 34-36, wherein the method further comprises mapping cross-reactive epitopes on one or more of the identified allergenic proteins and/or one or more cross-reactive allergenic proteins within the family.
Embodiment 38. The multiple allergen composition of embodiment 37, wherein the mapping of cross-reactive epitope comprises a method, the method further comprising: providing a providing a dataset comprising a plurality of peptide K-mers with a sliding window of 1 to 10 amino acids derived from all allergenic proteins within the protein family; providing another dataset comprising a plurality of experimentally-validated epitopes, wherein the experimentally-validated epitopes are T-cell epitopes and/or MHC-II-binding epitopes and/or B-cell epitopes; performing one or more bioinformatics steps to identify the presence or absence of experimentally-validated T-cell epitopes and/or MHC-II-binding epitopes and/or B-cell epitopes on each of the allergenic proteins within the protein family.
Embodiment 39. The method of embodiment 38, wherein the one or more bioinformatics steps comprise: performing an alignment algorithm on the dataset the plurality of peptide K-mers with a sliding window of 1 to 10 amino acids and the dataset comprising the plurality of experimentally-validated epitopes.
Embodiment 40. The method of embodiment 38 or 39, wherein the one or more bioinformatics steps comprises performing an alignment algorithm to map the experimentally-validated T-cell epitopes and/or MHC-II-binding epitopes and/or B-cell epitopes on each of the allergenic proteins within the protein family to identify cross-reactive epitopes (experimentally-validated T-cell epitopes and/or MHC-II-binding epitopes and/or B-cell epitopes) that are shared by or common to one or more identified allergenic proteins and one or more cross-reactive proteins within the protein family.
Embodiment 41. The methods of embodiment 39 or 40, wherein the alignment algorithm is BLASTP.
Embodiment 42. A computer-implemented method for identification of an allergenic protein that is cross-reactive with an identified allergenic protein present in a food source, comprising:
(a) receiving, in a computer processing system, a data input comprising a first dataset further comprising a plurality of full-length identified allergenic protein sequences and a second dataset comprising a plurality of full-length additional allergenic protein sequences within a protein family;
(b) applying an algorithm to compare each of the plurality of full-length additional allergenic protein sequences present in first dataset to one or more of the plurality of full-length identified allergenic protein sequences present in the second dataset, and determining homology (based pairwise identity and similarity) of each of the plurality of full-length additional allergenic protein sequences in first dataset to each of the plurality of full-length identified allergenic proteins sequences in the second dataset;
(c) applying an algorithm for determining a cross-reactivity score for each of the plurality of full-length additional allergenic protein sequences in the second dataset with respect to each full-length identified allergenic protein (a reference protein) sequence in the first dataset to predict cross-reactivity of each of the plurality of full-length additional allergenic protein sequences in the first dataset with respect to the reference protein; and
(d) identifying one or more full-length additional allergenic protein sequences from the second dataset as cross-reactive with one or more full-length Identified Allergenic Sequences in the first dataset, wherein the identified full-length cross-reactive allergenic protein is correlated as having at least 70% homology to a reference protein and/or a cross-reactivity score of >0.5 with respect to a reference protein.
Embodiment 43. A computer-implemented method for identification of cross-reactive epitopes shared by one or more identified allergenic protein present in a food source and one or more allergenic proteins within a protein family to which the one or more identified allergenic proteins belong, comprising:
(a) receiving, in a computer processing system, a data input comprising a first dataset further comprising a plurality of full-length allergenic protein sequences present in an identified protein family, wherein the plurality of full-length allergenic protein sequences comprise the one or more identified allergenic protein present in the food source, a second dataset comprising a plurality of overlapping peptide K-mers derived the plurality of full-length additional allergenic protein sequences from the first dataset; a third dataset comprising a plurality of validated T-cell epitopes; and/or a fourth dataset comprising a plurality of validated MHC-binding T-cell epitopes; and/or a fifth dataset comprising a plurality of validated B-cell epitopes;
(b) applying an algorithm to map the:
(i) plurality of validated T-cell epitopes in the third dataset on to the overlapping peptide K-mers of the second dataset to identify the presence of one or more validated T-cell epitopes in the second dataset;
(ii) plurality of validated MHC-binding T-cell epitopes in the fourth dataset on to the overlapping peptide K-mers of the second dataset to identify the presence of one or more validated MHC-binding T-cell epitopes in the second dataset;
(iii) plurality of validated B-cell epitopes in the sixth dataset on to the overlapping peptide K-mers of the second dataset to identify the presence of validated B-cell epitopes in the second dataset;
(c) applying an algorithm to the identified validated T-cell epitopes, WIC-binding T-cell epitopes, and/or the B-cell epitopes in (f) to each of the plurality of full-length allergenic proteins in the first dataset to identify the cross-reactive epitopes; and optionally,
(d) applying an algorithm to compare each of the plurality of full-length allergenic protein sequences present in the first dataset to each of the plurality of full-length allergenic protein sequences present in the first dataset, and determining homology (based on pairwise identity and similarity) of each of the plurality of full-length allergenic protein sequences in first dataset to each of the plurality of full-length allergenic protein sequences in the first dataset; wherein in each of the plurality of full-length allergenic proteins is a reference protein with respect to each of the plurality of full-length allergenic proteins in the first dataset;
(e) applying an algorithm for determining a cross-reactivity score for each of the plurality of full-length allergenic protein sequences in the first dataset with respect to each of the plurality of full-length allergenic protein sequence (each a reference protein) in the first dataset to identify cross-reactivity of each of the plurality of full-length allergenic protein sequences in the first dataset with respect to its reference protein;
(f) identifying one or more full-length allergenic protein sequences from the first dataset as cross-reactive with one or more full-length allergenic sequences in the first dataset, wherein the identified full-length cross-reactive allergenic protein has at least 70% homology to a reference protein and/or a cross-reactivity score of ≥0.5 with respect to a reference protein.
Embodiment 44. The method of embodiment 43, wherein the method further comprises correlating the identified cross-reactive epitopes of Step (g) to the identified cross-reactive allergenic proteins.
Embodiment 45. The method of embodiment 43 or 44, wherein the overlapping peptide K-mers range from 5 amino acids to 200 amino acids in length with a sliding window of 1 to 5 amino acids, from 7 to 150 amino acids in length with a sliding window of 1 to 5 amino acids, from 8 to 30 amino acids in length with a sliding window of 1 to 5 amino acids, from 9 amino acids to 25 amino acids in length with a sliding window of 1 to 5 amino acids.
Embodiment 46. The method of any one of embodiments 43-45, wherein the overlapping peptide K-mers is 14-mer or 15 mer with a sliding window of 1 to 5 amino acids.
Embodiment 47. The method of any one of embodiments 43-46, further comprises rank ordering each of the identified cross-reactive epitopes by
(a) the number of MHC molecules (and/or variants thereof) that each cross-reactive epitope binds; and/or
(b) the number of allergenic proteins in the first dataset carrying each cross-reactive epitope;
Embodiment 48. The method of any one of embodiments 43-47, further comprising selecting cross-reactive epitopes that are creative with 1 or more, 2 or more, 3 or more, 4 or more, 5 or more, 6 or more, 7 or more, 8 or more, 9 or more or 10 or more MHC molecules (and/or variants thereof).
Embodiment 49. The method of any one of embodiments 43-48, further comprising selecting cross-reactive epitopes that bind 1 to 150 MHC molecules.
Embodiment 50. The method of embodiment 49, wherein cross-reactive epitopes bind to at least 1 MHC molecule, at least 5 WIC molecules, at least 10 WIC molecules, at least 25 MHC molecules, at least 30 WIC molecules, at least 40 MHC molecules, at least 50 MHC molecules, at least 60 WIC molecules, at least 70 WIC molecules, at least 80 MHC molecules, at least 90 MHC molecules, or at least 100 WIC molecules (and variants thereof).
Embodiment 51. A composition comprising one or more cross-reactive allergenic protein or cross-reactive epitope identified by a method of any one of the embodiments 1-50.
Embodiment 52. The composition of embodiment 56, wherein the composition is a single allergen composition or a multiple allergen composition.
Embodiment 53. The multiple allergen composition of embodiment 51 or 52 wherein the multiple allergen composition comprises two or more allergenic proteins or two or more cross-reactive epitopes.
Embodiment 54. The multiple allergen composition of any one of embodiments 51-53, wherein the composition comprises:
(a) one or more identified allergenic proteins and one or more cross-reactive allergenic proteins; or
(b) two or more identified allergenic proteins and one or more cross-reactive allergenic proteins; or
(c) one or more of identified allergenic proteins provided in Table 2 and two or more cross-reactive allergenic proteins; or
(d) identified allergenic proteins provided in Table 2; or
(e) two or more cross-reactive allergenic proteins selected from the group of allergenic proteins provided in Table 4; or
(f) two or more cross-reactive allergenic proteins from Cupin family (Table 10); or
(g) three or more Cupin family proteins; or
(h) one or more proteins or cross-reactive epitope peptides selected from the group consisting of Ber_e_2, Car_i_2, Fag_e_1, Fag_e_13S, Fag_t_13S_globulin, Jug_n_4, Lup_a_vicilin, Lup_an_1, Pin_k_2, Pis_s_1, Pis_s_2, Pis_v_2, Ses_i_3, Ses_i_6, Ses_i_7, Sin_a_2, Vig_r_2, Car_i_4, Gly_m_5, Gly_m_6, Jug_r_4, Len_c_1 and Pru_du_6, or any combination thereof; or
(i) one or more identified proteins from Table 2 and one or more cross-reactive allergenic proteins from Cupin family; or
(j) one or more cross-reactive epitopes, wherein the one or more cross-reactive epitopes is T-cell epitope or an MHC-binding epitope or a B-cell epitope, or any combination thereof.
(k) one or more epitopes in a Cupin protein; or
(l) one or more cross-reactive epitopes of Tables 13-23; or
(m) at least four cross-reactive epitopes; or
(n) one or more food sources comprising one or more cross-reactive allergenic proteins or cross-reactive peptides; or
(o) one or more food sources comprising one or more identified allergenic proteins and one or more food sources comprising one or more cross-reactive allergenic proteins.
Embodiment 55. A multiple allergen oral immunotherapeutic composition comprising 3 or more Cupin family proteins and a pharmaceutically acceptable carrier, wherein the upon administration of the oral immunotherapeutic composition to a subject having an allergy or at risk of having an allergy to one or more the 3 or more Cupin family proteins, the subject has boosted resistance to the protein as compared to treatment with a single Cupin family protein alone; and/or is also treated for a different allergen in the Cupin family protein.
Embodiment 56. The multiple allergen oral immunotherapeutic composition of embodiment 55, wherein the Cupin family proteins are selected from the group provided in Table 5.
Embodiment 57. The multiple allergen oral immunotherapeutic composition of embodiment 55 or 56, wherein the Cupin family proteins is selected from the group provided in Table 10.
Embodiment 58. A multiple allergen oral immunotherapeutic composition comprising 12 or more cross-reactive epitopes from 3 or more Cupin family proteins and a pharmaceutically acceptable carrier, wherein each Cupin family protein provides at least 4 cross-reactive epitopes, and wherein the upon administration of the oral immunotherapeutic composition to a subject having an allergy or at risk of having an allergy to one or more the 3 or more Cupin family proteins, the subject has boosted resistance to the protein as compared to treatment with a single Cupin family protein alone; and/or is also treated a different allergen in the Cupin family protein.
Embodiment 59. A multiple allergen composition of any one of embodiments 51-58, wherein the cross-reactive allergenic proteins and/or cross-reactive epitopes are identified by any of the method of any one of embodiments 34-50.
Embodiment 60. The composition of embodiment 54 or 55, comprising at least 4 cross-reactive epitopes from one cross-reactive allergen selected from the allergenic proteins selected from an identified protein family provided in Table 3.
Embodiment 61. The composition of embodiment 60 comprising at least 4 cross-reactive epitopes from one cross-reactive allergen selected from Cupin family (Table 10).
Embodiment 62. A composition of any one of embodiments 51-61 that is formulated as an oral composition, a parenteral composition, a nasal composition, or a topical composition.
Embodiment 63. A cross-reactive epitope identified by the methods of any one of embodiments 41-50.
Embodiment 64. A cross-reactive epitope selected from Tables 13-23.
Embodiment 65. A vector comprising one or more cross-reactive epitopes of any one of embodiments 51-64.
Embodiment 66. A vector comprising at least 4 cross-reactive epitopes identified by the methods of any one of embodiments 34-50.
Embodiment 63. A host cell comprising the cross-reactive epitope of any one of embodiments 34-66.
Embodiment 64. A mouse comprising one or more cross-reactive epitopes of any one of embodiments 34-66.
Embodiment 65. A vaccine comprising one or more cross-reactive epitopes identified by a method of any one of embodiments 34-50.
Embodiment 66. A vaccine comprising one or more cross-reactive epitopes selected from Table 13-23.
Embodiment 67. A vaccine comprising at least 4 cross-reactive epitopes selected from Table 13-23.
Embodiment 68. A composition comprising a vaccine of embodiment 66 or 67.
Embodiment 69. A composition comprising a host cell comprising a vector of embodiment 65 or 66.
Embodiment 70. An antibody that binds to a cross-reactive epitope identified by the methods of any one of embodiments 34-50.
Embodiment 71. An antibody that binds to a cross-reactive MHC-binding cross-reactive epitope provided in Tables 13-23.
Embodiment 72. The antibody of embodiment 70 or 71, wherein the antibody is a specific IgE, IgG, IgA, IgM or IgD.
Embodiment 73. A composition comprising an antibody of any one of embodiments 70-72.
Embodiment 74. A method of preparing a multiple allergen oral immunotherapeutic composition that is capable of treating an allergy to one or more cross-reactive allergenic proteins comprising:
(a) identifying one or more cross-reactive allergenic proteins and/or at least four cross-reactive epitopes per protein on one or more cross-reactive allergenic protein that are cross-reactive allergenic with one or more identified allergenic protein in the multiple allergen oral immunotherapeutic composition by a method of any of the preceding embodiments (1-50); and
(b) selecting as components in the multiple allergen oral immunotherapeutic composition:
(i) two or more identified allergenic proteins, wherein the two or more allergenic proteins have at least 70% homology and/or a cross-reactive score of >0.5 with the one or more identified cross-reactive allergenic proteins; or
(ii) or more cross-reactive epitopes from step (a).
Embodiment 75. A method of preparing a multiple allergen oral immunotherapeutic composition that is capable of parallel tolerization of a subject having an existing food allergy and has an unidentified or undiagnosed allergy or at risk of having a second allergy to one or more cross-reactive allergenic proteins comprising:
(a) identifying one or more cross-reactive allergenic proteins and/or four or more cross-reactive epitopes per protein on one or more cross-reactive allergenic proteins that are cross-reactive with one or more identified allergenic proteins in the food to which the subject has an existing allergy by a method of any of the embodiments 1-50; and
(b) selecting as components in the multiple allergen oral immunotherapeutic composition
(i) the one or more cross-reactive allergenic proteins, wherein the one or more allergenic proteins have at least 70% homology and/or a cross-reactive score of ≥0.5 with the one or more identified cross-reactive allergenic proteins; and/or
(ii) four or more cross-reactive epitopes from step (a).
Embodiment 76. A method of embodiment 74 or 75, wherein the one or more identified allergenic proteins and/or one or more cross-reactive allergenic proteins belong to an identified protein family provided in Table 3.
Embodiment 77. The method of any one of embodiments 74-76, wherein the one or more identified allergenic proteins is selected from the group consisting of a shrimp allergen, a cod allergen, a salmon allergen, a hen's egg allergen, a cow's milk allergen, a peanut allergen, a sesame allergen, a soy allergen, a pecan allergen, a cashew allergen, a hazelnut allergen, a walnut allergen, a pistachio allergen, an almond allergen, and a wheat allergen.
Embodiment 78. The method of any one of embodiments 74-75, wherein the identified allergenic protein is selected from Table 2.
Embodiment 79. A multiple allergen oral immunotherapeutic composition comprising 3 or more Cupin family proteins and a pharmaceutically acceptable carrier, wherein the upon administration of the oral immunotherapeutic composition to a subject having an allergy or at risk of having an allergy to one or more the 3 or more Cupin family proteins, the subject has boosted resistance to the protein as compared to treatment with a single Cupin family protein alone; and/or is also treated for a different allergen in the Cupin family protein.
Embodiment 80. The multiple allergen oral immunotherapeutic composition of embodiment 79, wherein the Cupin family proteins are selected from the group provided in Table 5.
Embodiment 81. The multiple allergen oral immunotherapeutic composition of embodiment 79 or 80, wherein the Cupin family proteins is selected from the group provided in Table 10.
Embodiment 82. A method for identifying one or more specific antibodies useful for detection of an allergenic food antigen, comprising identifying one or more cross-reactive epitopes according to a method of any one of embodiments 1-50; and identifying the specific antibodies that bind to the identified cross-reactive B-cell epitopes.
Embodiment 83. The method of embodiment 82, wherein the one or specific antibodies is detected in an individual, a biological sample or a cell.
Embodiment 84. The method of embodiment 82 or 83, wherein the one or more specific antibodies bind to a cross-reactive allergenic protein or an identified allergenic protein that is immobilized to a solid support or a substrate.
Embodiment 85. The method of any one of embodiments 82-84, wherein the allergenic food antigen is from a food source provided in Table 1 or Table 8.
Embodiment 86. The method of embodiment 82, wherein the allergenic food antigen is a shrimp allergen, a cod allergen, a salmon allergen, a hen's egg allergen, a cow's milk allergen, a peanut allergen, a sesame allergen, a soy allergen, a pecan allergen, a cashew allergen, a hazelnut allergen, a walnut allergen, a pistachio allergen, an almond allergen, or a wheat allergen.
Embodiment 87. The method of any one of embodiments 82-86, wherein the allergenic food antigen is from identified protein family provided in Table 3.
Embodiment 88. The method of any one of embodiments 82-87, wherein the allergenic food antigen is a cross-reactive allergenic protein in Cupin protein family.
Embodiment 89. The method of embodiment 88, wherein the cross-reactive allergenic protein provided in Table 10.
Embodiment 90. The method of any one of embodiments 82-90, wherein one or more specific antibodies bind to a cross-reactive epitope identified by a method of any of the embodiments 1-41 immobilized on a solid support or a substrate.
Embodiment 91. The method of any one of embodiments 82-90, wherein one or more cross-reactive epitope identified by a method of any one of embodiments 1-50 is in a solution.
Embodiment 92. The method of any one of embodiments 82-90, wherein the one or more cross-reactive epitopes is from Tables 13-23.
Embodiment 93. The method of any one of embodiment 82-91, wherein the biological sample or the cell is immobilized on a solid support or substrate.
Embodiment 94. The method of any one of embodiments 82-93, wherein the specific antibody is IgE, IgG, IgA, IgM or IgD.
Embodiment 95. A method for detecting the presence of specific food antigens, comprising:
(a) providing one or more specific antibodies to a cross-reactive B-cell epitope;
(b) contacting a biological sample or a cell that may have an allergenic protein under conditions sufficient to permit binding of the specific antibodies to the cross-reactive B-cell epitope; and
(c) detecting the binding of the specific antibodies to identify specific food antigens.
Embodiment 96. The method of embodiment 95, wherein the specific antibody is an IgE, IgG, IgA, IgM or IgD.
Embodiment 97. The method of embodiment 95 or 96, wherein the specific food antigen is a food antigen from Tables 4-7 or 10-12.
Embodiment 98. The method of any one of embodiments 95-97, wherein the specific antibodies bind to a cross-reactive epitope identified by a method of any of the embodiments 1-50.
Embodiment 99. The method of any one of embodiments 95-98, wherein the specific antibodies bind to a cross-reactive epitope provided in Tables 13-23.
Embodiment 100. The method of any one of embodiments 95-99, wherein the specific antibody is immobilized on a solid support or substrate.
Embodiment 101. The method of any one of embodiments 84, 90, 93, or 100, wherein the solid support or substrate is a membrane, a filter, a chip, a slides (such as a microscopic slide), a wafer, a fiber, a magnetic or a non-magnetic bead, a gel, a tubing, a plate (such as a microtiter plate), microtiter strips, a polymer, a microparticle, a sphere, a microsphere, a nanosphere, a particle, a microparticle, a nanoparticle, or a capillary.
Embodiment 101. The method of embodiment 100, wherein the solid support or a substrate is an array (such as a microarray).
Embodiment 102. The method of embodiment 100 or 101, wherein the specific antibodies immobilized on the solid support or substrate are multiplexed.
Embodiment 103. The method of any one of embodiments 100-102, wherein six or more specific antibodies are immobilized on the solid support or a substrate.
Embodiment 104. The method of any one of embodiments 83-103, wherein the biological sample is selected from the group consisting of whole blood, serum, plasma, sputum, blood cells (e.g., peripheral blood mononuclear cells (PBMC), T-cells, B-cells, basophils, etc.), immune cells, tissue samples, biopsy samples, urine, tears, peritoneal fluid, pleural fluid, breast duct fluid, breast exudate, breast milk, breast fluids, saliva, semen, mucous, lymph, cytosol, ascites, amniotic fluid, bladder washes, and bronchioalveolar lavage.
Embodiment 105. A method of treating a subject for two or more allergies, comprising:
(a) identifying the subject as having an allergy to a single food having one or more identified allergenic proteins;
(b) assessing the subject for another allergy by the presence of IgE antibodies or T-cells that bind to one or more immunologically cross reactive proteins with one or more identified allergenic proteins, and if one or more additional allergies to other proteins are present; and
(c) administering a multiple allergen oral immunotherapy to the subject.
Embodiment 106. The method of embodiment 105, wherein the multiple allergen oral immunotherapy does not contain one or more of the immunologically cross reactive proteins.
Embodiment 107. The method of embodiment 105 or 106, wherein identifying the patient is identifying the patient's clinical history of a single food allergy.
Embodiment 108. The method of any one of embodiments 105-107, wherein the single food allergy is an allergy to a food source provided in Table 1.
Embodiment 109. The method of any one of embodiments 105-108, wherein the subject's allergy to another allergenic protein is not a protein included in the multiple allergen oral immunotherapeutic composition.
Embodiment 110. The method of any one of embodiments 105-109, wherein the multiple allergen oral immunotherapy comprises a shrimp allergen, a cod allergen, a salmon allergen, a hen's egg allergen, a cow's milk allergen, a peanut allergen, a sesame allergen, a soy allergen, a pecan allergen, a cashew allergen, a hazelnut allergen, a walnut allergen, a pistachio allergen, an almond allergen, and a wheat allergen.
Embodiment 111. The method of any one of embodiments 105-110, wherein the multiple allergen oral immunotherapy comprises the identified allergenic proteins of Table 2.
Embodiment 112. The method of any one of embodiments 105-111, wherein the one or more additional allergy is an allergy to one or more food sources provided in Table 8.
Embodiment 113. The method of any one of embodiments 105-112, wherein the one or more immunologically cross-reactive allergenic proteins are selected from the group provided in Table 4.
Embodiment 114. The method of any one of embodiments 106-113, wherein the one or more immunologically cross-reactive allergenic proteins are selected from the group provided in Tables 10-12.
Embodiment 115. A method for detecting development of clinical tolerance to and/or monitoring the efficacy of, a multiple-allergen immunotherapeutic composition in a subject being administered the multiple allergen immunotherapeutic composition and in need thereof, comprising:
(a) obtaining a biological sample from the subject at one or more intervals of administration;
(b) contacting the biological sample with one or more identified allergenic proteins present in the multiple allergen immunotherapeutic composition, one more immunologically cross reactive proteins and/or one or more cross-reactive epitopes;
(c) measuring the presence of or binding to IgE, IgG, and/or T-cells to the one or more identified allergenic proteins, one or more immunologically cross-reactive proteins and/or one or more cross-reactive epitopes;
(d) comparing, to assess the efficacy of the multiple allergen immunotherapeutic composition,
(i) the amount of unbound one or more identified allergenic proteins, unbound one or more immunologically allergenic proteins, or unbound one or more cross-reactive epitopes, and/or
(ii) the concentration of the IgE, IgG or T-cells with a previous measurement of the IgE, IgG and/or T-cells; and
where clinical tolerance is indicated when the amount of unbound one or more identified allergenic proteins present in the multiple allergen immunotherapy, unbound one more immunologically cross reactive proteins, or unbound one or more cross-reactive epitopes in the biological sample, or the concentration of the IgE, IgG or T-cells, is less than previously identified number of the one or more identified allergenic proteins, immunologically cross reactive proteins, and/or cross-reactive epitopes, or previous concentration of IgE, IgG or T-cell epitopes.
Embodiment 116. The method of embodiment 115, wherein the multiple allergen immunotherapeutic composition is a multiple allergen oral immunotherapeutic composition.
Embodiment 117. The method of embodiment 116, wherein the immunologically cross reactive proteins or the cross-reactive epitopes are immobilized on a solid support or substrate.
Embodiment 118. A method of assessing the presence or absence of cross-reactive IgE antibodies or cross-reactive T-cells in a biological sample, comprising:
(a) contacting the biological sample with one or more identified allergenic proteins;
(b) measuring the binding of IgE antibodies or T-cells to the one or more identified allergenic proteins;
(c) selecting the allergenic proteins that bind to the IgE antibodies or T-cells to obtain one or more binding allergenic proteins;
(d) comparing the similarity between the binding allergenic proteins and one or more untested allergenic proteins to assess the presence of one or more cross-reactive IgE antibodies or cross-reactive T-cells.
Embodiment 119. A method of assessing the presence or absence of cross-reactive IgE antibodies or cross-reactive T-cells in a biological sample, comprising:
(a) contacting the biological sample with one or more cross-reactive MHC-II-binding T-cell epitopes;
(b) measuring the binding of IgE antibodies or T-cells to the one or more cross-reactive MHC-II binding T-cell epitopes;
(c) selecting the cross-reactive that bind to the IgE antibodies or T-cells to obtain one or more binding cross-reactive MHC-II-binding epitopes;
(d) comparing the similarity between the binding cross-reactive MHC-II-binding epitopes and one or more untested cross-reactive MHC-II-binding epitopes to assess the presence of one or more cross-reactive IgE antibodies or cross-reactive T-cells.
Embodiment 120. A method for testing whether a patient is a candidate for a multiple allergen oral immunotherapy, comprising: assessing the patient for an unidentified allergy by detecting the presence of IgE antibodies or T-cell epitopes in a patient's biological sample that bind to one or more immunologically cross reactive proteins with one or more identified allergenic proteins, and if an unidentified allergy to other proteins is present; and if present, the patient is a candidate for administration of a multiple allergen oral immunotherapy.
Embodiment 121. A kit for a biological cross-reactivity IgE and/or IgG assay comprising six or more distinct proteins each bound to a solid support or a substrate, wherein the 6 or more distinct proteins are selected from the group consisting of Table 2, Table 4, and/or Table 10 proteins.
Embodiment 122. A detection kit comprising 6 or more specific IgE and/or IgG antibodies each bound to a solid support or substrate wherein the 6 or more specific IgE are antibodies to B-cell epitopes identified by a method of any of the embodiments 1-50.
Embodiment 123. The kit of embodiment 121 or 122, wherein each solid support is a bead.
Embodiment 124. The kit of any one of embodiments 121-123, further comprising an IgE and/or IgG specific labeling reagent and the distinct proteins.
Embodiment 125. The kit of any one of embodiments 121-124, further comprising one or more of a binding buffer, a wash buffer, and a detection buffer.
Embodiment 126. A kit comprising a single allergen or multiple allergen immunotherapeutic composition of any one of embodiments 51-73 and a pharmaceutical carrier.

The foregoing examples are presented herein for illustrative purposes only, and should not be construed as limiting in any way

### EXAMPLES

### Example 1. Allergenic Proteins and Protein Families

Identification of all known food allergenic proteins in each of the fifteen food sources listed in Table 1 was performed using the name and allergen identification numbers and Genbank numbers as keywords. AllFam allergen Database was used for initial screening. AllFam allergen database hosted by Medical University of Vienna available on the worldwide web at meduniwien.ac.at/allfam/ is a resource for classifying allergens into protein families. The allergenic proteins in the 15 food sources are listed in Table 2.

Next, all protein families to which allergenic proteins in the 15 food sources belonged were identified and reviewed (Table 2 and Table 3). The allergen database and Genbank were screened to ensure collection of all known allergenic proteins in all known allergenic food sources. Then, protein (amino acid) sequences for: (1) allergenic proteins in Table 2, and (2) all the additional allergenic proteins belonging to each of the identified protein families were retrieved from Genbank and Swiss Prot databases in FASTA format and stored locally. Review of the additional allergenic proteins for each of the identified protein families revealed that these additional allergenic proteins (Table 4) were present in additional food sources (Table 8) that are either known to be allergenic or based on this analysis could be potentially cross-reactive allergenic proteins.

FIG. 1 shows an exemplary method for the identification of food sources and protein families that contain potentially cross-reactive allergenic proteins.

Surprisingly, there was very little overlap between allergenic protein families or allergenic proteins between animal food sources and plant food sources. Further, allergenic proteins belonged to a small number of protein families, but each protein family spanned a wide variety of food sources.

For these studies, amino acid sequences for each protein family were set up in individual datasets. For example, a dataset containing amino acid sequences of Cupin family proteins in a FASTA format was created. Similarly, datasets were created for each of the remaining protein families. Another dataset included amino acid sequences of the allergenic proteins from the 15 food sources of Table 1.

### Example 2. Protein Sequence Homology Search

Homology between allergenic protein sequences within each identified protein family was evaluated. For this step, full-length amino acid sequences for allergenic proteins within each of the protein families were retrieved from GenBank and SWISS-PROT in FASTA format using FTP transfer protocols. Duplicates, shorter isoforms, and partial sequences were identified and removed from further analyses in this study.

Homology was determined using one or more bioinformatic steps using full-length protein sequences. Sequence similarity searching is a method of searching sequence databases by using alignment to a query sequence. By statistically assessing how well database and query sequences match one can infer homology and transfer information to the query sequence. BLAST and FASTA algorithms are some of the bioinformatic tools that were used to identify identity and similarity of each amino acid residue in the full-length sequences of allergenic proteins.

Allergenic proteins within a protein family were compared with other proteins within the same protein family. Pair-wise sequence alignment and multiple sequence alignment of the protein sequences were performed using the program Clustal-Omega for the following protein families: Cupin, Tropomyosin, EF Hand family, Profilin and Bet v 1. Sequence identity and similarity (and thus homology) were determined at the SIAS server using default parameters.

Pairwise alignments were performed using the k-tuple method in Clustal-Omega, Sampling method: mBed to speed, Matrix: Gonnet PAM 250 matrix; gap open penalty: 6 bits, gap extension: 1 bit. Multiple sequence alignments were produced using the HHalign package, which aligns two profile hidden Markov models (HMM). The guide tree was next constructed using the UPGMA method (Daugelaite et al. (2013) “An Overview of Multiple Sequence Alignments and Cloud Computing in Bioinformatics” International Scholarly Research Notices, vol. 2013, Article ID 615630).

### Example 3. Determination of Cross-Reactivity Score of Allergenic Proteins within a Protein Family

A cross-reactivity score for allergenic proteins within a protein family was calculated with respect to other or additional allergenic proteins in the same protein family. Cross-reactivity for each protein with respect to a “reference protein” was used to predict the likelihood of cross-reactivity between a protein and its reference protein. The A-RISC cross-reactivity score is believed to be predictive of a cross-reactive allergenic protein's ability to induce an immune response (such as induction of cross-reactive antibodies such as IgE and IgG (e.g., IgG4)) in an individual, a biological sample or a cell. In this Example, a cross-reactivity score for allergenic proteins within the Cupin protein family was calculated with respect to other or additional allergenic proteins within the protein family using the methods described by Chruszcz et al. PLoS ONE 13(11): e208276.

Briefly, A-RISC cross reactivity score for allergenic protein within the Cupin protein family with respect to other or additional allergenic Cupin protein family members was determined. First, identity and similarity were determined between full-length allergenic proteins in the Cupin protein family as calculated by SIAS server ((Immunomedicine Group: Tools >>SIAS (ucm.es)) using default parameters. Then a cross-reactivity score for each allergenic protein with respect to every other or additional allergenic protein in the Cupin protein family was calculated using the following algorithm: (I+S)/2.

The SIAS webpage divides amino acids into the following similarity groups: aromatic, aliphatic, positively charged, negatively charged, small with hydroxyl group and neutral polar. Sequence similarities and identities calculated with SIAS were stored and used for the generation of various plots as well as for cross-reactivity score calculations.

In addition to the Cupin family, cross-reactivity scores for Tropomyosin, EF Hand, Profilin, Bet v 1 protein and profilin families were also determined.

In each case, the cross-reactivity scores were categorized into 4 levels as shown in Table 9. A-RISC Cross-reactivity scores of ≥0.5-1 (medium high to high likelihood of cross-reactivity with a reference protein) was deemed a minimal cross-reactivity score (medium-high to high) indicative of a cross-reactive allergenic protein.

Cross-reactivity analyses and correlation were performed for allergenic proteins present in the indicated protein families (including allergenic proteins from food sources not listed on Table 1, for example, from sources listed in Table 8 which also includes certain non-food allergen sources).

If an allergenic protein in a protein family was found to have at least 70% full-length homology (homology cutoff or minimum acceptable homology) to a reference protein and/or a cross-reactivity score (A-RISC score) of ≥0.5 (cross-reactivity cutoff or cutoff or minimum acceptable cross-reactivity score), then such an allergenic protein was identified as a cross-reactive allergenic protein with respect to its reference protein (and vice versa).

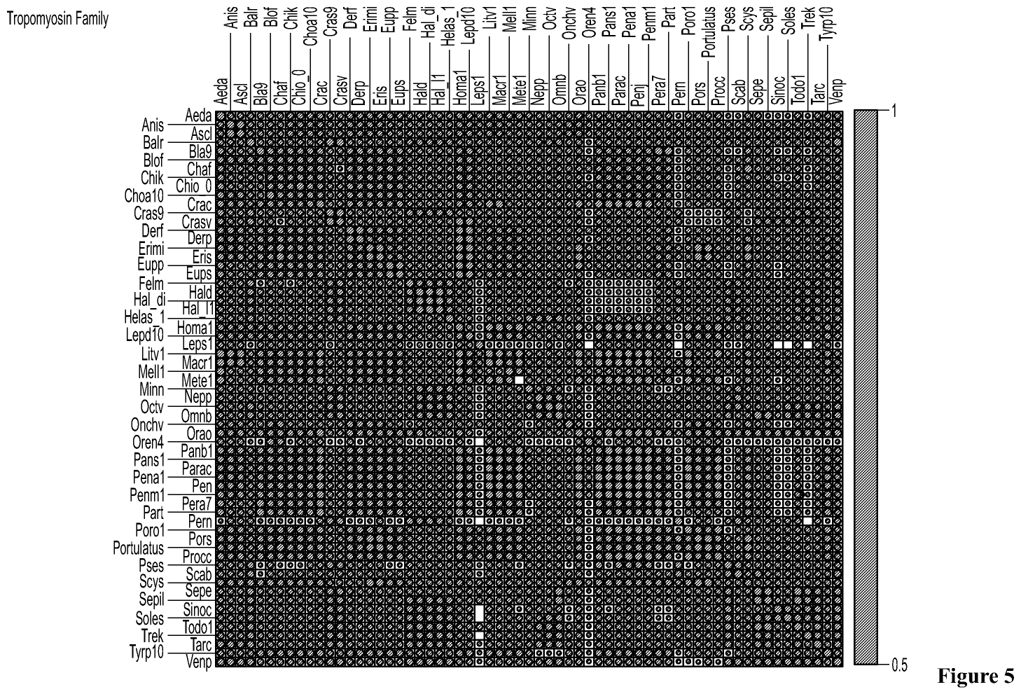

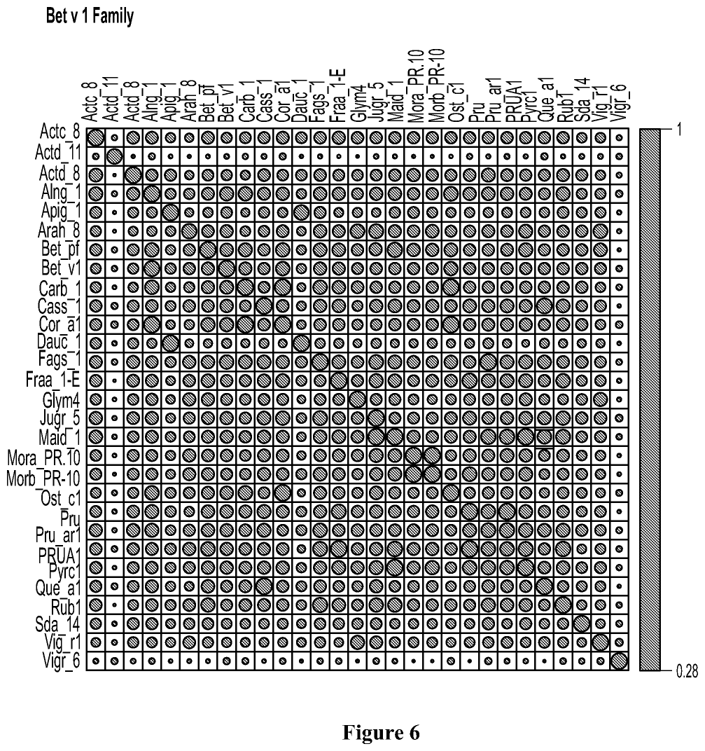

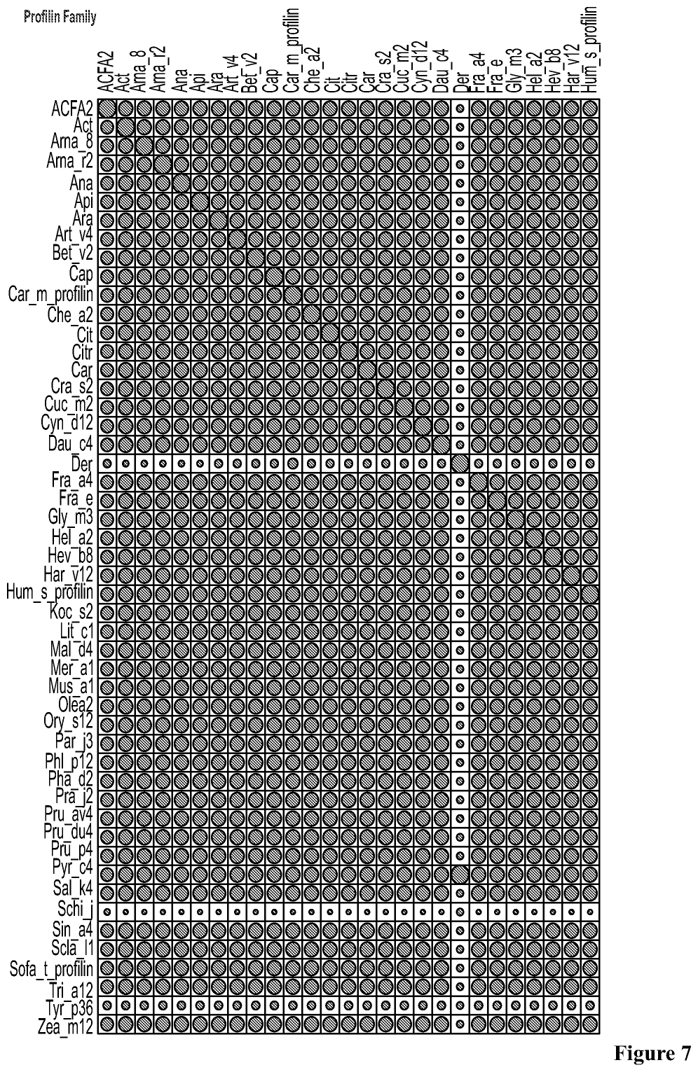

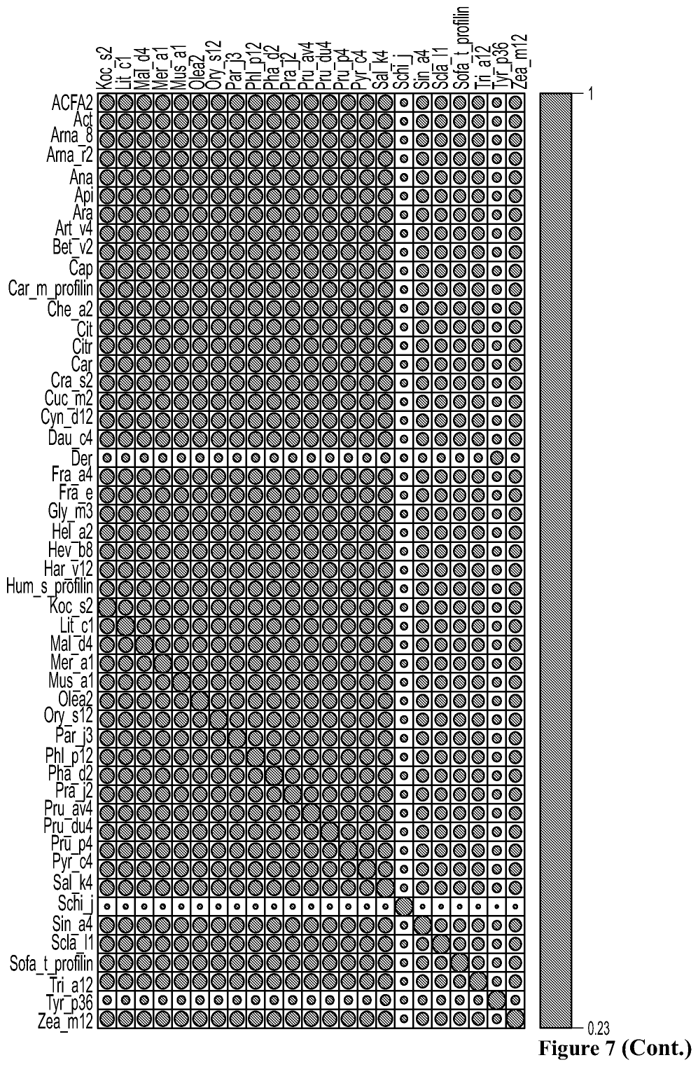

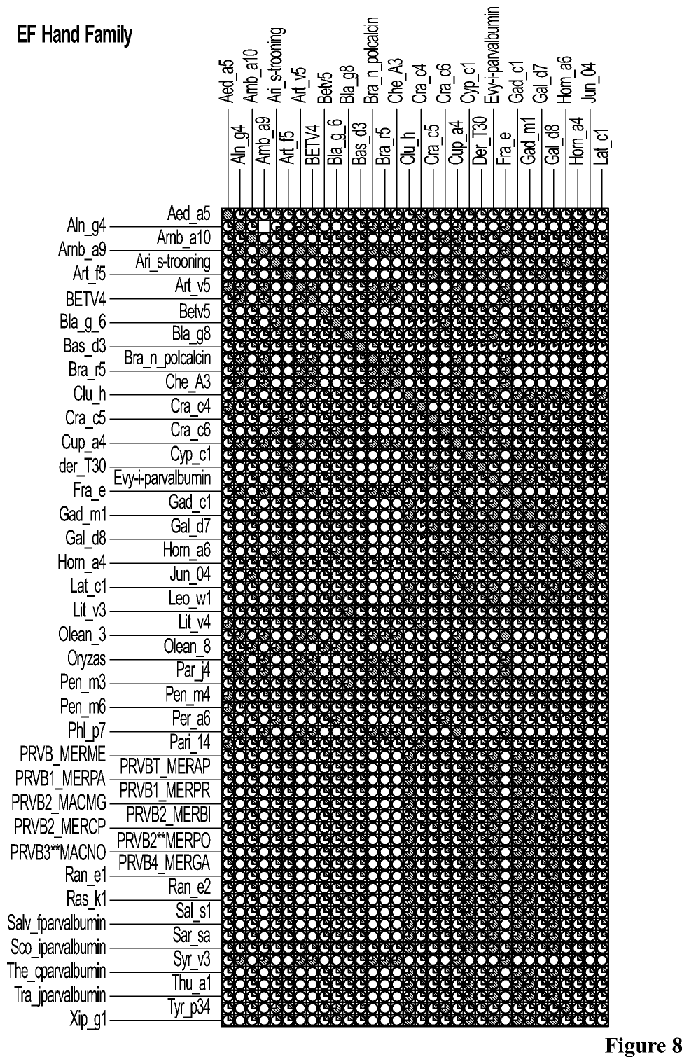

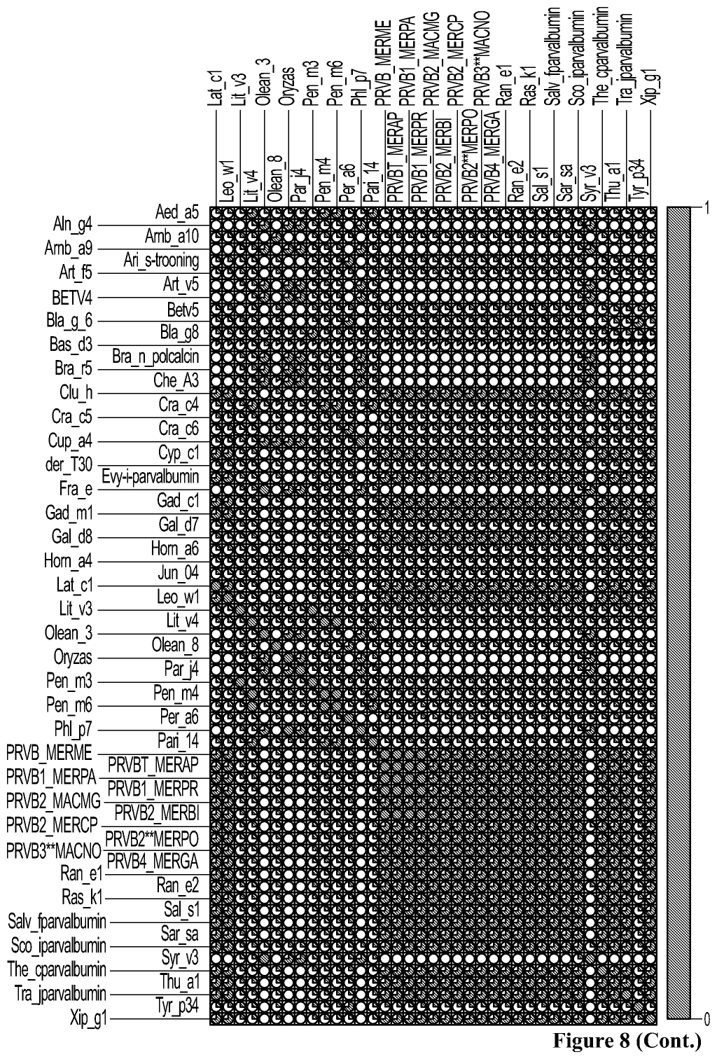

Allergenic proteins present in Cupin, Bet v 1, Profilin, Tropomyosin and EF Hand protein families were identified (See Table 4) and homology and cross-reactivity was calculated. 2-dimensional matrices illustrating the pair-wise comparison of each allergenic protein within each protein family with respect to other or additional allergenic proteins within the family based on homology and cross-reactivity are shown for the Cupin family (FIG. 4), Tropomyosin family (FIG. 5), Bet v 1 family (FIG. 6), Profilin family (FIG. 7), and EF Hand family (FIG. 8). Larger dots indicate greater homology and cross-reactivity potential.

Exemplary cross-reactive proteins that belong to the Cupin protein family and have a cross-reactivity score of ≥0.5 with respect to a reference protein are listed in Table 10. Cross-reactive allergenic proteins with a cross-reactivity score of ≥0.7 are predicted to have a high probability of cross-reactivity to the reference protein. Further, it is noted that that cross-reactivity was observed not only between food allergenic proteins but also between food-allergenic proteins and non-food allergenic proteins. Similarly, cross-reactive allergenic proteins in Tropomyosin (Table 11) and Bet v 1 (Table 12) protein families, each having a full-length homology of ≥70% (based on pairwise identity and similarity) and/or cross-reactivity score of ≥0.5 with respect to at least one allergenic protein within the protein family were identified.

The methods disclosed herein may be used to identify additional allergenic proteins and cross-reactive allergenic proteins.

### Example 4. Determination of T-Cell Epitopes

Highly immunogenic proteins contain many T-cell epitopes or concentrated clusters of T-cell epitopes within their amino acid sequences, whereas non-immunogenic proteins tend to contain fewer epitopes. Several T-cell epitope-mapping tools are known in the art. For this Example, T-cell epitope mapping was performed using the experimentally-validated T-cell epitope peptides and epitope prediction tools available on the IEDB server available on the world wide web at iedb.org.

Briefly, all known experimentally-validated T-cell epitopes (approximately 10,000 T-cell epitopes) were first downloaded using the Tepitool available on the world wide web at tools.iedb.org/tepitool from the IEDB database and stored locally for use in this study.

An in silico library containing approximately 9440 peptides (15-mer amino acid length overlapping peptides with a sliding window of 1 amino acid) was generated from all (37) allergenic proteins present in the Cupin protein family using bioinformatics tools used in the art. The entire dataset of 15-mers was then screened in an unbiased and unsupervised manner against the approximately 10,000 experimentally-validated T-cell epitopes downloaded and stored locally by use of one or more bioinformatics tools. In this Example, the BLAST program with default parameters was used to identify “hits” against experimentally-validated T-cell epitope peptides to identify and map predicted T-cell epitopes on allergenic proteins in the Cupin protein family. A homology cutoff or minimal acceptable homology parameter was set for ≥80% identity and the resulting epitope peptide hits (approximately 7162) were collected and stored locally. Thus, linear T-cell epitopes were determined in this Example for all Cupin family allergenic proteins.

These approximately 7162 hits were identified as T-cell epitopes based on homology to experimentally-validated T-cell epitopes and include redundant hits arising from the screening of the entire dataset containing 15-mers derived from all 37 Cupin protein family allergenic proteins at the same time in an unbiased and unsupervised manner (data not shown). The 7162 identified T-cell epitopes were then further screened against experimentally-validated epitopes that bind to at least one MHC class II molecule as described below in Example 5.

### Example 5. MHC Class H-Binding Epitope Peptides

Major histocompatibility complex class II (MHC-II or HLA-II) molecules are expressed on the surface of professional antigen-presenting cells where they display T-cell epitope peptides to T-helper cells in conjunction with MHC-II molecules, which orchestrate the onset and outcome of many host immune responses, including T-cell activation and proliferation, B-cell activation and antibody (e.g., IgG, IgE, etc.) production, etc. The aim of this Example was to identify which of the approximately 7162 in silico predicted T-cell epitopes from allergenic proteins in the Cupin protein family identified in Example 4 would bind to at least one of the MHC-II molecules.

In this study, the 7162 T-cell epitope hits from the Cupin protein family from Example 4 were in silico tested for predicted binding to at least one of the MHC Class II molecules (HLA-DP, HLA-DM, HLA-DOA, HLA-DOB, HLA-DQ, and HLA-DR and their variants) using prediction tools and algorithms available in the art. This MHC-II binding T-cell epitope screening study was performed on the IEDB server available on the world wide web at tools.iedb.org/mhcii using the default IEDB Recommended 2.20 method. This method uses the Consensus approach, combining NN-align, SMM-align, CombLib and Sturniolo where any corresponding predictor is available for the molecule, otherwise NetMHCIIpan is used. The Consensus method is further described in Wang et al. (2010) BMC Bioinformatics 11:568 and Wang et al. (2008) PLoS Comput Biol. 4(4):e1000048. Homology based on pairwise identify and coverage was used for identification of MHC-II binding epitopes that are highly likely to be present in Cupin proteins. Acceptable minimum homology was set for at least 70% identity and at least 80% coverage over the peptide.

To classify the 7162 T-cell epitope peptides into binders and non-binders, a binding affinity threshold was selected. A binding affinity threshold of 500 nm Kd in the context of the present disclosure was considered acceptable, thus classifying all T-cell epitope peptides with an IC50 binding affinity ≤500 nm as MHC-II-binders. In this Example, a more stringent binding affinity of IC50 of ≤100 was also selected to identify medium to strong binders for further validation of the ˜7162 T-cell epitopes' binding to one or more of the MHC-II molecules. Approximately 4354 positive experimentally-validated MHC-II-binding epitope hits were obtained using IC₅₀ of ≤100 and stored locally for further use. The resulting ˜4354 hits (again including redundant hits due to presence of multiple redundant T-cell epitope peptide hits from Example 4 in the dataset) are expected to bind to one or more the MHC-II molecules with medium to strong binding affinity.

The MHC-II binding epitope peptide hits were next used for back-probing or mapping to the full length amino acid sequences of each allergenic protein in the Cupin family using the algorithm BLASTP to confirm the presence, and alignment, of the MHC-II-binding epitopes on each Cupin family allergenic protein. The threshold used was a minimum of 80% identity and 70% coverage of the length of the peptide. The MHC-II-binding epitope peptide hits were mapped to Cupin protein family members in food sources, including food sources other than the 15 food sources listed in Table 1, as well as non-food sources.

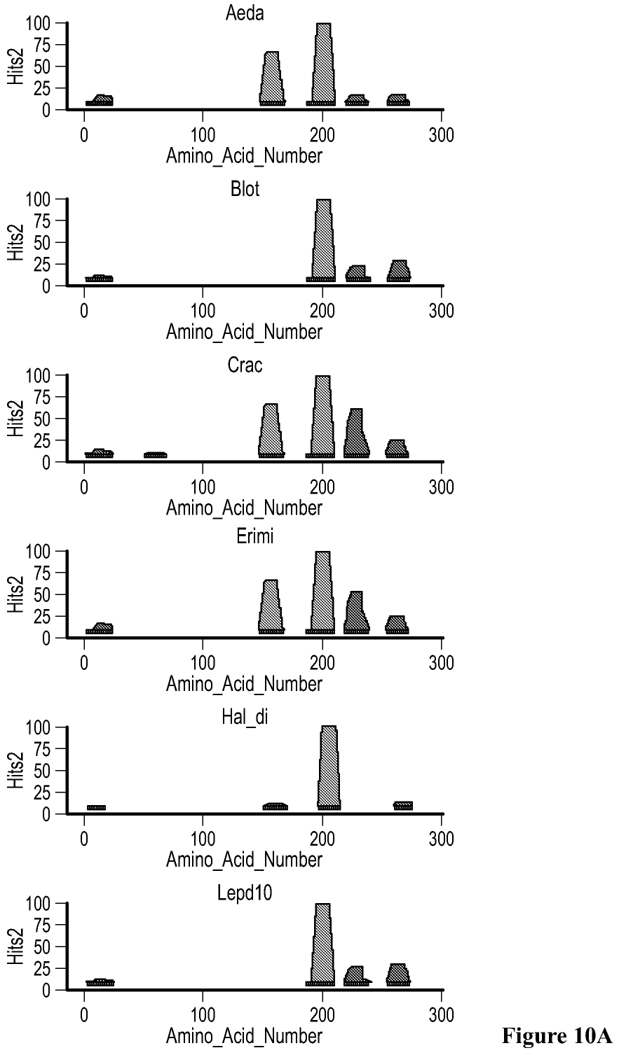

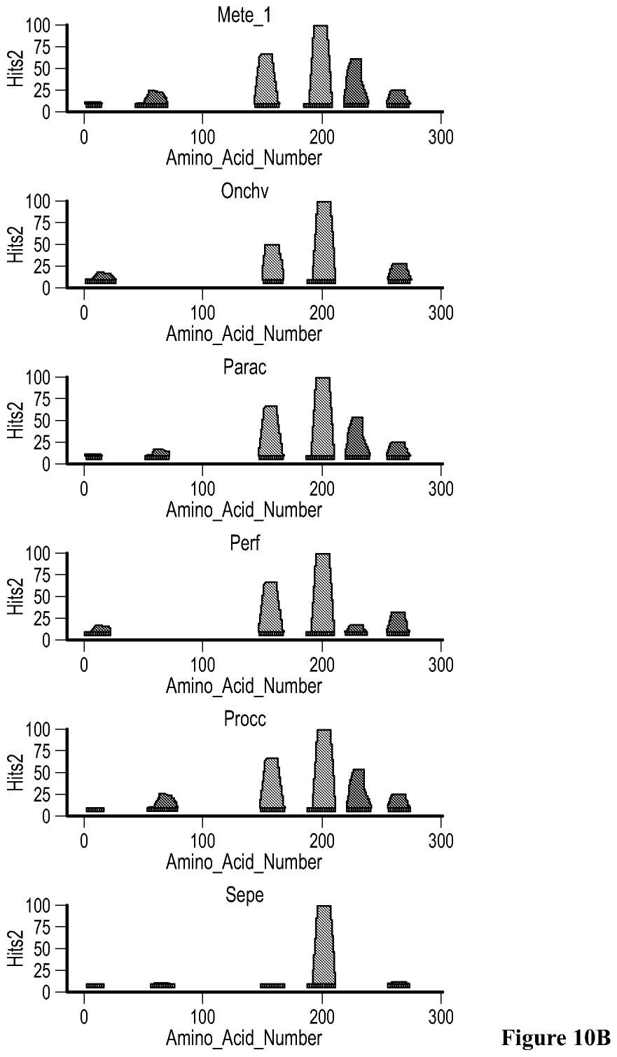

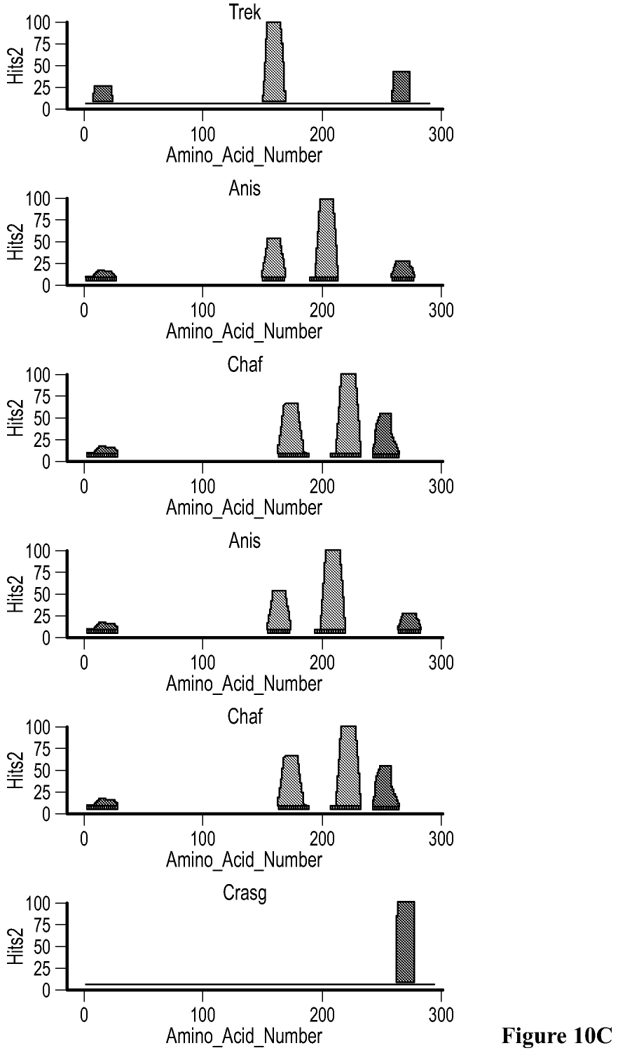

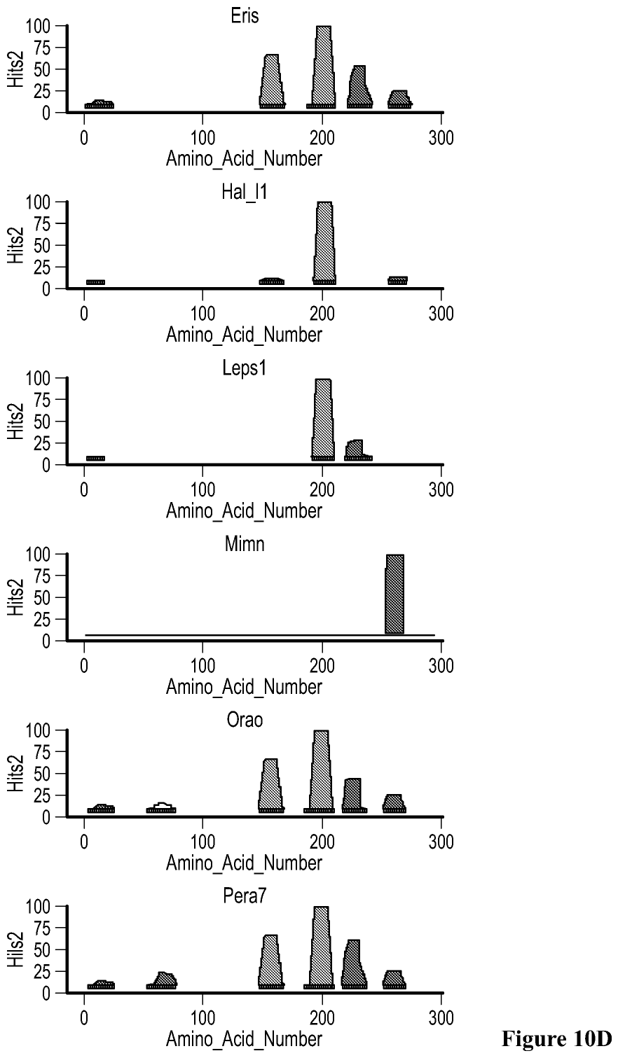

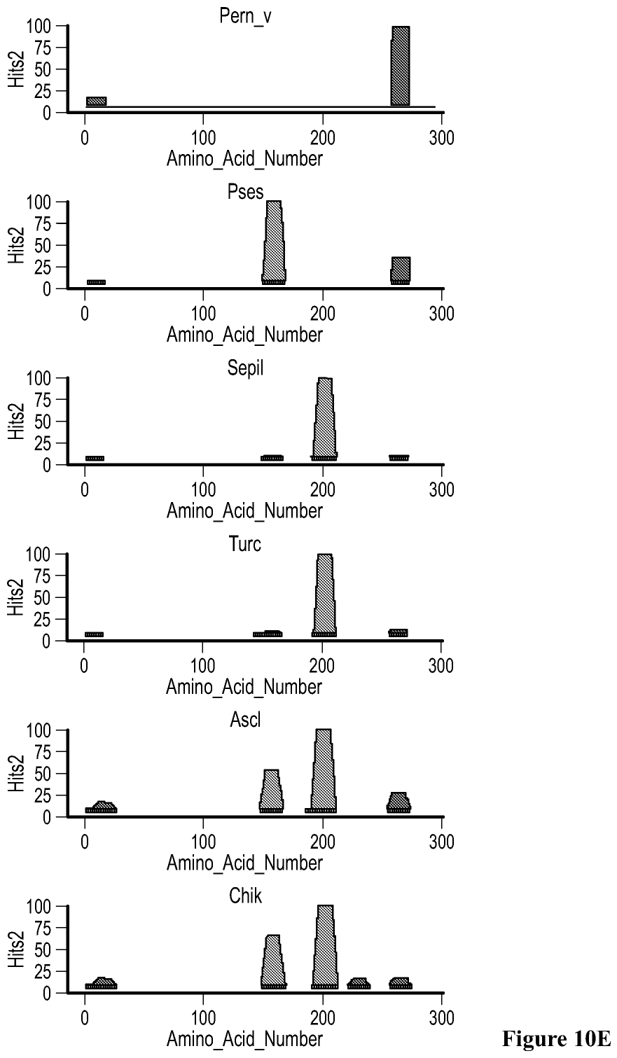

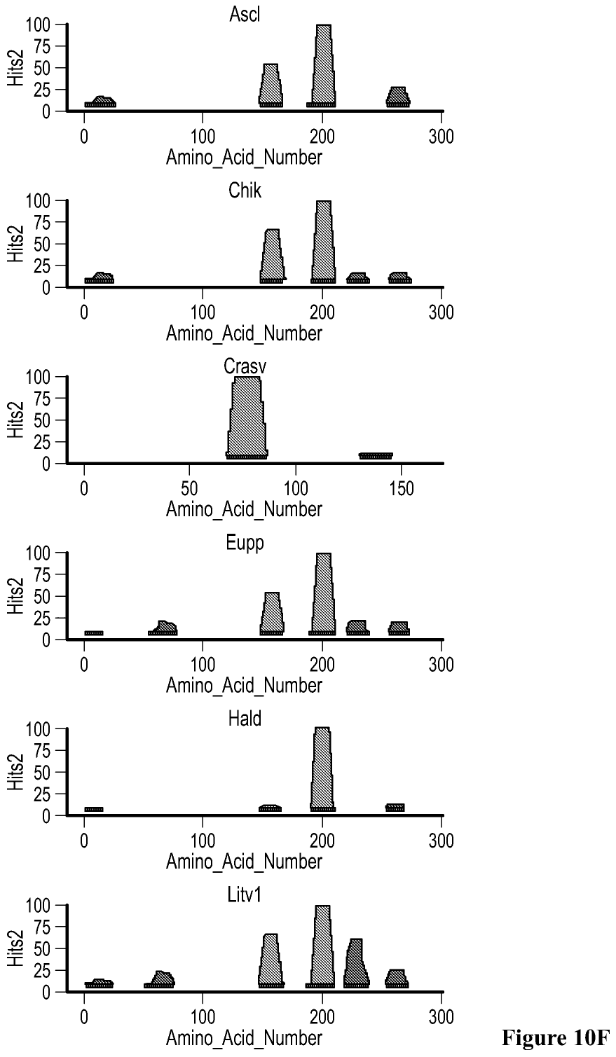

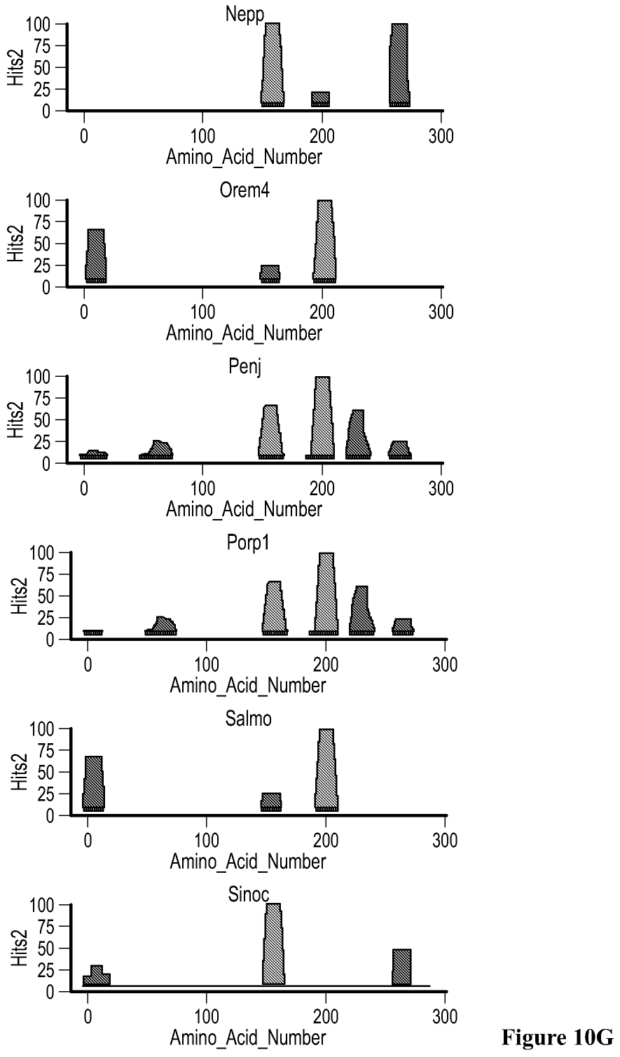

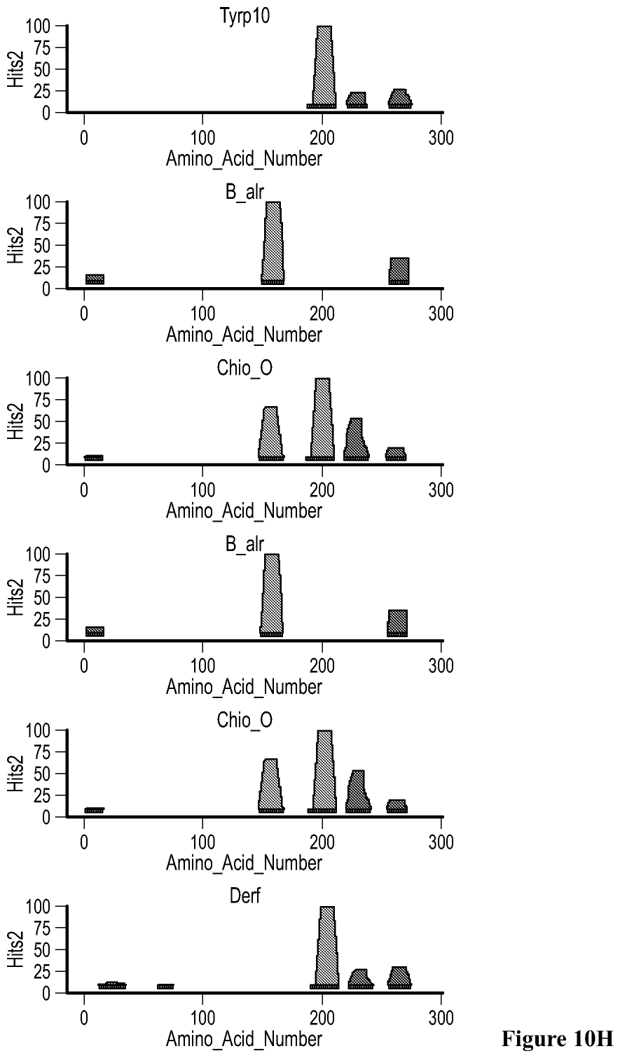

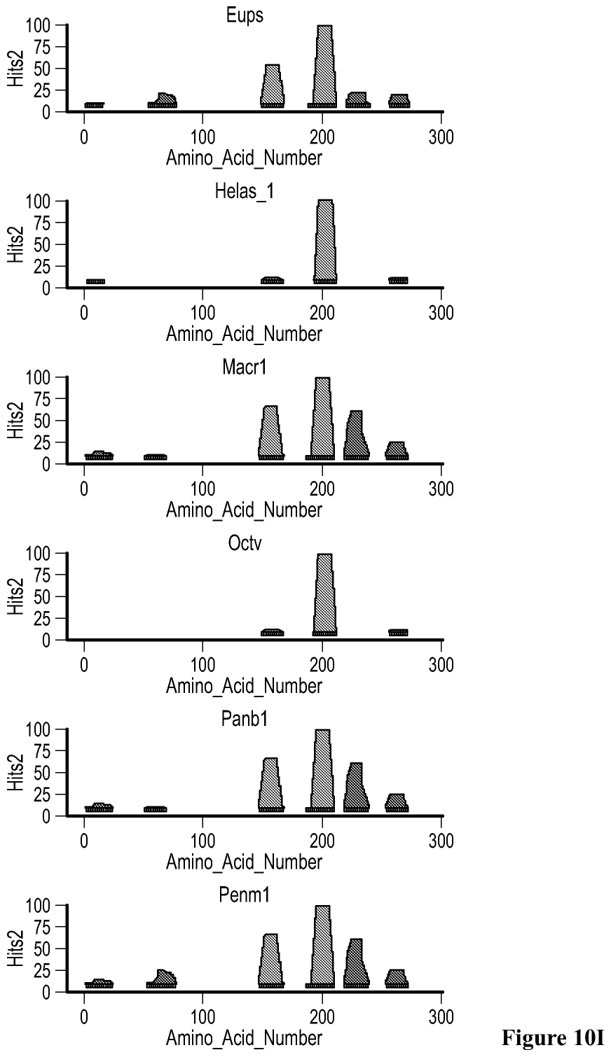

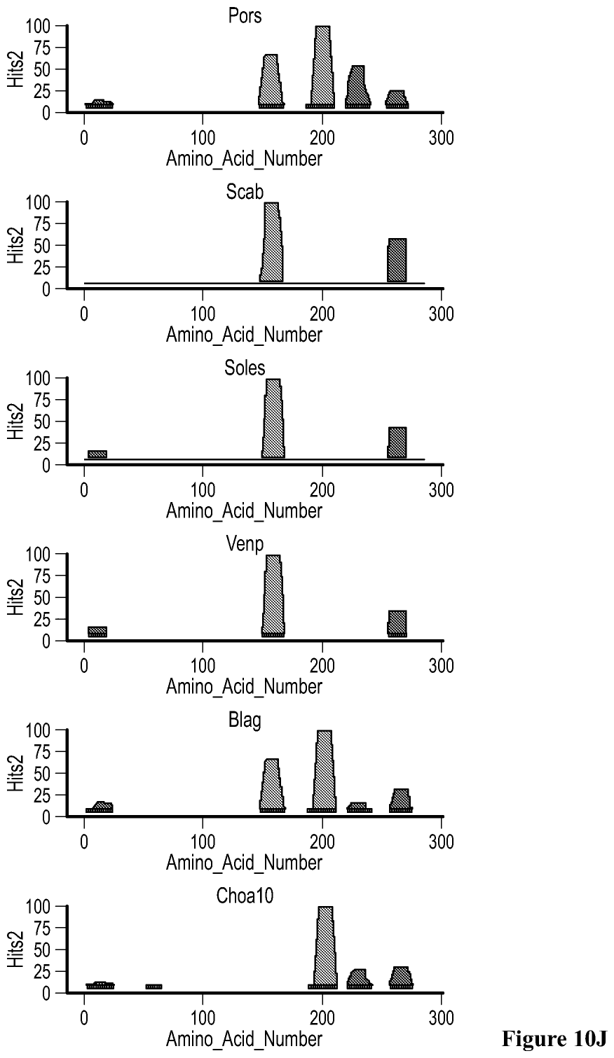

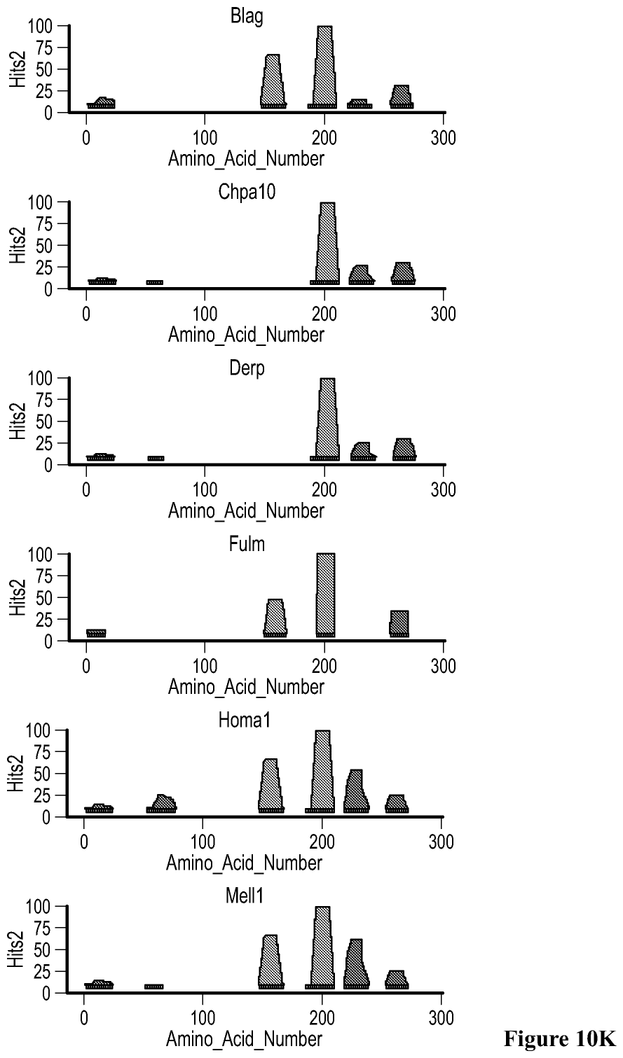

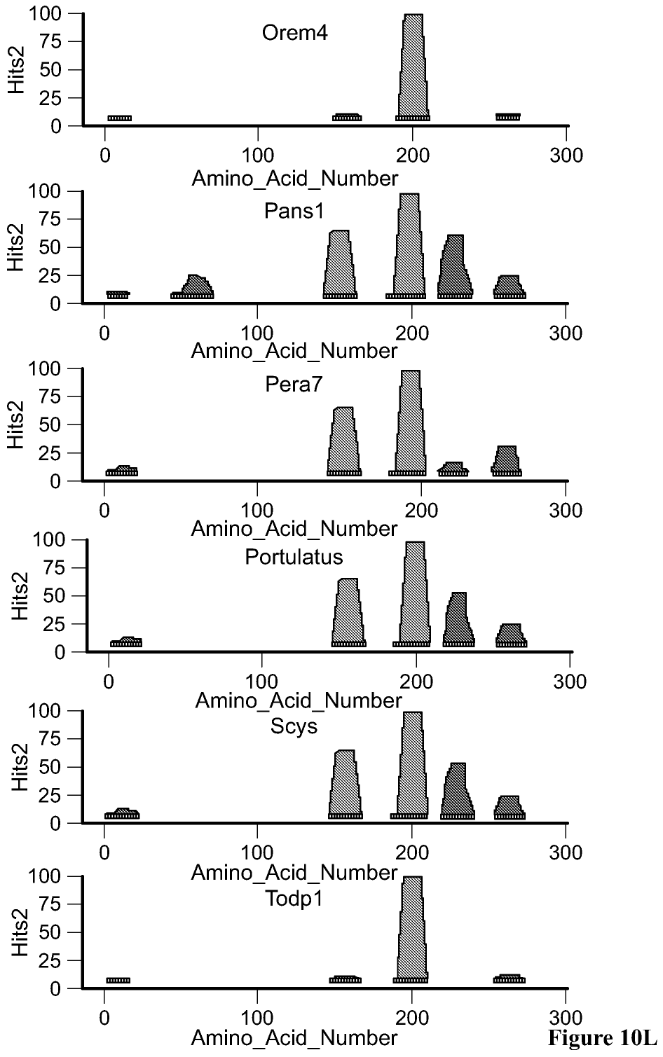

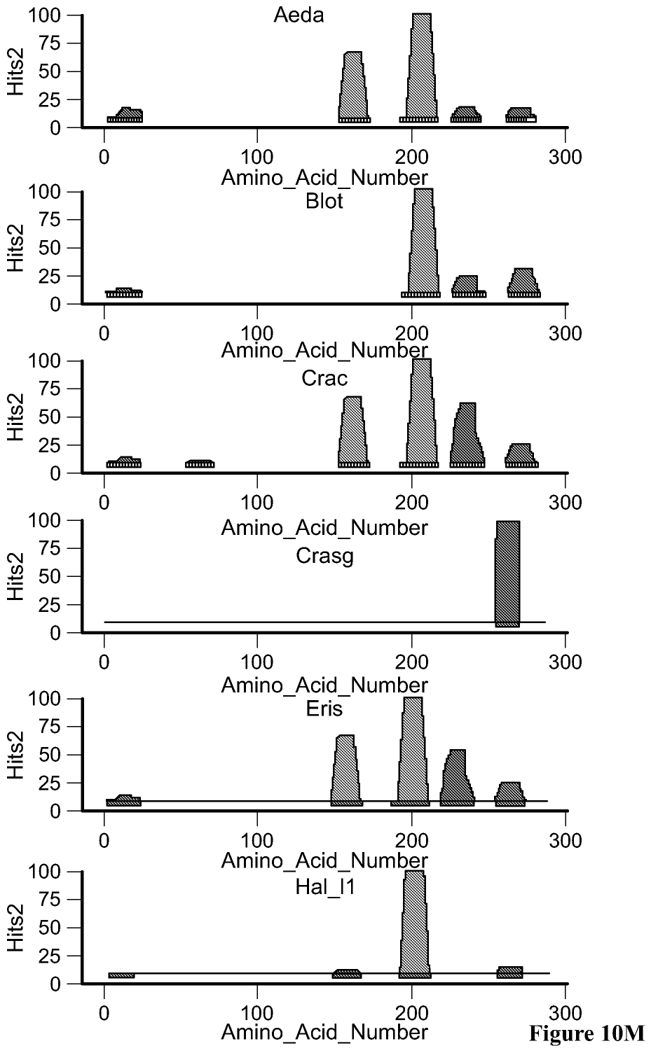

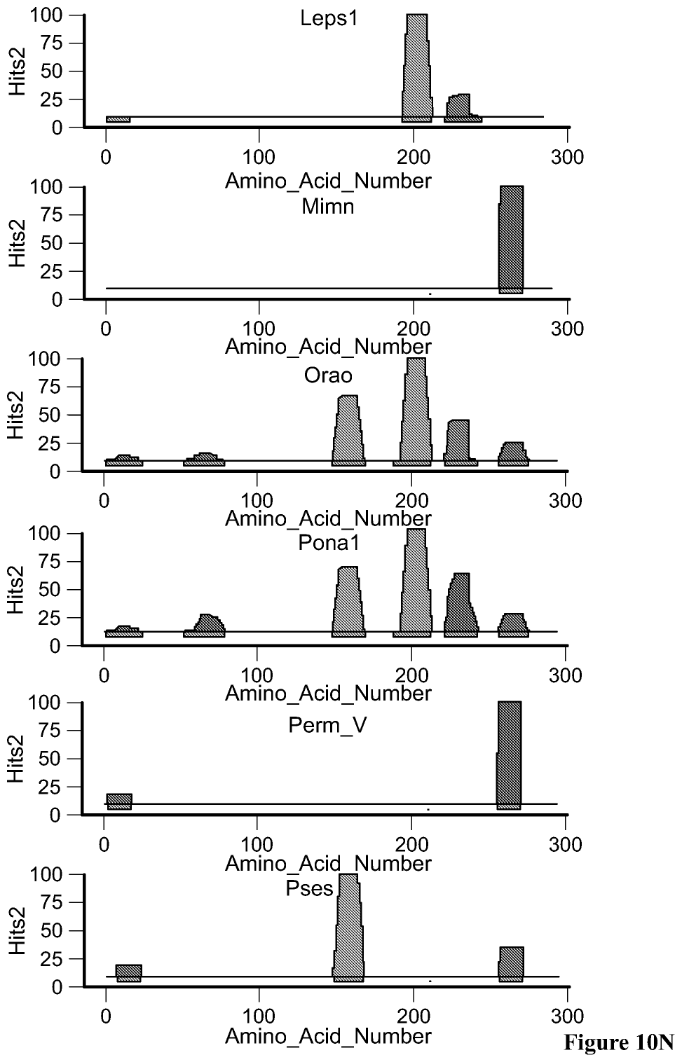

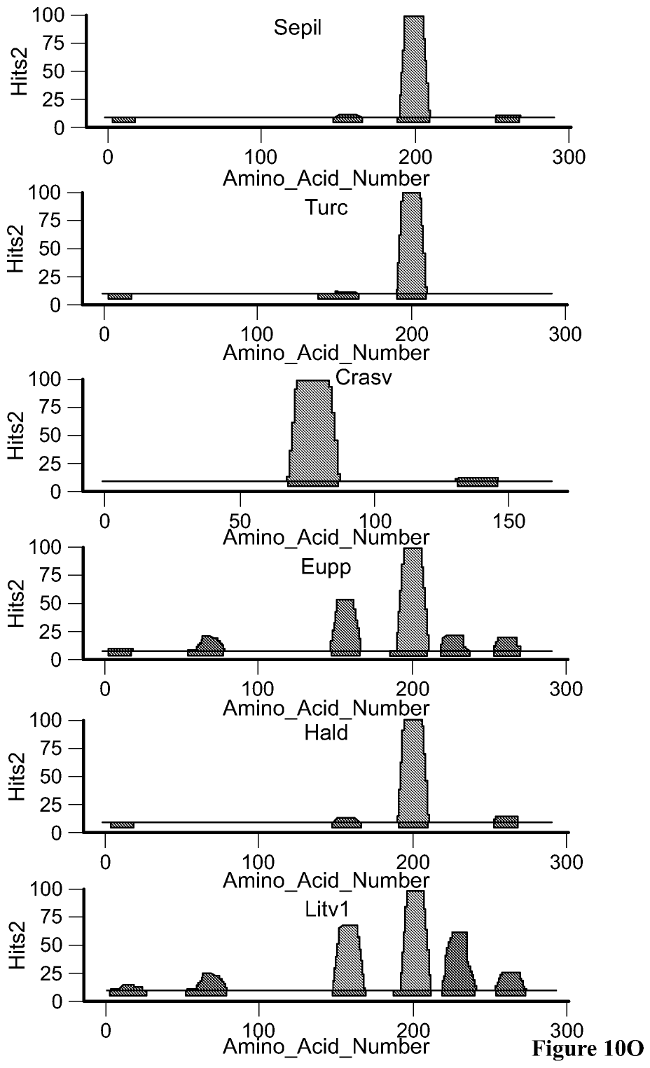

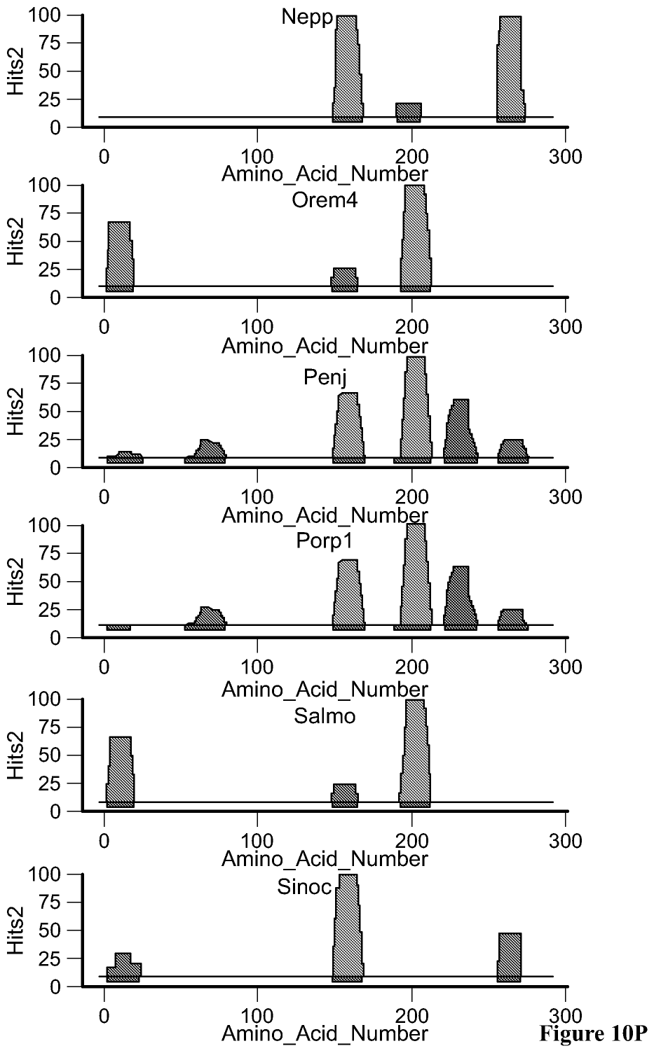

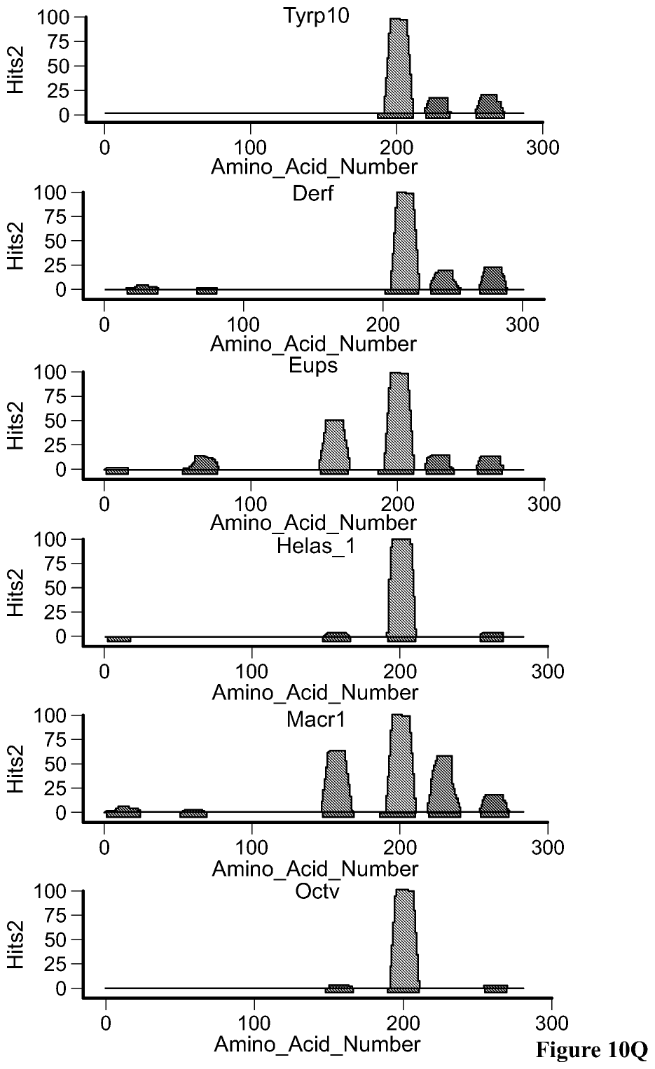

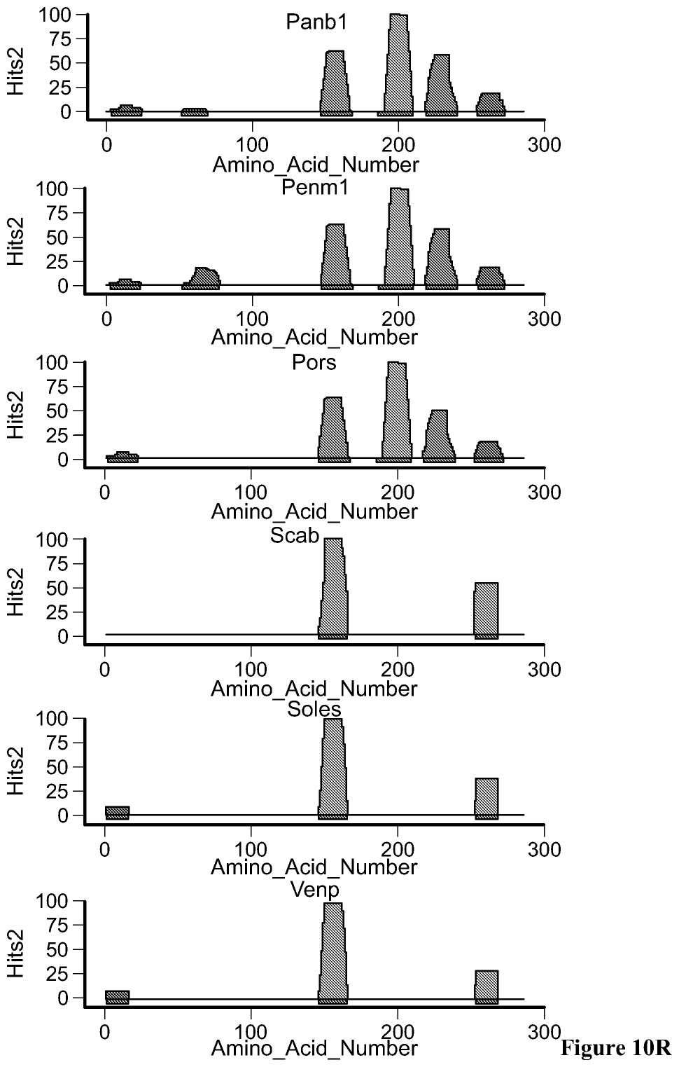

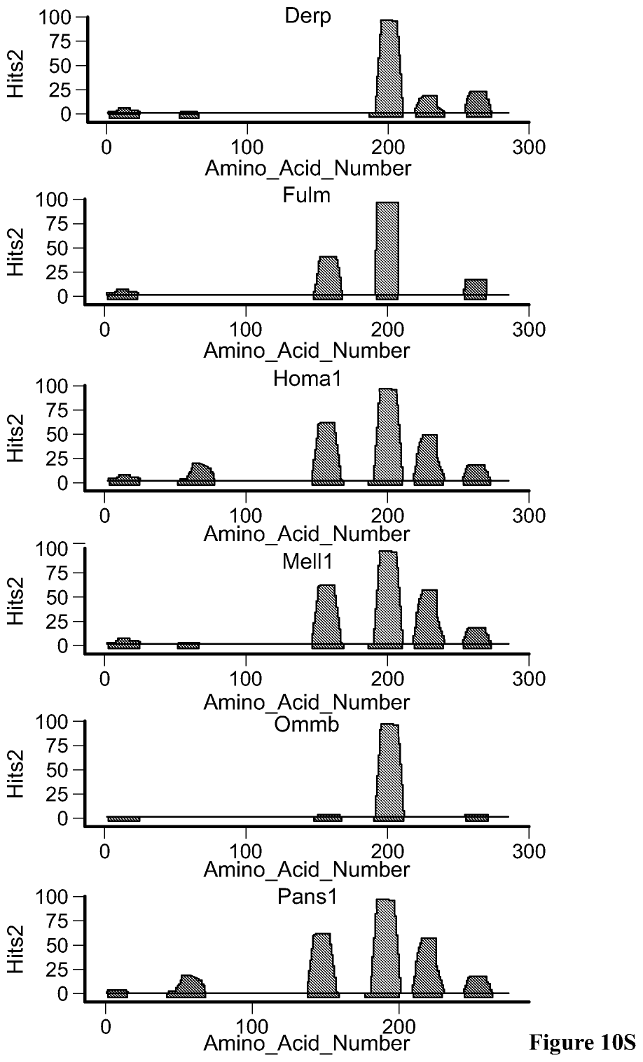

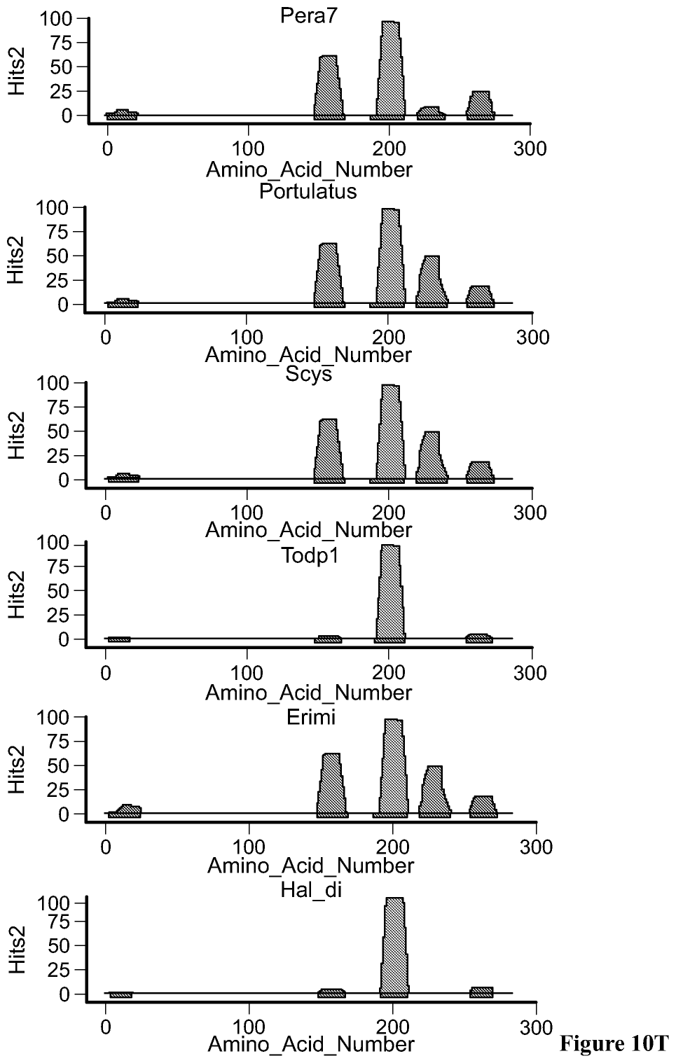

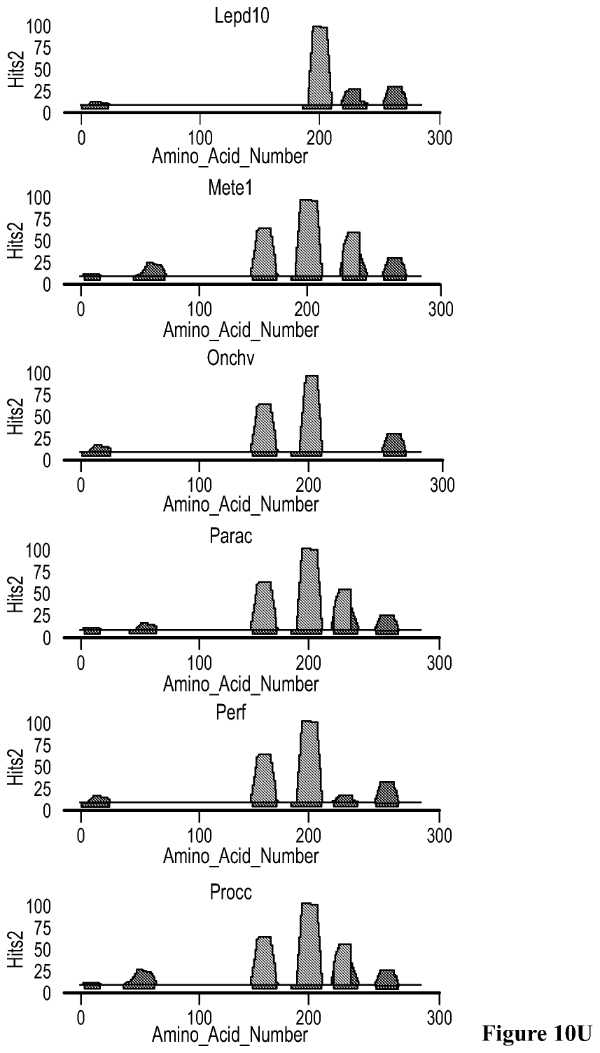

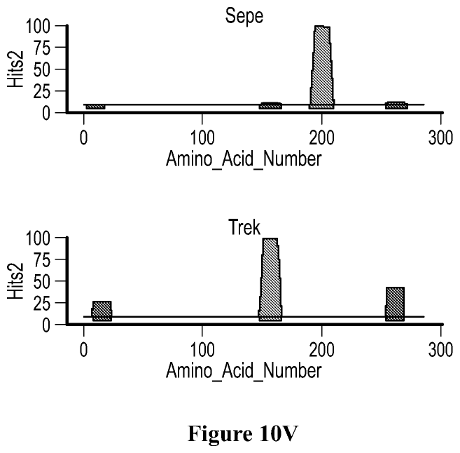

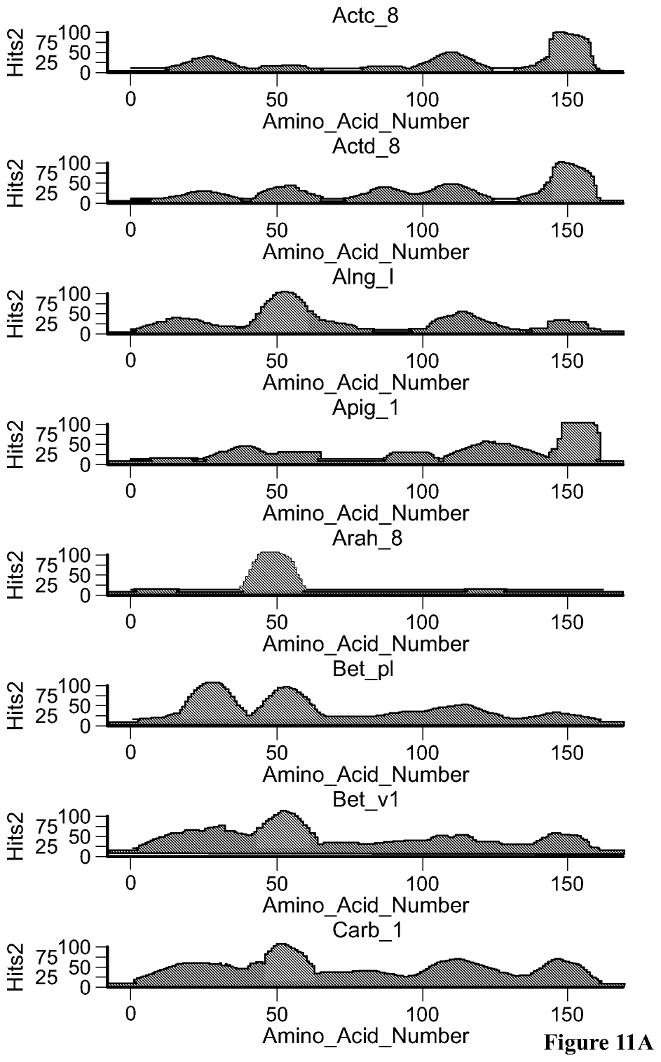

Next, the MHC-II-binding T-cell epitope peptides were mapped in silico to each Cupin family allergenic protein using the BLASTP alignment algorithm. The frequency of MHC-II-binding T-cell epitope peptide hits against each Cupin allergenic protein on the Y-axis and epitope location was mapped along the full-length protein. FIG. 9 shows the epitope cluster map results for each Cupin allergenic protein. The degree of cross-reactivity of each allergenic protein in the Cupin family is provided based on the frequency of hits (labeled on the y-axis for the graphs in FIG. 9 as “Hits2”, similar to the y-axis label in FIGS. 10-11) of MHC-II binding T-cell epitope peptides based on the height of the peaks (number of hits on Y-axis) and the location of MHC-II-binding T-cell epitope peptides along each full-length protein (amino acid position X-axis). Higher peaks reflect a greater frequency of hits for the epitope peptides, and a greater likelihood of the epitope being present on two or more Cupin protein family members, and a greater likelihood of cross-reactivity between them. As can be seen from FIG. 9, the results for ara h 1, a highly allergenic protein present in peanuts showing high frequency of MHC-II-binding T-cell epitope peptide hits, are consistent with those described previously, showing the presence of HLA-II restricted core epitope peptides located from 206-215, 213-225, 353-371, 409-425, 416-427, 436-452, 442-458, 451-470 and 452-470, and 507-524 (Prickett et al. (2013) Clin. & Expt. Allergy 43, 684-697).

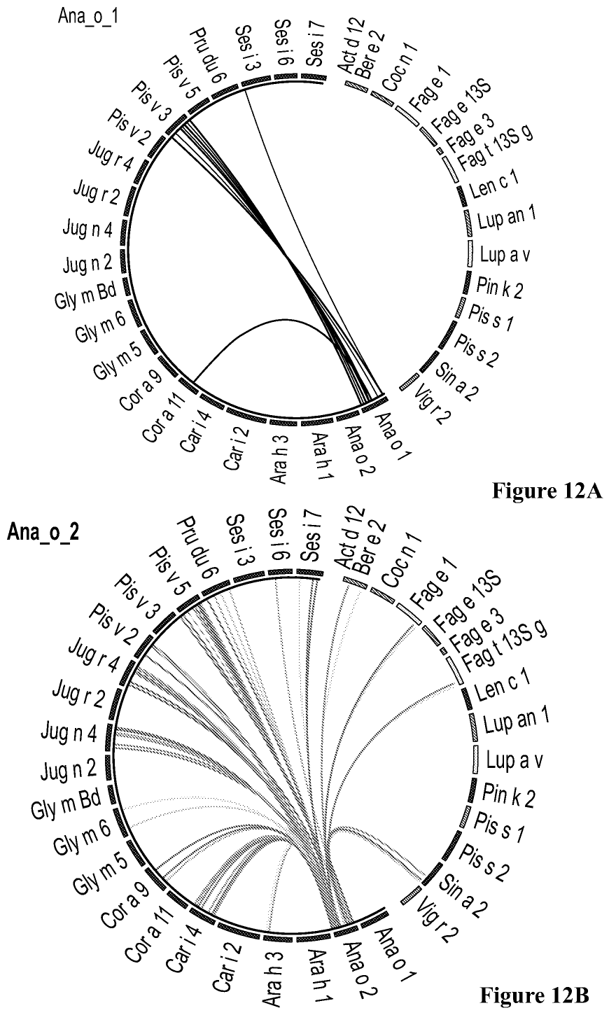

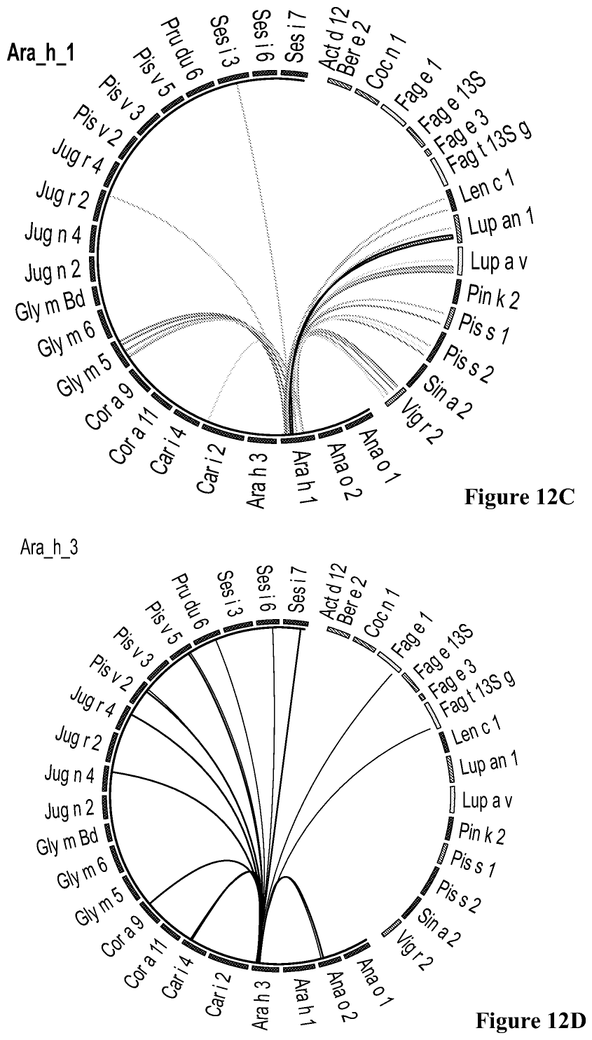

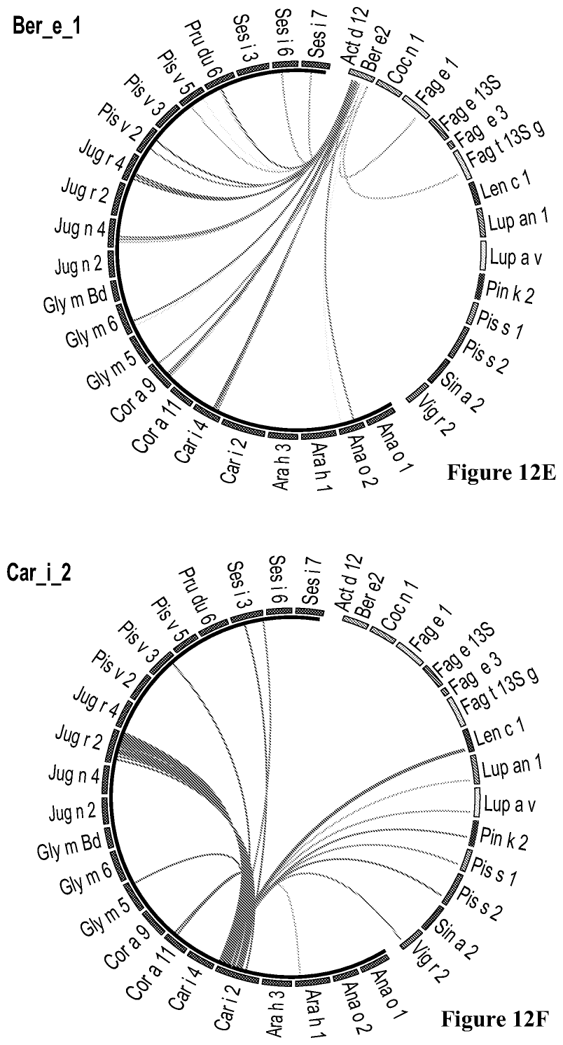

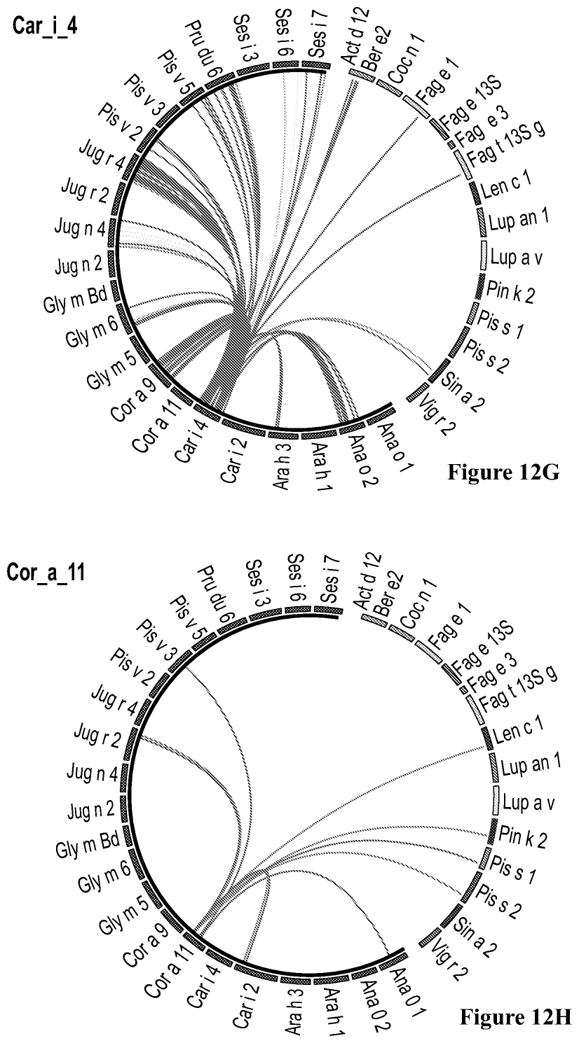

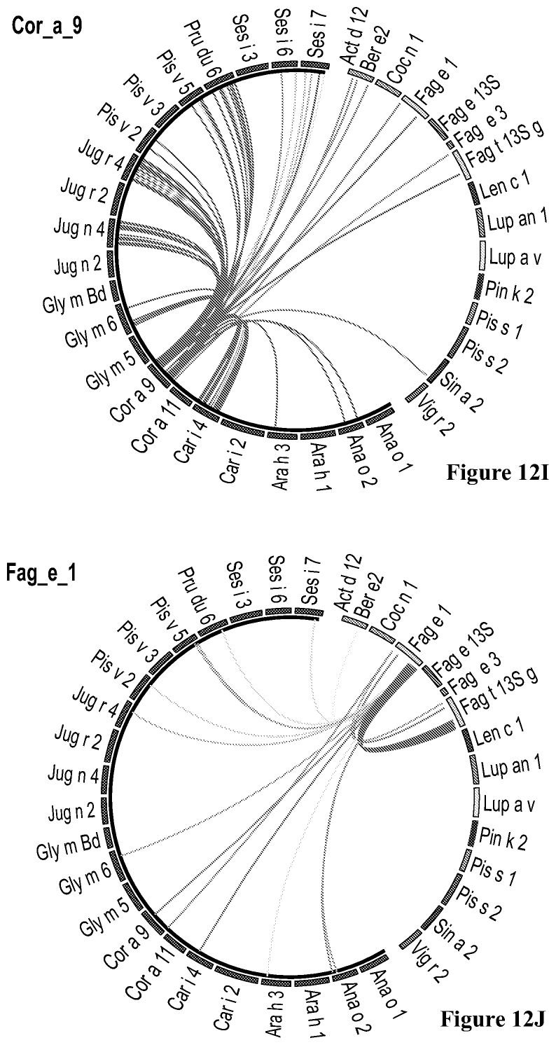

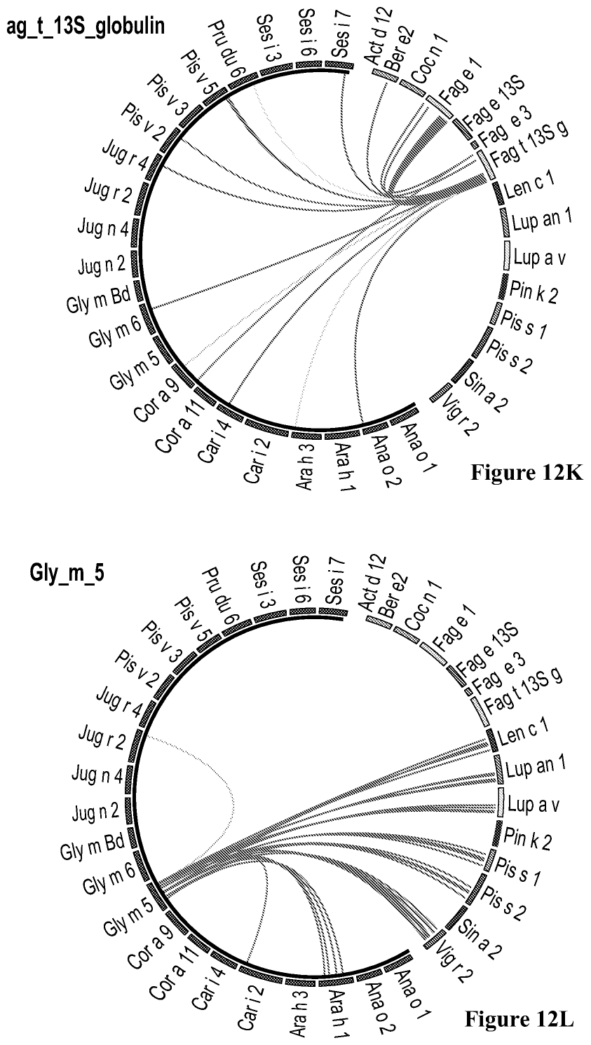

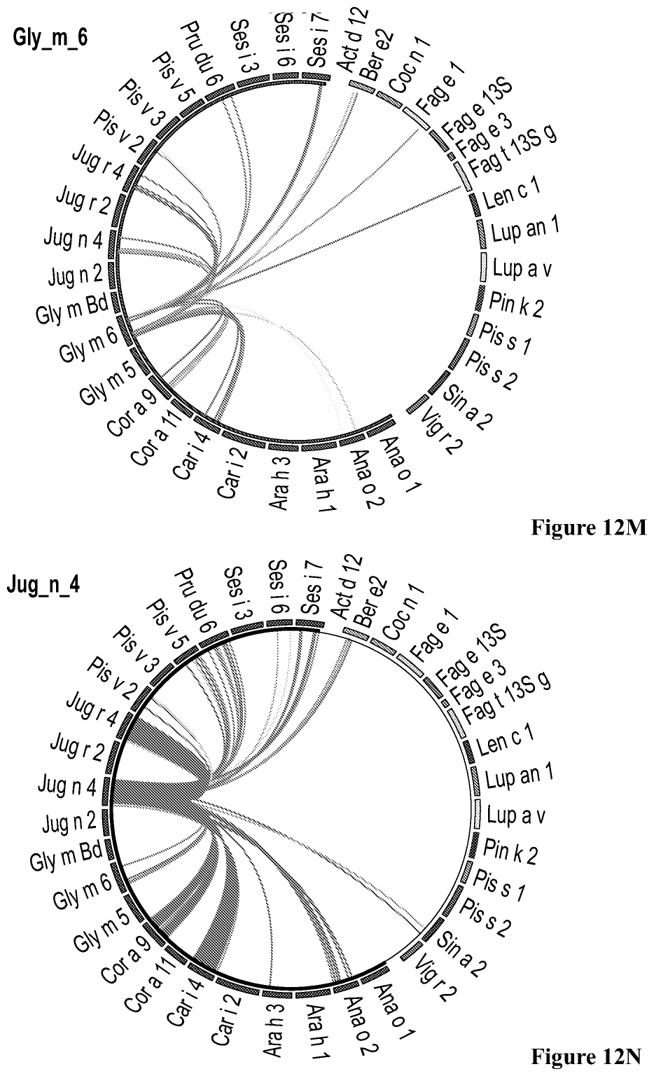

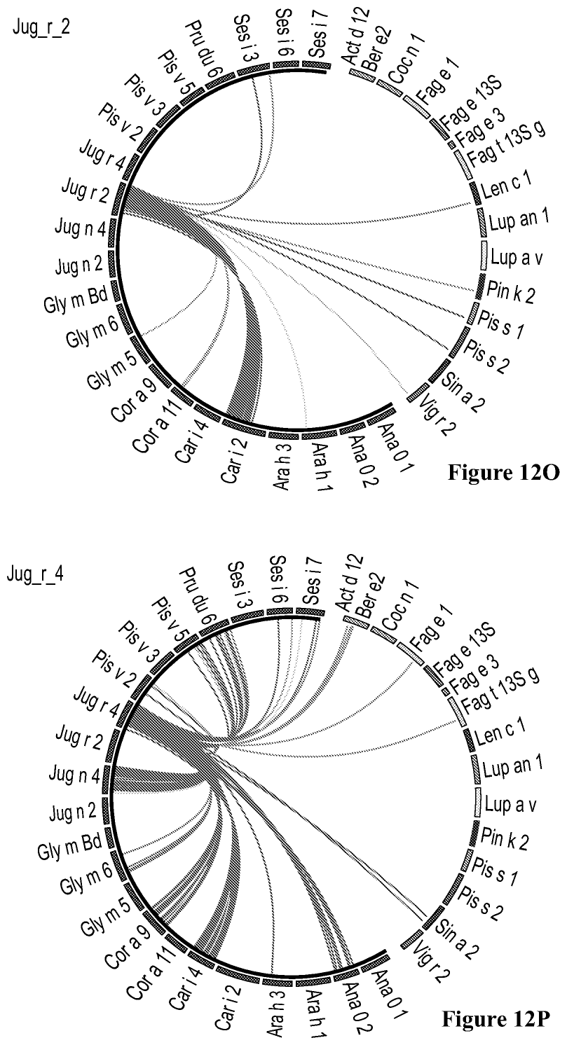

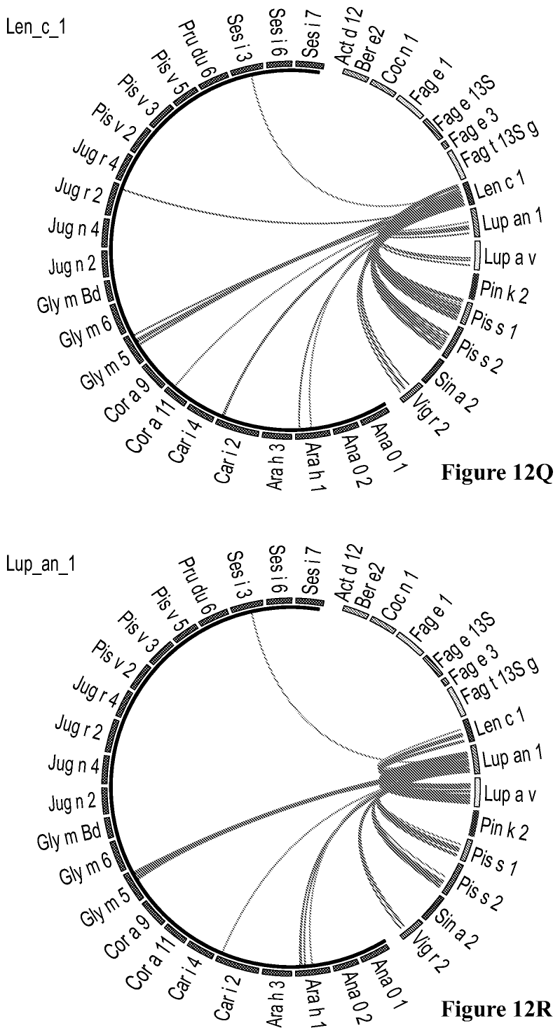

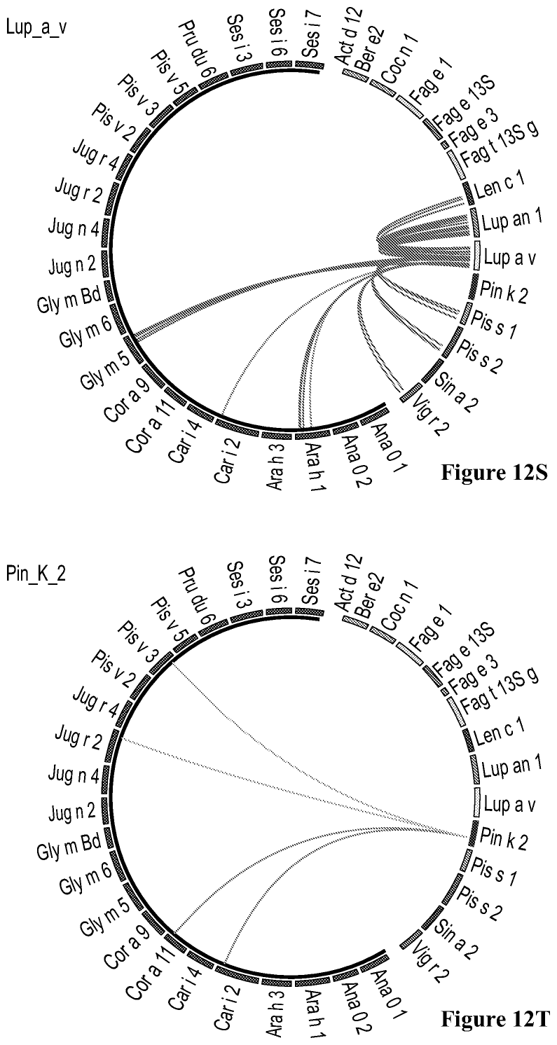

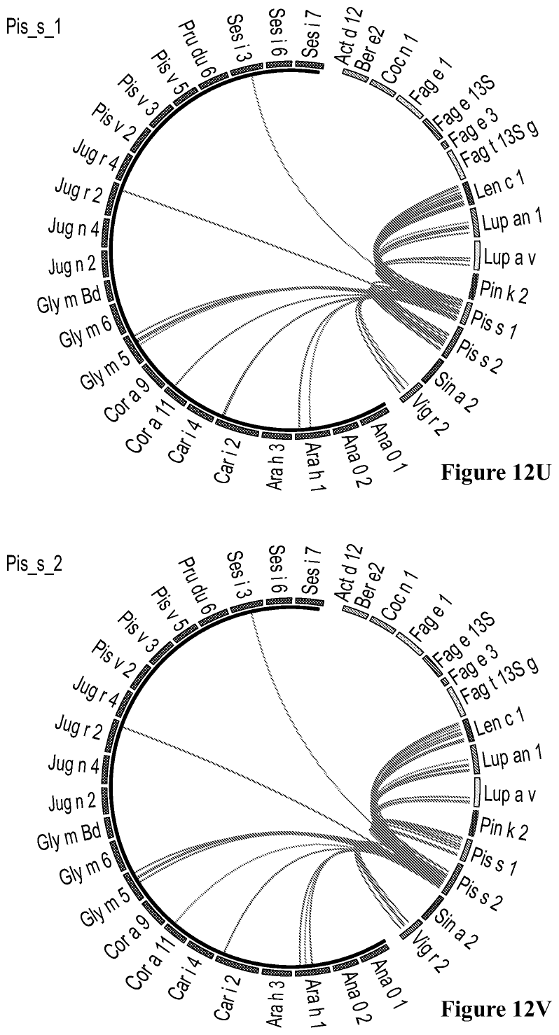

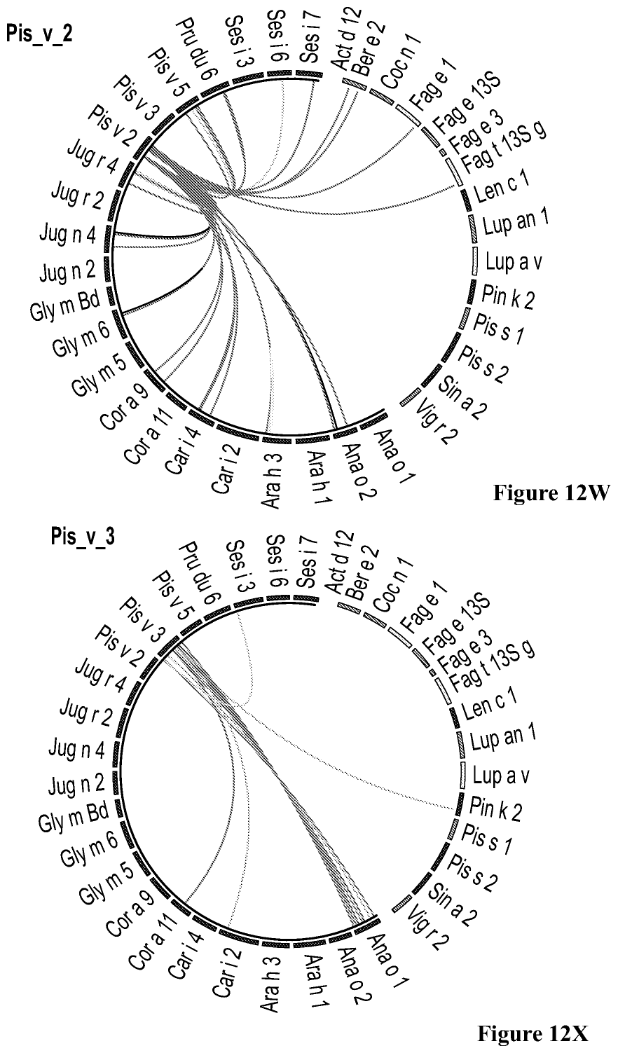

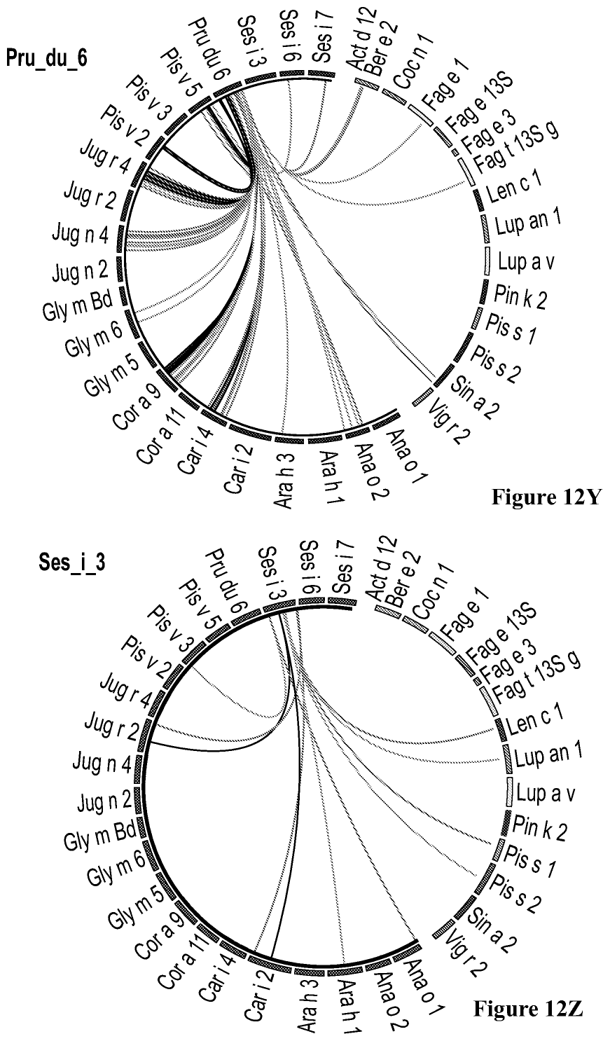

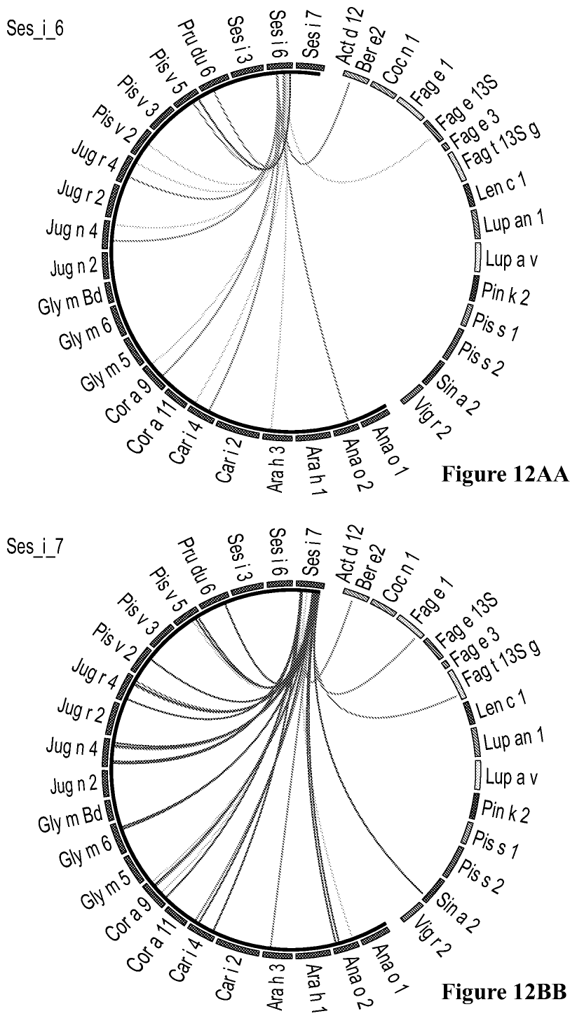

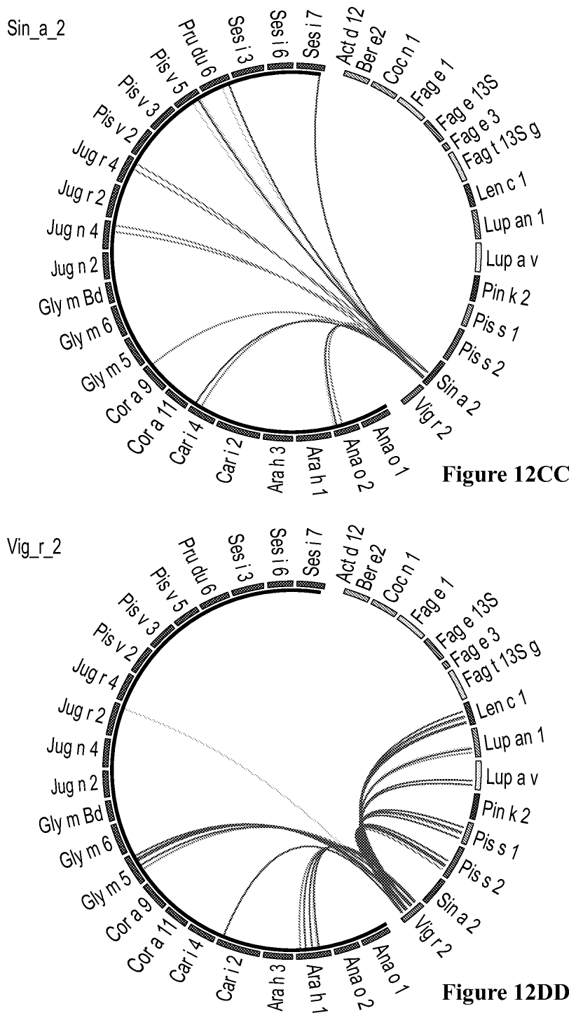

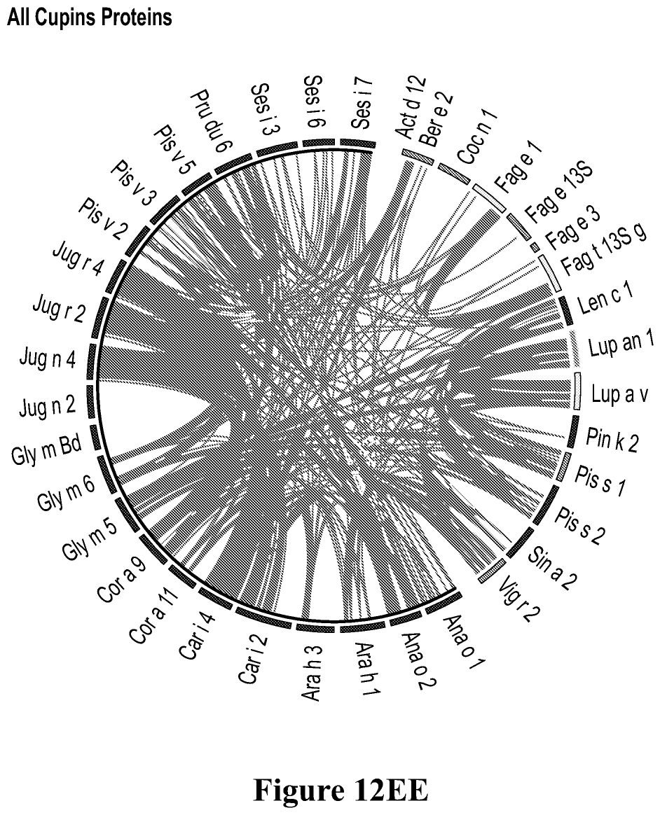

These results are also depicted as circos plots (FIG. 12). The individual MHC-II-binding T-cell epitopes present in a protein are connected to epitopes present on one or more of other proteins within the family. Proteins from some food sources, e.g., coconut had very few hits (data not shown) whereas other proteins had significantly more cross-reactivity with other allergenic proteins within the Cupin family.

Next, the identified MHC-II binding T-cell epitopes were analyzed to identify cross-reactive MHC-II binding T-cell epitopes. Predicted MHC-II-binding epitopes that were shared by or common to two or more allergenic proteins within the Cupin protein family were identified as cross-reactive peptides. The greater the number of each epitope sequence hits across the allergenic proteins within the protein family, the greater the cross-reactivity between the proteins. As can be seen from Tables 13-18, the presence and location of MHC-II-binding T-cell epitopes on allergenic proteins within a protein family can be identified using the methods of the present disclosure and cross-reactive epitopes.

MHC-binding T-cell epitopes were identified using the methods described above for the following Cupin proteins: Ber_e_2, Car_i_2, Cor_a_11, Fag_e_1, Fag_e_13S, Fag_t_13S_globulin, Jug_n_4, Lup_a_vicilin, Lup_an_1, Pin_k_2, Pis_s_1, Pis_s_2, Pis_v_2, Pis_v_3, Pis_v_5, Ses_i_3, Ses_i_6, Ses_i_7, Sin_a_2, and Vig_r_2 (See Tables 13-23). Further, these MHC-II-binding T-cell epitopes are cross-reactive with the other proteins within the Cupin protein family, including the peanut allergenic proteins ara h1 and ara h3 with the potential to induce allergy or sensitization in subjects to the food sources containing these cross-reactive allergenic proteins.

MHC-II or HLA molecules and their variants to which each T-cell epitope peptide bound were also identified based on the experimentally validated T-cell epitopes screened from IEDB. It is significant that these identified MHC-II-binding T-cell epitopes exhibit MHC degeneracy in their binding. The greater the number of MHC-II or HLA-II molecule variants that each T-cell epitope binds, the greater the number of individuals or subjects in which allergen cross-reactivity can be expected to occur, inducing immune response. As seen in FIG. 9, many such epitopes are present in clusters when viewed on the full-length amino acid sequence.

Results show that the Cupin allergenic proteins have multiple MHC-II-binding T-cell epitopes that bind large numbers of HLA variants ranging from 2-100 HLA molecule variants.

Further, MHC-II-binding T-cell epitopes may be prioritized based on cross-reactivity within proteins in a protein family. For example, Cupin allergenic proteins can be ranked ordered based on the number of other proteins that share the same epitope and/or the number of MHC molecules bound as found in this Example. A scoring system was used that assigned a cross-reactivity score (different from the score used with full-length proteins). A cross-reactivity scoring algorithm was applied to each identified MHC-binding T-cell epitope to allow rank ordering of the MHC-binding T-cell epitopes. The cross-reactivity score is calculated as:

$$\\ Cross ‐ reactivity score = ( \frac{{\sum }_{i = 1}^{l} h}{l} )$$

where, l is length of peptide (≤15) or number of amino acids in a cross-reactive site (≤15 amino acids), and h=number of times the given amino acid in the cross-reactive sites matches with high-confidence Kmers, given that these Kmers must also be matching with at least one other allergen.

A cross-reactive site is a region (length ≤15 amino acids) in any allergenic protein sequence that matches (using BLAST) with high-confidence Kmer(s) in such a way that the same given Kmer also matches with another allergen too. The number of redundant high-confidence Kmers that matches (using BLAST) a given region of a protein sequence can be determined. Thus, if a given amino acid is covered by large number of Kmer sequences then h of that amino acid must be large. So a cross-reactivity score of a given cross-reactive site is the sum of h-scores of its amino acids divided by length of the site, i.e., the mean of h-scores.

Higher cross-reactivity scores are expected to correspond to a higher tendency of an allergenic MHC-II-binding T-cell epitope peptides on one protein to cross-react with other proteins having the same (or nearly identical) epitope. Selection of the epitopes may be made based on other criteria in addition to or instead of the rank ordering done in this Example.

Tables 13-18 disclose cross-reactive MHC-II-binding epitope peptides identified by the methods disclosed herein for Cupin family proteins. The epitope peptide sequence and the number of distinct other Cupin proteins that each epitope peptide in a Cupin protein “hits” are shown. The various MHC-II molecules (and/or their variants) to which each of the identified cross-reactive MHC-II binding epitope peptides binds is shown in Tables 13-18.

For example, results show that Jug_r_4 allergenic protein present in English walnuts includes the MHC-II-binding T-cell epitope having the amino acid sequence (5′-HWNLNAHSVVYALRG-3′) corresponding to amino acid sequences from 377 to 391 of the Jug_r_4 protein. This epitope has “hits” to 10 distinct allergenic proteins (Pru_du_6; Gly_m_6; Pis_v_2; Cor_a_9; Jug_n_4; Pis_v_5; Ana_o_2; Car_i_4; Ses_i_7; Ara_h_3) belonging to various food sources (almond, soybean, pistachio, hazelnut, black walnut, cashew, pecan, sesame, and peanut). Thus, the Jug_r_4 protein (or a peptide comprising the epitope) comprising the WIC-II-binding T-cell epitope (5′-HWNLNAHSVVYALRG-3′) is cross-reactive with 10 other Cupin proteins (Pru_du_6; Gly_m_6; Pis_v_2; Cor_a_9; Jug_n_4; Pis_v_5; Ana_o_2; Car_i_4; Ses_i_7; and Ara_h_3 proteins), and peptides comprising sequences corresponding to the Jug_r_4 MHC-II-binding T-cell epitope (5′-HWNLNAHSVVYALRG-3′).

Further, the WIC-II-binding T-cell epitope (5′-HWNLNAHSVVYALRG-3′) is expected to bind to 25 different MHC-II molecules/variants in subjects having any of the following HLA haplotypes: HLA-DQA1*01:02/DQB1*06:02;HLA-DRB3*02:02;HLA-DRB1*13:02;HLA-DQA1*05:01/DQB1*03:01;HLA-DRB1*07:01;HLA-DRB1*09:01;HLA-DQA1*04:01/DQB1*04:02;HLA-DQA1*03:01/DQB1*03:02;HLA-DRB1*01:01;HLA-DRB3*01:01;HLA-DQA1*01:01/DQB1*05:01;HLA-DRB1*15:01;HLA-DRB1*04:01;HLA-DRB1*12:01;HLA-DRB1*08:02;HLA-DRB1*04:05;HLA-DQA1*05:01/DQB1*02:01;HLA-DRB4*01:01;HLA-DRB5*01:01;HLA-DRB1*11:01;HLA-DPA1*01:03/DPB1*02:01;HLA-DRB1*03:01;HLA-DPA1*03:01/DPB1*04:02;HLA-DPA1*02:01/DPB1*01:01;HLA-DPA1*02:01/DPB1*05:01.

The presence, location, and frequency of cross-reactive MHC-II-binding T-cell epitope hits can be determined for each allergenic protein present in the other identified protein families provided in Tables 3-4.

Similar methods were carried out for the Tropomyosin and Bet v 1 protein families, with results shown in Tables 20 and 22.

### Example 6. Identification of Cross-Reactive B-Cell Epitopes

B-cell epitopes can be linear epitopes or conformational (discontinuous). However, IgE-binding B-cell epitopes from food allergens are typically linear B-cell epitopes. In this Example, linear B-cell epitopes located in the allergenic proteins present in the Cupin protein family were identified. Briefly, all known experimentally-validated B-cell epitopes were first downloaded from the IEDB database available on the world wide web at iedb.org, and stored locally for use in this study (see tools.immuneepitope.org/main/html/bcell tools.html available on the world wide web).

A set of peptides (15-mer amino acid length overlapping peptides with a sliding window of 1 amino acid) were generated in silico from 37 protein family member sequences present within the Cupin protein family. The entire dataset of 15-mers was then screened in an unbiased and unsupervised manner against the experimentally-validated B-cell epitopes that were downloaded from IEDB database and stored locally. In this study, BLASTP algorithm with default parameters was used to screen for “hits” against the experimentally-validated B-cell epitope peptides. Cupin 15-mers that had a minimal acceptable homology of >80% identity and >70% coverage over the peptide length with a validated B-cell epitope were identified and stored locally in an in silico library. Approximately 2636 hits were identified as B-cell epitopes from the screening of the entire dataset containing 15-mers derived from all 37 Cupin protein family allergenic proteins at the same time in an unbiased and unsupervised manner.

Next, cross-reactivity of each identified B-cell epitope in the in silico library was determined. In order for an identified B-cell epitope to be characterized as a cross-reactive B-cell epitope, the B-cell cell epitope needs to present on at least 2 distinct full-length Cupin proteins. Accordingly, the approximately 2636 identified B-cell epitopes were each mapped back to the individual full length Cupin proteins as described above in Example 5. BLASTP algorithm was used for alignment of the peptide sequences. The B-cell epitope peptide sequence, and the number of Cupin proteins on which each B-cell epitope was present are provided in Table 19. Data shows that certain B-cell epitopes were shared by as many as 11 Cupin proteins. For example, Ana_o_2 B-cell epitope peptide: DNGIEETICTMRLK corresponding to 269-283 amino acid on the Ana_o_2 protein is highly cross-reactive with Sin_a_2; Ses_i_6; Jug_r_4; Gly_m_6; Pis_v_5; Pis_v_2; Car_i_4; Jug_n_4; Cor_a_9; Ses_i_7; Pru_du_6.

A comparison of the identified cross-reactive MHC-II-binding T-cell epitopes of Example 5 and the identified cross-reactive B-cells epitopes was performed by alignment (BLAST algorithm) of the B-cell epitopes with the MHC-II-binding T-cell epitopes to identify the cross-reactive epitope peptide sequences that included both T-cell epitopes and B-cell epitopes, thus identifying any overlap or adjacency of T-cells epitopes and B-cells epitopes on the Cupin allergenic proteins. Of the approximately 2636 B-cell epitopes, 1225 epitopes included both MHC-II-binding T-cell and B-cell epitope sequences.

Similar methods were carried out for the Tropomyosin and Bet v 1 protein families, with results shown in Tables 21 and 23.

### Example 7. Method of Obtaining Structural Models of Cross-Reactive Allergenic Proteins and/or Reference Allergenic Proteins and Prediction of Surface Exposed Amino Acids

The MHC-II-binding T-cell epitope peptides are supplemented with additional T-cell epitopes. To obtain a structural model of an allergenic protein (e.g., a cross-reactive allergenic protein) multiple structural representatives of the allergenic protein are retrieved from NCBI NR database and aligned. From the alignment a profile Hidden Markov Model is generated using the program HMMer3. The HMM is used to detect homologous structures in the PDB with a minimum e-value of 1×10⁻³. The HMM is used to align the structures' sequence to a reference sequence. An expected minimum threshold of 30% identity between at least one member of the allergenic protein or a T-cell epitope thereof is set to assert sufficient homology. No more than 10% insertions/deletions in the local alignment region are permitted. Predicting surface exposure of amino acids from protein sequence of the cross-reactive allergenic protein is performed using the method described in Holbrook et al. (1990) Protein Eng. 1990, 3(8):659-65).

### Example 8. In Vitro Assay for MHC-II Binding of T-Cell Epitopes

Peptides, for example, MHC-II-binding T-cell epitopes identified in Examples 1-5, are assayed for in vitro MHC-II binding. A competitive MHC Class II molecule-binding assay is used, wherein each peptide is analyzed for its ability to displace a known control binder from each of the human MHC Class II HLA-DP, HLA-DM, HLA-DOA, HLA-DOB, HLA-DQ, and HLA-DR. The MHC-II molecule and control peptides, for example with MHC Class II HLA-DRB1* allotypes (covering in total about 63% of the allotypes found in Caucasian population) are used in this Example. Each of the identified MHC-II-binding T-cell epitope peptides derived from Cupin, Tropomyosin, or Bet v 2 protein families are analyzed in a competition assay and screened for relative binding compared to a control peptide under stringent and less stringent conditions. Solubility of the identified MHC-II-binding T-cell epitope peptides is an essential criterion for development as a therapeutic or diagnostic. Each of the peptides is prescreened for solubility in aqueous, acidic milieu. Due to the nature of the competitive assay, the data for each are presented as a ratio of its own IC50 to that of the control peptide. Lower affinity binders, medium affinity binders and high affinity binders to the MHC-II molecules are determined and ranked. Each epitope's ability to bind the maximum number of MHC-II molecules is determined and ranked for “pan” MHC-II phenotype.

### Example 9. T-Cell Proliferation Assays

T-cell proliferation assays are conducted with full-length allergenic proteins (for example, shown in Tables 10-12) or allergenic peptides (for example, shown in Tables 13-18, 20, and 22). For example, in one experiment (A), six distinct allergenic proteins from Table 2 are selected (for example, ara_1, ara_h_3, car_i_2, car_i_4, Jug_n_1 and Jug_r_4 full-length protein) digested in simulated gastric juices (with enzymes) and immobilized on a microarray bead. In another experiment (B), five cross-reactive MHC-II-binding peptides from 6 distinct Cupin allergenic proteins shown in from Tables 13-18, 20, and 22 are immobilized on a microarray bead. Peptide microarrays are prepared using a commercial microarray system such as PEPperCHIP® Custom Peptide Microarrays as described by Ehlers et al. Clin. Exp. Allergy. Vol. 50(12), December 2020, 1415-1423.

Biological samples (blood) from subjects having an allergy to at least one of the allergens (for example, peanut, pecan and black walnut, or English walnut) are obtained and PBMCs are cultured in RPMI-1640 containing 2 mM L-glutamine, 100 IU/mL, penicillin-streptomycin and 5% heat-inactivated human AB serum (Sigma). The PBMCs are contacted with the microarray with (A) the allergenic proteins, and the microarray with (B) the MHC-II-binding peptides.

Antigen-induced T-cell proliferation is assessed by 3H-Thymidine (3H-TdR) uptake assays as described in J. Allergy Clin Immunol 2011, 127:608-615, e1-5. A stimulation Index (SI; cpm antigen-stimulated T-cells/cpm unstimulated T-cells)≥2 is considered positive. HLA-II restriction of epitope recognition by T-cells is assessed using monoclonal antibodies directed against HLA-DR (L243), HLA-DQ (SVP-L3), HLA-DP (B7/21) to block epitope presentation. To allow detection of peptide-induced CD4+ T-cell proliferation within whole PBMC, 7-day cultures of CFSE-labeled PBMC are set up. At least 10,000 CD4+ T-cells are analyzed per sample and SI calculated as percentage of CD4+CFSElo (proliferated) cells with antigen (background). Expression of markers for T-regs or suppressor T-cells (such as Foxp3+ and others) are performed to identify the T-cell type. The detection threshold for a specific response in this assay is assessed by expanding peptide-specific T-cells from proliferated CD4+ cells and Foxp3+ cells over a range of SI values. Specific T-cells are generated from divided T-cells with SI from 3 experiments.

### Example 10. Basophil Activation Assays

Basophils play a key role in allergy. Upon specific recognition of allergens by surface IgE, basophils release potent mediators, accompanied by mechanistically distinct surface expression of activation markers CD63 and CD203c. The DURAClone IF Basophil Activation panel assesses CD63 and CD203c, indicating IgE-mediated activation. DURAClone IF Basophil Activation assay from Beckman coulter is used in accordance with manufacturer's instructions to determine activation of basophils present in a biological sample (e.g., PBMCs) in response to cross-reactive allergenic proteins or MHC-II-binding T-cell epitope peptides (for example, those identified in Examples 1-5).

### Example 11. Microarray Assay for Detection of IgE Antibody and IgG Antibody

MHC-II binding T-cell peptides obtained from Example 5 are used to prepare a peptide microarray using a commercial microarray system such as PEPperCHIP® Custom Peptide Microarrays as described by Ehlers et al. (2020) Clin. Exp. Allergy. Vol. 50(12), 1415-1423.

The following sets of 5 exemplary MHC-II binding T-cell epitope peptides from 7 allergenic proteins may be used in the array. The peptide epitopes' corresponding amino acid position on the full-length proteins is provided.

Walnut Jug_r_4 MHC-II binding Epitope Peptides: HWNLNAHSVVYALRG (aa 377-391 aa), VPHWNLNAHSVVYAL (aa 375-389 aa), HTLPVLRWLQLSAER (aa 351-aa 365), DIIAFPAGVAHWSYN (aa 145-aa157), LLLPQYSNAPQLVYI (aa 83-97 aa).

Pecan Car_i_4 MHC-II binding Epitope Peptides: HWNLNAHSVVYALRG (aa 378-392 aa), AGVAHWCYNDGSSPV (aa 152-166 aa), NLPILRWLQLSAERG (aa 354-367 aa), DIIAFPAGVAHWCYN (aa 146-159 aa), PAGVAHWCYNDGSSP (aa 151-165 aa).

Hazelnut Cor_a_9 MHC-II binding Epitope Peptides: PHWNLNAHSVVYAIR (aa 382-395 aa), EGDIIALPAGVAHWC (aa 151-166 aa), LPAGVAHWCYNDGDS (aa 157-172 aa), LLLPQYSNAPELIYI (aa 85-99 aa), HFREGDIIALPAGVA (aa 148-162 aa), AGRTSAIRALPDDVL (aa 455-469 aa)

Mung Bean Vig_r_2 MHC-II binding Epitope Peptides: FGINAENNQRNFLAG (aa 379-393 aa), GSLLLPHYNSKAIVI (aa 300-314 aa), GINAENNQRNFLAGE (aa 380-394), SLLLPHYNSKAIVIL (aa 301-315 aa), QRNFLAGEKDNVISE (aa 387-400 aa)

Peanut Ara_h_3 MHC-II binding Epitope Peptides: HYNTNAHSIIYALRG (aa 407-418 aa), LLILRWLGLSAEYGN (aa 383-395 aa); GHVLVVPQNFAVAGK (aa 443-455 aa); AHSIIYALRGRAHVQ (aa 412-424 aa); ALRGRAHVQVVDSNG (aa 418-431 aa)

Cashew Ana_o_2 MHC-II binding Epitope Peptides: PAGVAHWCYNEGNSP (aa 138-152 aa); PHWNLNSHSIIYGCK (aa 332-346 aa); GDIIAIPAGVAHWCY (aa 132-146 aa); LLLPQYSNAPQLIYV (aa 72-86 aa); NGIEETICTMRLKEN (aa 270-282 aa)

Common Buckwheat Fag_e_1 MHC-II binding Epitope Peptides: RINTVNSNNLPILEF (aa 406-420 aa); QILVVPQGFAVVLKA (476 aa-488 aa); PRAGRINTVNSNNLP (402 aa-416 aa); TSVLRAIPVEVLANS (515 aa-529 aa); TKLILSFSLCLMVLS (2 aa-16 aa)

Briefly, a microarray with synthetic 15-mer peptides among those listed above is commercially obtained (PEPperPRINT). The peptide length of 15-20 amino acids is in accordance with the experience of PEPperPRINT to provide sufficient sensitivity without significant formation of secondary structures. All peptides are printed in triplicates with a linker consisting of 2 ß-alanine and one aspartic acid. This linker is chosen to circumvent the binding of negatively charged fluorescent dyes to positively charged amino acids which are close to the array surface. Subjects' sera are diluted 1:4 in WSUB and incubated overnight.

For detecting bound-specific IgE and IgG4, a biotinylated anti-IgE antibody (clone MHE-18 1:5000, BioLegend) and simultaneously a biotinylated anti-human IgG4 coupled with Neutravidin DyLight 680 (clone HP6025, 1:5000, Southern Biotech/Thermo Fisher Scientific) is applied on the microarray and incubated for one hour at room temperature. Bound biotinylated human anti-IgE antibodies is visualized by adding Neutravidin DyLight 800 (1:5000, Thermo Fisher) for one hour at room temperature. After extensive washing and drying, the microarray slides are scanned at a wavelength of 700 nm for IgG4 and 800 nm for IgE (intensity: 8.5) and the focus is set to 0.8 mm and the resolution to 21 μm.

For data evaluation, the fluorescent signals for each peptide are obtained using the Pepslide Analyzer Software (SICASYS) with the fixed-spot adjustment and the logarithmic signal-to-noise ratios (S) is computed as described by Ehlers et al. Epitopes are defined as recognition of 2-4 contiguous peptides with a median z-score ≥3.0 and the amino acid residues will be counted based on the amino acid sequence without signal peptide.

### Example 12. T-Cell Epitope Peptide-Specific T-Cells

9-mer and 15-mer peptides comprising MHC-II binding epitopes are synthesized using a standard solid phase synthesis method and purified by reversed phase high performance liquid chromatography (HPLC). The purity (>90%) and the identity of the peptides are determined by analytical HPLC and mass spectrometry analysis, respectively. Peptides are dissolved in dimethylsulfoxide at 20 mg/ml and stored at −80° C.

HLA-II restricted cell lines are purchased from International Histocompatibility Working Group and American Type Culture Collection (Rockville, Md.) and are cultured under the recommendations of their respective depositors.

Human leukocyte antigen (HLA)-transfected C1R cells are used as stimulator cells. cDNA encoding an open reading frame of HLA class II (HLA-DRB3*02:02; HLA-DRB1*13:02;HLA-DRB1*07:01; or HLA-DRB1*09:01) is amplified by PCR and inserted into an expression vector. C1R cells are transfected with HLA class II expression vector and cultured in presence of G418 (Invitrogen, Carlsbad, Calif) for 14 days. G418-resistant single cell and feeder cells were plated into 96 well cell culture plate (Corning, Inc., Corning, N.Y.) containing culture medium supplemented with G418 and further cultured for 30 days. The expression of transfected HLA class II on the C1R cells was confirmed by flow cytometry analysis.

Monocyte-derived dendritic cells (DCs) are used as antigen-presenting cells to induce cytotoxic T lymphocyte (CTL) which respond against peptide presented on HLA class II. DCs are generated in vitro (Uchida N et al. Clin Cancer Res. 2004; 10:8577-86). Peripheral blood mononuclear cells (PBMCs) are isolated from blood of healthy volunteer by Ficoll-Paque PLUS (GE Healthcare). Monocytes (adherent cells in PBMCs) are cultured to induce into DCs in the presence of 1000 IU/ml of granulocyte-macrophage colony-stimulating factor (R&D Systems, Minneapolis, Minn.) and 1000 IU/ml of interleukin (IL)-4 (R&D System) in AIM-V Medium (Invitrogen) containing 2% heat-inactivated human serum (AIM-V/2% HS medium). After seven days of culture, monocyte-derived DCs are pulsed with 20 μg/ml of the synthesized peptide in the presence of 3 μg/ml of β-2-microglobulin for 3 hr at 37° C. in AIM-V Medium. These peptide-pulsed DCs are inactivated by X ray-irradiation (20 Gy) and mixed at a 1:20 ratio with autologous CD8+ T cells obtained from PBMCs by using CD8 Positive Isolation Kit (Thermo Fisher Scientific, Carlsbad, Calif). These cultures are set up in 48 well cell culture plate (Corning). Each well contain 1.5×104 peptide-pulsed DCs, 3×105 CD8+ T cells and 10 ng/ml of IL-7 (R&D System) in 0.5 ml of AIM-V/2% HS medium. The day after next (day 2), IL-2 (Novartis) is added to the culture at final concentration of 20 IU/ml. On day 7 and day 14, CD8+ T cells are further stimulated with autologous peptide-pulsed DCs. DCs are prepared each time in the same way as described above. Peptide specific IFN-γ production of CD8+ T cells is tested by ELISPOT assay on day 21 (Watanabe T et al. Cancer Sci. 2005; 96:498-506; Suda et al. Cancer Sci. 2006; 97:411-9; incorporated by reference in their entireties).

After limiting dilution, CD8+ T cells are expanded using the Rapid Expansion Method. CD8+ T cells are cultured with feeder cells (5×106 cells each) and 40 ng/ml of anti-CD3 antibody in 25 ml of AIM-V/5% HS medium. Next day (day 1), 3000 IU of IL-2 are added to the culture. The half volume of culture medium is exchanged with fresh AIM-V/5% HS medium containing 60 IU/ml of IL-2 on day 5, 8 and 11. Peptide specific IFN-γ production of CD8+ T cells was tested by ELISA between day 14 and day 16.

To examine peptide specific IFN-γ production of CD8+ T cells, ELISPOT assay or ELISA are performed. Peptide-pulsed HLA class II expressing C1R cells (1×104 cells) are prepared as stimulator cells. CD8+ T cells are used as responder cells. IFN-γ ELISPOT assay and IFN-gamma ELISA are performed under the manufacturer's procedure (BD Biosciences, Calif.).

TCR sequences are determined (Fang H et al. Oncoimmunology. 2015; 3:e968467). Total RNA is extracted from expanded or dextramer-positive T cells. cDNAs with common 5′-RACE adapter are synthesized using SMART library construction kit (Clontech, Mountain View, Calif). The fusion PCR is performed to amplify TCRA or TCRB cDNAs using a forward primer corresponding to the SMART adapter sequence and reverse primers corresponding to the constant region of each of TCRA or TCRB. After adding the Illumina index sequences with barcode using the Nextera Index kit (Illumina, San Diego, Calif.), the prepared libraries are sequenced by 300-bp paired-end reads on the MiSeq (Illumina). Obtained sequence reads are analyzed using Tcrip software. The sequence is also confirmed by Sanger sequence using fusion PCR products as a template (Thermo Scientific).

Both TCRA and TCRB sequences are codon-optimized and cloned into pMP71-PRE; incorporated by reference in their entireties). To maximize TCR expression, modified murine TCRA and TCRB constant domains are used. Transient retroviral supernatants are generated and PBMCs from donors are transduced. The expression of the TCR is evaluated with anti-human TCRβV antibodies. Only transduced TCR-engineered T cells are transduced using the staining with APC-conjugated anti-mouse TCRf3 monoclonal antibody (H57-597, eBioscience, San Diego, Calif) at a proper condition for TCR-engineered T cells for FOXM1 and UBE2T followed by the incubation with anti-APC microbeads (Miltenyi Biotec, Bergisch Gladbach, Germany) according to the manufacturer's instructions. To increase the number of T cells transduced with desired TCRs which are not occupied with antibodies, conditions are based on the peak fluorescence intensity and the number of sorted cells by comparing the five different conditions of antibody dilution. 1:2000 (0.1 μg/mL) to 1:4000 (0.05 μg/mL) ratios of antibody staining is used for sorting of TCR-engineered T cells for FOXM1 and UBE2T.

CTL clones are induced that are specific to HLA-DRB3*02:02; HLA-DRB1*13:02; HLA-DRB1*07:01; or HLA-DRB1*09:01 restricted peptides. The CTLs are captured with HLA dextramer with each peptide, and TCR sequences for these cells are determined. Using the HLA expressing cells with or without peptide, all CTL clones are evaluated for peptide specific IFN-γ production by ELISA assays.

### Example 13. Vaccine

The Examples above identified, for example, allergenic proteins ara h1 and ara h 3 present in peanuts and cross-reactive allergenic protein ber e 2 present in brazil nuts and cross-reactive epitopes (MHC-binding T-cell epitopes) located on ara h1, ara h 3, and ber e 2 (see Tables 13-23).

A library containing all cross-reactive MHC-II binding epitopes shared by or common to ara h1, ara h3 and ber e 2 are expressed by mammalian, phage, hyperphage, or yeast display. Epitope that bind to broadly neutralizing epitopes are selected. Additional rounds are further condcuted in the presence of heat, intercalating agents and other protein folding selective pressures to evaluate diversity. A final set of 100 cross-reactive epitopes exhibiting maximal epitope dispersion (most different from one another) are selected. The set of 100 members are then expressed and tested for folding and binding to broadly neutralizing antibodies in a terminal expression system for production. A population of epitopes are purified without His or other tags using broadly neutralizing antibody affinity columns, beads, etc. The resulting pure pools are combined into a single ventivalent (20×), quinquavalent (50×), and centivalent (100×) pools composed of equal composition of each antigen. The performance of the vaccine is validated using FACS and an animal model.

For FACS, a population of cells containing known broadly neutralizing antibodies (a library of antibodies with spike in broadly neutralizing B-cells, or a population of B-cells from an individual known to carry broadly neutralizing B-cells) is sorted using a plurality of labeled cross-reactive epitopes as a detection agent.

Animal models (mice, ferrets, primates, or human) are provided either a single allergen, a multi-allergen, or a saline/adjuvant control. Multivalent doses are made such that individual peptides are below the minimum concentration required for an effective immune response. Animals are subjected to cross-reactive allergen challenge against peanut allergens ara h1 and ara h3 and brazil nut allergen ber e 1 or whole/complete peanut and brazil nut. Responses are assays by ELISA, FACS and high throughput sequencing of the BCR and TCR responding populations.

### Example 14. Prevention and Suppression of Food Allergy With Food Allergen-Derived Cross-Reactive Peptides

Multiple food allergy is being more and more commonly observed in subjects. Subjects presenting with allergies to two or more foods, such as English or Persian walnut, black walnut, pecans, pistachio, peanuts, almonds, sesame, cashew, hazelnut, and soybean are recruited.

Suitable peptides for use in subjects are selected based on cross-reactivity of peptides. Peptides having a minimal acceptable homology (at least 70% identity over 80% coverage) to MHC-II-binding T-cell epitopes are also acceptable for use in such treatment. For example, peptides comprising MHC-II-binding T-cell epitopes that a present on at least 2 distinct allergenic proteins are selected. For example, use of the methods of the present disclosure (for example, as described in Examples 1-5) identified a cross-reactive peptide that includes an MHC-II-binding T-cell epitope (amino acid sequence 5′-HWNLNAHSVVYALRG-3′) derived from English walnut as cross-reactive with 10 additional allergenic proteins within the Cupin protein family from various food sources (Pru_du_6 (almond), gly m 6 (soybean), Pis_v_2 (pistachio), Pis_v_5 (pistachio), Cor_a_9 (hazelnut), Jug_n_4 (black walnut), Ana_o_2 (cashew), Car_i_4 (pecan), Ses_i_7 (sesame), and Ara_h_3 (peanut)) (Tables 13-18, 20, and 22).

The cross-reactive peptide having the amino acid sequence 5′-HWNLNAHSVVYALRG-3′ corresponding to: amino residues 377 to 388 on the walnut (Juglans regia) allergenic protein Jug_r_4 and containing an MHC-II-binding T-cell epitope, amino acid residues 378-389 on the pecan (Carya illinoinensis) allergenic protein Car_1_4; 380-391 on the black walnut (Juglans nigra) Jug_n_4, and to amino acid residues 382-393 on the hazelnut (Coryllus avellane) allergenic protein Cor_a_9 (wherein the MHC-II-binding T-cell peptide epitope on Cor_a_9 has the amino acid sequence of HWNLNAHSVVYAIR having an extremely high homology to peptide sequences of Jug_r_4, Car_i_4, and Jug_n_4 with a single amino acid difference) is selected.

Mouse Study: A composition comprising 50 ug of the cross-reactive peptide comprising the MHC-II-binding T-cell epitope 5′-HWNLNAHSVVYALRG-3′ and a pharmaceutical acceptable carrier is administered orally to mice for 4 weeks. Two weeks after the last dose, blood is drawn from a peripheral vein and CD4+ T cells are purified by cell sorting on magnetic beads. CD4+ T-cells are added to culture medium in which histocompatible dendritic cells, used as antigen presenting cells, are incubated with either the peptide sequence or with tetanus toxoid as a control for 2 hr at room temperature. The cells are then washed and purified CD4+ T-cells are added to culture medium. Such CD4+ T-cells induce apoptosis of dendritic cells presenting the Cor_i_9 antigen, but not of dendritic cells presenting the tetanus toxoid protein. Incubation of CD4+ T-cells with dendritic cells loaded with the hazelnut Cor_i_9 antigen results in dendritic cell apoptosis.

Single Allergen Subcutaneous Immunotherapy: The cross-reactive peptide comprising the Jug_r_4 MHC-II-binding T-cell epitope 5′-HWNLNAHSVVYALRG-3′ is prepared for single allergen oral immunotherapy. Subjects presenting allergic reactions upon exposure to walnut and hazelnut food antigens by ingestion of walnuts and hazelnuts are treated with subcutaneous immunotherapy with a polypeptide comprising the Jug_r_4 5′-HWNLNAHSVVYALRG-3′ and are expected to subsequently fail to react to both walnuts and hazelnuts.

Multiple Allergen Oral Immunotherapy: Subjects presenting allergic reactions upon exposure to peanuts and a suspected allergy to one or more of the food allergens: almond, soybean, pistachio, hazelnut, English walnut, black walnut, cashew, pecan, sesame, and peanut are identified and administered multiple allergen oral immunotherapeutic compositions comprising equal measures proteins or peptides including of each of the MHC-II-binding epitope peptides: 5′-HWNLNAHSVVYALRG-3′ (present in English walnut, pecan, and black walnut); 5′-HWNLNAHSVVYAIRG-3′ present in hazelnuts (Cor_a_9); 5′-HWNFNAHSIVYGCKG-3 present in pistachio (Pis_v_5); 5′-HWNLNAHSIIYITRG-3′ present in sesame (Ses_i_7); and 5′-GALLLPHFNSKAIVI-3′ present in soy (Gly_m_5).

Vaccine: Vaccination with a composition containing the MHC-II-binding T-cell epitope peptides in Table 24 modified to contain a redox moiety is expected to elicit cytolytic regulatory T-cells that reduce or eliminate the symptoms related to walnuts and those resulting from ingestion of at least one of the food sources containing cross-reactive allergenic proteins Pru_du_6 (almond), gly m 6 (soybean), Pis_v_2 (pistachio), Pis_v_5 (pistachio), Cor_a_9 (hazelnut), Jug_n_4 (black walnut), Ana_o_2 (cashew), Car_i_4 (pecan), Ses_i_7 (sesame), and Ara_h_3 (peanut).

### Example 15. Method for Identifying Allergen-Specific B Cells

Labeling Cross-reactive B-cell epitope: Recombinant peptide comprising a cross-reactive B-cell epitope (e.g., on peanut Ara h 3 and cashew Ana_o_2) identified in Example 6 above is biotinylated at lysine residues using a water-soluble biotin-XX sulfosuccinimidyl ester according to the manufacturer's protocol (Invitrogen/Thermo Fisher Scientific, CA) to produce a labelled polypeptide comprising the B-cell epitope peptide. Fluorescent multimers are formed by means of the stepwise addition of Alexa Fluor 488 streptavidin (Invitrogen) to the biotinylated allergen until a 1:4 molar ratio is reached, thereby fluorescently labelling the allergen.

Identifying Allergen-Positive Circulating B Cells from Subjects: The identification of cross-reactive allergenic protein (or polypeptide)-positive circulating B cells from subjects is carried out as previously described (Patil, S. U. et al. (2015) J. Allergy Clin. Immunol. 136(1): 125-134). Briefly, PBMCs are isolated by density gradient centrifugation (Ficoll-Paque Plus; GE Healthcare) from peripheral blood harvested from a subject. The PBMCs are stained with CD3-allophycocyanin (APC; clone OKT3; eBioscience), CD14-APC (clone 61D3, eBioscience), CD16-APC (clone CB16, eBioscience), CD19-APC-Cy7 (clone SJ25C1; BD Biosciences), CD27-phycoerythrin (clone M-T271; BD PharMingen), CD38-Violet 421 (clone HIT2, BD Biosciences), IgM-phycoerythrin-Cy5 (clone G20-127; BD PharMingen), and fluorescently-labelled allergen (e.g., Alexa Fluor 488-Ara h 3 multimer). Alexa Fluor 488⁺ B cells (allergen-positive B cells) are then identified and isolated from the rest of the stained PBMC population by flow cytometry using a FACS AriaIII instrument. Data is analyzed with FlowJo 8.8.7 software. Normalization of Alexa Fluor 488 is performed with Quantum Alexa Fluor 488 Molecules of Equivalent Soluble Fluorochrome (MESF) Beads (Bangs Laboratories). Single allergen-positive CD1913 cells, identified by FACS analysis, are sorted into individual wells of a 96 well plate (Eppendorf) containing 10 μL of first-strand buffer (Invitrogen) and 20 Units of recombinant RNasin Ribonuclease Inhibitor (Promega), and are frozen at −80° C.

Single-Cell RT-PCR and Immunoglobulin Gene Amplification: Immunoglobulin genes are amplified from the frozen B cells using a nested RT-PCR protocol as follows: 3 μL of NP40 and 150 ng of random hexamers are added to each well containing the frozen B cells, and the cells are lysed by heat treating the samples at 65° C. for 10 minutes, 25° C. for 3 minutes, and then a 4° C. incubation. The lysed cells are then subjected to a reverse transcription reaction using first-strand buffer (Invitrogen), 0.1 mM dithiothreitol (Invitrogen), 2.5 mM deoxynucleotide triphosphates (Invitrogen) and SuperScript III Reverse Transcriptase (Invitrogen) according to the manufacturer's protocol. The resulting cDNA is then amplified by two rounds of nested PCR amplification. In the first round, 5 μL of template cDNA is amplified with Taq DNA polymerase (Invitrogen) according to the manufacturer's protocol. In the second round, 3 μL of the Taq-amplified template is amplified with Pfu DNA polymerase (Invitrogen) according to the manufacturer's protocol. A sample taken from the amplification products of the second PCR step is run on a 1.5% agarose gel to determine whether immunoglobulin heavy and light chains are successfully/adequately amplified. Successfully amplified products are then sequenced.

Recombinant Antibody Production: Paired immunoglobulin heavy and light chains are selected after sequencing, and the DNA encoding the selected heavy and light chains (amplified in the second nested PCR step) is purified with the QIAquick 96 PCR Purification Kit (Qiagen). NEB® 5-alpha competent E. coli (New England Biolabs) are transformed with the heavy and light chain ligation products at 42° C. for 30 seconds, grown for 1.5 hours at 37° C., followed by selection on LB plates with 100 μg/mL ampicillin. Ampicillin-resistant clones are selected and screened for vector insertion of the amplified immunoglobulin heavy and light chains. Vectors harboring the insertions are then amplified and purified from overnight liquid cultures (LB with 100 μg/mL ampicillin) of selected colonies using the QIAprep Spin Miniprep Kit (Qiagen). Plasmid DNA is then sequenced with colony PCR primers to determine similarity to previous sequences. Plasmid DNA (25 ng) from selected heavy and light chains are transfected into HEK293T cells using GenJet In Vitro DNA Transfection Reagent (SignaGen, Rockville, Md.) according to the manufacturer's protocol. Antibodies are purified from the transfected HEK293T cells according to standard techniques.

Recombinant Antibody Characterization: Allergen specificity is then validated by ELISA assay (ImmunoCAP) for the recombinant antibodies purified from the transfected HEK293T cells according to the manufacturer's protocol. For immunoblot inhibition experiments, recombinant cross-reactive proteins (e.g., Jug_r_4, Ara_h_1, Ara_h_3, Car_i_4, Jug_n_4, Cor_a_9) are loaded onto a gel at concentration of 2-3 μg of protein or peptides thereof/cm of gel, Immunoblotting experiments are then performed as is well-known in the art using the recombinant antibodies developed using the methods described above. Membranes are then incubated with fluorescent or radiolabeled goat anti-human IgE and exposed to Kodak Imaging film for 12 days or through fluorescent microscopy or other fluorescence detection systems. A pool of subjects' sera is preabsorbed with cross-reactive proteins (e.g., Jug_r_4, Ara_h_1, Ara_h_3, Car_i_4, Jug_n_4, Cor_a_9) and whole peanut flour protein extract as control.

### Example 16. Peptides for Antibody Preparation

Synthesis of cross-reactive allergenic peptide haptens: 0.5 g of p-carboxybenzaldehyde is dissolved in 10 ml of water, 3 ml of HCl solution with a concentration of 1 mol/L is added to obtain a mixed solution 1; and DMF and 1.02 g of MHC-II-binding peptides of Cupin allergenic proteins provided below are added to the mixed solution while magnetically stirring, and then the mixed solution is stirred magnetically at 60° C. and refluxed overnight to obtain a mixed solution 2; the obtained mixed solution 2 is centrifuged, washed and dried to obtain a crude product; and column chromatography separation is carried out on the crude product to obtain the Cupin allergenic cross-reactive epitope-haptens.

Walnut Jug_r_4 Peptides: HWNLNAHSVVYALR (aa 377-388 aa), VPHWNLNAHSVVYA (aa 375-389 aa), HTLPVLRWLQLSAE (aa 351-aa 365), DIIAFPAGVAHWSY (aa 145-aa157), LLLPQYSNAPQLVY (aa 83-97 aa).

Pecan Car_i_4 Peptides: HWNLNAHSVVYALR (aa 378-389 aa), AGVAHWCYNDGSSP (aa 152-166 aa), LPILRWLQLSAERG (aa 354-366 aa), DIIAFPAGVAHWCY (aa 146-159 aa), PAGVAHWCYNDGSS (aa 151-165 aa).

Hazelnut Cor_a_9 Peptides: HWNLNAHSVVYAIR (aa 382-393 aa), GDIIALPAGVAHWC (aa 152-166 aa), PAGVAHWCYNDGDS (aa 158-172 aa), LLLPQYSNAPELIY (aa 85-99 aa), FREGDIIALPAGVA (aa 149-163), AGRTSAIRALPDDV (aa 455-469 aa).

Mung Bean Vig_r_2: FGINAENNQRNFLA (aa 379-392 aa), GSLLLPHYNSKAIV (aa 300-313 aa), GINAENNQRNFLAG (aa 380-394), SLLLPHYNSKAIVI (aa 301-315 aa), QRNFLAGEKDNVIS (aa 387-400 aa).

Peanut Ara_h_3 MHC-II binding Epitope Peptides: HYNTNAHSIIYALR (aa 407-418 aa), LLILRWLGLSAEYG (aa 383-395 aa); GHVLVVPQNFAVAG (aa 443-455 aa); AHSIIYALRGRAHV (aa 412-424 aa); ALRGRAHVQVVDSN (aa 418-431 aa).

Cashew Ana_o_2 MHC-II binding Epitope Peptides: AGVAHWCYNEGNSP (aa 139-153 aa); EIWNLNSHSITYGCK (aa 333-344 aa); GDIIAIPAGVAHWC (aa 132-146 aa); LLLPQYSNAPQLIY (aa 72-86 aa); NGIEETICTMRLKE (aa 270-282 aa).

Common Buckwheat Fag_e_1 MHC-II binding Epitope Peptides: RINTVNSNNLPILE (aa 406-420 aa); QILVVPQGFAVVLK (476 aa-488 aa); PRAGRINTVNSNNL (402 aa-416 aa); SVLRAIPVEVLANS (516 aa-529 aa); TKLILSFSLCLMVL (2 aa-16 aa)

Synthesis of a Jug_r_4 Complete Antigen: 4.5 mg of a Jug_r_4 hapten, 2.0 mg of EDC (1-(3-dimethylaminopropyl)-3-ethylcarbodiimide hydrochloride) and 1.0 mg of NHS (N-hydroxysuccinimide) are dissolved in DMF (N,N-dimethylformamide), and stirred for activation at room temperature for 4 h to obtain a solution A; 5 mg of KLH is dissolved in 2 mL of the CB solution with a concentration of 0.05 mol/L and a pH of 9.6 to obtain a solution B; and the solution A is dropwise added to the solution B, and stirred at room temperature for reaction overnight to obtain a Jug_r_4 complete antigen.

Synthesis of Coating Antigens (Cross-Reactive Epitope Peptides): 5 mg of thiamethoxam hapten (TMX-COOH) and 4.8 mg of N-hydroxysuccinimide (NETS) are dissolved in 300 μL of anhydrous N,N-dimethylformamide (DMF), and the solution is stirred at room temperature to react for 10 min to obtain a TMX-COOH solution; 7.6 mg of 1-(3-dimethylaminopropyl)-3-ethylcarbodiimide hydrochloride (EDC) is dissolved in 100 μL of anhydrous DMF and then added to the TMX-COOH solution, and stirred at room temperature to react for 6-8 h to obtain a solution A; 10 mg of each WIC-II-binding epitope peptide is diluted with 1 mL of a phosphate buffer solution (PBS) with a concentration of 0.01 mmol/L and a pH of 7.4 to obtain a solution B; the solution A is slowly added dropwise to the solution B to react to obtain a reaction solution; the reaction solution is dialyzed with the PBS solution to remove the unreacted small molecular hapten to obtain a coating antigen (TMX-COOH-epitope peptide).

### Example 17. Preparation of a Hybridoma Cell Strain Secreting a Cross-Reactive Allergenic Polypeptide Monoclonal Antibody

Acquisition of Immunity in Animals: Healthy 6-8 week old BALB/c mice are selected for immunization. After a Cupin Jug_r_4 cross-reactive complete antigen (1 mg/mL) is emulsified uniformly with an equal amount of a Freund's adjuvant, BALB/c mice are immunized with the emulsified Jug_r_4 complete antigen by subcutaneous injection at a dose of 100 μL for each mouse. The first immunization is performed with a complete Freund's adjuvant, the booster immunization is performed with an incomplete Freund's adjuvant, and the immunization dose for the rush immunization is half of the dose for previous immunization; the Freund's adjuvants are directly used through intraperitoneal injection after being mixed with normal saline; the immunization interval of the immunizations is three weeks. After the third immunization, blood samples are taken at intervals of one week to detect serum titer and inhibitory effect; the mice with the best inhibitory effect are selected and are subject to rush immunization 21 days after the fifth immunization.

Cell Fusion: Three days after the rush immunization, cell fusion is carried out according to the conventional PEG (polyethylene glycol, with a molecular weight of 4000) method. The specific steps are as follows: (1) The mouse spleens are aseptically taken, ground and screened through a 200-mesh cell sieve to obtain a spleen cell suspension, and cells are counted; (2) SP2/0 cells are collected, suspended in a RPMI-1640 basal medium, and subjected to cell counting; and (3) Spleen cells and SP2/0 cells are mixed at a ratio of 1:10, centrifuged and fused with 50% PEG for 1 min, then the RPMI-1640 basal medium is added from slow to fast, the solution is centrifuged and then suspended in an RPMI-1640 screening medium containing 20% fetal bovine serum and 2% 50×HAT, and then added to a 96-well cell culture plate and cultured in an incubator at 37° C. in a 5% CO2 atmosphere.

Cell Screening and Cell Strain Establishment: The medium of the fused cells is semi-changed with an RPMI-1640 screening medium on the third day of cell fusion, and then completely changed with an RPMI-1640 transition medium containing 20% fetal bovine serum and 1% 100.times.HT on the 5th day. The cell supernatant is taken for screening on the 7th day. The screening is divided into two steps: the first step is to select positive cell wells by ic-ELISA, the second step is to use a cross-reactive peptide as a standard and measure the inhibitory effect of the positive cells by ic-ELISA. Cell wells with a good inhibitory effect to the cross-reactive peptide standard are selected and subcloned by a limiting dilution method. The same method is used for detection and repeated three times to obtain a cell strain.

Preparation and Identification of a Jug_r_4 cross-reactive peptide Monoclonal Antibody: 8-10 weeks old BALB/c mice are taken and each intraperitoneally injected with 1 mL of sterile paraffin oil, and 7 days later, intraperitoneally injected with a 1×106 hybridoma cell strain; ascites is collected from the 7th day and purified by an octanoic acid-ammonium sulfate method. Under an acidic condition, n-octanoic acid can precipitate heterologous proteins other than IgG immunoglobulin in ascites, then centrifugation is performed to remove the precipitate; the IgG monoclonal antibody is then precipitated with an ammonium sulfate solution with the equivalent saturability, centrifuged to remove the supernatant, dissolved in 0.01 M PBS solution (with a pH of 7.4), and dialyzed for desalting to finally obtain the purified monoclonal antibody, and the purified monoclonal antibody is stored at −20° C. By using an indirect competitive ELISA, the IC₅₀ of the monoclonal antibody is determined to be 2 μg/L, indicating a good sensitivity to Jug_r_4 cross-reactive peptide. Thus, the monoclonal antibody can be used for Jug_r_4 cross-reactive peptide immunoassay. Further, the lymphocytes harvested from the immunized host, or the hybridoma lines can be the source to derive antibody variable region sequences then used to make recombinant proteins.

### Example 18. Allergenic Potency of Multiple Allergen Oral Immunotherapeutic Compositions

A competitive ELISA is performed to measure the potency of various multiple allergen oral immunotherapeutic compositions. ELISA is based on coating allergens (full-length allergenic proteins and MHC-II-binding epitopes or polypeptides comprising such

MHC-II-binding epitopes) on multi-well plates and then allowing allergen-specific IgE from pooled human biological samples (e.g., serum) containing cross-reactive IgE antibodies to bind to the coated allergenic proteins and MHC-II binding epitope peptides on the multi-well plates and detecting bound IgE in the biological sample and detecting bound IgE with anti-IgE-alkaline phosphatase which converts pNPP to become detectable at 405 nm. Absorbance at 405 nm is proportional to the amounts of allergens present in the multiple allergen oral immunotherapeutic compositions.

In one study, potency is measured for a multiple allergen oral immunotherapeutic compositions comprising walnut Jug_r_4, Pecan Car_i_4, Hazelnut Cor_a_9, Mung Bean Vig_r_2, Peanut Ara_h_3, Cashew Ana_o_2 and Fag_e_1.

Walnut Jug_r_4 full-length proteins and MHC-II binding epitope peptides used for coating the multi-well plates are: HWNLNAHSVVYALR (aa 377-388 aa), VPHWNLNAHSVVYA (aa 375-389 aa), HTLPVLRWLQLSAE (aa 351-aa 365), DIIAFPAGVAHWSY (aa 145-aa157), LLLPQYSNAPQLVY (aa 83-97 aa).

Pecan Car_i_4 full-length proteins and MHC-II binding epitope peptides used for coating the multi-well plates are: HWNLNAHSVVYALR (aa 378-389 aa), AGVAHWCYNDGSSP (aa 152-166 aa), LPILRWLQLSAERG (aa 354-366 aa), DIIAFPAGVAHWCY (aa 146-159 aa), PAGVAHWCYNDGSS (aa 151-165 aa).

Hazelnut Cor_a_9 full-length proteins and MHC-II binding epitope peptides used for coating the multi-well plates are: HWNLNAHSVVYAIR (aa 382-393 aa), GDIIALPAGVAHWC (aa 152-166 aa), PAGVAHWCYNDGDS (aa 158-172 aa), LLLPQYSNAPELIY (aa 85-99 aa), FREGDIIALPAGVA (aa 149-163), AGRTSAIRALPDDV (aa 455-469 aa)

Mung Bean Vig_r_2 full-length proteins and MHC-II binding epitope peptides used for coating the multi-well plates are: FGINAENNQRNFLA (aa 379-392 aa), GSLLLPHYNSKAIV (aa 300-313 aa), GINAENNQRNFLAG (aa 380-394), SLLLPHYNSKAIVI (aa 301-315 aa), QRNFLAGEKDNVIS (aa 387-400 aa)

Peanut Ara_h_3 full-length proteins and MHC-II binding epitope peptides used for coating the multi-well plates are: HYNTNAHSIIYALR (aa 407-418 aa), LLILRWLGLSAEYG (aa 383-395 aa); GHVLVVPQNFAVAG (aa 443-455 aa); AHSIIYALRGRAHV (aa 412-424 aa); ALRGRAHVQVVDSN (aa 418-431 aa).

Cashew Ana_o_2 full-length proteins and MHC-II binding epitope peptides used for coating the multi-well plates are: AGVAHWCYNEGNSP (aa 139-153 aa); HWNLNSHSIIYGCK (aa 333-344 aa); GDIIAIPAGVAHWC (aa 132-146 aa); LLLPQYSNAPQLIY (aa 72-86 aa); NGIEETICTMRLKE (aa 270-282 aa).

Common Buckwheat Fag_e_1 full-length proteins and MHC-II binding epitope peptides used for coating the multi-well plates are: RINTVNSNNLPILE (aa 406-420 aa); QILVVPQGFAVVLK (476 aa-488 aa); PRAGRINTVNSNNL (402 aa-416 aa); SVLRAIPVEVLANS (516 aa-529 aa); TKLILSFSLCLMVL (2 aa-16 aa).

As controls, protein extracts from each of the food allergens: walnut, pecan, hazelnut, mung bean, peanut, cashew, and buckwheat are also coated on the microarray slides.

In a second study, potency of a multiple allergen oral immunotherapeutic compositions comprising a shrimp allergen, a cod allergen, a salmon allergen, a hen's egg allergen, a cow's milk allergen, a peanut allergen, a sesame allergen, a soy allergen, a pecan allergen, a cashew allergen, a hazelnut allergen, a walnut allergen, a pistachio allergen, an almond allergen, and a wheat allergen selected from Table 2 are evaluated. Crude extracts, recombinant or isolated full-length proteins and/or polypeptides comprising at least 4 epitopes from each allergen are selected and coated to multi-well plate to perform ELISA as described herein.

ELISA coating allergens, reference standards, quality control, and standard curve are each produced from the allergens. Reference standards are prepared with each allergenic protein, and the MHC-II-binding epitope peptides and from each food source flour prior to being extracted. Positive IgE pooled human sera for each allergen is generated by poling sera from subjects that are IgE positive for each allergen. The specific IgE for each peptide and full-length allergenic protein is determined.

Blocking buffer (5% FBS, 1% Tween 20) is prepared in PBS and stored in 4° C. for up to a month. Washing Buffer (1×PBS, 0.05% Tween 20) is prepared and stored at room temperature for up to a month.

Coating allergen samples are prepared by adding allergen extracts, recombinant full-length proteins and peptides to 50 mM sodium carbonate/sodium bicarbonate pH 9.2 and allowing adherence to a Greiner high binding plate overnight at 4° C. or at room temperature for 2 hours. Plates are washed 3 times with washing buffer, before being blocked with blocking buffer for an hour at room temperature, and washed 3 more time with washing buffer. Wells are finally emptied by flicking off and ready for sample loading.

The Positive pooled human sera are diluted 1: 40 in blocking buffer and incubated for 12 hours at 4° C. Diluted antihuman IgE-AP conjugate (1L500) is added to each well and the multi-well plates are incubated for 4 hours at room temperature. The plate contents are discarded and the multi-well plates are washed 3 times with wash buffer. pNPP substrate in TRIS buffer is added and the color reaction allowed to develop for 15-30 minutes at room temperature before being stopped by the addition of 50 mCL 3M NaOH. The multi-well plates are read at absorbance wavelength 405 nm.

Potencies of the multiple allergen oral immunotherapeutic compositions are provided.

### Example 19. Preparation of an Exemplary Multiple Allergen Oral Immunotherapeutic Composition

Three Cupin proteins that are cross-reactive with each other are selected from Table 10.

For example, Ber_e_1, Car_i_4, and Jug_n_4 are selected for preparation of a multiple allergen oral immunotherapy that includes full-length cross-reactive allergenic proteins.

Each of the 3 selected Cupin proteins are recombinantly synthesized in small batches as described below. Small scale recombinant proteins (≥75% purity, endotoxin level <10 EU/mg) are expressed in E. coli and/or in insect cells as a custom service by a commercial vendor (e.g., GenScript, NJ, USA) using codon optimized DNA constructs. Selected proteins are further expressed in a human embryonic kidney (HEK293) suspension cell line (Freestyle™ 293 Expression System, Thermo Fisher, MA, USA), according to the manufacturer's instructions. Briefly: 30 μg transfection grade, codon optimized plasmids encoding the protein of interest (made as a custom service by Genscript, NJ, USA), are mixed with 60 μl 293Fectin™, and incubated for 25 min. This mixture is added to 30 ml suspension culture of HEK293 cells with a cell density of 1×10⁶ cell/ml. The culture is incubated in 125 ml disposable, polycarbonate, Erlenmeyer flasks with vent caps (Corning, N.Y.) in a 37° C. incubator having a humidified atmosphere with 8% CO2 and orbital shaking at 125 rpm for 2-5 days before harvesting. Recombinant proteins secreted into the medium are harvested by sedimentation of the HEK293 cells at 100 g for 5 min. The cell supernatants are subsequently sterilized through a low protein binding Millex-GP 0.45 um filter (Millipore, MA, USA).

Equal amounts (1:1:1) of each of the three proteins Ber_e_1, Car_i_4, and Jug_n_4 on a w/w basis are lyophilized and packed neat into Vcaps vegetarian capsule (available from Capsugel) to create a multiple allergen oral composition comprising each cross-reactive allergenic protein at 300 mg/capsule.

### Example 20. Preparation of an Exemplary Multiple Allergen Oral Immunotherapeutic Composition

Peptide Synthesis and HPLC purification. Peptides are selected from Tables 13-23. Briefly peptides are synthesized on a Milligen/Biosearch 9600 peptide synthesizer using a 4-mthylbenzhydrylamine resin as the solid support (substitution 0.54 mm/g). The Fmoc/t-butyl synthetic methods is employed using 4(hydroxymethyl) phenoxyacetic acid as the linker. After the final deprotection step, protecting groups and peptide resin bond are cleaved with 90% TFA, 5% anisole, 3% tioanisole, 2% ethanediol. Crude peptide is purified by semi-preparative HPLC using a Vydac C4 (10 mm×25 cm) column at 32.5° C. buffers are 0.1% TFA in H₂O and 0.1% TFA in acetonitrile. Peptides incorporate a degenerate MHC as provided herein.

For example, the following cross-reactive MHC-II-binding T-cell epitope peptides are selected for preparation of an allergen oral immunotherapeutic vaccine composition.

Ber_e_2 14-mer peptide AIPAGVALWCYNDG—cross-reactive with five Cupin proteins Ana_o_2;Jug_n_4;Jug_r_4;Cor_a_9;Car_i_4 and capable of binding 25 MHC variants: HLA-DQA1*05:01/DQB1*03:01;HLA-DQA1*01:02/DQB1*06:02;HLA-DQA1*04:01/DQB1*04:02;HLA-DQA1*03:01/DQB1*03:02;HLA-DRB1*09:01;HLA-DQA1*01:01/DQB1*05:01;HLA-DQA1*05:01/DQB1*02:01;HLA-DRB1*01:01;HLA-DRB1*07:01;HLA-DRB5*01:01;HLA-DRB1*15:01;HLA-DPA1*01:03/DPB1*02:01;HLA-DRB1*11:01;HLA-DRB1*08:02;HLA-DRB1*12:01;HLA-DPA1*02:01/DPB1*01:01;HLA-DRB3*02:02;HLA-DRB1*04:01;HLA-DRB1*04:05;HLA-DPA1*03:01/DPB1*04:02;HLA-DPA1*02:01/DPB1*05:01;HLA-DRB4*01:01;HLA-DRB3*01:01;HLA-DRB1*13:02;HLA-DRB1*03:01;HLA-DRB1*09:01;HLA-DRB3*02:02;HLA-DPA1*01/DPB1*04:01;HLA-DQA1*05:01/DQB1*03:01.

Ber_e_2 14-mer peptide AIPAGVALWCYNDG—cross-reactive with 5 Cupin proteins Ana_o_2;Jug_n_4;Jug_r_4;Cor_a_9;Car_i_4 and is capable of binding 29 MHC variants: HLA-DQA1*05:01/DQB1*03:01;HLA-DQA1*01:02/DQB1*06:02;HLA-DQA1*04:01/DQB1*04:02;HLA-DQA1*03:01/DQB1*03:02;HLA-DRB1*09:01;HLA-DQA1*01:01/DQB1*05:01;HLA-DQA1*05:01/DQB1*02:01;HLA-DRB1*01:01;HLA-DRB1*07:01;HLA-DRB5*01:01;HLA-DRB1*15:01;HLA-DPA1*01:03/DPB1*02:01;HLA-DRB1*11:01;HLA-DRB1*08:02;HLA-DRB1*12:01;HLA-DPA1*02:01/DPB1*01:01;HLA-DRB3*02:02;HLA-DRB1*04:01;HLA-DRB1*04:05;HLA-DPA1*03:01/DPB1*04:02;HLA-DPA1*02:01/DPB1*05:01;HLA-DRB4*01:01;HLA-DRB3*01:01;HLA-DRB1*13:02;HLA-DRB1*03:01;HLA-DRB1*09:01;HLA-DRB3*02:02;HLA-DPA1*01/DPB1*04:01;HLA-DQA1*05:01/DQB1*03:01

Ber_e_2 14-mer peptide RGILGVLMPGCPET—cross-reactive with 4 Cupin proteins Jug_r_4; Cor_a_9; Car_i_4; and Jug_n_4 is capable of binding 79 MHC variants.

Ber_e_2 14-mer peptide PRAGRLTTVNSLKV—cross-reactive with 2 Cupin proteins Fag_t_13 S_globulin; and Fag_e_1 is capable of binding 80 MHC variants.

Briefly, 20 mg/ml acidified peptide solution (0.1 mg/mL) is loaded on to a Sephadex G-25 column and 5 ml fraction are eluted with 0.1 M HoAc. Peptide samples are measured spectrophotometrically at 235 nm and absorbance values plotted over time. Samples with absorbance over 0.1 and eluting before DTT are collected, pooled and lyophilized. Capillary zone electrophoresis is performed and sample is voltage separated. Mass spectrometry is performed and circular dichroism is measured at ambient temperature. Mean residue ellipticity (mdeg) is calculated. Fast atom bombardment mass spectrometry measurement is conducted. Peptides are dissolved in minimal amount of water and 100 mg/mm S-tBu solution is added. Peptide is placed under vacuum and precipitated by 2-mercaptoethanol in a 55° C. water bath while stirring. Solution is filtered and filtrate is rotary evaporated, acidified with 0.1% TFA in water and lyophilized. Equal amounts of lyophilized epitope peptides is placed in a glass vial and sealed until further use.

An oil-in-water emulsion is prepared by providing polysorbate 80 as surfactant and combined with lipids (6-10 carbon fatty acid esters of glycerol and 1,2-propanediol) and then further combining the mixture with distilled water to prepare the oil-in-water emulsion. Adjuvant (Aluminum hydroxide (alum)) and anti-oxidant (α-tocopherol) is also included in the emulsion and mixed to homogeneity and provided as a component in a kit that also includes the vial containing the lyophilized epitope peptides, and instructions for use. The lyophilized epitope peptides are combined with the emulsion of oil-in-water by injecting using a syringe or any other suitable means to create an allergen oral vaccine composition such that each epitope peptide is present in the final composition in equal amounts on a w/w basis and is about 500 nmol/mL.

### Example 21. Correlating Cross-Reactivity Between Allergic Proteins with High Sequence Similarity

In another example, forty-two human serum samples with self-reported clinical allergies were obtained and analyzed in triplicate on an ALEX2 chip (developed by Macro Array Diagnostics), which enables an ELISA-based multiplex assay for the detection of IgE adsorption to allergen components and extracts. The ALEX2 chip contains ˜300 allergenic proteins, including ten recombinant allergens from the Bet v 1 family, 12 from the Cupin family and 5 from the Tropomyosin family. The assay detects the binding of IgE to these allergens in an ELISA-based multiplex fashion. The dynamic range of the chip spans a little over two orders of magnitude, 0.3-50 kUA/L.

Based on the assay, twenty-two (22) samples had a positive value for the Bet v 1 allergen from the forty two samples run on the ALEX2 chip. Considering there are 9 other allergenic proteins from the Bet v 1 family on this ALEX2 chip, and based on the cross-reactivity predicted by the computational methods described herein, a correlation between the reactivity observed for these 22 samples was expected. As seen in tables 25 (Bet v 1 family) and 26 (Tropomyosin family) below, a high sequence similarity between allergenic proteins generally corresponds to a high degree of correlation between the IgE levels. The observed high correlation between a pattern on the chip and a pattern predicted computationally therefore showed that a computation method (as described herein) can predict potential cross-reactivity between allergenic proteins with a very high degree of certainty.

TABLE 25
Bet v 1 Family

| | | Pearson | % sequence |
|---|---|---|---|
| | | Correlation | similarity |
| | | coefficient | based on |
| Allergen | Common | based on | RISC plots to |
| name | Name | ALEX data | Bet v 1 |
| Bet v 1 | Silver birch | 1.00 | 100 |
| Cor a 1.0103 | Hazel | 0.78 | 82 |
| Cor a 1.0401 | Hazelnut | 0.71 | 81.25 |
| Fag s 1 | Beech | 0.67 | 76.25 |
| Mal d 1 | Apple | 0.62 | 68.55 |
| Ara h 8 | Peanut | 0.55 | 63.05 |
| Gly m 4 | Soy | 0.52 | 62.02 |
| Aln g 1 | Alder | 0.52 | 88.12 |
| Fra a 1 + 3 | Strawberry | 0.50 | 68.12 |
| Api g 1 | Celery | 0.42 | 57.79 |
| Dau c 1 | Carrot | 0.37 | 51.25 |

TABLE 26
Tropomyosin Family

| | | Pearson | % sequence |
|---|---|---|---|
| | | Correlation | similarity |
| | | coefficient | based on |
| Allergen | | based on | RISC plots |
| name | Common Name | ALEX data | to Penm1 |
| Penm1 | Black-Tiger shrimp | 1.00 | 100.00 |
| Pera7 | American cockroach | 0.99 | 82.04 |
| Blot | Blomia tropicalis | 0.98 | 80.28 |
| Derp | European house dust | 0.99 | 80.28 |
| | mite | | |
| Anis | Anisakis simplex | 0.99 | 72.88 |

### INCORPORATION BY REFERENCE

All publications and patents mentioned herein, including those items listed below, are hereby incorporated by reference in their entirety for all purposes as if each individual publication or patent was specifically and individually incorporated by reference. In case of conflict, the present application, including any definitions herein, will control.

### EQUIVALENTS

While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

Lengthy table referenced here
US20240230671A9-20240711-T00001
Please refer to the end of the specification for access instructions.

Lengthy table referenced here
US20240230671A9-20240711-T00002
Please refer to the end of the specification for access instructions.

Lengthy table referenced here
US20240230671A9-20240711-T00003
Please refer to the end of the specification for access instructions.

Lengthy table referenced here
US20240230671A9-20240711-T00004
Please refer to the end of the specification for access instructions.

Lengthy table referenced here
US20240230671A9-20240711-T00005
Please refer to the end of the specification for access instructions.

Lengthy table referenced here
US20240230671A9-20240711-T00006
Please refer to the end of the specification for access instructions.

Lengthy table referenced here
US20240230671A9-20240711-T00007
Please refer to the end of the specification for access instructions.

Lengthy table referenced here
US20240230671A9-20240711-T00008
Please refer to the end of the specification for access instructions.

Lengthy table referenced here
US20240230671A9-20240711-T00009
Please refer to the end of the specification for access instructions.

Lengthy table referenced here
US20240230671A9-20240711-T00010
Please refer to the end of the specification for access instructions.

Lengthy table referenced here
US20240230671A9-20240711-T00011
Please refer to the end of the specification for access instructions.

Lengthy table referenced here
US20240230671A9-20240711-T00012
Please refer to the end of the specification for access instructions.

Lengthy table referenced here
US20240230671A9-20240711-T00013
Please refer to the end of the specification for access instructions.

Lengthy table referenced here
US20240230671A9-20240711-T00014
Please refer to the end of the specification for access instructions.

Lengthy table referenced here
US20240230671A9-20240711-T00015
Please refer to the end of the specification for access instructions.

Lengthy table referenced here
US20240230671A9-20240711-T00016
Please refer to the end of the specification for access instructions.

Lengthy table referenced here
US20240230671A9-20240711-T00017
Please refer to the end of the specification for access instructions.

Lengthy table referenced here
US20240230671A9-20240711-T00018
Please refer to the end of the specification for access instructions.

Lengthy table referenced here
US20240230671A9-20240711-T00019
Please refer to the end of the specification for access instructions.

Lengthy table referenced here
US20240230671A9-20240711-T00020
Please refer to the end of the specification for access instructions.

Lengthy table referenced here
US20240230671A9-20240711-T00021
Please refer to the end of the specification for access instructions.

Lengthy table referenced here
US20240230671A9-20240711-T00022
Please refer to the end of the specification for access instructions.

Lengthy table referenced here
US20240230671A9-20240711-T00023
Please refer to the end of the specification for access instructions.

Lengthy table referenced here
US20240230671A9-20240711-T00024
Please refer to the end of the specification for access instructions.

LENGTHY TABLES
The patent application contains a lengthy table section. A copy of the table is available in electronic form from the USPTO web site (https://seqdata.uspto.gov/?pageRequest=docDetail&DocID=US20240230671A9). An electronic copy of the table will also be available from the USPTO upon request and payment of the fee set forth in 37 CFR 1.19(b)(3).

What is claimed is:

1. A method of treating a subject for two or more allergies, the method comprising:
(a) identifying the subject as having a first allergy to a first allergenic protein;
(b) identifying the subject as having a second allergy to a second allergenic protein that is immunologically cross-reactive with the first allergenic protein; and
(c) administering a multiple allergen oral immunotherapy to the subject.

2. The method of claim 2, wherein identifying the subject as having the second allergy comprises assessing the presence of IgE antibodies in the subject that bind to the first allergenic protein.

3. The method of claim 1 or 2, wherein identifying the subject as having the second allergy comprises assessing the presence of a T-cell epitope within the first allergenic protein.

4. The method of any one of claims 1-3, wherein the multiple allergen oral immunotherapy does not contain the second allergenic protein.

5. The method of any one of claims 1-4, wherein identifying the subject as having the first allergy comprises evaluating the subject's clinical history of allergy.

6. The method of any one of claims 1-5, wherein identifying the subject as having the second allergy does not include evaluating the subject's clinical history of allergy.

7. The method of any one of claims 1-6, wherein the first allergy is a shrimp, cod, salmon, hen's egg, cow's milk, peanut, sesame, soy, pecan, cashew, hazelnut, walnut, pistachio, almond, or wheat allergy.

8. The method of any one of claims 1-7, wherein the multiple allergen oral immunotherapy comprises two or more of: a shrimp allergen, a cod allergen, a salmon allergen, a hen's egg allergen, a cow's milk allergen, a peanut allergen, a sesame allergen, a soy allergen, a pecan allergen, a cashew allergen, a hazelnut allergen, a walnut allergen, a pistachio allergen, an almond allergen, and a wheat allergen.

9. The method of any one of claims 1-8, wherein the multiple allergen oral immunotherapy comprises a shrimp allergen, a cod allergen, a salmon allergen, a hen's egg allergen, a cow's milk allergen, a peanut allergen, a sesame allergen, a soy allergen, a pecan allergen, a cashew allergen, a hazelnut allergen, a walnut allergen, a pistachio allergen, an almond allergen, and a wheat allergen.

10. The method of any one of claims 1-9, wherein the multiple allergen oral immunotherapy comprises one or more allergenic proteins listed in Table 2.

11. The method of any one of claims 1-10, wherein the second allergy is selected from the group consisting of: a prawn, kiwi, brazil nut, lobster, celery, coconut, crab, apple, buckwheat, clam, pear, lentil, mussel, tomato, garden pea, oyster, stone fruit (e.g., apricot, peach, cherry), yellow mustard, abalone, birch tree, mung bean, squid, alder tree, lupin, octopus, white oak tree, Korean pine, house dust mite, cockroach, and yellow fever mosquito allergy.

12. The method of any one of claims 1-11, wherein the first and/or second allergenic protein is selected from the group consisting of proteins listed in Table 4.

13. The method of any one of claims 1-12, wherein the first and/or second allergenic protein is selected from the group consisting of proteins listed in Tables 5-7 and Tables 10-12.

14. A method of treating an allergy to kiwifruit, brazil nut, coconut, buckwheat, lentil, garden pea, yellow mustard, mung bean, lupin, and/or Korean pine in a subject in need thereof, the method comprising administering to the subject a multiple allergen oral immunotherapy comprising a soybean, sesame, pistachio, pecan, hazelnut, walnut, cashew, and/or almond allergen.

15. The method of claim 14, wherein the multiple allergen oral immunotherapy does not comprise a kiwifruit, brazil nut, coconut, buckwheat, lentil, garden pea, yellow mustard, mung bean, lupin, and/or Korean pine allergen.

16. A method for treating a subject in need thereof having an allergy to an allergenic protein listed in the first column of any one of Tables 10-12, the method comprising administering to the subject a multiple allergen oral immunotherapy comprising an allergenic protein listed in the corresponding row of the second column of Tables 10-12.

17. The method of claim 16, wherein the multiple allergen oral immunotherapy does not comprise the allergenic protein listed in the first column of Tables 10-12.

18. A method for treating a subject in need thereof having an allergy to an allergenic peptide listed in the third column of any one of Tables 13-23, the method comprising administering to the subject a multiple allergen oral immunotherapy comprising an allergenic protein listed in the corresponding row of the sixth column of Tables 13-23.

19. The method of claim 18, wherein the multiple allergen oral immunotherapy does not comprise the allergenic peptide listed in the third column of Tables 13-23.

20. A method for detecting development of tolerance to and/or monitoring the efficacy of a multiple allergen oral immunotherapy in a subject being administered the multiple allergen oral immunotherapy and in need thereof, the method comprising:
(a) obtaining a biological sample from the subject;
(b) contacting the biological sample with a reference allergenic protein present in the multiple allergen oral immunotherapy and/or an other protein that is immunologically cross reactive to the reference allergenic protein;
(c) measuring an amount of IgE, IgG, or T-cells that bind to the reference allergenic protein and/or the other protein; and
(d) comparing the amount of IgE, IgG or T-cells with a previous measurement of the amount of IgE, IgG or T-cells;
wherein tolerance and/or efficacy is indicated when the amount of IgE, IgG or T-cells, is less than the previous amount of IgE, IgG or T-cells.

21. The method of claim 20, wherein the reference allergenic protein is selected from the group consisting of a shrimp allergen, a cod allergen, a salmon allergen, a hen's egg allergen, a cow's milk allergen, a peanut allergen, a sesame allergen, a soy allergen, a pecan allergen, a cashew allergen, a hazelnut allergen, a walnut allergen, a pistachio allergen, an almond allergen, and a wheat allergen.

22. The method of claim 20 or 21, wherein the reference allergenic protein is listed in Table 2.

23. The method of any one of claims 20-22, wherein the other protein is in the same protein family as the reference allergenic protein.

24. The method of claim 23, wherein the protein family is listed in Table 3.

25. The method of any one of claims 20-24, wherein the other protein is identified by the method of any one of claims 48-67, or is listed in Table 4, Tables 5-7, or Tables 10-12.

26. The method of any one of claims 20-25, wherein the reference allergenic protein and/or the other protein are coupled to a solid support to form one or more protein-solid support complexes.

27. The method of claim 26, wherein the solid support is a membrane, a filter, a chip, a slide (such as a microscopic slide), an array (such as a microarray), a wafer, a fiber, a magnetic or a non-magnetic bead, a gel, a tubing, a plate (such as a microtiter plate), a microtiter strip, a polymer, a microparticle, a sphere, a microsphere, a nanosphere, a particle, a microparticle, a nanoparticle, or a capillary.

28. The method of claim 26 or 27, wherein the protein-solid support complexes are bound to a labeling reagent.

29. A method for detecting development of tolerance to a protein listed in the second column of any one of Tables 10-12, and/or monitoring the efficacy of a multiple allergen oral immunotherapy in treating an allergy to a protein listed in the second column of any one of Tables 10-12, the method comprising:
(a) obtaining a biological sample from the subject;
(b) contacting the biological sample with a protein listed in the corresponding row of the first column of Tables 10-12;
(c) measuring an amount of IgE, IgG, or T-cells that bind to the protein listed in the corresponding row of the first column of Tables 10-12; and
(d) comparing the amount of IgE, IgG or T-cells with a previous measurement of the amount of IgE, IgG or T-cells;
wherein tolerance and/or efficacy is indicated when the amount of IgE, IgG or T-cells, is less than the previous amount of IgE, IgG or T-cells.

30. A method for detecting development of tolerance to a peptide listed in the third column of any one of Tables 13-23, and/or monitoring the efficacy of a multiple allergen oral immunotherapy in treating an allergy to a peptide listed in the third column of any one of Tables 13-23, the method comprising:
(a) obtaining a biological sample from the subject;
(b) contacting the biological sample with a protein listed in the corresponding row of the sixth column of Tables 13-23;
(c) measuring an amount of IgE, IgG, or T-cells that bind to the protein listed in the corresponding row of the sixth column of Tables 13-23; and
(d) comparing the amount of IgE, IgG or T-cells with a previous measurement of the amount of IgE, IgG or T-cells;
wherein tolerance and/or efficacy is indicated when the amount of IgE, IgG or T-cells, is less than the previous amount of IgE, IgG or T-cells.

31. A detection kit for a biological cross-reactivity IgE assay comprising six or more distinct proteins each bound to a solid support, wherein the six or more distinct proteins are selected from the group consisting of proteins listed in Table 2.

32. A detection kit for a biological cross-reactivity IgE assay comprising six or more distinct proteins each bound to a solid support, wherein the six or more distinct proteins are selected from the group consisting of proteins listed in Tables 10-12.

33. A detection kit for a biological cross-reactivity IgE assay comprising six or more distinct peptides each bound to a solid support, wherein the six or more distinct peptides are selected from the group consisting of peptides listed in Tables 13-23.

34. The detection kit of any one of claims 31-33, wherein the solid support is a membrane, a filter, a chip, a slide (such as a microscopic slide), an array (such as a microarray) a wafer, a fiber, a magnetic or a non-magnetic bead, a gel, a tubing, a plate (such as a microtiter plate), a microtiter strip, a polymer, a microparticle, a sphere, a microsphere, a nanosphere, a particle, a microparticle, a nanoparticle, or a capillary.

35. The detection kit of any one of claims 31-34, further comprising an IgE or IgG labeling reagent.

36. The detection kit of claim 35, further comprising one or more of a binding buffer, a wash buffer, and a detection buffer.

37. The detection kit of any one of claims 31-36, further comprising instructions for use.

38. A method of assessing the presence or absence of cross-reactive IgE antibodies or T-cells in a biological sample, the method comprising:
(a) contacting the biological sample with a reference allergenic protein;
(b) measuring the binding of IgE antibodies or T-cells to the reference allergenic proteins; and/or;
(c) identifying any proteins that are immunologically cross-reactive to the reference allergenic protein;
to assess the presence of one or more cross-reactive IgE antibodies or T-cells.

39. The method of claim 38, wherein step (c) comprises the method of any one of claims 48-67.

40. The method of claim 38 or 39, wherein the method does not comprise measuring the binding of IgE antibodies or T-cells to proteins that are immunologically cross-reactive to the reference allergenic protein.

41. The method of any one of claims 38-40, wherein the reference allergenic protein is immobilized on a substrate.

42. The method of claim 41, wherein the substrate is membrane, a filter, a chip, a slide (such as a microscopic slide), an array (such as a microarray) a wafer, a fiber, a magnetic or a non-magnetic bead, a gel, a tubing, a plate (such as a microtiter plate), a microtiter strip, a polymer, a microparticle, a sphere, a microsphere, a nanosphere, a particle, a microparticle, a nanoparticle, or a capillary.

43. The method of claims 20-42, wherein the biological sample is selected from the group consisting of whole blood, serum, plasma, sputum, blood cells (e.g., peripheral blood mononuclear cells (PBMC), T-cells, B-cells, basophils, etc.), immune cells, a tissue sample, a biopsy sample, urine, tears, peritoneal fluid, pleural fluid, breast duct fluid, breast exudate, breast milk, breast fluids, saliva, semen, mucous, lymph, cytosol, ascites, amniotic fluid, bladder washes, and bronchioalveolar lavage.

44. A method for testing whether a subject is a candidate for a multiple allergen oral immunotherapy, the method comprising:
detecting in a biological sample from the subject the presence of IgE antibodies or T-cells that bind to a reference allergenic protein in the multiple allergen oral immunotherapy and an other protein that is immunologically cross-reactive with the reference protein,
wherein if IgE antibodies or T-cells are present in the biological sample, the subject is a candidate for the multiple allergen oral immunotherapy.

45. The method of claim 44, wherein the subject had not been identified as having an allergy to the other protein.

46. A method for diagnosing a subject as having an allergy to an allergic protein listed in the first column of any one of Tables 10-12, the method comprising evaluating the subject, or a biological sample from the subject, for an allergic response to an allergenic protein listed in the corresponding row of the second column of Tables 10-12.

47. A method for diagnosing a subject as having an allergy to an allergic peptide listed in the third column of any one of Tables 13-23, the method comprising evaluating the subject, or a biological sample from the subject, for an allergic response to an allergenic peptide listed in the corresponding row of the second column of Tables 13-23.

48. A method for identifying a protein that is immunologically cross-reactive to a reference allergenic protein, the method comprising:
(a) correlating the reference allergenic protein to an other protein that is in the same protein domain family; and/or
(b) calculating pairwise identity and/or similarity between an amino acid sequence of the reference allergenic protein and an other protein,
so as to determine whether the other protein is cross-reactive to the reference allergenic protein.

49. The method of claim 48, wherein step (b) comprises:
(i) providing a dataset comprising the amino acid sequence of the reference allergenic protein and the other protein;
(ii) inputting the dataset into a computer readable medium;
(iii) performing a bioinformatic step to calculate pairwise identity and similarity between the reference allergenic protein and the other protein; and/or
(iv) identifying the other protein as cross-reactive to the reference allergenic protein based on pairwise identity and similarity between the reference allergenic protein and the other protein.

50. The method of claim 49, wherein the bioinformatic step comprises applying an algorithm to determine homology between the reference allergenic protein and the other protein based on pairwise identity and similarity, wherein the algorithm is selected from the group consisting of GAP, BESTFIT, FASTA, TFASTA, BLAST, BLASTP, TBLASTN, FASTDB, ALIGN, CLUSTALW, and CLUSTAL-Omega, including any version thereof.

51. The method of claim 49 or 50, wherein the bioinformatic step comprises applying an algorithm comprising (identity+similarity)/2 to determine a cross-reactivity score.

52. The method of claim 51, wherein the other protein is considered cross-reactive to the reference allergenic protein if the other protein has a cross-reactivity score of ≥0.5 with respect to the reference allergenic protein.

53. The method of any one of claims 48-52, wherein the other protein is considered cross-reactive to the reference allergenic protein if the other protein has ≥70% homology to the reference allergenic protein.

54. The method of any one of claims 48-53, wherein the amino acid sequence is a full-length amino acid sequence.

55. The method of any one of claims 48-54, where the method comprises, prior to step (a), identifying the reference allergenic protein as a protein in an allergenic food source.

56. The method of any one of claims 48-55, where the method comprises, prior to step (a), mapping the reference allergenic protein to a protein domain family.

57. The method of any one of claims 48-56, wherein the method further comprises identifying a T-cell epitope peptide, a B-cell epitope peptide, and/or an MHC-binding epitope peptide in the allergenic protein that is cross-reactive to a T-cell epitope peptide, a B-cell epitope peptides and/or an MHC-binding epitope in the other protein.

58. The method of claim 57, wherein the method further comprises determining the MHC binding potential of the T-cell or B-cell peptide.

59. The method of any one of claims 48-58, wherein the reference allergenic protein is present in a multiple allergen oral immunotherapy.

60. The method of any one of claims 48-59, wherein the reference allergenic protein is selected from the group consisting of a shrimp allergen, a cod allergen, a salmon allergen, a hen's egg allergen, a cow's milk allergen, a peanut allergen, a sesame allergen, a soy allergen, a pecan allergen, a cashew allergen, a hazelnut allergen, a walnut allergen, a pistachio allergen, an almond allergen, and a wheat allergen.

61. The method of any one of claims 48-60, wherein the protein family is selected from the group consisting of: Tropomyosin, EF hand family (Beta-parvalbumin), ATP:guanido phosphotransferase (Arginine Kinase), Triosephosphate isomerase, Fructose bisphosphate aldolase class I, Alpha/beta casein, Lipocalin, Kappa-casein, Transferrin, C-type lysozyme/alpha-lactalbumin family, Serum albumin, Enolase, Serpin serine protease inhibitor, Kazal-type serine protease inhibitor, Collagen, Lipoprotein, Prolamin superfamily, Cupin, Profilin, Bet v 1 family, Oleosin, Isoflavone reductase family, Plant defensin, 60S acidic ribosomal protein, Heat shock protein Hsp70, Fe/Mn superoxide dismutase, Papain-like cysteine protease, Thaumatin-like protein, Unclassified, GILT family, Hevein-like and class I/II chitinase, Zinc finger, C3HC4 type (RING finger), SCAI family, Peroxidase, Serpin serine protease inhibitor, Thionin, Thioredoxin, Transcription elongation factor 1, Lipid transfer protein, and Triosephosphate isomerase.

62. The method of claim 61, wherein the protein family is selected from the group consisting of: Tropomyosin, Cupin, Profilin, Bet v 1, and EF hand.

63. The method of claim 62, wherein the protein family is Cupin.

64. The method of any one of claims 48-63, wherein the reference allergenic protein is selected from the group consisting of allergenic proteins listed in Table 2.

65. The method of any one of claims 48-64, wherein the other protein is selected from the group consisting of proteins listed in Table 4.

66. The method of any one of claims 48-65, wherein the other protein is selected from the group consisting of proteins listed in Tables 5-7 or Tables 10-12.

67. The method of any one of claims 48-66, wherein the method comprises identifying proteins that are immunologically cross-reactive to each reference allergenic protein present in a food source to generate an allergenic map (AllerMap) of the food source.

68. A method of treating an allergy to one or more of soybean, sesame, pistachio, pecan, hazelnut, walnut, cashew, peanut and almond in a subject in need thereof, the method comprising administering to the subject a multiple allergen oral immunotherapy composition comprising two or more of soybean, sesame, pistachio, pecan, hazelnut, walnut, cashew, peanut, almond, kiwifruit, brazil nut, coconut, buckwheat, lentil, garden pea, yellow mustard, mung bean, lupin, and/or korean pine, optionally wherein the multiple allergen oral immunotherapy composition does not comprise soybean, sesame, pistachio, pecan, hazelnut, walnut, cashew, peanut and/or almond.

69. A method of treating an allergy to soybean in a subject in need thereof, the method comprising administering to the subject a multiple allergen oral immunotherapy composition comprising one or more of kiwifruit, brazil nut, coconut, buckwheat, lentil, garden pea, yellow mustard, mung bean, lupin, and korean pine, optionally wherein the multiple allergen oral immunotherapy composition does not comprise soybean.

70. A method of treating an allergy to soybean in a subject in need thereof, the method comprising administering to the subject a multiple allergen oral immunotherapy composition comprising two or more of sesame, pistachio, pecan, hazelnut, walnut, cashew, peanut, almond, kiwifruit, brazil nut, coconut, buckwheat, lentil, garden pea, yellow mustard, mung bean, lupin, and korean pine, optionally wherein the multiple allergen oral immunotherapy composition does not comprise soybean.

71. A method of treating an allergy to sesame in a subject in need thereof, the method comprising administering to the subject a multiple allergen oral immunotherapy composition comprising one or more of kiwifruit, brazil nut, coconut, buckwheat, lentil, garden pea, yellow mustard, mung bean, lupin, and korean pine, optionally wherein the multiple allergen oral immunotherapy composition does not comprise sesame.

72. A method of treating an allergy to sesame in a subject in need thereof, the method comprising administering to the subject a multiple allergen oral immunotherapy composition comprising two or more of soybean, pistachio, pecan, hazelnut, walnut, cashew, peanut, almond, kiwifruit, brazil nut, coconut, buckwheat, lentil, garden pea, yellow mustard, mung bean, lupin, and korean pine, optionally wherein the multiple allergen oral immunotherapy composition does not comprise sesame.

73. A method of treating an allergy to pistachio in a subject in need thereof, the method comprising administering to the subject a multiple allergen oral immunotherapy composition comprising one or more of kiwifruit, brazil nut, coconut, buckwheat, lentil, garden pea, yellow mustard, mung bean, lupin, and korean pine, optionally wherein the multiple allergen oral immunotherapy composition does not comprise pistachio.

74. A method of treating an allergy to pistachio in a subject in need thereof, the method comprising administering to the subject a multiple allergen oral immunotherapy composition comprising two or more of soybean, sesame, pecan, hazelnut, walnut, cashew, peanut, almond, kiwifruit, brazil nut, coconut, buckwheat, lentil, garden pea, yellow mustard, mung bean, lupin, and korean pine, optionally wherein the multiple allergen oral immunotherapy composition does not comprise pistachio.

75. A method of treating an allergy to pecan in a subject in need thereof, the method comprising administering to the subject a multiple allergen oral immunotherapy composition comprising one or more of kiwifruit, brazil nut, coconut, buckwheat, lentil, garden pea, yellow mustard, mung bean, lupin, and korean pine, optionally wherein the multiple allergen oral immunotherapy composition does not comprise pecan.

76. A method of treating an allergy to pecan in a subject in need thereof, the method comprising administering to the subject a multiple allergen oral immunotherapy composition comprising two or more of soybean, sesame, pistachio, hazelnut, walnut, cashew, peanut, almond, kiwifruit, brazil nut, coconut, buckwheat, lentil, garden pea, yellow mustard, mung bean, lupin, and korean pine, optionally wherein the multiple allergen oral immunotherapy composition does not comprise pecan.

77. A method of treating an allergy to hazelnut in a subject in need thereof, the method comprising administering to the subject a multiple allergen oral immunotherapy composition comprising one or more of kiwifruit, brazil nut, coconut, buckwheat, lentil, garden pea, yellow mustard, mung bean, lupin, and korean pine, optionally wherein the multiple allergen oral immunotherapy composition does not comprise hazelnut.

78. A method of treating an allergy to hazelnut in a subject need thereof, the method comprising administering to the subject a multiple allergen oral immunotherapy composition comprising two or more of soybean, sesame, pistachio, pecan, walnut, cashew, peanut, almond, kiwifruit, brazil nut, coconut, buckwheat, lentil, garden pea, yellow mustard, mung bean, lupin, and korean pine, optionally wherein the multiple allergen oral immunotherapy composition does not comprise hazelnut.

79. A method of treating an allergy to walnut in a subject in need thereof, the method comprising administering to the subject a multiple allergen oral immunotherapy composition comprising one or more of kiwifruit, brazil nut, coconut, buckwheat, lentil, garden pea, yellow mustard, mung bean, lupin, and korean pine, optionally wherein the multiple allergen oral immunotherapy composition does not comprise walnut.

80. A method of treating an allergy to walnut in a subject need thereof, the method comprising administering to the subject a multiple allergen oral immunotherapy composition comprising two or more of soybean, sesame, pistachio, pecan, hazelnut, cashew, peanut, almond, kiwifruit, brazil nut, coconut, buckwheat, lentil, garden pea, yellow mustard, mung bean, lupin, and korean pine, optionally wherein the multiple allergen oral immunotherapy composition does not comprise walnut.

81. A method of treating an allergy to cashew in a subject in need thereof, the method comprising administering to the subject a multiple allergen oral immunotherapy composition comprising one or more of kiwifruit, brazil nut, coconut, buckwheat, lentil, garden pea, yellow mustard, mung bean, lupin, and korean pine, optionally wherein the multiple allergen oral immunotherapy composition does not comprise cashew.

82. A method of treating an allergy to cashew in a subject need thereof, the method comprising administering to the subject a multiple allergen oral immunotherapy composition comprising two or more of soybean, sesame, pistachio, pecan, hazelnut, walnut, peanut, almond, kiwifruit, brazil nut, coconut, buckwheat, lentil, garden pea, yellow mustard, mung bean, lupin, and korean pine, optionally wherein the multiple allergen oral immunotherapy composition does not comprise cashew.

83. A method of treating an allergy to peanut in a subject in need thereof, the method comprising administering to the subject a multiple allergen oral immunotherapy composition comprising one or more of kiwifruit, brazil nut, coconut, buckwheat, lentil, garden pea, yellow mustard, mung bean, lupin, and korean pine, optionally wherein the multiple allergen oral immunotherapy composition does not comprise peanut.

84. A method of treating an allergy to peanut in a subject need thereof, the method comprising administering to the subject a multiple allergen oral immunotherapy composition comprising two or more of soybean, sesame, pistachio, pecan, hazelnut, walnut, cashew, almond, kiwifruit, brazil nut, coconut, buckwheat, lentil, garden pea, yellow mustard, mung bean, lupin, and korean pine, optionally wherein the multiple allergen oral immunotherapy composition does not comprise peanut.

85. A method of treating an allergy to almond in a subject in need thereof, the method comprising administering to the subject a multiple allergen oral immunotherapy composition comprising one or more of kiwifruit, brazil nut, coconut, buckwheat, lentil, garden pea, yellow mustard, mung bean, lupin, and korean pine, optionally wherein the multiple allergen oral immunotherapy composition does not comprise almond.

86. A method of treating an allergy to almond in a subject need thereof, the method comprising administering to the subject a multiple allergen oral immunotherapy composition comprising two or more of soybean, sesame, pistachio, pecan, hazelnut, walnut, cashew, peanut, kiwifruit, brazil nut, coconut, buckwheat, lentil, garden pea, yellow mustard, mung bean, lupin, and korean pine, optionally wherein the multiple allergen oral immunotherapy composition does not comprise almond.

87. A method of treating an allergy to an Ana o 1 protein, or a food source comprising the Ana o 1 protein, in a subject in need thereof, the method comprising administering to the subject: a Cor a 11 protein, or a food source comprising the Cor a 11 protein; a Pis v 3 protein, or a food source comprising the Pis v 3 protein; and/or a Ses i 3 protein, or a food source comprising the Ses i 3 protein; optionally wherein the method does not comprise administering to the subject the Ana o 1 protein or a food source comprising the Ana o 1 protein.

88. A method of treating an allergy to an Ana o 2 protein, or a food source comprising the Ana o 2 protein, in a subject in need thereof, the method comprising administering to the subject: an Ara h 3 protein, or a food source comprising the Ara h 3 protein; a Car i 4 protein, or a food source comprising the Car i 4 protein; a Cor a 9 protein, or a food source comprising the Cor a 9 protein; a Gly m 6 protein, or a food source comprising the Gly m 6 protein; a Jug n 4 protein, or a food source comprising the Jug n 4 protein; a Jug r 4 protein, or a food source comprising the Jug r 4 protein; a Pis v 2 protein, or a food source comprising the Pis v 2 protein; a Pis v 5 protein, or a food source comprising the Pis v 5 protein; a Pru du 6 protein, or a food source comprising the Pru du 6 protein; a Ses i 6 protein, or a food source comprising the Ses i 6 protein; a Ses i 7 protein, or a food source comprising the Ses i 7 protein; a Ber e 2 protein, or a food source comprising the Ber e 2 protein; a Fag e 1 protein, or a food source comprising the Fag e 1 protein; a Fag t 13s g protein, or a food source comprising the Fag t 13s g protein; and/or a Sin a 2 protein, or a food source comprising the Sin a 2 protein; optionally wherein the method does not comprise administering to the subject the Ana o 2 protein or a food source comprising the Ana o 2 protein.

89. A method of treating an allergy to an Ara h 1 protein, or a food source comprising the Ara h 1 protein, in a subject in need thereof, the method comprising administering to the subject: a Car i 2 protein, or a food source comprising the Car i 2 protein; a Gly m 5 protein, or a food source comprising the Gly m 5 protein; a Jug r 2 protein, or a food source comprising the Jug r 2 protein; a Ses i 3 protein, or a food source comprising the Ses i 3 protein; a Len c 1 protein, or a food source comprising the Len c 1 protein; a Lup an 1 protein, or a food source comprising the Lup an 1 protein; a Lup a v protein, or a food source comprising the Lup a v protein; a Pis s 1 protein, or a food source comprising the Pis s 1 protein; a Pis s 2 protein, or a food source comprising the Pis s 2 protein; and/or a Vig r 2 protein, or a food source comprising the Vig r 2 protein; optionally wherein the method does not comprise administering to the subject the Ara h 1 protein or a food source comprising the Ara h 1 protein.

90. A method of treating an allergy to an Ara h 3 protein, or a food source comprising the Ara h 3 protein, in a subject in need thereof, the method comprising administering to the subject: a Car i 4 protein, or a food source comprising the Car i 4 protein; a Cor a 9 protein, or a food source comprising the Cor a 9 protein; a Jug n 4 protein, or a food source comprising the Jug n 4 protein; a Jug r 4 protein, or a food source comprising the Jug r 4 protein; a Pis v 2 protein, or a food source comprising the Pis v 2 protein; a Pis v 5 protein, or a food source comprising the Pis v 5 protein; a Pru du 6 protein, or a food source comprising the Pru du 6 protein; a Ses i 6 protein, or a food source comprising the Ses i 6 protein; a Ses i 7 protein, or a food source comprising the Ses i 7 protein; a Fag e 1 protein, or a food source comprising the Fag e 1 protein; a Fag t 13s g protein, or a food source comprising the Fag t 13s g protein; and/or an Ana o 2 protein, or a food source comprising the Ana o 2; optionally wherein the method does not comprise administering to the subject the Ara h 3 protein or a food source comprising the Ara h 3 protein.

91. A method of treating an allergy to a Ber e 2 protein, or a food source comprising the Ber e 2 protein, in a subject in need thereof, the method comprising administering to the subject: a Fag e 1 protein, or a food source comprising the Fag e 1 protein; a Fag t 13s g protein, or a food source comprising the Fag t 13s g protein; an Ana o 2 protein, or a food source comprising the Ana o 2 protein; a Car i 4 protein, or a food source comprising the Car i 4 protein; a Cor a 9 protein, or a food source comprising the Cor a 9 protein; a Gly m 6 protein, or a food source comprising the Gly m 6 protein; a Jug n 4 protein, or a food source comprising the Jug n 4 protein; a Jug r 4 protein, or a food source comprising the Jug r 4 protein; a Pis v 2 protein, or a food source comprising the Pis v 2 protein; a Pis v 5 protein, or a food source comprising the Pis v 5 protein; a Pru du 6 protein, or a food source comprising the Pru du 6 protein; a Ses i 6 protein, or a food source comprising the Ses i 6 protein; and/or a Ses i 7 protein, or a food source comprising the Ses i 7 protein; optionally wherein the method does not comprise administering to the subject the Ber e 2 protein or a food source comprising the Ber e 2 protein.

92. A method of treating an allergy to a Car i 2 protein, or a food source comprising the Car i 2 protein, in a subject in need thereof, the method comprising administering to the subject: a Cor a 11 protein, or a food source comprising the Cor a 11 protein; a Gly m 5 protein, or a food source comprising the Gly m 5 protein; a Jug r 2 protein, or a food source comprising the Jug r 2 protein; a Pis v 3 protein, or a food source comprising the Pis v 3 protein; a Ses i 3 protein, or a food source comprising the Ses i 3 protein; a Len c 1 protein, or a food source comprising the Len c 1 protein; a Lup an 1 protein, or a food source comprising the Lup an 1 protein; a Lup a v protein, or a food source comprising the Lup a v protein; a Pin k 2 protein, or a food source comprising the Pin k 2 protein; a Pis s 2 protein, or a food source comprising the Pis s 2 protein; a Vig r 2 protein, or a food source comprising the Vig r 2 protein; and/or an Ara h 1 protein, or a food source comprising the Ara h 1 protein; optionally wherein the method does not comprise administering to the subject the Car i 2 protein or a food source comprising the Car i 2 protein.

93. A method of treating an allergy to a Car i 4 protein, or a food source comprising the Car i 4 protein, in a subject in need thereof, the method comprising administering to the subject: a Cor a 9 protein, or a food source comprising the Cor a 9 protein; a Gly m 6 protein, or a food source comprising the Gly m 6 protein; a Jug n 4 protein, or a food source comprising the Jug n 4 protein; a Jug r 4 protein, or a food source comprising the Jug r 4 protein; a Pis v 2 protein, or a food source comprising the Pis v 2 protein; a Pis v 5 protein, or a food source comprising the Pis v 5 protein; a Pru du 6 protein, or a food source comprising the Pru du 6 protein; a Ses i 6 protein, or a food source comprising the Ses i 6 protein; a Ses i 7 protein, or a food source comprising the Ses i 7 protein; a Ber e 2 protein, or a food source comprising the Ber e 2 protein; a Fag e 1 protein, or a food source comprising the Fag e 1 protein; a Fag t 13s g protein, or a food source comprising the Fag t 13s g protein; a Sin a 2 protein, or a food source comprising the Sin a 2 protein; an Ana o 2 protein, or a food source comprising the Ana o 2 protein; and/or an Ara h 3 protein, or a food source comprising the Ara h 3 protein; optionally wherein the method does not comprise administering to the subject the Car i 4 protein or a food source comprising the Car i 4 protein.

94. A method of treating an allergy to a Cor a 11 protein, or a food source comprising the Cor a 11 protein, in a subject in need thereof, the method comprising administering to the subject: a Jug r 2 protein, or a food source comprising the Jug r 2 protein; a Pis v 3 protein, or a food source comprising the Pis v 3 protein; a Len c 1 protein, or a food source comprising the Len c 1 protein; a Pin k 2 protein, or a food source comprising the Pin k 2 protein; a Pis s 1 protein, or a food source comprising the Pis s 1 protein; a Pis s 2 protein, or a food source comprising the Pis s 2 protein; an Ana o 1 protein, or a food source comprising the Ana o 1 protein; and/or a Car i 2 protein, or a food source comprising the Car i 2 protein; optionally wherein the method does not comprise administering to the subject the Cor a 11 protein or a food source comprising the Cor a 11 protein.

95. A method of treating an allergy to a Cor a 9 protein, or a food source comprising the Cor a 9 protein, in a subject in need thereof, the method comprising administering to the subject: a Gly m 6 protein, or a food source comprising the Gly m 6 protein; a Jug n 4 protein, or a food source comprising the Jug n 4 protein; a Jug r 4 protein, or a food source comprising the Jug r 4 protein; a Pis v 2 protein, or a food source comprising the Pis v 2 protein; a Pis v 5 protein, or a food source comprising the Pis v 5 protein; a Pru du 6 protein, or a food source comprising the Pru du 6 protein; a Ses i 6 protein, or a food source comprising the Ses i 6 protein; a Ses i 7 protein, or a food source comprising the Ses i 7 protein; a Ber e 2 protein, or a food source comprising the Ber e 2 protein; a Fag e 1 protein, or a food source comprising the Fag e 1 protein; a Fag t 13s g protein, or a food source comprising the Fag t 13s g protein; a Sin a 2 protein, or a food source comprising the Sin a 2 protein; an Ana o 2 protein, or a food source comprising the Ana o 2 protein; an Ara h 3 protein, or a food source comprising the Ara h 3 protein; and/or a Car i 4 protein, or a food source comprising the Car i 4 protein; optionally wherein the method does not comprise administering to the subject the Cor a 9 protein or a food source comprising the Cor a 9 protein.

96. A method of treating an allergy to a Fag e 1 protein, or a food source comprising the Fag e 1 protein, in a subject in need thereof, the method comprising administering to the subject: a Fag t 13s g protein, or a food source comprising the Fag t 13s g protein; an Ana o 2 protein, or a food source comprising the Ana o 2 protein; an Ara h 3 protein, or a food source comprising the Ara h 3 protein; a Car i 4 protein, or a food source comprising the Car i 4 protein; a Cor a 9 protein, or a food source comprising the Cor a 9 protein; a Gly m 6 protein, or a food source comprising the Gly m 6 protein; a Jug r 4 protein, or a food source comprising the Jug r 4 protein; a Pis v 2 protein, or a food source comprising the Pis v 2 protein; a Pis v 5 protein, or a food source comprising the Pis v 5 protein; a Pru du 6 protein, or a food source comprising the Pru du 6 protein; a Ses i 7 protein, or a food source comprising the Ses i 7 protein; and/or a Ber e 2 protein, or a food source comprising the Ber e 2 protein; optionally wherein the method does not comprise administering to the subject the Fag e 1 protein or a food source comprising the Fag e 1 protein.

97. A method of treating an allergy to a Fag t 13s g protein, or a food source comprising the Fag t 13s g protein, in a subject in need thereof, the method comprising administering to the subject: an Ana o 2 protein, or a food source comprising the Ana o 2 protein; an Ara h 3 protein, or a food source comprising the Ara h 3 protein; a Car i 4 protein, or a food source comprising the Car i 4 protein; a Cor a 9 protein, or a food source comprising the Cor a 9 protein; a Gly m 6 protein, or a food source comprising the Gly m 6 protein; a Jug r 4 protein, or a food source comprising the Jug r 4 protein; a Pis v 2 protein, or a food source comprising the Pis v 2 protein; a Pis v 5 protein, or a food source comprising the Pis v 5 protein; a Pru du 6 protein, or a food source comprising the Pru du 6 protein; a Ses i 7 protein, or a food source comprising the Ses i 7 protein; a Ber e 2 protein, or a food source comprising the Ber e 2 protein; and/or a Fag e 1 protein, or a food source comprising the Fag e 1 protein; optionally wherein the method does not comprise administering to the subject the Fag t 13s g protein or a food source comprising the Fag t 13s g protein.

98. A method of treating an allergy to a Gly m 5 protein, or a food source comprising the Gly m 5 protein, in a subject in need thereof, the method comprising administering to the subject: a Jug r 2 protein, or a food source comprising the Jug r 2 protein; a Len c 1 protein, or a food source comprising the Len c 1 protein; a Lup an 1 protein, or a food source comprising the Lup an 1 protein; a Lup a v protein, or a food source comprising the Lup a v protein; a Pis s 1 protein, or a food source comprising the Pis s 1 protein; a Pis s 2 protein, or a food source comprising the Pis s 2 protein; a Vig r 2 protein, or a food source comprising the Vig r 2 protein; an Ara h 1 protein, or a food source comprising the Ara h 1 protein; and/or a Car i 2 protein, or a food source comprising the Car i 2 protein; optionally wherein the method does not comprise administering to the subject the Gly m 5 protein or a food source comprising the Gly m 5 protein.

99. A method of treating an allergy to a Gly m 6 protein, or a food source comprising the Gly m 6 protein, in a subject in need thereof, the method comprising administering to the subject: a Jug n 4 protein, or a food source comprising the Jug n 4 protein; a Jug r 4 protein, or a food source comprising the Jug r 4 protein; a Pis v 2 protein, or a food source comprising the Pis v 2 protein; a Pru du 6 protein, or a food source comprising the Pru du 6 protein; a Ses i 7 protein, or a food source comprising the Ses i 7 protein; a Ber e 2 protein, or a food source comprising the Ber e 2 protein; a Fag e 1 protein, or a food source comprising the Fag e 1 protein; a Fag t 13s g protein, or a food source comprising the Fag 13s g protein; an Ana o 2 protein, or a food source comprising the Ana o 2 protein; a Car i 4 protein, or a food source comprising the Car i 4 protein; and/or a Cor a 9 protein, or a food source comprising the Cor a 9 protein; optionally wherein the method does not comprise administering to the subject the Gly m 6 protein or a food source comprising the Gly m 6 protein.

100. A method of treating an allergy to a Jug n 4 protein, or a food source comprising the Jug n 4 protein, in a subject in need thereof, the method comprising administering to the subject: a Jug r 4 protein, or a food source comprising the Jug r 4 protein; a Pis v 2 protein, or a food source comprising the Pis v 2 protein; a Pis v 5 protein, or a food source comprising the Pis v 5 protein; a Pru du 6 protein, or a food source comprising the Pru du 6 protein; a Ses i 6 protein, or a food source comprising the Ses i 6 protein; a Ses i 7 protein, or a food source comprising the Ses i 7 protein; a Ber e 2 protein, or a food source comprising the Ber e 2 protein; a Sin a 2 protein, or a food source comprising the Sin a 2 protein; an Ana o 2 protein, or a food source comprising the Ana o 2 protein; an Ara h 3 protein, or a food source comprising the Ara h 3 protein; a Car i 4 protein, or a food source comprising the Car i 4 protein; a Cor a 9 protein, or a food source comprising the Cor a 9 protein; and/or a Gly m 6 protein, or a food source comprising the Gly m 6 protein; optionally wherein the method does not comprise administering to the subject the Jug n 4 protein or a food source comprising the Jug n 4 protein.

101. A method of treating an allergy to a Jug r 2 protein, or a food source comprising the Jug r 2 protein, in a subject in need thereof, the method comprising administering to the subject: a Ses i 3 protein, or a food source comprising the Ses i 3 protein; a Len c 1 protein, or a food source comprising the Len c 1 protein; a Pin k 2 protein, or a food source comprising the Pin k 2 protein; a Pis s 1 protein, or a food source comprising the Pis s 1 protein; a Pis s 2 protein, or a food source comprising the Pis s 2 protein; a Vig r 2 protein, or a food source comprising the Vig r 2 protein; an Ara h 1 protein, or a food source comprising the Ara h 1 protein; a Car i 2 protein, or a food source comprising the Car i 2 protein; a Cor a 11 protein, or a food source comprising the Cor a 11 protein; and/or a Gly m 5 protein, or a food source comprising the Gly m 5 protein; optionally wherein the method does not comprise administering to the subject the Jug r 2 protein or a food source comprising the Jug r 2 protein.

102. A method of treating an allergy to a Jug r 4 protein, or a food source comprising the Jug r 4 protein, in a subject in need thereof, the method comprising administering to the subject: a Pis v 2 protein, or a food source comprising the Pis v 2 protein; a Pis v 5 protein, or a food source comprising the Pis v 5 protein; a Pru du 6 protein, or a food source comprising the Pru du 6 protein; a Ses i 6 protein, or a food source comprising the Ses i 6 protein; a Ses i 7 protein, or a food source comprising the Ses i 7 protein; a Ber e 2 protein, or a food source comprising the Ber e 2 protein; a Fag e 1 protein, or a food source comprising the Fag e 1 protein; a Fag t 13s g protein, or a food source comprising the Fag t 13s g protein; a Sin a 2 protein, or a food source comprising the Sin a 2 protein; an Ana o 2 protein, or a food source comprising the Ana o 2 protein; an Ara h 3 protein, or a food source comprising the Ara h 3 protein; a Car i 4 protein, or a food source comprising the Car i 4 protein; a Cor a 9 protein, or a food source comprising the Cor a 9 protein; a Gly m 6 protein, or a food source comprising the Gly m 6 protein; and/or a Jug n 4 protein, or a food source comprising the Jug n 4 protein; optionally wherein the method does not comprise administering to the subject the Jug r 4 protein or a food source comprising the Jug r 4 protein.

103. A method of treating an allergy to a Len c 1 protein, or a food source comprising the Len c 1 protein, in a subject in need thereof, the method comprising administering to the subject: a Lup an 1 protein, or a food source comprising the Lup an 1 protein; a Lup a v protein, or a food source comprising the Lup a v protein; a Pis s 1 protein, or a food source comprising the Pis s 1 protein; a Pis s 2 protein, or a food source comprising the Pis s 2 protein; a Vig r 2 protein, or a food source comprising the Vig r 2 protein; an Ara h 1 protein, or a food source comprising the Ara h 1 protein; a Car i 2 protein, or a food source comprising the Car i 2 protein; a Cor a 11 protein, or a food source comprising the Cor a 11 protein; a Gly m 5 protein, or a food source comprising the Gly m 5 protein; a Jug r 2 protein, or a food source comprising the Jug r 2 protein; and/or a Ses i 3 protein, or a food source comprising the Ses i 3 protein; optionally wherein the method does not comprise administering to the subject the Len c 1 protein or a food source comprising the Len c 1 protein.

104. A method of treating an allergy to a Lup an 1 protein, or a food source comprising the Lup an 1 protein, in a subject in need thereof, the method comprising administering to the subject: a Lup a v protein, or a food source comprising the Lup a v protein; a Pis s 1 protein, or a food source comprising the Pis s 1 protein; a Pis s 2 protein, or a food source comprising the Pis s 2 protein; a Vig r 2 protein, or a food source comprising the Vig r 2 protein; an Ara h 1 protein, or a food source comprising the Ara h 1 protein; a Car i 2 protein, or a food source comprising the Car i 2 protein; a Gly m 5 protein, or a food source comprising the Gly m 5 protein; a Ses i 3 protein, or a food source comprising the Ses i 3 protein; and/or a Len c 1 protein, or a food source comprising the Len c 1 protein; optionally wherein the method does not comprise administering to the subject the Lup an 1 protein or a food source comprising the Lup an 1 protein.

105. A method of treating an allergy to a Lup a v protein, or a food source comprising the Lup a v protein, in a subject in need thereof, the method comprising administering to the subject: a Pis s 1 protein, or a food source comprising the Pis s 1 protein; a Pis s 2 protein, or a food source comprising the Pis s 2 protein; a Vig r 2 protein, or a food source comprising the Vig r 2 protein; an Ara h 1 protein, or a food source comprising the Ara h 1 protein; a Car i 2 protein, or a food source comprising the Car i 2 protein; a Gly m 5 protein, or a food source comprising the Gly m 5 protein; a Len c 1 protein, or a food source comprising the Len c 1 protein; and/or a Lup an 1 protein, or a food source comprising the Lup an 1 protein; optionally wherein the method does not comprise administering to the subject the Lup a v protein or a food source comprising the Lup a v protein.

106. A method of treating an allergy to a Pin k 2 protein, or a food source comprising the Pin k 2 protein, in a subject in need thereof, the method comprising administering to the subject: a Car i 2 protein, or a food source comprising the Car i 2 protein; a Cor a 11 protein, or a food source comprising the Cor a 11 protein; a Jug r 2 protein, or a food source comprising the Jug r 2 protein; and/or a Pis v 3 protein, or a food source comprising the Pis v 3 protein; optionally wherein the method does not comprise administering to the subject the Pin k 2 protein or a food source comprising the Pin k 2 protein.

107. A method of treating an allergy to a Pis s 1 protein, or a food source comprising the Pis s 1 protein, in a subject in need thereof, the method comprising administering to the subject: a Pis s 2 protein, or a food source comprising the Pis s 2 protein; a Vig r 2 protein, or a food source comprising the Vig r 2 protein; an Ara h 1 protein, or a food source comprising the Ara h 1 protein; a Car i 2 protein, or a food source comprising the Car i 2 protein; a Cor a 11 protein, or a food source comprising the Cor a 11 protein; a Gly m 5 protein, or a food source comprising the Gly m 5 protein; a Jug r 2 protein, or a food source comprising the Jug r 2 protein; a Ses i 3 protein, or a food source comprising the Ses i 3 protein; a Len c 1 protein, or a food source comprising the Len c 1 protein; a Lup an 1 protein, or a food source comprising the Lup an 1 protein; and/or a Lup a v protein, or a food source comprising the Lup a v protein; optionally wherein the method does not comprise administering to the subject the Pis s 1 protein or a food source comprising the Pis s 1 protein.

108. A method of treating an allergy to a Pis s 2 protein, or a food source comprising the Pis s 2 protein, in a subject in need thereof, the method comprising administering to the subject: a Vig r 2 protein, or a food source comprising the Vig r 2 protein; an Ara h 1 protein, or a food source comprising the Ara h 1 protein; a Car i 2 protein, or a food source comprising the Car i 2 protein; a Cor a 11 protein, or a food source comprising the Cor a 11 protein; a Gly m 5 protein, or a food source comprising the Gly m 5 protein; a Jug r 2 protein, or a food source comprising the Jug r 2 protein; a Ses i 3 protein, or a food source comprising the Ses i 3 protein; a Len c 1 protein, or a food source comprising the Len c 1 protein; a Lup an 1 protein, or a food source comprising the Lup an 1 protein; a Lup a v protein, or a food source comprising the Lup a v protein; and/or a Pis s 1 protein, or a food source comprising the Pis s 1 protein; optionally wherein the method does not comprise administering to the subject the Pis s 2 protein or a food source comprising the Pis s 2 protein.

109. A method of treating an allergy to a Pis v 2 protein, or a food source comprising the Pis v 2 protein, in a subject in need thereof, the method comprising administering to the subject: a Pis v 5 protein, or a food source comprising the Pis v 5 protein; a Pru du 6 protein, or a food source comprising the Pru du 6 protein; a Ses i 6 protein, or a food source comprising the Ses i 6 protein; a Ses i 7 protein, or a food source comprising the Ses i 7 protein; a Ber e 2 protein, or a food source comprising the Ber e 2 protein; a Fag e 1 protein, or a food source comprising the Fag e 1 protein; a Fag t 13s g protein, or a food source comprising the Fag t 13s g protein; an Ana o 2 protein, or a food source comprising the Ana o 2 protein; an Ara h 3 protein, or a food source comprising the Ara h 3 protein; a Car i 4 protein, or a food source comprising the Car i 4 protein; a Cor a 9 protein, or a food source comprising the Cor a 9 protein; a Gly m 6 protein, or a food source comprising the Gly m 6 protein; a Jug n 4 protein, or a food source comprising the Jug n 4 protein; and/or a Jug r 4 protein, or a food source comprising the Jug r 4 protein; optionally wherein the method does not comprise administering to the subject the Pis v 2 protein or a food source comprising the Pis v 2 protein.

110. A method of treating an allergy to a Pis v 3 protein, or a food source comprising the Pis v 3 protein, in a subject in need thereof, the method comprising administering to the subject: a Ses i 3 protein, or a food source comprising the Ses i 3 protein; a Pin k 2 protein, or a food source comprising the Pin k 2 protein; an Ana o 1 protein, or a food source comprising the Ana o 1 protein; a Car i 2 protein, or a food source comprising the Car i 2 protein; and/or a Cor a 11 protein, or a food source comprising the Cor a 11 protein; optionally wherein the method does not comprise administering to the subject the Pis v 3 protein or a food source comprising the Pis v 3 protein.

111. A method of treating an allergy to a Pru du 6 protein, or a food source comprising the Pru du 6 protein, in a subject in need thereof, the method comprising administering to the subject: a Ses i 6 protein, or a food source comprising the Ses i 6 protein; a Ses i 7 protein, or a food source comprising the Ses i 7 protein; a Ber e 2 protein, or a food source comprising the Ber e 2 protein; a Fag e 1 protein, or a food source comprising the Fag e 1 protein; a Fag t 13s g protein, or a food source comprising the Fag t 13s g protein; a Sin a 2 protein, or a food source comprising the Sin a 2 protein; an Ana o 2 protein, or a food source comprising the Ana o 2 protein; an Ara h 3 protein, or a food source comprising the Ara h 3 protein; a Car i 4 protein, or a food source comprising the Car i 4 protein; a Cor a 9 protein, or a food source comprising the Cor a 9 protein; a Gly m 6 protein, or a food source comprising the Gly m 6 protein; a Jug n 4 protein, or a food source comprising the Jug n 4 protein; a Jug r 4 protein, or a food source comprising the Jug r 4 protein; a Pis v 2 protein, or a food source comprising the Pis v 2 protein; and/or a Pis v 5 protein, or a food source comprising the Pis v 5 protein; optionally wherein the method does not comprise administering to the subject the Pru du 6 protein or a food source comprising the Pru du 6 protein.

112. A method of treating an allergy to a Ses i 3 protein, or a food source comprising the Ses i 3 protein, in a subject in need thereof, the method comprising administering to the subject: a Len c 1 protein, or a food source comprising the Len c 1 protein; a Lup an 1 protein, or a food source comprising the Lup an 1 protein; a Pis s 1 protein, or a food source comprising the Pis s 1 protein; a Pis s 2 protein, or a food source comprising the Pis s 2 protein; an Ana o 1 protein, or a food source comprising the Ana o 1 protein; an Ara h 1 protein, or a food source comprising the Ara h 1 protein; a Car i 2 protein, or a food source comprising the Car i 2 protein; a Jug r 2 protein, or a food source comprising the Jug r 2 protein; and/or a Pis v 3 protein, or a food source comprising the Pis v 3 protein; optionally wherein the method does not comprise administering to the subject the Ses i 3 protein or a food source comprising the Ses i 3 protein.

113. A method of treating an allergy to a Ses i 6 protein, or a food source comprising the Ses i 6 protein, in a subject in need thereof, the method comprising administering to the subject: a Ber e 2 protein, or a food source comprising the Ber e 2 protein; a Fag t 13s g protein, or a food source comprising the Fag t 13s g protein; an Ana o 2 protein, or a food source comprising the Ana o 2 protein; an Ara h 3 protein, or a food source comprising the Ara h 3 protein; a Car i 4 protein, or a food source comprising the Car i 4 protein; a Cor a 9 protein, or a food source comprising the Cor a 9 protein; a Jug n 4 protein, or a food source comprising the Jug n 4 protein; a Jug r 4 protein, or a food source comprising the Jug r 4 protein; a Pis v 2 protein, or a food source comprising the Pis v 2 protein; a Pis v 5 protein, or a food source comprising the Pis v 5 protein; and/or a Pru du 6 protein, or a food source comprising the Pru du 6 protein; optionally wherein the method does not comprise administering to the subject the Ses i 6 protein or a food source comprising the Ses i 6 protein.

114. A method of treating an allergy to a Ses i 7 protein, or a food source comprising the Ses i 7 protein, in a subject in need thereof, the method comprising administering to the subject: a Ber e 2 protein, or a food source comprising the Ber e 2 protein; a Fag e 1 protein, or a food source comprising the Fag e 1 protein; a Fag t 13s g protein, or a food source comprising the Fag t 13s g protein; a Sin a 2 protein, or a food source comprising the Sin a 2 protein; an Ana o 2 protein, or a food source comprising the Ana o 2 protein; an Ara h 3 protein, or a food source comprising the Ara h 3 protein; a Car i 4 protein, or a food source comprising the Car i 4 protein; a Cor a 9 protein, or a food source comprising the Cor a 9 protein; a Gly m 6 protein, or a food source comprising the Gly m 6 protein; a Jug n 4 protein, or a food source comprising the Jug n 4 protein; a Jug r 4 protein, or a food source comprising the Jug r 4 protein; a Pis v 2 protein, or a food source comprising the Pis v 2 protein; a Pis v 5 protein, or a food source comprising the Pis v 5 protein; and/or a Pru du 6 protein, or a food source comprising the Pru du 6 protein; optionally wherein the method does not comprise administering to the subject the Ses i 7 protein or a food source comprising the Ses i 7 protein.

115. A method of treating an allergy to a Sin a 2 protein, or a food source comprising the Sin a 2 protein, in a subject in need thereof, the method comprising administering to the subject: an Ana o 2 protein, or a food source comprising the Ana o 2 protein; a Car i 4 protein, or a food source comprising the Car i 4 protein; a Cor a 9 protein, or a food source comprising the Cor a 9 protein; a Jug n 4 protein, or a food source comprising the Jug n 4 protein; a Jug r 4 protein, or a food source comprising the Jug r 4 protein; a Pis v 5 protein, or a food source comprising the Pis v 5 protein; a Pru du 6 protein, or a food source comprising the Pru du 6 protein; and/or a Ses i 7 protein, or a food source comprising the Ses i 7 protein; optionally wherein the method does not comprise administering to the subject the Sin a 2 protein or a food source comprising the Sin a 2 protein.

116. A method of treating an allergy to a Vig r 2 protein, or a food source comprising the Vig r 2 protein, in a subject in need thereof, the method comprising administering to the subject: an Ara h 1 protein, or a food source comprising the Ara h 1 protein; a Car i 2 protein, or a food source comprising the Car i 2 protein; a Gly m 5 protein, or a food source comprising the Gly m 5 protein; a Jug r 2 protein, or a food source comprising the Jug r 2 protein; a Len c 1 protein, or a food source comprising the Len c 1 protein; a Lup an 1 protein, or a food source comprising the Lup an 1 protein; a Lup a v protein, or a food source comprising the Lup a v protein; a Pis s 1 protein, or a food source comprising the Pis s 1 protein; and/or a Pis s 2 protein, or a food source comprising the Pis s 2 protein; optionally wherein the method does not comprise administering to the subject the Vig r 2 protein or a food source comprising the Vig r 2 protein.

117. A method of treating an allergy to one or more of salmon, shrimp, prawn, crab, lobster, crayfish, mussel, scallop, oyster, clam, abalone, squid, octopus, and nautilus in a subject in need thereof, the method comprising administering to the subject a multiple allergen oral immunotherapy composition comprising two or more of salmon, shrimp, prawn, crab, lobster, crayfish, mussels, scallop, oyster, clam, abalone, squid, octopus, and/or nautilus, optionally wherein the multiple allergen oral immunotherapy composition does not comprise one or more of salmon, shrimp, prawn, crab, lobster, crayfish, mussels, scallop, oyster, clam, abalone, squid, octopus, and nautilus.

118. A method of treating an allergy to salmon in a subject in need thereof, the method comprising administering to the subject a multiple allergen oral immunotherapy composition comprising one or more of shrimp, prawn, crab, lobster, crayfish, mussel, scallop, oyster, clam, abalone, squid, octopus, and nautilus, optionally wherein the multiple allergen oral immunotherapy composition does not comprise salmon.

119. A method of treating an allergy to shrimp in a subject in need thereof, the method comprising administering to the subject a multiple allergen oral immunotherapy composition comprising one or more of salmon, prawn, crab, lobster, crayfish, mussel, scallop, oyster, clam, abalone, squid, octopus, and nautilus, optionally wherein the multiple allergen oral immunotherapy composition does not comprise shrimp.

120. A method of treating an allergy to prawn in a subject in need thereof, the method comprising administering to the subject a multiple allergen oral immunotherapy composition comprising one or more of salmon, shrimp, crab, lobster, crayfish, mussel, scallop, oyster, clam, abalone, squid, octopus, and nautilus, optionally wherein the multiple allergen oral immunotherapy composition does not comprise prawn.

121. A method of treating an allergy to crab in a subject in need thereof, the method comprising administering to the subject a multiple allergen oral immunotherapy composition comprising one or more of salmon, shrimp, prawn, lobster, crayfish, mussel, scallop, oyster, clam, abalone, squid, octopus, and nautilus, optionally wherein the multiple allergen oral immunotherapy composition does not comprise crab.

122. A method of treating an allergy to lobster in a subject in need thereof, the method comprising administering to the subject a multiple allergen oral immunotherapy composition comprising one or more of salmon, shrimp, prawn, crab, crayfish, mussel, scallop, oyster, clam, abalone, squid, octopus, and nautilus, optionally wherein the multiple allergen oral immunotherapy composition does not comprise lobster.

123. A method of treating an allergy to crayfish in a subject in need thereof, the method comprising administering to the subject a multiple allergen oral immunotherapy composition comprising one or more of salmon, shrimp, prawn, crab, lobster, mussel, scallop, oyster, clam, abalone, squid, octopus, and nautilus, optionally wherein the multiple allergen oral immunotherapy composition does not comprise crayfish.

124. A method of treating an allergy to mussel in a subject in need thereof, the method comprising administering to the subject a multiple allergen oral immunotherapy composition comprising one or more of salmon, shrimp, prawn, crab, lobster, crayfish, scallop, oyster, clam, abalone, squid, octopus, and nautilus, optionally wherein the multiple allergen oral immunotherapy composition does not comprise mussel.

125. A method of treating an allergy to scallop in a subject in need thereof, the method comprising administering to the subject a multiple allergen oral immunotherapy composition comprising one or more of salmon, shrimp, prawn, crab, lobster, crayfish, mussel, oyster, clam, abalone, squid, octopus, and nautilus, optionally wherein the multiple allergen oral immunotherapy composition does not comprise scallop.

126. A method of treating an allergy to oyster in a subject in need thereof, the method comprising administering to the subject a multiple allergen oral immunotherapy composition comprising one or more of salmon, shrimp, prawn, crab, lobster, crayfish, mussel, scallop, clam, abalone, squid, octopus, and nautilus, optionally wherein the multiple allergen oral immunotherapy composition does not comprise oyster.

127. A method of treating an allergy to clam in a subject in need thereof, the method comprising administering to the subject a multiple allergen oral immunotherapy composition comprising one or more of salmon, shrimp, prawn, crab, lobster, crayfish, mussel, scallop, oyster, abalone, squid, octopus, and nautilus, optionally wherein the multiple allergen oral immunotherapy composition does not comprise clam.

128. A method of treating an allergy to abalone in a subject in need thereof, the method comprising administering to the subject a multiple allergen oral immunotherapy composition comprising one or more of salmon, shrimp, prawn, crab, lobster, crayfish, mussel, scallop, oyster, clam, squid, octopus, and nautilus, optionally wherein the multiple allergen oral immunotherapy composition does not comprise abalone.

129. A method of treating an allergy to squid in a subject in need thereof, the method comprising administering to the subject a multiple allergen oral immunotherapy composition comprising one or more of salmon, shrimp, prawn, crab, lobster, crayfish, mussel, scallop, oyster, clam, abalone, octopus, and nautilus, optionally wherein the multiple allergen oral immunotherapy composition does not comprise squid.

130. A method of treating an allergy to octopus in a subject in need thereof, the method comprising administering to the subject a multiple allergen oral immunotherapy composition comprising one or more of salmon, shrimp, prawn, crab, lobster, crayfish, mussel, scallop, oyster, clam, abalone, squid, and nautilus, optionally wherein the multiple allergen oral immunotherapy composition does not comprise octopus.

131. A method of treating an allergy to nautilus in a subject in need thereof, the method comprising administering to the subject a multiple allergen oral immunotherapy composition comprising one or more of salmon, shrimp, prawn, crab, lobster, crayfish, mussel, scallop, oyster, clam, abalone, squid, and octopus, optionally wherein the multiple allergen oral immunotherapy composition does not comprise nautilus.

132. A method of treating an allergy to one or more of cod, salmon, shrimp, and carp in a subject in need thereof, the method comprising administering to the subject a multiple allergen oral immunotherapy composition comprising two or more of cod, salmon, shrimp, and carp, optionally wherein the multiple allergen oral immunotherapy composition does not comprise one or more of cod, salmon, shrimp, and carp.

133. A method of treating an allergy to cod in a subject in need thereof, the method comprising administering to the subject a multiple allergen oral immunotherapy composition comprising one or more of salmon, shrimp, and carp, optionally wherein the multiple allergen oral immunotherapy composition does not comprise cod.

134. A method of treating an allergy to salmon in a subject in need thereof, the method comprising administering to the subject a multiple allergen oral immunotherapy composition comprising two or more of cod, salmon, shrimp, and carp, optionally wherein the multiple allergen oral immunotherapy composition does not comprise salmon.

135. A method of treating an allergy to shrimp in a subject in need thereof, the method comprising administering to the subject a multiple allergen oral immunotherapy composition comprising two or more of cod, salmon, shrimp, and carp, optionally wherein the multiple allergen oral immunotherapy composition does not comprise shrimp.

136. A method of treating an allergy to carp in a subject in need thereof, the method comprising administering to the subject a multiple allergen oral immunotherapy composition comprising two or more of cod, salmon, shrimp, and carp, optionally wherein the multiple allergen oral immunotherapy composition does not comprise carp.

137. A method of treating an allergy to one or more of egg, milk, and cheese in a subject in need thereof, the method comprising administering to the subject a multiple allergen oral immunotherapy composition comprising two or more of egg, milk, and cheese, optionally wherein the multiple allergen oral immunotherapy composition does not comprise one or more of egg, milk, and cheese.

138. A method of treating an allergy to egg in a subject in need thereof, the method comprising administering to the subject a multiple allergen oral immunotherapy composition comprising one or both of milk and cheese, optionally wherein the multiple allergen oral immunotherapy composition does not comprise egg.

139. A method of treating an allergy to milk in a subject in need thereof, the method comprising administering to the subject a multiple allergen oral immunotherapy composition comprising one or both of milk and cheese, optionally wherein the multiple allergen oral immunotherapy composition does not comprise milk.

140. A method of treating an allergy to cheese in a subject in need thereof, the method comprising administering to the subject a multiple allergen oral immunotherapy composition comprising one or both of milk and cheese, optionally wherein the multiple allergen oral immunotherapy composition does not comprise cheese.

141. A method of treating an allergy to one or more of wheat, corn, barley, and oat in a subject in need thereof, the method comprising administering to the subject a multiple allergen oral immunotherapy composition comprising two or more of wheat, corn, barley, and oat, optionally wherein the multiple allergen oral immunotherapy composition does not comprise one or more of wheat, corn, barley, and oat.

142. A method of treating an allergy to wheat in a subject in need thereof, the method comprising administering to the subject a multiple allergen oral immunotherapy composition comprising one or more of corn, barley, and oat, optionally wherein the multiple allergen oral immunotherapy composition does not comprise wheat.

143. A method of treating an allergy to corn in a subject in need thereof, the method comprising administering to the subject a multiple allergen oral immunotherapy composition comprising two or more of wheat, corn, barley, and oat, optionally wherein the multiple allergen oral immunotherapy composition does not comprise corn.

144. A method of treating an allergy to barley in a subject in need thereof, the method comprising administering to the subject a multiple allergen oral immunotherapy composition comprising two or more of wheat, corn, barley, and oat, optionally wherein the multiple allergen oral immunotherapy composition does not comprise barley.

145. A method of treating an allergy to oat in a subject in need thereof, the method comprising administering to the subject a multiple allergen oral immunotherapy composition comprising two or more of wheat, corn, barley, and oat, optionally wherein the multiple allergen oral immunotherapy composition does not comprise oat.

146. A method of identifying an IgE or IgG antibody useful for detection of a cross-reactive allergenic protein, the method comprising:
(a) identifying a cross-reactive T-cell or B-cell epitope peptide within the cross-reactive allergenic protein; and
(b) identifying an IgE or IgG antibody that binds to the identified cross-reactive T-cell or B-cell epitope peptide.

147. The method of claim 146, wherein (a) comprises:
(i) identifying a T-cell or B-cell epitope peptide within a reference allergenic protein; and/or
(ii) calculating pairwise identity and/or similarity between the T-cell or B-cell epitope peptide and a T-cell or B-cell epitope from an other protein,
so as to determine whether the T-cell or B-cell from the other protein is cross-reactive to the T-cell or B-cell from the reference allergenic protein.

148. The method of claim 147, wherein (i) comprises: selecting a peptide within the reference allergenic protein; and calculating pairwise identity and/or similarity between the peptide and a known T-cell or B-cell epitope, so as to determine whether the peptide is a T-cell or B-cell epitope.

149. The method of any one of claims 146-148, further comprising determining the MHC binding potential of a T-cell epitope.

150. The method of any one of claims 146-149, wherein the method further comprises mapping the cross-reactive T-cell or B-cell epitope peptide to the full-length sequence of the cross-reactive allergenic protein.

151. The method of any one of claims 146-150, wherein the cross-reactive allergenic protein and/or the reference protein is selected from the group consisting of a shrimp allergen, a cod allergen, a salmon allergen, a hen's egg allergen, a cow's milk allergen, a peanut allergen, a sesame allergen, a soy allergen, a pecan allergen, a cashew allergen, a hazelnut allergen, a walnut allergen, a pistachio allergen, an almond allergen, and a wheat allergen.

152. The method of any one of claims 147-151, wherein the T-cell or B-cell epitope peptide within a reference allergenic protein and the T-cell or B-cell from the other protein are from the same protein family, and the protein family is selected from the group consisting of: Tropomyosin, EF hand family (Beta-parvalbumin), ATP:guanido phosphotransferase (Arginine Kinase), Triosephosphate isomerase, Fructose bisphosphate aldolase class I, Alpha/beta casein, Lipocalin, Kappa-casein, Transferrin, C-type lysozyme/alpha-lactalbumin family, Serum albumin, Enolase, Serpin serine protease inhibitor, Kazal-type serine protease inhibitor, Collagen, Lipoprotein, Prolamin superfamily, Cupin, Profilin, Bet v 1 family, Oleosin, Isoflavone reductase family, Plant defensin, 60S acidic ribosomal protein, Heat shock protein Hsp70, Fe/Mn superoxide dismutase, Papain-like cysteine protease, Thaumatin-like protein, Unclassified, GILT family, Hevein-like and class I/II chitinase, Zinc finger, C3HC4 type (RING finger), SCAI family, Peroxidase, Serpin serine protease inhibitor, Thionin, Thioredoxin, Transcription elongation factor 1, and Triosephosphate isomerase.

153. The method of claim 152, wherein the protein family is selected from the group consisting of: Tropomyosin, Cupin, Profilin, Bet v 1, and EF hand.

154. The method of claim 153, wherein the protein family is Cupin.

155. The method of any one of claims 146-154, wherein the cross-reactive allergenic protein and/or the reference protein is selected from the group consisting of allergenic proteins listed in Table 2.

156. The method of any one of claims 146-155, wherein the cross-reactive allergenic protein and/or the reference protein is selected from the group consisting of proteins listed in Table 4.

157. The method of any one of claims 146-156, wherein the cross-reactive allergenic protein and/or the reference protein is selected from the group consisting of proteins listed in Tables 5-7 or Tables 10-12.

158. An IgE or IgG antibody identified by the method of any one of claims 146-157.

159. An IgE antibody to a cross-reactive epitope sequence selected from the group consisting of: SEQ ID NOs: 1-11742.

160. An IgG antibody to a cross-reactive epitope sequence selected from the group of SEQ ID NOs: SEQ ID NOs: 1-11742.

161. A method for detecting the presence or amount of a cross-reactive allergenic protein, the method comprising:
(a) providing an IgE or IgG antibody to a cross-reactive B-cell or T-cell epitope sequence within the cross-reactive allergenic protein;
(b) contacting a sample (e.g., a biological sample) that may have the cross-reactive allergenic protein under conditions sufficient to permit binding of the IgE or IgG antibody; and
(c) detecting binding of the IgE or IgG antibody, thereby to identify the presence or amount of the cross-reactive allergenic protein.

162. The method of claim 161, wherein the cross-reactive allergenic protein is a kiwifruit, prawn, kiwi, brazil nut, lobster, celery, coconut, crab, apple, buckwheat, clam, pear, lentil, mussel, tomato, garden pea, oyster, stone fruit (e.g., apricot, peach, cherry), yellow mustard, abalone, birch tree, mung bean, squid, alder tree, lupin, octopus, white oak tree, Korean pine, house dust mite, cockroach, or yellow fever mosquito cross-reactive allergenic protein.

163. The method of any one of claims 146-162, wherein the IgE or IgG antibody binds a cross-reactive epitope sequence selected from the group consisting of: SEQ ID NOs: 1-11742.

164. The method of any one of claims 146-163, wherein the IgE or IgG antibody is immobilized on a substrate.

165. The method of claim 164, wherein the substrate is a membrane, a filter, a chip, a slide (such as a microscopic slide), an array (such as a microarray), a wafer, a fiber, a magnetic or a non-magnetic bead, a gel, a tubing, a plate (such as a microtiter plate), microtiter strips, a polymer, a microparticle, a sphere, a microsphere, a nanosphere, a particle, a microparticle, a nanoparticle, or a capillary.

166. A detection kit comprising six or more IgE or IgG antibodies of any one of claims 158-160, each bound to a solid support.

167. The kit of claim 166, wherein each solid support is a membrane, a filter, a chip, a slide (such as a microscopic slide), an array (such as a microarray), a wafer, a fiber, a magnetic or a non-magnetic bead, a gel, a tubing, a plate (such as a microtiter plate), microtiter strips, a polymer, a microparticle, a sphere, a microsphere, a nanosphere, a particle, a microparticle, a nanoparticle, or a capillary.

168. The detection kit of claim 166 or 167, further comprising an IgE or IgG labeling reagent.

169. The detection kit of any one of claims 166-168, further comprising one or more of a binding buffer, a wash buffer, and a detection buffer.

170. The detection kit of any one of claims 166-169, further comprising instructions for use.

171. A method for preparing a multiple allergen oral immunotherapy composition that is capable of treating an allergy to a reference allergenic protein that is not present in the multiple allergen oral immunotherapy, the method comprising:
(a) calculating pairwise identity and/or similarity between an amino acid sequence of the reference allergenic protein and an other protein, so as to determine whether the other protein is cross-reactive to the reference allergenic protein; and
(b) selecting the other allergenic protein as a component in the multiple allergen oral immunotherapy if the other protein is cross-reactive to the reference allergenic protein.

172. The method of claim 171, wherein step (b) comprises:
(i) providing a dataset comprising the amino acid sequence of the reference allergenic protein and the other protein;
(ii) inputting the dataset into a computer readable medium;
(iii) performing a bioinformatic step to calculate pairwise identity and similarity between the reference allergenic protein and the other protein; and/or
(iv) identifying the other protein as cross-reactive to the reference allergenic protein based on pairwise identity and similarity between the reference allergenic protein and the other protein.

173. The method of claim 172, wherein the bioinformatic step comprises applying an algorithm to determine homology between the reference allergenic protein and the other protein based on pairwise identity and similarity, wherein the algorithm is selected from the group consisting of GAP, BESTFIT, FASTA, TFASTA, BLAST, BLASTP, TBLASTN, FASTDB, ALIGN, CLUSTALW, and CLUSTAL-Omega, including any version thereof.

174. The method of claim 172 or 173, wherein the bioinformatic step comprises applying an algorithm comprising (identity+similarity)/2 to determine a cross-reactivity score.

175. The method of claim 174, wherein the other protein is considered cross-reactive to the reference allergenic protein if the other protein has a cross-reactivity score of ≥0.5 with respect to the reference allergenic protein.

176. The method of any one of claims 171-175, wherein the other protein is considered cross-reactive to the reference allergenic protein if the other protein has ≥70% homology to the reference allergenic protein.

177. The method of any one of claims 171-176, wherein the amino acid sequence is a full-length amino acid sequence.

178. The method of any one of claims 171-177, where the method comprises, prior to step (a), identifying the reference allergenic protein as a protein in an allergenic food source.

179. The method of any one of claims 171-178, where the method comprises, prior to step (a), mapping the reference allergenic protein to a protein domain family.

180. The method of any one of claims 171-179, wherein the method further comprises identifying a T-cell epitope peptide, a B-cell epitope peptide, and/or an MHC-binding epitope peptide in the reference allergenic protein that is cross-reactive to a T-cell epitope peptide, a B-cell epitope peptides and/or an MHC-binding epitope in the other protein.

181. The method of claim 180 wherein the method further comprises determining the MHC binding potential of the T-cell or B-cell peptide.

182. The method of any one of claims 171-181, wherein the reference allergenic protein and the other protein are from the same protein family, and the protein family is selected from the group consisting of: Tropomyosin, EF hand family (Beta-parvalbumin), ATP:guanido phosphotransferase (Arginine Kinase), Triosephosphate isomerase, Fructose bisphosphate aldolase class I, Alpha/beta casein, Lipocalin, Kappa-casein, Transferrin, C-type lysozyme/alpha-lactalbumin family, Serum albumin, Enolase, Serpin serine protease inhibitor, Kazal-type serine protease inhibitor, Collagen, Lipoprotein, Prolamin superfamily, Cupin, Profilin, Bet v 1 family, Oleosin, Isoflavone reductase family, Plant defensin, 60S acidic ribosomal protein, Heat shock protein Hsp70, Fe/Mn superoxide dismutase, Papain-like cysteine protease, Thaumatin-like protein, Unclassified, GILT family, Hevein-like and class I/II chitinase, Zinc finger, C3HC4 type (RING finger), SCAI family, Peroxidase, Serpin serine protease inhibitor, Thionin, Thioredoxin, Transcription elongation factor 1, and Triosephosphate isomerase.

183. The method of claim 182, wherein the protein family is selected from the group consisting of: Tropomyosin, Cupin, Profilin, Bet v 1, and EF hand.

184. The method of claim 183, wherein the protein family is Cupin.

185. The method of any one of claims 171-184, wherein the other allergenic protein is selected from the group consisting of a shrimp allergen, a cod allergen, a salmon allergen, a hen's egg allergen, a cow's milk allergen, a peanut allergen, a sesame allergen, a soy allergen, a pecan allergen, a cashew allergen, a hazelnut allergen, a walnut allergen, a pistachio allergen, an almond allergen, and a wheat allergen.

186. The method of any one of claims 171-185, wherein the other allergenic protein is selected from the group consisting of allergenic proteins listed in Table 2.

187. The method of any one of claims 171-186, wherein the reference protein is selected from the group consisting of proteins listed in Table 4.

188. The method of any one of claims 171-187, wherein the reference protein is selected from the group consisting of proteins listed in Tables 5-7 or Tables 10-12.

189. A multiple allergen oral immunotherapeutic composition prepared by the method of any one of claims 171-188.

190. A multiple allergen oral immunotherapy composition comprising:
(a) three or more Cupin family proteins; and
(b) a pharmaceutically acceptable excipient;
wherein upon administration of the oral immunotherapy composition to a subject having an allergy or at risk of having an allergy to one or more of the three or more Cupin family proteins, the subject has increased resistance to the protein as compared to treatment with one Cupin family protein alone; and/or is also treated for an allergy to a different Cupin family protein.

191. The multiple allergen oral immunotherapy composition of claim 190, wherein the three or more Cupin family proteins and/or the different Cupin family member are selected from the group consisting of proteins listed in Table 5 and Table 10.

192. The multiple allergen oral immunotherapy composition of claim 190 or 191, wherein the Cupin family proteins are selected from Pru du 6, Gly m 5, Gly m 6, and Gly m Bd, and upon administration the subject is also effectively treated for a Pis s 1, a Pis s 2 allergy and/or has more effective treatment for a Jug n 4 protein allergy as compared to administering a treatment with Jug n 4 alone.

193. A multiple allergen oral immunotherapy composition comprising:
(a) three or more Tropomyosin family proteins; and
(b) a pharmaceutically acceptable excipient;
wherein upon administration of the oral immunotherapy composition to a subject having an allergy or at risk of having an allergy to one or more of the three or more Tropomyosin family proteins, the subject has increased resistance to the protein as compared to treatment with one Tropomyosin family protein alone; and/or is also treated for an allergy to a different Tropomyosin family protein.

194. The multiple allergen oral immunotherapy composition of claim 193, wherein the three or more Tropomyosin family proteins and/or the different Tropomyosin family member are selected from the group consisting of proteins listed in Table 6 and Table 11.

195. A multiple allergen oral immunotherapy composition comprising:
(a) three or more Bet v 1 family proteins; and
(b) a pharmaceutically acceptable excipient;
wherein upon administration of the oral immunotherapy composition to a subject having an allergy or at risk of having an allergy to one or more of the three or more Bet v 1 family proteins, the subject has increased resistance to the protein as compared to treatment with one Bet v 1 family protein alone; and/or is also treated for an allergy to a different Bet v 1 family protein.

196. The multiple allergen oral immunotherapy composition of claim 195, wherein the three or more Bet v 1 family proteins and/or the different Bet v 1 family member are selected from the group consisting of proteins listed in Table 7 and Table 12.

197. An allergen immunotherapeutic composition comprising at least 4 peptides, wherein the at least 4 peptides are each (i) from 9 to 25 amino acids in length, (ii) selected from the group consisting of a T-cell epitope peptide, a B-cell epitope peptide, and an WIC-binding peptide, and (iii) derived from an allergenic protein in a protein family selected from the group consisting of Tropomyosin, EF hand family (Beta-parvalbumin), ATP:guanido phosphotransferase (Arginine Kinase), Triosephosphate isomerase, Fructose bisphosphate aldolase class I, Alpha/beta casein, Lipocalin, Kappa-casein, Transferrin, C-type lysozyme/alpha-lactalbumin family, Serum albumin, Enolase, Serpin serine protease inhibitor, Kazal-type serine protease inhibitor, Collagen, Lipoprotein, Prolamin superfamily, Cupin, Profilin, Bet v 1 family, Oleosin, Isoflavone reductase family, Plant defensin, 60S acidic ribosomal protein, Heat shock protein Hsp70, Fe/Mn superoxide dismutase, Papain-like cysteine protease, Thaumatin-like protein, Unclassified, GILT family, Hevein-like and class I/II chitinase, Zinc finger, C3HC4 type (RING finger), SCAI family, Peroxidase, Serpin serine protease inhibitor, Thionin, Thioredoxin, Transcription elongation factor 1, and Triosephosphate isomerase.

198. The allergen immunotherapeutic composition of claim 197, wherein the at least 4 peptides are each 15 amino acids in length.

199. The allergen immunotherapeutic composition of claim 197 or 198, wherein the at least 4 peptides are each derived from a protein listed in Table 4.

200. The allergen immunotherapeutic composition of any one of claims 197-199, wherein the at least 4 peptides are each derived from a protein listed in Tables 5-7 or Tables 10-12.

201. The allergen immunotherapeutic composition of any of claims 197-200, wherein the at least 4 peptides selected from the group consisting of peptides comprising the amino acid sequence of any one of SEQ ID NOs: 1-11742 or any fragments thereof.

202. The allergen immunotherapeutic composition of any of claims 195-197, wherein the at least 4 peptides selected from the group consisting of peptides consisting of the amino acid sequence of any one of SEQ ID NOs: 1-11742.
